SEQUENCE LISTING (264 sequences)

SEQ ID NO: 1 - DNA, 54 bp, synthetic construct
  source 1..54: mol_type = other DNA
  modified_base 1: mod_base = OTHER; note = 5' 6-Carboxyfluorescein modified nucleotide
gatcacttagagcaatcggcccagactgagcacgtgatggcagagtactaggat

SEQ ID NO: 2 - DNA, 54 bp, synthetic construct
  source 1..54: mol_type = other DNA
atcctagtactctgccatcacgtgctcagtctgggccgattgctctaagtgatc

SEQ ID NO: 3 - DNA, 58 bp, synthetic construct
  source 1..58: mol_type = other DNA
ggatcctaatacgactcactataggcccagactgagcacgtgagttttagagctagaa

SEQ ID NO: 4 - DNA, 76 bp, synthetic construct
  source 1..76: mol_type = other DNA
gcaccgactcggtgccactttttcaagttgataacggactagccttattttaacttgctatttctagctctaaaac

SEQ ID NO: 5 - DNA, 24 bp, synthetic construct
  source 1..24: mol_type = other DNA
ggatcctaatacgactcactatag

SEQ ID NO: 6 - DNA, 16 bp, synthetic construct
  source 1..16: mol_type = other DNA
aaaagcaccgactcgg

SEQ ID NO: 7 - DNA, 38 bp, synthetic construct
  source 1..38: mol_type = other DNA
aaaagagcacgagattgcagagtactgcaccgactcgg

SEQ ID NO: 8 - DNA, 41 bp, synthetic construct
  source 1..41: mol_type = other DNA
aaaaactgagcacgagattgcagagtactgcaccgactcgg

SEQ ID NO: 9 - DNA, 48 bp, synthetic construct
  source 1..48: mol_type = other DNA
aaaagagcacgagattgcagagtactagatgacgtagcaccgactcgg

SEQ ID NO: 10 - DNA, 51 bp, synthetic construct
  source 1..51: mol_type = other DNA
aaaaactgagcacgagattgcagagtactagatgacgtagcaccgactcgg

SEQ ID NO: 11 - DNA, 15 bp, synthetic construct
  source 1..15: mol_type = other DNA
  modified_base 1: mod_base = OTHER; note = 5' phosphate modified nucleotide
agattgcagagtact

SEQ ID NO: 12 - DNA, 25 bp, synthetic construct
  source 1..25: mol_type = other DNA
  modified_base 1: mod_base = OTHER; note = 5' phosphate modified nucleotide
agattgcagagtactagatgacgta

SEQ ID NO: 13 - DNA, 30 bp, synthetic construct
  source 1..30: mol_type = other DNA
  modified_base 1: mod_base = OTHER; note = 5' phosphate modified nucleotide
agattgcagagtactagatgacgtacgcag

SEQ ID NO: 14 - DNA, 50 bp, synthetic construct
  source 1..50: mol_type = other DNA
  modified_base 1: mod_base = OTHER; note = 5' phosphate modified nucleotide
agattgcagagtactagatgacgtacgcagaccaagaaccgcaagatgcg

SEQ ID NO: 15 - DNA, 70 bp, synthetic construct
  source 1..70: mol_type = other DNA
  modified_base 1: mod_base = OTHER; note = 5' phosphate modified nucleotide
agattgcagagtactagatgacgtacgcagaccaagaaccgcaagatgcgacggtgtacaagtaattgtc

SEQ ID NO: 16 - DNA, 90 bp, synthetic construct
  source 1..90: mol_type = other DNA
  modified_base 1: mod_base = OTHER; note = 5' phosphate modified nucleotide
agattgcagagtactagatgacgtacgcagaccaagaaccgcaagatgcgacggtgtacaagtaattgtcaacagaccatcgtgttttca

SEQ ID NO: 17 - DNA, 24 bp, synthetic construct
  source 1..24: mol_type = other DNA
agtactctgcaatctcgtgcattt

SEQ ID NO: 18 - DNA, 25 bp, synthetic construct
  source 1..25: mol_type = other DNA
agtactctgcaatctcgtgcttttt

SEQ ID NO: 19 - DNA, 26 bp, synthetic construct
  source 1..26: mol_type = other DNA
agtactctgcaatctcgtgctctttt

SEQ ID NO: 20 - DNA, 27 bp, synthetic construct
  source 1..27: mol_type = other DNA
agtactctgcaatctcgtgctcatttt

SEQ ID NO: 21 - DNA, 28 bp, synthetic construct
  source 1..28: mol_type = other DNA
agtactctgcaatctcgtgctcagattt

SEQ ID NO: 22 - DNA, 29 bp, synthetic construct
  source 1..29: mol_type = other DNA
agtactctgcaatctcgtgctcagttttt

SEQ ID NO: 23 - DNA, 39 bp, synthetic construct
  source 1..39: mol_type = other DNA
ctgcgtacgtcatctagtactctgcaatctcgtgcattt

SEQ ID NO: 24 - DNA, 59 bp, synthetic construct
  source 1..59: mol_type = other DNA
cgcatcttgcggttcttggtctgcgtacgtcatctagtactctgcaatctcgtgcattt

SEQ ID NO: 25 - DNA, 41 bp, synthetic construct
  source 1..41: mol_type = other DNA
ctgcgtacgtcatctagtactctgcaatctcgtgctctttt

SEQ ID NO: 26 - DNA, 60 bp, synthetic construct
  source 1..60: mol_type = other DNA
cgcatcttgcggttcttggtctgcgtacgtcatctagtactctgcaatctcgtgctcttt

SEQ ID NO: 27 - DNA, 26 bp, synthetic construct
  source 1..26: mol_type = other DNA
  misc_feature 1..20: note = DNA
  misc_feature 21..22: note = RNA
  misc_feature 23..26: note = DNA
agtactctgcaatctcgtgctctttt

SEQ ID NO: 28 - DNA, 26 bp, synthetic construct
  source 1..26: mol_type = other DNA
  misc_feature 1..19: note = DNA
  misc_feature 20..22: note = RNA
  misc_feature 23..26: note = DNA
agtactctgcaatctcgtgctctttt

SEQ ID NO: 29 - DNA, 26 bp, synthetic construct
  source 1..26: mol_type = other DNA
  misc_feature 1..18: note = DNA
  misc_feature 19..22: note = RNA
  misc_feature 23..26: note = DNA
agtactctgcaatctcgtgctctttt

SEQ ID NO: 30 - DNA, 26 bp, synthetic construct
  source 1..26: mol_type = other DNA
  misc_feature 1..17: note = DNA
  misc_feature 18..22: note = RNA
  misc_feature 23..26: note = DNA
agtactctgcaatctcgtgctctttt

SEQ ID NO: 31 - DNA, 26 bp, synthetic construct
  source 1..26: mol_type = other DNA
  misc_feature 1..16: note = DNA
  misc_feature 17..22: note = RNA
  misc_feature 23..26: note = DNA
agtactctgcaatctcgtgctctttt

SEQ ID NO: 32 - DNA, 34 bp, synthetic construct
  source 1..34: mol_type = other DNA
  modified_base 1: mod_base = OTHER; note = 5' 6-Carboxyfluorescein modified nucleotide
gatcacttagagcaatcggcccagactgagcacg

SEQ ID NO: 33 - DNA, 20 bp, synthetic construct
  source 1..20: mol_type = other DNA
tgatggcagagtactaggat

SEQ ID NO: 34 - DNA, 42 bp, synthetic construct
  source 1..42: mol_type = other DNA
ctgcgtacgtcatctagtactctgcaatctcgtgctcatttt

SEQ ID NO: 35 - DNA, 43 bp, synthetic construct
  source 1..43: mol_type = other DNA
ctgcgtacgtcatctagtactctgcaatctcgtgctcagattt

SEQ ID NO: 36 - DNA, 44 bp, synthetic construct
  source 1..44: mol_type = other DNA
ctgcgtacgtcatctagtactctgcaatctcgtgctcagttttt

SEQ ID NO: 37 - DNA, 45 bp, synthetic construct
  source 1..45: mol_type = other DNA
ctgcgtacgtcatctagtactctgcaatctcgtgctcagtcattt

SEQ ID NO: 38 - DNA, 46 bp, synthetic construct
  source 1..46: mol_type = other DNA
ctgcgtacgtcatctagtactctgcaatctcgtgctcagtcttttt

SEQ ID NO: 39 - DNA, 47 bp, synthetic construct
  source 1..47: mol_type = other DNA
ctgcgtacgtcatctagtactctgcaatctcgtgctcagtctgtttt

SEQ ID NO: 40 - DNA, 15 bp, synthetic construct
  source 1..15: mol_type = other DNA
  modified_base 1: mod_base = OTHER; note = 5' phosphate modified nucleotide
ggatcgcagaggaaa

SEQ ID NO: 41 - DNA, 30 bp, synthetic construct
  source 1..30: mol_type = other DNA
  modified_base 1: mod_base = OTHER; note = 5' phosphate modified nucleotide
ggatcgcagaggaaaggaagccctgcttcc

SEQ ID NO: 42 - DNA, 26 bp, synthetic construct
  source 1..26: mol_type = other DNA
  misc_feature 1..15: note = DNA
  misc_feature 16..22: note = RNA
  misc_feature 23..26: note = DNA
agtactctgcaatctcgtgctctttt

SEQ ID NO: 43 - DNA, 26 bp, synthetic construct
  source 1..26: mol_type = other DNA
  misc_feature 1..21: note = DNA
  misc_feature 22: note = RNA
  misc_feature 23..26: note = DNA
agtactctgcaatctcgtgctctttt

SEQ ID NO: 44 - DNA, 26 bp, synthetic construct
  source 1..26: mol_type = other DNA
agtactctgcaatctcgtgctctttt

SEQ ID NO: 45 - DNA, 117 bp, synthetic construct
  source 1..117: mol_type = other DNA
  misc_feature 1..96: note = RNA
  misc_feature 97..116: note = DNA
  misc_feature 117: note = RNA
ggcccagactgagcacgtgagttttagagctagaaatagcaagttaaaataaggctagtccgttatcaacttgaaaaagtggcaccgagtcggtgctttcctctgcgatcccgtgct

SEQ ID NO: 46 - DNA, 118 bp, synthetic construct
  source 1..118: mol_type = other DNA
  misc_feature 1..96: note = RNA
  misc_feature 97..117: note = DNA
  misc_feature 118: note = RNA
ggcccagactgagcacgtgagttttagagctagaaatagcaagttaaaataaggctagtccgttatcaacttgaaaaagtggcaccgagtcggtgctttcctctgcgatcccgtgctt

SEQ ID NO: 47 - DNA, 119 bp, synthetic construct
  source 1..119: mol_type = other DNA
  misc_feature 1..96: note = RNA
  misc_feature 97..118: note = DNA
  misc_feature 119: note = RNA
ggcccagactgagcacgtgagttttagagctagaaatagcaagttaaaataaggctagtccgttatcaacttgaaaaagtggcaccgagtcggtgctttcctctgcgatcccgtgctct

SEQ ID NO: 48 - DNA, 119 bp, synthetic construct; identical to SEQ ID NO: 47
  source 1..119: mol_type = other DNA
  misc_feature 1..96: note = RNA
  misc_feature 97..116: note = DNA
  misc_feature 117..119: note = RNA

SEQ ID NO: 49 - DNA, 119 bp, synthetic construct; identical to SEQ ID NO: 47
  source 1..119: mol_type = other DNA
  misc_feature 1..96: note = RNA
  misc_feature 97..115: note = DNA
  misc_feature 116..119: note = RNA

SEQ ID NO: 50 - DNA, 119 bp, synthetic construct; identical to SEQ ID NO: 47
  source 1..119: mol_type = other DNA
  misc_feature 1..96: note = RNA
  misc_feature 97..114: note = DNA
  misc_feature 115..119: note = RNA

SEQ ID NO: 51 - DNA, 119 bp, synthetic construct; identical to SEQ ID NO: 47
  source 1..119: mol_type = other DNA
  misc_feature 1..96: note = RNA
  misc_feature 97..113: note = DNA
  misc_feature 114..119: note = RNA

SEQ ID NO: 52 - DNA, 119 bp, synthetic construct; identical to SEQ ID NO: 47
  source 1..119: mol_type = other DNA
  misc_feature 1..96: note = RNA
  misc_feature 97..112: note = DNA
  misc_feature 113..119: note = RNA

SEQ ID NO: 53 - DNA, 119 bp, synthetic construct; identical to SEQ ID NO: 47
  source 1..119: mol_type = other DNA
  misc_feature 1..96: note = RNA
  misc_feature 97..111: note = DNA
  misc_feature 112..119: note = RNA

SEQ ID NO: 54 - DNA, 134 bp, synthetic construct
  source 1..134: mol_type = other DNA
  misc_feature 1..96: note = RNA
  misc_feature 97..126: note = DNA
  misc_feature 127..134: note = RNA
ggcccagactgagcacgtgagttttagagctagaaatagcaagttaaaataaggctagtccgttatcaacttgaaaaagtggcaccgagtcggtgcggaagcagggcttcctttcctctgcgatcccgtgctct

SEQ ID NO: 55 - RNA, 119 nt, synthetic construct
  source 1..119: mol_type = other RNA
ggcccagactgagcacgtgagttttagagctagaaatagcaagttaaaataaggctagtccgttatcaacttgaaaaagtggcaccgagtcggtgctttcctctgcgatcccgtgctct

SEQ ID NO: 56 - DNA, 117 bp, synthetic construct
  source 1..117: mol_type = other DNA
  misc_feature 1..96: note = RNA
  misc_feature 97..106: note = DNA
  misc_feature 107..117: note = RNA
ggcccagactgagcacgtgagttttagagctagaaatagcaagttaaaataaggctagtccgttatcaacttgaaaaagtggcaccgagtcggtgctctgcgatcccgtgctctttt

SEQ ID NO: 57 - DNA, 117 bp, synthetic construct; identical to SEQ ID NO: 56
  source 1..117: mol_type = other DNA
  misc_feature 1..102: note = RNA
  misc_feature 103..110: note = DNA
  misc_feature 111..117: note = RNA

SEQ ID NO: 58 - protein, 1166 aa, Shigella flexneri
MKAGEESVAQVSGVREQAILTQAIRSELKTQGVLGHPEVTMTALSPVWLDSRSRYLRDMYRPGMVMEQWNPETRSHDRYVTERVTAQSHSLTLRNAQGETQVVRISSLDSSWSLFRPEKMPVADGERLRVTGKIPGLRVSGGDRLQVASVSEDAMTVVVPGRAEPATLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFADLGGEINAQIKRGDLLYVDVAKGYGTGLLVSRASYEAEKSILRHILEGKEAVTPLMERVPGELMEKLTSGQRAATRMILETSDRFTVVQGYAGVGKTTQFRAVMSAVNMLPESERPRVVGLGPTHRAVGEMRSAGVDAQTLASFLHDTQLQQRSGETPDFSNTLFLLDESSMVGNTDMARAYALIAAGGGRAVASGDTDQLQAIAPGQPFRLQQTRSAADVVIMKEIVRQTPELREAVYSLINRDVERALSGLERVKPSQVPRLEGAWAPEHSVTEFSHSQEAKLAEAQQKAMLKGEAFPDVPMTLYEAIVRDYTGRTPEAREQTLIVTHLNEDRRVLNSMIHDAREKAGELGKVQVMVPVLNTANIRDGELRRLSTWENNPDALALVDNVYHRIAGISKDDGLITLQDAEGNTRLISPREAVAEGVTLYTPDTIRVGTGDRIRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVFEPKPDREVMNAERLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTEQAVREIAGQERDRAAITEREAALPESVLREPQRVREAVREVARENLLQERLQQMERDMVRDLQKEKTPGGD

SEQ ID NO: 59 - protein, 351 aa, Nostoc sp.
MVSTLDDTKRNAIAEKLADAKLLQELIIENQERFLRESTDNEISNRIRDFLEDDRKNLGIIETVIVQYGIQKEPRQTVREMVDQVRQLMQGSQLNFFEKVAQHELLKHKQVMSGLLVHKAAQKVGADVLAAIGPLNTVNFENRAHQEQLKGILEILGVRELTGQDADQGIWGRVQDAIAAFSGAVGSAVTQGSDKQDMNIQDVIRMDHNKVNILFTELQQSNDPQKIQEYFGQIYKDLTAHAEAEEEVLYPRVRSFYGEGDTQELYDEQSEMKRLLEQIKAISPSAPEFKDRVRQLADIVMDHVRQEESTLFAAIRNNLSSEQTEQWATEFKAAKSKIQQRLGGQATGAGV

SEQ ID NO: 60 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 61 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 62 - protein, 1629 aa, Francisella sp.
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDAIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 63 - protein, 575 aa, unidentified [sequence omitted]

SEQ ID NO: 64 - protein, 174 aa, African swine fever virus
MLTLIQGKKIVNHLRSRLAFEYNGQLIKILSKNIVAVGSLRREEKMLNDVDLLIIVPEKKLLKHVLPNIRIKGLSFSVKVCGERKCVLFIEWEKKTYQLDLFTALAEEKPYAIFHFTGPVSYLIRIRAALKKKNYKLNQYGLFKNQTLVPLKITTEKELIKELGFTYRIPKKRL

SEQ ID NO: 65 - protein, 575 aa, Homo sapiens
MDPRGILKAFPKRQKIHADASSKVLAKIPRREEGEEAEEWLSSLRAHVVRTGIGRARAELFEKQIVQHGGQLCPAQGPGVTHIVVDEGMDYERALRLLRLPQLPPGAQLVKSAWLSLCLQERRLVDVAGFSIFIPSRYLDHPQPSKAEQDASIPPGTHEALLQTALSPPPPPTRPVSPPQKAKEAPNTQAQPISDDEASDGEETQVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVSQEENGQQQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 66 - protein, 335 aa, Homo sapiens
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 67 - protein, 876 aa, Bacillus subtilis
MKNKLVLIDGNSVAYRAFFALPLLHNDKGIHTNAVYGFTMMLNKILAEEQPTHILVAFDAGKTTFRHETFQDYKGGRQQTPPELSEQFPLLRELLKAYRIPAYELDHYEADDIIGTMAARAEREGFAVKVISGDRDLTQLASPQVTVEITKKGITDIESYTPETVVEKYGLTPEQIVDLKGLMGDKSDNIPGVPGIGEKTAVKLLKQFGTVENVLASIDEIKGEKLKENLRQYRDLALLSKQLAAICRDAPVELTLDDIVYKGEDREKVVALFQELGFQSFLDKMAVQTDEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAK

SEQ ID NO: 68 - protein, 928 aa, Escherichia coli [sequence omitted]

SEQ ID NO: 69 - protein, 606 aa, Escherichia coli
MVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 70 - protein, 704 aa, unidentified
MLVSDIEANNLLEKVTKFHCGVIYDYRDGEYHSYRPGDFGAYLDALEAEVKRGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDVEHRAAWLLAKQERNGFPFDTKAIEELYVELAARRSELLRNLTETFGSWYQPKGGTEMFCHPRTGKPLPKYPRIKIPKVGGIFKKPKNKAQREGREPCELDTREYVAGAPYTPVEHVVFNPSSRDHIQKKLQEAGWVPTKFTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQMAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWIHDEIQVACRTEEIAKTVIEVAQEAMRWVGEHWNFRCLLDTEGKMGANWKECH

SEQ ID NO: 71 - protein, 898 aa, unidentified
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 72 - protein, 855 aa, unidentified
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFDSETSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 73 - protein, 704 aa, unidentified
MIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 74 - protein, 876 aa, Geobacillus stearothermophilus
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 75 - protein, 598 aa, Homo sapiens
MSASAQQLAEELQIFGLDCEEALIEKLVELCVQYGQNEEGMVGELIAFCTSTHKVGLTSEILNSFEHEFLSKRLSKARHSTCKDSGHAGARDIVSIQELIEVEEEEEILLNSYTTPSKGSQKRAISTPETPLTKRSVSTRSPHQLLSPSSFSPSATPSQKYNSRSNRGEVVTSFGLAQGVSWSGRGGAGNISLKVLGCPEALTGSYKSMFQKLPDIREVLTCKIEELGSELKEHYKIEAFTPLLAPAQEPVTLLGQIGCDSNGKLNNKSVILEGDREHSSGAQIPVDLSELKEYSLFPGQVVIMEGINTTGRKLVATKLYEGVPLPFYQPTEEDADFEQSMVLVACGPYTTSDSITYDPLLDLIAVINHDRPDVCILFGPFLESKHEQVENCLLTSPFEDIFKQCLRTIIEGTRSSGSHLVFVPSLRDVHHEPVYPQPPFSYSDLSREDKKQVQFVSEPCSLSINGVIFGLTSTDLLFHLGAEEISSSSGTSDRFSRILKHILTQRSYYPLYPPQEDMAIDYESFYVYAQLPVTPDVLIIPSELRYFVKDVLGCVCVNPGRLTKGQVGGTFARLYLRRPAADGAERQSPCIAVQVVRI

SEQ ID NO: 76 - protein, 1235 aa, Human alphaherpesvirus 1
MFSGGGGPLSPGGKSAARAASGFFAPAGPRGAGRGPPPCLRQNFYNPYLAPVGTQQKPTGPTQRHTYYSECDEFRFIAPRVLDEDAPPEKRAGVHDGHLKRAPKVYCGGDERDVLRVGSGGFWPRRSRLWGGVDHAPAGFNPTVTVFHVYDILENVEHAYGMRAAQFHARFMDAITPTGTVITLLGLTPEGHRVAVHVYGTRQYFYMNKEEVDRHLQCRAPRDLCERMAAALRESPGASFRGISADHFEAEVVERTDVYYYETRPALFYRVYVRSGRVLSYLCDNFCPAIKKYEGGVDATTRFILDNPGFVTFGWYRLKPGRNNTLAQPRAPMAFGTSSDVEFNCTADNLAIEGGMSDLPAYKLMCFDIECKAGGEDELAFPVAGHPEDLVIQISCLLYDLSTTALEHVLLFSLGSCDLPESHLNELAARGLPTPVVLEFDSEFEMLLAFMTLVKQYGPEFVTGYNIINFDWPFLLAKLTDIYKVPLDGYGRMNGRGVFRVWDIGQSHFQKRSKIKVNGMVNIDMYGIITDKIKLSSYKLNAVAEAVLKDKKKDLSYRDIPAYYAAGPAQRGVIGEYCIQDSLLVGQLFFKFLPHLELSAVARLAGINITRTIYDGQQIRVFTCLLRLADQKGFILPDTQGRFRGAGGEAPKRPAAAREDEERPEEEGEDEDEREEGGGEREPEGARETAGRHVGYQGARVHDPTSGFHVNPVVGFDFASLYPSIIQAHNLCFSTLSLRADAVAHLEAGKDYLEIEVGGRRLFFVKAHVRESLLSILLRDWLAMRKQIRSRIPQSSPEEAVLLDKQQAAIKVVCNSVYGFTGVQHGLLPCLHVAATVTTIGREMLLATREYVHARWAAFEQLLADFPEAADMRAPGPYSMRIIYGDTDSIFVLCRGLTAAGLTAMGDKMASHISRALFLPPIKLECEKTFTKLLLIAKKKYIGVIYGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSHADPPGGASKPRKLLVSELAEDPAYAIAHGVALNTDYYFSHLLGAACVTFKALFGNNAKITESLLKRFIPEVWHPPDDVAARLRAAGFGAVGAGATAEETRRMLHRAFDTLA

SEQ ID NO: 77 - protein, 7 aa, Simian virus 40
PKKKRKV

SEQ ID NO: 78 - protein, 17 aa, unidentified
KRTADGSEFEPKKKRKV

SEQ ID NO: 79 - protein, 16 aa, unidentified
KRPAATKKAGQAKKKK

SEQ ID NO: 80 - protein, 3000 aa, synthetic construct
  VARIANT 1..3000: note = This sequence may encompass 1-1000 GGS repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 81 - protein, 5000 aa, synthetic construct
  VARIANT 1..5000: note = This sequence may encompass 1-1000 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 82 - protein, 5000 aa, synthetic construct
  VARIANT 1..5000: note = This sequence may encompass 1-1000 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 83 - protein, 7 aa, synthetic construct
MSRPDPA

SEQ ID NO: 84 - protein, 10 aa, synthetic construct
MKIIEQLPSA

SEQ ID NO: 85 - protein, 10 aa, synthetic construct
VRHKLKRVGS

SEQ ID NO: 86 - protein, 12 aa, synthetic construct
SIVAQLSRPDPA

SEQ ID NO: 87 - protein, 12 aa, synthetic construct
GHGTGSTGSGSS

SEQ ID NO: 88 - protein, 12 aa, synthetic construct
GSAGSAAGSGEF

SEQ ID NO: 89 - protein, 15 aa, synthetic construct
VPFLLEPDNINGKTC

SEQ ID NO: 90 - protein, 16 aa, synthetic construct
SGSETPGTSESATPES

SEQ ID NO: 91 - protein, 33 aa, synthetic construct
SGGSSGGSSGSETPGTSESATPESSGGSSGGSS

SEQ ID NO: 92 - protein, 1368 aa, Streptococcus pyogenes [sequence omitted]

SEQ ID NO: 93 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 94 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 95 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 96 - protein, 1368 aa, Streptococcus pyogenes
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKELQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 97 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 98 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDAILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 99 - protein, 575 aa, synthetic construct
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 100 - protein, 575 aa, synthetic construct
MKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 101 - protein, 575 aa, synthetic construct
MKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAPACYDRIIYCDTDSIHLTGTEIPDVIKDIVDPKKLGYWAHESTFKRAKYLRQKTYIQDIYMKEVDGELVEGSPDDYTDIKLSVKCAGMTDKIKKEVTFENFKVGFSRKMKPKPVQVPGGVVLVDDTFTIK

SEQ ID NO: 102 - protein, 565 aa, synthetic construct
MKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIK

SEQ ID NO: 103 - protein, 575 aa, synthetic construct
MKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIK

SEQ ID NO: 104 - protein, 575 aa, synthetic construct
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGARKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 105 - protein, 575 aa, synthetic construct
MKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGARKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIK

SEQ ID NO: 106 - protein, 575 aa, synthetic construct
MKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGARKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIK

SEQ ID NO: 107 - protein, 664 aa, synthetic construct
MLVSAIAANNLLEKVTKFHCGVIYDYRDGEYHSYRPGDFGAYLDALEAEVKRGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDVEHRAAWLLAKQERNGFPFDTKAIEELYVELAARRSELLRNLTETFGSWYQPKGGTEMFCHPRTGKPLPKYPRIKIPKVGGIFKKPKNKAQREGREPCELDTREYVAGAPYTPVEHVVFNPSSRDHIQKKLQEAGWVPTKFTDKGAPVVDDEVLEGVRVDDPEKQAAIDLIKEYLMIQKRIGQSAEGDKAWLRYVAEDGKIHGSVNPNGAVTGRATHAFPNLAQILRCLAHFMARFDNGEYAHEILNGDIHTKNQMAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWIHDEIQVACRTEEIAKTVIEVAQEAMRWVGEHWNFRCLLDTEGKMGANWKECH

SEQ ID NO: 108 - protein, 896 aa, synthetic construct
MKEFYISIETVGNNIVERYIDENGKERTREVEYLPTMFRHCKEESKYKDIYGKNCAPQKFMKDARDWMKRMEDIGLEALGMNDFKLAYISDTYGSEIVYDRKFVRVANCAIAVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 109 - protein, 855 aa, synthetic construct
MKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGA

SEQ ID NO: 110 - protein, 705 aa, synthetic construct
MIVSAIAEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 111 - protein, 678 aa, Murine leukemia virus
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 112 - protein, 1356 aa, Murine leukemia virus
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 113 - DNA, 57 bp, synthetic construct
  source 1..57: mol_type = other DNA
acactctttccctacacgacgctcttccgatctaacggatggatgtggcgcaggtag

SEQ ID NO: 114 - DNA, 57 bp, synthetic construct
  source 1..57: mol_type = other DNA
acactctttccctacacgacgctcttccgatctaagtgatggatgtggcgcaggtag

SEQ ID NO: 115 - DNA, 57 bp, synthetic construct
  source 1..57: mol_type = other DNA
acactctttccctacacgacgctcttccgatctagctgatggatgtggcgcaggtag

SEQ ID NO: 116 - DNA, 57 bp, synthetic construct
  source 1..57: mol_type = other DNA
acactctttccctacacgacgctcttccgatctataggatggatgtggcgcaggtag

SEQ ID NO: 117 - DNA, 57 bp, synthetic construct
  source 1..57: mol_type = other DNA
acactctttccctacacgacgctcttccgatctcggagatggatgtggcgcaggtag

SEQ ID NO: 118 - DNA, 57 bp, synthetic construct
  source 1..57: mol_type = other DNA
acactctttccctacacgacgctcttccgatctcttggatggatgtggcgcaggtag

SEQ ID NO: 119 - DNA, 57 bp, synthetic construct
  source 1..57: mol_type = other DNA
acactctttccctacacgacgctcttccgatctgaacgatggatgtggcgcaggtag

SEQ ID NO: 120 - DNA, 57 bp, synthetic construct
  source 1..57: mol_type = other DNA
acactctttccctacacgacgctcttccgatctgagtgatggatgtggcgcaggtag

SEQ ID NO: 121 - DNA, 57 bp, synthetic construct
  source 1..57: mol_type = other DNA
acactctttccctacacgacgctcttccgatctgcaggatggatgtggcgcaggtag

SEQ ID NO: 122 - DNA, 57 bp, synthetic construct
  source 1..57: mol_type = other DNA
acactctttccctacacgacgctcttccgatctgtaagatggatgtggcgcaggtag

SEQ ID NO: 123 - DNA, 57 bp, synthetic construct
  source 1..57: mol_type = other DNA
acactctttccctacacgacgctcttccgatcttacagatggatgtggcgcaggtag

SEQ ID NO: 124 - DNA, 57 bp, synthetic construct
  source 1..57: mol_type = other DNA
acactctttccctacacgacgctcttccgatcttctagatggatgtggcgcaggtag

SEQ ID NO: 125 - DNA, 56 bp, synthetic construct
  source 1..56: mol_type = other DNA
gactggagttcagacgtgtgctcttccgatctacagaggcgtatcatttcgcggat

SEQ ID NO: 126 - DNA, 56 bp, synthetic construct
  source 1..56: mol_type = other DNA
gactggagttcagacgtgtgctcttccgatctagaaaggcgtatcatttcgcggat

SEQ ID NO: 127 - DNA, 56 bp, synthetic construct
  source 1..56: mol_type = other DNA
gactggagttcagacgtgtgctcttccgatctctaaaggcgtatcatttcgcggat

SEQ ID NO: 128 - DNA, 56 bp, synthetic construct
  source 1..56: mol_type = other DNA
gactggagttcagacgtgtgctcttccgatctctgcaggcgtatcatttcgcggat

SEQ ID NO: 129 - DNA, 56 bp, synthetic construct
  source 1..56: mol_type = other DNA
gactggagttcagacgtgtgctcttccgatctgcggaggcgtatcatttcgcggat

SEQ ID NO: 130 - DNA, 56 bp, synthetic construct
  source 1..56: mol_type = other DNA
gactggagttcagacgtgtgctcttccgatctggataggcgtatcatttcgcggat

SEQ ID NO: 131 - DNA, 56 bp, synthetic construct
  source 1..56: mol_type = other DNA
gactggagttcagacgtgtgctcttccgatcttcaaaggcgtatcatttcgcggat

SEQ ID NO: 132 - DNA, 56 bp, synthetic construct
  source 1..56: mol_type = other DNA
gactggagttcagacgtgtgctcttccgatctttccaggcgtatcatttcgcggat

SEQ ID NO: 133 - DNA, 60 bp, synthetic construct
  source 1..60: mol_type = other DNA
acactctttccctacacgacgctcttccgatctaacggcatggatgagagaagcctggag

SEQ ID NO: 134 - DNA, 60 bp, synthetic construct
  source 1..60: mol_type = other DNA
acactctttccctacacgacgctcttccgatctaagtgcatggatgagagaagcctggag

SEQ ID NO: 135 - DNA, 60 bp, synthetic construct
  source 1..60: mol_type = other DNA
acactctttccctacacgacgctcttccgatctagctgcatggatgagagaagcctggag

SEQ ID NO: 136 - DNA, 60 bp, synthetic construct
  source 1..60: mol_type = other DNA
acactctttccctacacgacgctcttccgatctataggcatggatgagagaagcctggag

SEQ ID NO: 137 - DNA, 60 bp, synthetic construct
  source 1..60: mol_type = other DNA
acactctttccctacacgacgctcttccgatctcggagcatggatgagagaagcctggag

SEQ ID NO: 138 - DNA, 60 bp, synthetic construct
  source 1..60: mol_type = other DNA
acactctttccctacacgacgctcttccgatctcttggcatggatgagagaagcctggag

SEQ ID NO: 139 - DNA, 60 bp, synthetic construct
  source 1..60: mol_type = other DNA
acactctttccctacacgacgctcttccgatctgaacgcatggatgagagaagcctggag

SEQ ID NO: 140 - DNA, 60 bp, synthetic construct
  source 1..60: mol_type = other DNA
acactctttccctacacgacgctcttccgatctgagtgcatggatgagagaagcctggag

SEQ ID NO: 141 - DNA, 60 bp, synthetic construct
  source 1..60: mol_type = other DNA
acactctttccctacacgacgctcttccgatctgcaggcatggatgagagaagcctggag

SEQ ID NO: 142 - DNA, 60 bp, synthetic construct
  source 1..60: mol_type = other DNA
acactctttccctacacgacgctcttccgatctgtaagcatggatgagagaagcctggag

SEQ ID NO: 143 - DNA, 60 bp, synthetic construct
  source 1..60: mol_type = other DNA
acactctttccctacacgacgctcttccgatcttacagcatggatgagagaagcctggag

SEQ ID NO: 144 - DNA, 60 bp, synthetic construct
  source 1..60: mol_type = other DNA
acactctttccctacacgacgctcttccgatcttctagcatggatgagagaagcctggag

SEQ ID NO: 145 - DNA, 56 bp, synthetic construct
  source 1..56: mol_type = other DNA
gactggagttcagacgtgtgctcttccgatctacagcccagccaaacttgtcaacc

SEQ ID NO: 146 - DNA, 56 bp, synthetic construct
  source 1..56: mol_type = other DNA
gactggagttcagacgtgtgctcttccgatctagaacccagccaaacttgtcaacc

SEQ ID NO: 147 - DNA, 56 bp, synthetic construct
  source 1..56: mol_type = other DNA
gactggagttcagacgtgtgctcttccgatctctaacccagccaaacttgtcaacc

SEQ ID NO: 148 - DNA, 56 bp, synthetic construct
  source 1..56: mol_type = other DNA
gactggagttcagacgtgtgctcttccgatctctgccccagccaaacttgtcaacc

SEQ ID NO: 149 - DNA, 56 bp, synthetic construct
  source 1..56: mol_type = other DNA
gactggagttcagacgtgtgctcttccgatctgcggcccagccaaacttgtcaacc

SEQ ID NO: 150 - DNA, 56 bp, synthetic construct
  source 1..56: mol_type = other DNA
gactggagttcagacgtgtgctcttccgatctggatcccagccaaacttgtcaacc

SEQ ID NO: 151 - DNA, 56 bp, synthetic construct
  source 1..56: mol_type = other DNA
gactggagttcagacgtgtgctcttccgatcttcaacccagccaaacttgtcaacc

SEQ ID NO: 152 - DNA, 56 bp, synthetic construct
  source 1..56: mol_type = other DNA
gactggagttcagacgtgtgctcttccgatctttcccccagccaaacttgtcaacc

SEQ ID NO: 153 - DNA, 53 bp, synthetic construct
  source 1..53: mol_type = other DNA
acactctttccctacacgacgctcttccgatcttgctgaacatgcggaagcgg

SEQ ID NO: 154 - DNA, 56 bp, synthetic construct
  source 1..56: mol_type = other DNA
gactggagttcagacgtgtgctcttccgatcttttccagccttgggcatagtcagg

SEQ ID NO: 155 - DNA, 54 bp, synthetic construct
  source 1..54: mol_type = other DNA
acactctttccctacacgacgctcttccgatctttcacagccaacgactccggc

SEQ ID NO: 156 - DNA, 68 bp, synthetic construct
  source 1..68: mol_type = other DNA
gactggagttcagacgtgtgctcttccgatctgcatatgaggtgaaaacactgctttagtaaaaatgg

SEQ ID NO: 157 - DNA, 51 bp, synthetic construct
  source 1..51: mol_type = other DNA
acactctttccctacacgacgctcttccgatctaggtcccctgggaggggc

SEQ ID NO: 158 - DNA, 51 bp, synthetic construct
  source 1..51: mol_type = other DNA
gactggagttcagacgtgtgctcttccgatctagcctgctgacgctgcctt

SEQ ID NO: 159 - DNA, 57 bp, synthetic construct
  source 1..57: mol_type = other DNA
acactctttccctacacgacgctcttccgatcttccgctaaaggctgctctctcaac

SEQ ID NO: 160 - DNA, 54 bp, synthetic construct
  source 1..54: mol_type = other DNA
gactggagttcagacgtgtgctcttccgatcttgtgactgatgctgtggcaggt

SEQ ID NO: 161 - DNA, 59 bp, synthetic construct
  source 1..59: mol_type = other DNA
acactctttccctacacgacgctcttccgatctccattatctaatggacgaccccaggg

SEQ ID NO: 162 - DNA, 54 bp, synthetic construct
  source 1..54: mol_type = other DNA
gactggagttcagacgtgtgctcttccgatctacgtacagctgcccatccttcc

SEQ ID NO: 163 - DNA, 63 bp, synthetic construct
  source 1..63: mol_type = other DNA
acactctttccctacacgacgctcttccgatcttgaagccaacccacacagtatacctatagt

SEQ ID NO: 164 - DNA, 61 bp, synthetic construct
  source 1..61: mol_type = other DNA
gactggagttcagacgtgtgctcttccgatctgaagcattttcgattccgtgacttggaag

SEQ ID NO: 165 - DNA, 57 bp, synthetic construct
  source 1..57: mol_type = other DNA
acactctttccctacacgacgctcttccgatctctgaacactcctcaaacggtcccc

SEQ ID NO: 166 - DNA, 56 bp, synthetic construct
  source 1..56: mol_type = other DNA
gactggagttcagacgtgtgctcttccgatctctttctcaaggggctgctgtgagg

SEQ ID NO: 167 - DNA, 60 bp, synthetic construct
  source 1..60: mol_type = other DNA
acactctttccctacacgacgctcttccgatctaacgggggagacttggtattttgttca

SEQ ID NO: 168 - DNA, 60 bp, synthetic construct
  source 1..60: mol_type = other DNA
acactctttccctacacgacgctcttccgatctaagtggggagacttggtattttgttca

SEQ ID NO: 169 - DNA, 60 bp, synthetic construct
  source 1..60: mol_type = other DNA
acactctttccctacacgacgctcttccgatctagctggggagacttggtattttgttca

SEQ ID NO: 170 - DNA, 60 bp, synthetic construct
  source 1..60: mol_type = other DNA
acactctttccctacacgacgctcttccgatctatagggggagacttggtattttgttca

SEQ ID NO: 171 - DNA, 60 bp, synthetic construct
  source 1..60: mol_type = other DNA
acactctttccctacacgacgctcttccgatctcggaggggagacttggtattttgttca

SEQ ID NO: 172 - DNA, 60 bp, synthetic construct
  source 1..60: mol_type = other DNA
acactctttccctacacgacgctcttccgatctcttgggggagacttggtattttgttca

SEQ ID NO: 173 - DNA, 60 bp, synthetic construct
  source 1..60: mol_type = other DNA
acactctttccctacacgacgctcttccgatctgaacggggagacttggtattttgttca

SEQ ID NO: 174 - DNA, 60 bp, synthetic construct
  source 1..60: mol_type = other DNA
acactctttccctacacgacgctcttccgatctgagtggggagacttggtattttgttca

SEQ ID NO: 175 - DNA, 60 bp, synthetic construct
  source 1..60: mol_type = other DNA
acactctttccctacacgacgctcttccgatctgcagggggagacttggtattttgttca

SEQ ID NO: 176 - DNA, 60 bp, synthetic construct
  source 1..60: mol_type = other DNA
acactctttccctacacgacgctcttccgatctgtaaggggagacttggtattttgttca

SEQ ID NO: 177 - DNA, 60 bp, synthetic construct
  source 1..60: mol_type = other DNA
acactctttccctacacgacgctcttccgatcttacaggggagacttggtattttgttca

SEQ ID NO: 178 - DNA, 60 bp, synthetic construct
  source 1..60: mol_type = other DNA
acactctttccctacacgacgctcttccgatcttctaggggagacttggtattttgttca

SEQ ID NO: 179 - DNA, 53 bp, synthetic construct
  source 1..53: mol_type = other DNA
gactggagttcagacgtgtgctcttccgatctacagggagtgagcgcttcctg

SEQ ID NO: 180 - DNA, 53 bp, synthetic construct
  source 1..53: mol_type = other DNA
gactggagttcagacgtgtgctcttccgatctagaaggagtgagcgcttcctg

SEQ ID NO: 181 - DNA, 53 bp, synthetic construct
  source 1..53: mol_type = other DNA
gactggagttcagacgtgtgctcttccgatctctaaggagtgagcgcttcctg

SEQ ID NO: 182 - DNA, 53 bp, synthetic construct
  source 1..53: mol_type = other DNA
gactggagttcagacgtgtgctcttccgatctctgcggagtgagcgcttcctg

SEQ ID NO: 183 - DNA, 53 bp, synthetic construct
  source 1..53: mol_type = other DNA
gactggagttcagacgtgtgctcttccgatctgcggggagtgagcgcttcctg

SEQ ID NO: 184 - DNA, 53 bp, synthetic construct
  source 1..53: mol_type = other DNA
gactggagttcagacgtgtgctcttccgatctggatggagtgagcgcttcctg

SEQ ID NO: 185 - DNA, 53 bp, synthetic construct
  source 1..53: mol_type = other DNA
gactggagttcagacgtgtgctcttccgatcttcaaggagtgagcgcttcctg

SEQ ID NO: 186 - DNA, 53 bp, synthetic construct
  source 1..53: mol_type = other DNA
gactggagttcagacgtgtgctcttccgatctttccggagtgagcgcttcctg

SEQ ID NO: 187 - DNA, 127 bp, synthetic construct
  source 1..127: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = Nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..114: note = DNA
  misc_feature 115..127: note = RNA
ggcccagactgagcacgtgagttttagagctagaaatagcaagttaaaataaggctagtccgttatcaacttgaaaaagtggcaccgagtcggtgctttcctctgccatcccgtgctcagtctgttt

SEQ ID NO: 188 - DNA, 127 bp, synthetic construct; identical to SEQ ID NO: 187
  source 1..127: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = Nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..113: note = DNA
  misc_feature 114..127: note = RNA

SEQ ID NO: 189 - DNA, 127 bp, synthetic construct; identical to SEQ ID NO: 187
  source 1..127: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = Nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..112: note = DNA
  misc_feature 113..127: note = RNA

SEQ ID NO: 190 - DNA, 126 bp, synthetic construct
  source 1..126: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 123: mod_base = OTHER; note = Nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide
  misc_feature 1..96: note = RNA
  misc_feature 97..115: note = DNA
  misc_feature 116..126: note = RNA
ggaatcccttctgcagcaccgttttagagctagaaatagcaagttaaaataaggctagtccgttatcaacttgaaaaagtggcaccgagtcggtgcggaaaagcgatcgtgatgctgcagaaggga

SEQ ID NO: 191 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatcgtgatgctgcagaagg

SEQ ID NO: 192 - DNA, 122 bp, synthetic construct
  source 1..122: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = Nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage
  modified_base 122: mod_base = OTHER; note = 2'-O-methyl modified nucleotide
  misc_feature 1..96: note = RNA
  misc_feature 97..115: note = DNA
  misc_feature 116..122: note = RNA
ggaatcccttctgcagcaccgttttagagctagaaatagcaagttaaaataaggctagtccgttatcaacttgaaaaagtggcaccgagtcggtgcggaaaagcgatcgtgatgctgcagaa

SEQ ID NO: 193 - DNA, 122 bp, synthetic construct
  source 1..122: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = Nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage
  modified_base 122: mod_base = OTHER; note = 2'-O-methyl modified nucleotide
  misc_feature 1..96: note = RNA
  misc_feature 97..116: note = DNA
  misc_feature 117..122: note = RNA
ggcccagactgagcacgtgagttttagagctagaaatagcaagttaaaataaggctagtccgttatcaacttgaaaaagtggcaccgagtcggtgctttcctctgcgatcacgtgctcagtc

SEQ ID NO: 194 - DNA, 122 bp, synthetic construct; identical to SEQ ID NO: 193
  source 1..122: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = Nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage
  modified_base 122: mod_base = OTHER; note = 2'-O-methyl modified nucleotide
  misc_feature 1..96: note = RNA
  misc_feature 97..113: note = DNA
  misc_feature 114..122: note = RNA

SEQ ID NO: 195 - DNA, 122 bp, synthetic construct; identical to SEQ ID NO: 193
  source 1..122: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = Nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage
  modified_base 122: mod_base = OTHER; note = 2'-O-methyl modified nucleotide
  misc_feature 1..96: note = RNA
  misc_feature 97..112: note = DNA
  misc_feature 113..122: note = RNA

SEQ ID NO: 196 - DNA, 124 bp, synthetic construct; identical to SEQ ID NO: 191
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..118: note = DNA
  misc_feature 119..124: note = RNA

SEQ ID NO: 197 - DNA, 124 bp, synthetic construct; identical to SEQ ID NO: 191
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..116: note = DNA
  misc_feature 117..124: note = RNA

SEQ ID NO: 198 - DNA, 124 bp, synthetic construct; identical to SEQ ID NO: 191
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..114: note = DNA
  misc_feature 115..124: note = RNA

SEQ ID NO: 199 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccagatgctgcagaagg

SEQ ID NO: 200 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccagctgctgcagaagg

SEQ ID NO: 201 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccagttgctgcagaagg

SEQ ID NO: 202 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccaggcgctgcagaagg

SEQ ID NO: 203 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccaggagctgcagaagg

SEQ ID NO: 204 - DNA, 124 bp, synthetic construct
  source 1..124: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
  misc_feature 1..96: note = RNA
  misc_feature 97..115: note = DNA
  misc_feature 116..124: note = RNA
ggaatcccttctgcagcaccgttttagagctagaaatagcaagttaaaataaggctagtccgttatcaacttgaaaaagtggcaccgagtcggtgcggaaaagcgatccaggggctgcagaagg

SEQ ID NO: 205 - DNA, 120 bp, synthetic construct
  source 1..120: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 117: mod_base = OTHER; note = Nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide
  misc_feature 1..96: note = RNA
  misc_feature 97..110: note = DNA
  misc_feature 111..120: note = RNA
ggcccagactgagcacgtgagttttagagctagaaatagcaagttaaaataaggctagtccgttatcaacttgaaaaagtggcaccgagtcggtgctctgccatcaaagcgtgctcagtc

SEQ ID NO: 206 - DNA, 138 bp, synthetic construct
  source 1..138: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 135: mod_base = OTHER; note = Nucleotide with phosphorothioate linkage
  modified_base 136: mod_base = OTHER; note = 2'-O-methyl modified nucleotide with phosphorothioate linkage
  modified_base 137: mod_base = OTHER; note = 2'-O-methyl modified nucleotide with phosphorothioate linkage
  modified_base 138: mod_base = OTHER; note = 2'-O-methyl modified nucleotide
  misc_feature 1..96: note = RNA
  misc_feature 97..128: note = DNA
  misc_feature 129..138: note = RNA
ggcccagactgagcacgtgagttttagagctagaaatagcaagttaaaataaggctagtccgttatcaacttgaaaaagtggcaccgagtcggtgctggaggaagcagggcttcctttcctctgccacgtgctcagtc

SEQ ID NO: 207 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gtcaaccagtatcccggtgcgttttagagctagaaatagcaagttaaaataaggctagtccgttatcaacttgaaaaagtggcaccgagtcggtgctttt

SEQ ID NO: 208 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
ggggtcccaggtgctgacgtgttttagagctagaaatagcaagttaaaataaggctagtccgttatcaacttgaaaaagtggcaccgagtcggtgctttt

SEQ ID NO: 209 - DNA, 134 bp, synthetic construct
  source 1..134: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 131: mod_base = OTHER; note = Nucleotide with phosphorothioate linkage
  modified_base 132: mod_base = OTHER; note = 2'-O-methyl modified nucleotide with phosphorothioate linkage
  modified_base 133: mod_base = OTHER; note = 2'-O-methyl modified nucleotide with phosphorothioate linkage
  modified_base 134: mod_base = OTHER; note = 2'-O-methyl modified nucleotide
  misc_feature 1..96: note = RNA
  misc_feature 97..120: note = DNA
  misc_feature 121..134: note = RNA
aaataaggtaaagagagggagttttagagctagaaatagcaagttaaaataaggctagtccgttatcaacttgaaaaagtggcaccgagtcggtgctccctgggtccctgccaggtcccctctctttaccttat

SEQ ID NO: 210 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gcatagtcagggactctcgtgttttagagctagaaatagcaagttaaaataaggctagtccgttatcaacttgaaaaagtggcaccgagtcggtgctttt

SEQ ID NO: 211 - DNA, 131 bp, synthetic construct
  source 1..131: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 128: mod_base = OTHER; note = Nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide
  misc_feature 1..96: note = RNA
  misc_feature 97..119: note = DNA
  misc_feature 120..131: note = RNA
agtccctcattccttgggatgttttagagctagaaatagcaagttaaaataaggctagtccgttatcaacttgaaaaagtggcaccgagtcggtgctccaccacggctgtcacaaatcccaaggaatgagg

SEQ ID NO: 212 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gtattcacagccaacgactcgttttagagctagaaatagcaagttaaaataaggctagtccgttatcaacttgaaaaagtggcaccgagtcggtgctttt

SEQ ID NO: 213 - DNA, 129 bp, synthetic construct
  source 1..129: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 126: mod_base = OTHER; note = Nucleotide with phosphorothioate linkage
  modified_base 127: mod_base = OTHER; note = 2'-O-methyl modified nucleotide with phosphorothioate linkage
  modified_base 128: mod_base = OTHER; note = 2'-O-methyl modified nucleotide with phosphorothioate linkage
  modified_base 129: mod_base = OTHER; note = 2'-O-methyl modified nucleotide
  misc_feature 1..96: note = RNA
  misc_feature 97..119: note = DNA
  misc_feature 120..129: note = RNA
gtttcggcgccataaggtgggttttagagctagaaatagcaagttaaaataaggctagtccgttatcaacttgaaaaagtggcaccgagtcggtgcgcaaattgtcagacgacaaccaccttatggcgc

SEQ ID NO: 214 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
cgctgccttgccctcccagtgttttagagctagaaatagcaagttaaaataaggctagtccgttatcaacttgaaaaagtggcaccgagtcggtgctttt

SEQ ID NO: 215 - DNA, 127 bp, synthetic construct
  source 1..127: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = Nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..119: note = DNA
  misc_feature 120..127: note = RNA
gcccagagctggggcccccagttttagagctagaaatagcaagttaaaataaggctagtccgttatcaacttgaaaaagtggcaccgagtcggtgcgctcaccttcctgcagtcgtgggggccccag

SEQ ID NO: 216 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gaggaacgatttaagtcttggttttagagctagaaatagcaagttaaaataaggctagtccgttatcaacttgaaaaagtggcaccgagtcggtgctttt

SEQ ID NO: 217 - DNA, 126 bp, synthetic construct
  source 1..126: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 123: mod_base = OTHER; note = Nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide
  misc_feature 1..96: note = RNA
  misc_feature 97..113: note = DNA
  misc_feature 114..126: note = RNA
aaagagcatgatcacatgctgttttagagctagaaatagcaagttaaaataaggctagtccgttatcaacttgaaaaagtggcaccgagtcggtgcaaatatggcgtcaagcatgtgatcatgctc

SEQ ID NO: 218 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
ttatctaatggacgaccccagttttagagctagaaatagcaagttaaaataaggctagtccgttatcaacttgaaaaagtggcaccgagtcggtgctttt

SEQ ID NO: 219 - DNA, 125 bp, synthetic construct
  source 1..125: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 122: mod_base = OTHER; note = Nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide
  misc_feature 1..96: note = RNA
  misc_feature 97..113: note = DNA
  misc_feature 114..125: note = RNA
actgagggatgtctgaaggcgttttagagctagaaatagcaagttaaaataaggctagtccgttatcaacttgaaaaagtggcaccgagtcggtgcgccagtatccctggccttcagacatccct

SEQ ID NO: 220 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gcattttcgattccgtgactgttttagagctagaaatagcaagttaaaataaggctagtccgttatcaacttgaaaaagtggcaccgagtcggtgctttt

SEQ ID NO: 221 - DNA, 121 bp, synthetic construct
  source 1..121: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 118: mod_base = OTHER; note = Nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide
  misc_feature 1..96: note = RNA
  misc_feature 97..112: note = DNA
  misc_feature 113..121: note = RNA
ggagtgagggaaacggccccgttttagagctagaaatagcaagttaaaataaggctagtccgttatcaacttgaaaaagtggcaccgagtcggtgcgctggccctggggatgccgtttccc

SEQ ID NO: 222 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
agctgctcacttgagcctctgttttagagctagaaatagcaagttaaaataaggctagtccgttatcaacttgaaaaagtggcaccgagtcggtgctttt

SEQ ID NO: 223 - DNA, 118 bp, synthetic construct
  source 1..118: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = Nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide with phosphorothioate linkage
  modified_base 118: mod_base = OTHER; note = 2'-O-methyl modified nucleotide
  misc_feature 1..96: note = RNA
  misc_feature 97..108: note = DNA
  misc_feature 109..118: note = RNA
gtgctgaccatcgacgagaagttttagagctagaaatagcaagttaaaataaggctagtccgttatcaacttgaaaaagtggcaccgagtcggtgcagtccgtttcttgtcgatggtc

SEQ ID NO: 224 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
gacctcgggggggatagacagttttagagctagaaatagcaagttaaaataaggctagtccgttatcaacttgaaaaagtggcaccgagtcggtgctttt

SEQ ID NO: 225 - DNA, 128 bp, synthetic construct
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..114: note = DNA
  misc_feature 115..128: note = RNA
ggaatcccttctgcagcaccgttttagagctagaaatagcaagttaaaataaggctagtccgttatcaacttgaaaaagtggcaccgagtcggtgcggaaaagcgatcgtgatgctgcagaaggtttt

SEQ ID NO: 226 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Locked nucleic acid
  modified_base 99: mod_base = OTHER; note = Locked nucleic acid
  modified_base 101: mod_base = OTHER; note = Locked nucleic acid
  modified_base 103: mod_base = OTHER; note = Locked nucleic acid
  modified_base 105: mod_base = OTHER; note = Locked nucleic acid
  modified_base 107: mod_base = OTHER; note = Locked thymine
  modified_base 109: mod_base = OTHER; note = Locked nucleic acid
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..97: note = RNA
  misc_feature 98: note = DNA
  misc_feature 99: note = RNA
  misc_feature 100: note = DNA
  misc_feature 101: note = RNA
  misc_feature 102: note = DNA
  misc_feature 103: note = RNA
  misc_feature 104: note = DNA
  misc_feature 105: note = RNA
  misc_feature 106: note = DNA
  misc_feature 107: note = RNA
  misc_feature 108: note = DNA
  misc_feature 109: note = RNA
  misc_feature 110..114: note = DNA
  misc_feature 115..128: note = RNA

SEQ ID NO: 227 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 98: mod_base = OTHER; note = Locked nucleic acid
  modified_base 100: mod_base = OTHER; note = Locked nucleic acid
  modified_base 102: mod_base = OTHER; note = Locked nucleic acid
  modified_base 104: mod_base = OTHER; note = Locked nucleic acid
  modified_base 106: mod_base = OTHER; note = Locked nucleic acid
  modified_base 108: mod_base = OTHER; note = Locked nucleic acid
  modified_base 110: mod_base = OTHER; note = Locked thymine
  modified_base 112: mod_base = OTHER; note = Locked nucleic acid
  modified_base 114: mod_base = OTHER; note = Locked nucleic acid
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97: note = DNA
  misc_feature 98: note = RNA
  misc_feature 99: note = DNA
  misc_feature 100: note = RNA
  misc_feature 101: note = DNA
  misc_feature 102: note = RNA
  misc_feature 103: note = DNA
  misc_feature 104: note = RNA
  misc_feature 105: note = DNA
  misc_feature 106: note = RNA
  misc_feature 107: note = DNA
  misc_feature 108: note = RNA
  misc_feature 109: note = DNA
  misc_feature 110: note = RNA
  misc_feature 111: note = DNA
  misc_feature 112: note = RNA
  misc_feature 113: note = DNA
  misc_feature 114..128: note = RNA

SEQ ID NO: 228 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = Nucleotide with phosphorothioate linkage
  modified_base 99: mod_base = OTHER; note = Nucleotide with phosphorothioate linkage
  modified_base 100: mod_base = OTHER; note = Nucleotide with phosphorothioate linkage
  modified_base 101: mod_base = OTHER; note = Nucleotide with phosphorothioate linkage
  modified_base 102: mod_base = OTHER; note = Nucleotide with phosphorothioate linkage
  modified_base 103: mod_base = OTHER; note = Nucleotide with phosphorothioate linkage
  modified_base 104: mod_base = OTHER; note = Nucleotide with phosphorothioate linkage
  modified_base 105: mod_base = OTHER; note = Nucleotide with phosphorothioate linkage
  modified_base 106: mod_base = OTHER; note = Nucleotide with phosphorothioate linkage
  modified_base 107: mod_base = OTHER; note = Nucleotide with phosphorothioate linkage
  modified_base 108: mod_base = OTHER; note = Nucleotide with phosphorothioate linkage
  modified_base 109: mod_base = OTHER; note = Nucleotide with phosphorothioate linkage
  modified_base 110: mod_base = OTHER; note = Nucleotide with phosphorothioate linkage
  modified_base 111: mod_base = OTHER; note = Nucleotide with phosphorothioate linkage
  modified_base 112: mod_base = OTHER; note = Nucleotide with phosphorothioate linkage
  modified_base 113: mod_base = OTHER; note = Nucleotide with phosphorothioate linkage
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..114: note = DNA
  misc_feature 115..128: note = RNA

SEQ ID NO: 229 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 98: mod_base = OTHER; note = Nucleotide with phosphorothioate linkage
  modified_base 100: mod_base = OTHER; note = Nucleotide with phosphorothioate linkage
  modified_base 102: mod_base = OTHER; note = Nucleotide with phosphorothioate linkage
  modified_base 104: mod_base = OTHER; note = Nucleotide with phosphorothioate linkage
  modified_base 106: mod_base = OTHER; note = Nucleotide with phosphorothioate linkage
  modified_base 108: mod_base = OTHER; note = Nucleotide with phosphorothioate linkage
  modified_base 110: mod_base = OTHER; note = Nucleotide with phosphorothioate linkage
  modified_base 112: mod_base = OTHER; note = Nucleotide with phosphorothioate linkage
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97..114: note = DNA
  misc_feature 115..128: note = RNA

SEQ ID NO: 230 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 98: mod_base = OTHER; note = Locked nucleic acid with phosphorothioate linkage
  modified_base 100: mod_base = OTHER; note = Locked nucleic acid with phosphorothioate linkage
  modified_base 102: mod_base = OTHER; note = Locked nucleic acid with phosphorothioate linkage
  modified_base 104: mod_base = OTHER; note = Locked nucleic acid with phosphorothioate linkage
  modified_base 106: mod_base = OTHER; note = Locked nucleic acid with phosphorothioate linkage
  modified_base 108: mod_base = OTHER; note = Locked nucleic acid with phosphorothioate linkage
  modified_base 110: mod_base = OTHER; note = Locked thymine with phosphorothioate linkage
  modified_base 112: mod_base = OTHER; note = Locked nucleic acid with phosphorothioate linkage
  modified_base 115: mod_base = OTHER; note = 2'-O-methyl modified nucleotide
  modified_base 116: mod_base = OTHER; note = 2'-O-methyl modified nucleotide
  modified_base 117: mod_base = OTHER; note = 2'-O-methyl modified nucleotide
  modified_base 118: mod_base = OTHER; note = 2'-O-methyl modified nucleotide
  modified_base 119: mod_base = OTHER; note = 2'-O-methyl modified nucleotide
  modified_base 120: mod_base = OTHER; note = 2'-O-methyl modified nucleotide
  modified_base 121: mod_base = OTHER; note = 2'-O-methyl modified nucleotide
  modified_base 122: mod_base = OTHER; note = 2'-O-methyl modified nucleotide
  modified_base 123: mod_base = OTHER; note = 2'-O-methyl modified nucleotide
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified nucleotide
  misc_feature 1..96: note = RNA
  misc_feature 97: note = DNA
  misc_feature 98: note = RNA
  misc_feature 99: note = DNA
  misc_feature 100: note = RNA
  misc_feature 101: note = DNA
  misc_feature 102: note = RNA
  misc_feature 103: note = DNA
  misc_feature 104: note = RNA
  misc_feature 105: note = DNA
  misc_feature 106: note = RNA
  misc_feature 107: note = DNA
  misc_feature 108: note = RNA
  misc_feature 109: note = DNA
  misc_feature 110: note = RNA
  misc_feature 111: note = DNA
  misc_feature 112: note = RNA
  misc_feature 113..114: note = DNA
  misc_feature 115..128: note = RNA

SEQ ID NO: 231 - DNA, 128 bp, synthetic construct; identical to SEQ ID NO: 225
  source 1..128: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = Nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage
  modified_base 127: mod_base = OTHER; note = 2'-O-methyl modified extended nucleic acid with phosphorothioate linkage
  modified_base 128: mod_base = OTHER; note = 2'-O-methyl modified extended nucleic acid
  misc_feature 1..96: note = RNA
  misc_feature 97..114: note = DNA
  misc_feature 115..128: note = RNA

SEQ ID NO: 232 - RNA, 120 nt, synthetic construct
  source 1..120: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 117: mod_base = OTHER; note = Nucleotide with phosphorothioate linkage
  modified_base 118: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide
ggcccagactgagcacgtgagttttagagctagaaatagcaagttaaaataaggctagtccgttatcaacttgaaaaagtggcaccgagtcggtgctctgccatcaaagcgtgctcagtc

SEQ ID NO: 233 - RNA, 124 nt, synthetic construct
  source 1..124: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 121: mod_base = OTHER; note = Nucleotide with phosphorothioate linkage
  modified_base 122: mod_base = OTHER; note = 2'-O-methyl modified nucleotide with phosphorothioate linkage
  modified_base 123: mod_base = OTHER; note = 2'-O-methyl modified nucleotide with phosphorothioate linkage
  modified_base 124: mod_base = OTHER; note = 2'-O-methyl modified nucleotide
ggaatcccttctgcagcaccgttttagagctagaaatagcaagttaaaataaggctagtccgttatcaacttgaaaaagtggcaccgagtcggtgcggaaaagcgatcgtgatgctgcagaagg

SEQ ID NO: 234 - protein, 2014 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFEPKKKRKV

SEQ ID NO: 235 - protein, 1613 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSLTLIQGKKIVNHLRSRLAFEYNGQLIKILSKNIVAVGSLRREEKMLNDVDLLIIVPEKKLLKHVLPNIRIKGLSFSVKVCGERKCVLFIEWEKKTYQLDLFTALAEEKPYAIFHFTGPVSYLIRIRAALKKKNYKLNQYGLFKNQTLVPLKITTEKELIKELGFTYRIPKKRLSGGSKRTADGSEFEPKKKRKV

SEQ ID NO: 236 - protein, 2014 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDPRGILKAFPKRQKIHADASSKVLAKIPRREEGEEAEEWLSSLRAHVVRTGIGRARAELFEKQIVQHGGQLCPAQGPGVTHIVVDEGMDYERALRLLRLPQLPPGAQLVKSAWLSLCLQERRLVDVAGFSIFIPSRYLDHPQPSKAEQDASIPPGTHEALLQTALSPPPPPTRPVSPPQKAKEAPNTQAQPISDDEASDGEETQVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVSQEENGQQQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDWSGGSKRTADGSEFEPKKKRKV

SEQ ID NO: 237 - protein, 1774 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSESGGSKRTADGSEFEPKKKRKV

SEQ ID NO: 238 - protein, 2315 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKNKLVLIDGNSVAYRAFFALPLLHNDKGIHTNAVYGFTMMLNKILAEEQPTHILVAFDAGKTTFRHETFQDYKGGRQQTPPELSEQFPLLRELLKAYRIPAYELDHYEADDIIGTMAARAEREGFAVKVISGDRDLTQLASPQVTVEITKKGITDIESYTPETVVEKYGLTPEQIVDLKGLMGDKSDNIPGVPGIGEKTAVKLLKQFGTVENVLASIDEIKGEKLKENLRQYRDLALLSKQLAAICRDAPVELTLDDIVYKGEDREKVVALFQELGFQSFLDKMAVQTDEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAKSGGSKRTADGSEFEPKKKRKV

SEQ ID NO: 239 - protein, 2045 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSKRTADGSEFEPKKKRKV

SEQ ID NO: 240 - protein, 2143 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSLVSAIAANNLLEKVTKFHCGVIYDYRDGEYHSYRPGDFGAYLDALEAEVKRGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDVEHRAAWLLAKQERNGFPFDTKAIEELYVELAARRSELLRNLTETFGSWYQPKGGTEMFCHPRTGKPLPKYPRIKIPKVGGIFKKPKNKAQREGREPCELDTREYVAGAPYTPVEHVVFNPSSRDHIQKKLQEAGWVPTKFTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQMAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWIHDEIQVACRTEEIAKTVIEVAQEAMRWVGEHWNFRCLLDTEGKMGANWKECHSGGSKRTADGSEFEPKKKRKV

SEQ ID NO: 241 - protein, 2337 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCAIAVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGSGGSKRTADGSEFEPKKKRKV

SEQ ID NO: 242 - protein, 2294 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKIAVVDKALNNTRYDKHFQLYGEEVDVFHMCNEKLSGRLLKKHITIGTPENPFDPNDYDFVILVGAEPFLYFAGKKGIGDYTGKRVEYNGYANWIASISPAQLHFKPEMKPVFDATVENIHDIINGREKIAKAGDYRPITDPDEAEEYIKMVYNMVIGPVAFASATSALYCRDGYLLGVSISHQEYQGVYIDSDCLTEVAVYYLQKILDSENHTIVFHNLKFDMHFYKYHLGLTFDKAHKERRLHDTMLQHYVLDERRGTHGLKSLAMKYTDMGDYDFELDKFKDDYCKAHKIKKEDFTYDLIPFDIMWPYAAKDTDATIRLHNFFLPKIEKNEKLCSLYYDVLMPGCVFLQRVEDRGVPISIDRLKEAQYQLTHNLNKAREKLYTYPEVKQLEQDQNEAFNPNSVKQLRVLLFDYVGLTPTGKLTDTGADSTDAEALNELATQHPIAKTLLEIRKLTKLISTYVEKILLSIDADGCIRTGFHEHMTTSGRLSSSGKLNLQQLPRDESIIKGCVVAPPGYRVIAWDLTTAEVYYAAVLSGDRNMQQVFINMRNEPDKYPDFHSNIAHMVFKLQCEPRDVKKLFPALRQAAKAITFGILYGSGPAKVAHSVNEALLEQAAKTGEPFVECTVADAKEYIETYFGQFPQLKRWIDKCHDQIKNHGFIYSHFGRKRRLHNIHSEDRGVQGEEIRSGFNAIIQSASSDSLLLGAVDADNEIISLGLEQEMKIVMLVHDSVVAIVREDLIDQYNEILIRNIQKDRGISIPGCPIGIDSDSEAGGSRDYSCGKMKKQHPSIACIDDDEYTRYVKGVLLDAEFEYKKLAAMDKEHPDHSKYKDDKFIAVCKDLDNVKRILGASGGSKRTADGSEFEPKKKRKV

SEQ ID NO: 243 - protein, 2143 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSIVSAIAANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICHSGGSKRTADGSEFEPKKKRKV

SEQ ID NO: 244 - protein, 2315 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAKSGGSKRTADGSEFEPKKKRKV

SEQ ID NO: 245 - protein, 2037 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSSASAQQLAEELQIFGLDCEEALIEKLVELCVQYGQNEEGMVGELIAFCTSTHKVGLTSEILNSFEHEFLSKRLSKARHSTCKDSGHAGARDIVSIQELIEVEEEEEILLNSYTTPSKGSQKRAISTPETPLTKRSVSTRSPHQLLSPSSFSPSATPSQKYNSRSNRGEVVTSFGLAQGVSWSGRGGAGNISLKVLGCPEALTGSYKSMFQKLPDIREVLTCKIEELGSELKEHYKIEAFTPLLAPAQEPVTLLGQIGCDSNGKLNNKSVILEGDREHSSGAQIPVDLSELKEYSLFPGQVVIMEGINTTGRKLVATKLYEGVPLPFYQPTEEDADFEQSMVLVACGPYTTSDSITYDPLLDLIAVINHDRPDVCILFGPFLESKHEQVENCLLTSPFEDIFKQCLRTIIEGTRSSGSHLVFVPSLRDVHHEPVYPQPPFSYSDLSREDKKQVQFVSEPCSLSINGVIFGLTSTDLLFHLGAEEISSSSGTSDRFSRILKHILTQRSYYPLYPPQEDMAIDYESFYVYAQLPVTPDVLIIPSELRYFVKDVLGCVCVNPGRLTKGQVGGTFARLYLRRPAADGAERQSPCIAVQVVRISGGSKRTADGSEFEPKKKRKV

SEQ ID NO: 246 - protein, 2674 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSFSGGGGPLSPGGKSAARAASGFFAPAGPRGAGRGPPPCLRQNFYNPYLAPVGTQQKPTGPTQRHTYYSECDEFRFIAPRVLDEDAPPEKRAGVHDGHLKRAPKVYCGGDERDVLRVGSGGFWPRRSRLWGGVDHAPAGFNPTVTVFHVYDILENVEHAYGMRAAQFHARFMDAITPTGTVITLLGLTPEGHRVAVHVYGTRQYFYMNKEEVDRHLQCRAPRDLCERMAAALRESPGASFRGISADHFEAEVVERTDVYYYETRPALFYRVYVRSGRVLSYLCDNFCPAIKKYEGGVDATTRFILDNPGFVTFGWYRLKPGRNNTLAQPRAPMAFGTSSDVEFNCTADNLAIEGGMSDLPAYKLMCFDIECKAGGEDELAFPVAGHPEDLVIQISCLLYDLSTTALEHVLLFSLGSCDLPESHLNELAARGLPTPVVLEFDSEFEMLLAFMTLVKQYGPEFVTGYNIINFDWPFLLAKLTDIYKVPLDGYGRMNGRGVFRVWDIGQSHFQKRSKIKVNGMVNIDMYGIITDKIKLSSYKLNAVAEAVLKDKKKDLSYRDIPAYYAAGPAQRGVIGEYCIQDSLLVGQLFFKFLPHLELSAVARLAGINITRTIYDGQQIRVFTCLLRLADQKGFILPDTQGRFRGAGGEAPKRPAAAREDEERPEEEGEDEDEREEGGGEREPEGARETAGRHVGYQGARVHDPTSGFHVNPVVGFDFASLYPSIIQAHNLCFSTLSLRADAVAHLEAGKDYLEIEVGGRRLFFVKAHVRESLLSILLRDWLAMRKQIRSRIPQSSPEEAVLLDKQQAAIKVVCNSVYGFTGVQHGLLPCLHVAATVTTIGREMLLATREYVHARWAAFEQLLADFPEAADMRAPGPYSMRIIYGDTDSIFVLCRGLTAAGLTAMGDKMASHISRALFLPPIKLECEKTFTKLLLIAKKKYIGVIYGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSHADPPGGASKPRKLLVSELAEDPAYAIAHGVALNTDYYFSHLLGAACVTFKALFGNNAKITESLLKRFIPEVWHPPDDVAARLRAAGFGAVGAGATAEETRRMLHRAFDTLASGGSKRTADGSEFEPKKKRKV

SEQ ID NO: 247 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 248 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 249 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 250 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 251 - protein, 2090 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKGTGSGAWKEWLERKVGEGRARRLIEYFGSAGEVGKLVENAEVSKLLEVPGIGDEAVARKVPGSGGSKRTADGSEFESPKKKRKVGSGPAAKRVKLD

SEQ ID NO: 252 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 253 - protein, 2254 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSSGSETPGTSESATPESSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFESPKKKRKVGSGPAAKRVKLD

SEQ ID NO: 254 - protein, 2254 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCDFETTTKVEDCRVWAYGYMNIEDHSEYKIGNSLDEFMAWALKVQADLYFHNLKFDGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSSGGSSGSETPGTSESATPESSGGSSGGSMAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNESGGSKRTADGSEFESPKKKRKVGSGPAAKRVKLD

SEQ ID NO: 255 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGARKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 256 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGARKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 257 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTEIKNTSDGCRKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 258 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 259 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 260 - protein, 2028 aa, synthetic construct
MKRTADGSEFESPKKKRKVDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 261 - protein, 1781 aa, synthetic construct
MKRTADGSEFESPKKKRKVSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDFSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 262 - protein, 1781 aa, synthetic construct
MKRTADGSEFESPKKKRKVSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDAILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDFSGGSSGGSKRTADGSEFESPKKKRKVSGGSSGGSKHMPRKRYSCAFETTTKVEDCRVWAYGYMNIEDHSEYKIGNSLDEFMAWALKVQADLYFHNLKFAGAFIINWLERNGFKWSADGLPNTYNTIISRTGQWYMIDICLGYKGKRKIHTVIYDSLKKLPFPVKKIAKDCKLTVLKGDIDYHKERPVGYKITPEEYAYIKNDIQIIAEALLIQFKQGLDRMTAGSDSLKDFKDIITTKKFKKVFPTLSLGLDKKVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGCIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRPKTYIQDIYMKEVDGELVEGSPDDYTDIKLSVKCAGMTDKIKKEVTFENFKVGFSRKMKPKPVQVPGGVVLVDDTFTIKSGGSKRTADGSEFESPKKKRKVGSGPAAKRVKLD

SEQ ID NO: 263 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 264 - protein, 606 aa, Escherichia coli; identical to SEQ ID NO: 69

(19) United States
(12) Patent Application Publication
(10) Pub. No.: US 2026/0193622 A1
(43) Pub. Date: Jul. 9, 2026

(54) DNA-DEPENDENT DNA POLYMERASE COMPOSITIONS, METHODS, AND USES THEREOF

(71) Applicant: Revision Bio Corporation, Cambridge, MA (US)

(72) Inventors: Shantanu KUMAR, Hopkinton, MA (US); Jonathan Hsu, Cambridge, MA (US)

(21) Appl. No.: 19/572,051

(22) Filed: Mar. 19, 2026

Related U.S. Application Data
(63) Continuation of application No. PCT/US24/47687, filed on Sep. 20, 2024.
(60) Provisional application No. 63/584,641, filed on Sep. 22, 2023.

(51) Int. Cl. C12N 9/12; C12N 9/22; C12N 15/11; C12P 19/34

(52) U.S. Cl. CPC C12N 9/1252; C12N 9/226; C12N 15/11; C12P 19/34; C12Y 207/07007; C12N 2310/20

(57) ABSTRACT

DNA-dependent DNA polymerase compositions, guide polynucleotides, systems, methods, and uses thereof are provided. Methods of modifying a target nucleic acid and genetically modifying a cell are described. The methods can be used for biotechnology applications, therapeutic treatments, and for generating cell therapies for the treatment of various diseases and conditions. Also included are scaffolds and kits comprising the compositions and systems described herein.

### CROSS-REFERENCE TO RELATED APPLICATION

This application claims the benefit of and is a continuation of International Application No. PCT/US2024/047687, filed Sep. 20, 2024, which claims the benefit of priority to U.S. Provisional Patent Application No. 63/584,641, filed Sep. 22, 2023, the contents of which is incorporated herein by reference in its entirety.

### SEQUENCE LISTING

The instant application contains a Sequence Listing which has been submitted electronically in xml format and is hereby incorporated by reference in its entirety. Said xml copy, created on Sep. 19, 2024, is named 219001-701601_SL.xml and is 664,000 bytes in size.

### BACKGROUND

While many CRISPR systems have emerged as a useful tool for gene editing, such systems are limited by off-target effects, the length of insertion sequences, and inefficient nucleic acid synthesis with low fidelity. Therefore, there is a great unmet need for gene modification systems that can address these concerns.

### SUMMARY

Provided herein are compositions, wherein the compositions comprise: (a) a polynucleotide encoding a phi29 DNA-dependent DNA polymerase or a variant thereof; (b) a polynucleotide encoding a nickase; and (c) a guide polynucleotide comprising: (i) a targeting region that has complementarity to a target nucleic acid; (ii) a protein binding region, wherein the protein binding region comprises a secondary structure that binds to the nickase; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides.

Provided herein are compositions, wherein the compositions comprise: (a) an engineered fusion protein comprising: (i) a phi29 DNA-dependent DNA polymerase or a variant thereof; and (ii) a nickase, wherein the phi29 DNA-dependent DNA polymerase or a variant thereof is linked to the nickase, (b) a guide polynucleotide comprising: (i) a targeting region that has complementarity to a target nucleic acid; (ii) a protein binding region, wherein the protein binding region comprises a secondary structure that binds to the nickase; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and (iv) a hybridization region, wherein the hybridization region comprises: deoxyribonucleotides and ribonucleotides.

Provided herein are compositions, wherein the compositions comprise: (a) a polynucleotide encoding a DNA-dependent DNA polymerase or a variant thereof, (b) a polynucleotide encoding a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92, (c) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and (iv) a hybridization region, wherein the hybridization region comprises: deoxyribonucleotides and ribonucleotides.

Provided herein are compositions, wherein the compositions comprise: (a) an engineered fusion protein comprising: (i) a DNA-dependent DNA polymerase or a variant thereof, (ii) a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92, (b) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and (iv) a hybridization region, wherein the hybridization region comprises: deoxyribonucleotides and ribonucleotides.

Provided herein are compositions, wherein the compositions comprise: (a) a polynucleotide encoding a DNA-dependent DNA polymerase or a variant thereof, (b) a polynucleotide encoding a Streptococcus thermophilus Cas9 nickase or a variant thereof, wherein the variant comprises an amino acid substitution at position 599 as compared to SEQ ID NO: 97, (c) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides.

Provided herein are compositions, wherein the compositions comprise: (a) an engineered fusion protein comprising: (i) a DNA-dependent DNA polymerase or a variant thereof, and (ii) a Streptococcus thermophilus Cas9 nickase or a variant thereof, wherein the variant comprises an amino acid substitution at position 599 as compared to SEQ ID NO: 97, (b) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides.

Provided herein are engineered fusion proteins, wherein the engineered fusion proteins comprise: (a) a phi29 DNA-dependent DNA polymerase or a variant thereof, and (b) a nickase or a variant thereof.

Provided herein are systems for synthesizing a nucleic acid sequence, wherein the systems comprise: (a) an engineered protein construct comprising a nickase region; (b) a DNA-dependent DNA polymerase (DdDP) or a functional fragment thereof, and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region has complementarity to the target nucleic acid, wherein upon introduction to a cell or a cell-free system, the system synthesizes a nucleic acid sequence that is incorporated into the target nucleic acid.

Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: one or more ribonucleotides or one or more deoxyribonucleotides, wherein the hybridization region has complementarity to the target nucleic acid, wherein upon introduction to a cell or a cell-free system, the system synthesizes a nucleic acid sequence that is incorporated into the target nucleic acid.

Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: one or more deoxyribonucleotides, wherein the hybridization region has complementarity to the target nucleic acid, wherein upon introduction to a cell or a cell-free system, the system synthesizes a nucleic acid sequence that is incorporated into the target nucleic acid.

Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: a deoxyribonucleotide and a ribonucleotide, wherein the hybridization region has complementarity to the target nucleic acid, wherein upon introduction to a cell or a cell-free system, the system synthesizes a nucleic acid sequence that is incorporated into the target nucleic acid.

Provided herein are compositions, wherein the compositions comprise: a system provided herein, an engineered protein construct provided herein, a DNA-dependent DNA polymerase provided herein or a functional fragment thereof, or a guide polynucleotide provided herein.

Provided herein are compositions, wherein the compositions comprise: the systems provided herein; and a delivery vehicle.

Provided herein are polynucleotides, wherein the polynucleotides encode for a composition or a system provided herein, an engineered protein provided herein, or a guide polynucleotide provided herein.

Provided herein are sets of polynucleotides, wherein the sets of polynucleotides encode for a system provided herein, an engineered protein provided herein, or a guide polynucleotide provided herein.

Provided herein are nanoparticles, wherein the nanoparticles comprise: the polynucleotides provided herein, the sets of polynucleotides provided herein, the systems provided herein, the compositions provided herein, the cells provided herein, the vectors provided herein or any portion thereof.

Provided herein are vectors, wherein the vectors comprise: the polynucleotides provided herein.

Provided herein are cells, wherein the cells comprise: a system provided herein, a polynucleotide provided herein, a vector provided herein, a composition provided herein, an engineered protein provided herein, or a guide polynucleotide provided herein.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises ribonucleotides.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises deoxyribonucleotides and ribonucleotides.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region; and an engineered protein or a polynucleotide encoding the engineered protein, wherein the engineered protein comprises a nickase region; and a DNA-dependent DNA polymerase (DdDP) region.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises one or more ribonucleotides or one or more deoxyribonucleotides; and an engineered protein or a polynucleotide encoding the engineered protein, wherein the engineered protein comprises a nickase region; and a DNA-dependent DNA polymerase (DdDP) region.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises a deoxyribonucleotide and a ribonucleotide; and an engineered protein or a polynucleotide encoding the engineered protein, wherein the engineered protein comprises a nickase region; and a DNA-dependent DNA polymerase (DdDP) region.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises a deoxyribonucleotide and a ribonucleotide; and an engineered protein or a polynucleotide encoding the engineered protein, wherein the engineered protein comprises a nickase region; and a phi29 DNA-dependent DNA polymerase (DdDP) region.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises a deoxyribonucleotide and a ribonucleotide; and an engineered protein or a polynucleotide encoding the engineered protein, wherein the engineered protein comprises a nickase region, wherein the nickase region comprises a SpCas9 nickase, a St1Cas9 nickase, or a variant thereof, and a DNA-dependent DNA polymerase (DdDP) region.

Provided herein are methods of synthesizing a nucleic acid, wherein the methods comprise: contacting a cell or a cell-free system with: a system provided herein, a polynucleotide provided herein, a vector provided herein, a composition provided herein, an engineered protein provided herein, or a guide polynucleotide provided herein.

Provided herein are methods of synthesizing a nucleic acid, wherein the methods comprise: contacting a cell or a cell-free system with: (a) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration relative to the target nucleic acid; (iv) a hybridization region, wherein the hybridization region comprises a deoxyribonucleotide and a ribonucleotide; (b) an engineered protein or a polynucleotide encoding the engineered protein, wherein the engineered protein comprises: (i) a nickase region; and (ii) a DdDP region; wherein: the guide polynucleotide forms a complex with the engineered protein via the protein binding region, the targeting sequence forms a complex with a complementary strand of the target nucleic acid, the nickase region of the engineered protein generates a single strand break in the target nucleic acid to generate a leading strand, the hybridization region forms a complex with the leading strand, and wherein the guide polynucleotide associates with the DdDP region of the engineered protein, thereby synthesizing a nucleic acid.

Provided herein are methods, wherein the methods comprise: administering to a cell, a tissue, or a subject a system provided herein, a composition provided herein, a vector provided herein, a polynucleotide provided herein, or a cell provided herein, wherein the administering generates an alteration in a target nucleic acid.

Provided herein are methods, wherein the methods comprise: administering to a cell, a tissue, or a subject a system provided herein, a composition provided herein, a vector provided herein, a polynucleotide provided herein, thereby generating an alteration in a gene of the cell.

Provided herein are populations of cells made by the methods provided herein.

Provided herein are kits, wherein the kits comprise: a system provided herein, a composition provided herein, a vector provided herein, a polynucleotide provided herein, or a cell provided herein, packaging and materials therefor.

Provided herein are kits, wherein the kits comprise: a first container comprising: a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration nucleobase relative to the target nucleic acid; and (iv) a hybridization region, wherein the hybridization region comprises: a deoxyribonucleotide and a ribonucleotide, and a second container comprising: an engineered protein comprising a nickase operably linked to a DNA-dependent DNA polymerase.

Provided herein are scaffolds, wherein the scaffolds comprise: a system provided herein or a composition provided herein; and a surface, wherein the system or the composition are immobilized to the surface.

### A BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show a schematic an embodiment of the system provided herein. FIG. 1A shows a schematic of the DNA editing process using an engineered protein comprising a Cas9 nickase-DNA-dependent polymerase and a guide polynucleotide designed to form a complex with a double stranded target nucleic acid and edit the target nucleic acid by synthesizing a new DNA strand that is incorporated into the target nucleic acid sequence. FIG. 1B shows a schematic of the DNA-dependent DNA polymerase guide RNA (degRNA). The degRNA comprises a DNA-dependent DNA polymerase (DdDP) synthesis template region (DST) and a hybridization region (HR). Key: D is a deoxyribonucleotide (DNA) and R is a ribonucleotide (RNA).

FIG. 2 shows an image from a denaturing urea polyacrylamide gel showing activity of DNA editing by DNA-dependent DNA polymerase I (Klenow Fragment) with the DNA-dependent DNA polymerase synthesis template (DST) and hybridization region (HR) supplied in trans. The band in lane 1 represents the oligo ladder showing non-nicked non-target strand (54 nucleotides (nt)) (Oligo 1). The bands in lane 2 represent the Cas9 nickase (H840A) control using in vitro transcribed sgRNA. The bands in lane 3 represent the positive control with insertion of 15 nt DNA editing (49 nt) (Oligo 32, Oligo 19). The bands in Lanes 4-9 represent the editing products of DNA editing with DST and HR in trans with Oligo 17 (lane 4), Oligo 18 (lane 5), Oligo 19 (lane 6), Oligo 20 (lane 7), Oligo 21 (lane 8), Oligo 22 (lane 9), respectively, and all lanes using in vitro transcribed sgRNA. The bands in lane 10 represent positive control with insertion of 15 nt DNA editing (49 nt) (Oligo 32, Oligo 19) and spiked-in oligo ladder for non-nicked (Oligo 1, 54 nt) and pre-nicked (Oligo 32, 34 nt) DNA. The bands in lane 11-12 represent the products of DNA editing with DST and HR in trans with Oligo 25 (lane 11) and Oligo 26 (lane 12), respectively, with both lanes using in vitro transcribed sgRNA.

FIG. 3 shows an image from a denaturing urea polyacrylamide gel showing activity of DNA editing by DNA-dependent DNA polymerase I (Klenow Fragment) with the DNA-dependent DNA polymerase synthesis template (DST) and hybridization region (HR) incorporated into a synthetic guide RNA. The bands in lanes 1 and 12 represent the Oligo ladder showing non-nicked (Oligo 1, 54 nt) and pre-nicked (Oligo 32, 34 nt) DNA. The bands in lane 2 represent the editing products of Cas9 nickase (H840A) control using Guide 3. The bands in lanes 3-11 represent the editing products of DNA editing with Guide 1 (lane 3), Guide 2 (lane 4), Guide 3 (lane 5), Guide 4 (lane 6), Guide 5 (lane 7), Guide 6 (lane 8), Guide 7 (lane 9), Guide 8 (lane 10), Guide 9 (lane 11), respectively.

FIGS. 4A-4B show graphs of DNA-dependent DNA polymerases (DdDP) genome editor editing efficiency in human cells. FIG. 4A shows a graph of DNA-dependent DNA polymerase constructs assayed for precision genome editing activity in HEK293T cells at genomic target site, HEK site 3. FIG. 4B shows a graph of DNA-dependent DNA polymerase constructs assayed for precision genome editing activity in HEK293T cells at genomic target site, FANCF site 1. Three guide constructs (HEK site 3: SEQ ID NO: 187, SEQ ID NO: 188, SEQ ID NO: 189; FANCF site 1: SEQ ID NO: 190, SEQ ID NO: 191, SEQ ID NO: 192) were assayed for each target site as indicated by the key in the right corner. X-axis: DdDP-genome editor names (pRVB_1 to pRVB_13, SEQ ID NO: 234-SEQ ID NO: 246), Y-axis: Desired Editing Efficiency (%)

FIGS. 5A-5B shows graphs of Phi29 DNA polymerase genome editor editing efficiency. FIG. 5A shows a graph of Phi29 DNA polymerase variants that exhibit enhanced precision genome editing activity in HEK293T cells at genomic target site, HEK site 3 (SEQ ID NO: 255, SEQ ID NO: 250). FIG. 5B shows a graph of Phi29 DNA polymerase variants that exhibit enhanced precision genome editing activity in HEK293T cells at genomic target site, FANCF site 1 (SEQ ID NO: 255, SEQ ID NO: 250) compared to PE3 (HEK site 3: SEQ ID NO: 263, Guides: SEQ ID NO: 232, SEQ ID NO: 207; FANCF site 1: SEQ ID NO: 263, Guides: SEQ ID NO: 233, SEQ ID NO: 208). X-axis: DdDP-genome editor names (pRVB_14 (SEQ ID NO: 247), pRVB_22 (SEQ ID NO: 255), pRVB_17 (SEQ ID NO: 250), Y-axis: Desired Editing Efficiency (%).

FIGS. 6A-6B show graphs of guide RNA effects on the activity of DdDP genome editors. FIG. 6A shows a graph of various guide RNA constructs and DdDP genome editing system activity in HEK293T cells at genomic target site HEK site 3 using the pRVB_17 DdDP editor construct (SEQ ID NO: 250) and associated nicking gRNAs. FIG. 6B shows a graph of various guide RNA constructs and DdDP genome editing system activity in HEK293T cells at genomic target site, FANCF site 1. X-axis: guide RNA names (gRVB_7, gRVB_8, gRVB_9, gRVB_10, gRVB_11, gRVB_12, SEQ ID NO: 193-SEQ ID NO: 198, respectively); Y-axis: Desired Editing Efficiency (%).

FIG. 7 shows a graph of modified DdDP genome editor activity on substitution, insertion, and deletion edits in human cells. These experiments were performed in HEK293T cells using the pRVB_17 DdDP editor construct (SEQ ID NO: 250). X-axis: guide RNA names (gRVB_12, gRVB_13, gRVB_14, gRVB_15, gRVB_16, gRVB_17, gRVB_18, gRVB_19, gRVB_9, gRVB_20, SEQ ID NO: 198-SEQ ID NO: 205, SEQ ID NO: 195, SEQ ID NO: 206, respectively); Y-axis: Desired Editing Efficiency (%).

FIG. 8 shows a graph of DdDP genome editors assayed to install various human pathogenic variants across endogenous genomic loci in human cells. These experiments were performed in HEK293T cells using the pRVB_17 DdDP editor construct (SEQ ID NO: 250) with guide RNAs gRVB_23-36 (SEQ ID NO: 209-SEQ ID NO: 222). X-axis: gene target (Presenilin-1 (PSEN1), IDS, GATA2, DNA methyltransferase 1 (DNMT1), MYBPC3, PKD1, and TTR); Y-axis: Desired Editing Efficiency (%).

FIG. 9 shows a graph of DdDP genome editor activity in primary human hepatocytes at the genomic target site, FANCF site 1 (pRVB_17 (SEQ ID NO: 250), gRVB_12 (SEQ ID NO: 198), gRVB_22 (SEQ ID NO: 208)) as compared to PE3 (pRVB 30 (SEQ ID NO: 263), gRVB_47 (SEQ ID NO: 233), gRVB_22 (SEQ ID NO: 208)). X-axis: genome editor name (PE3 also referred to herein as pRVB_30, SEQ ID NO: 263; or pRVB_17, SEQ ID NO: 250); Y-axis: Desired Editing Efficiency (%).

FIG. 10 shows a graph of Phi29 DNA polymerase precision genome editing within the hybridization region (HR) of the guide RNA. The same guide RNAs were used (gRVB_37 (SEQ ID NO: 223), gRVB_38 (SEQ ID NO: 224). X-axis: DdDP genome editor name (3′ exonuclease-deficient DdDP editor pRVB_15 (SEQ ID NO: 248) was compared with pRVB_19 (SEQ ID NO: 252)); Y-axis: Desired Editing Efficiency (%).

Various aspects now will be described more fully hereinafter. Such aspects may, however, be embodied in many different forms and should not be construed as limited to the embodiments set forth herein.

### DETAILED DESCRIPTION OF THE INVENTION

Provided herein are compositions, kits, methods, and uses thereof for editing and synthesizing a target gene using a DNA-dependent DNA polymerase. Briefly, further described herein are: (1) gene-editing systems; (2) delivery vehicles and vectors; (3) cells and cell-free systems; (4) pharmaceutical compositions, dosing, and administration; (5) scaffolds and systems; (6) kits; (7) gene-editing activity; and (8) applications.

Provided herein are compositions and systems for gene editing and DNA synthesis that can be used in a variety of applications, such as gene-editing of microbial, mammalian, and plant cells, diagnostics, therapeutics, and biologics manufacturing. The compositions and systems provided herein include an engineered protein that comprises nickase activity and DNA-dependent DNA polymerase activity or a polynucleotide encoding the engineered protein. The engineered protein can bind to dsDNA and create a nick on the non-target DNA strand to synthesize DNA in a facile process.

The compositions and systems provided herein also include a guide polynucleotide that binds to the engineered protein and the target nucleic acid to promote loop formation between the cleaved target nucleic acid and the guide polynucleotide for high fidelity targeting and editing. The compositions and systems provided herein allow for controlled editing outcomes and have limited off-target effects. Furthermore, the compositions and systems provided herein are useful for targeted insertion of small and large nucleic acid sequences that can be synthesized and integrated into the target nucleic acid with precision.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to ±20% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “leading strand” and its grammatical equivalents refers to a single-stranded nucleic acid sequence (e.g., DNA) that binds to a hybridization region of a guide polynucleotide provided herein or the DNA-dependent DNA polymerase synthesis template (DST) of a guide polynucleotide provided herein.

As used herein, the term “complementary strand” and its grammatical equivalents refers to a single-stranded nucleic acid sequence (e.g., DNA) that comprises a protospacer adjacent motif (PAM), is complementary to the leading strand, and/or binds to a targeting region of a guide polynucleotide provided herein.

The term “effective amount” or “therapeutically effective amount” refers to an amount that is sufficient to achieve or at least partially achieve the desired effect.

### (1) Gene Editing Systems

Provided herein are systems, wherein the systems comprise: a guide polynucleotide and an engineered protein or a polynucleotide encoding the engineered protein. In some embodiments, the systems are provided for use in the synthesis of a nucleic acid. In some embodiments, the systems cleave a target nucleic acid. In some embodiments, the systems generate a single strand break in the target nucleic acid. In some embodiments, the systems synthesize a new nucleic acid for incorporation into the target nucleic acid, thereby replacing an abnormal nucleic acid sequence relative to a reference sequence.

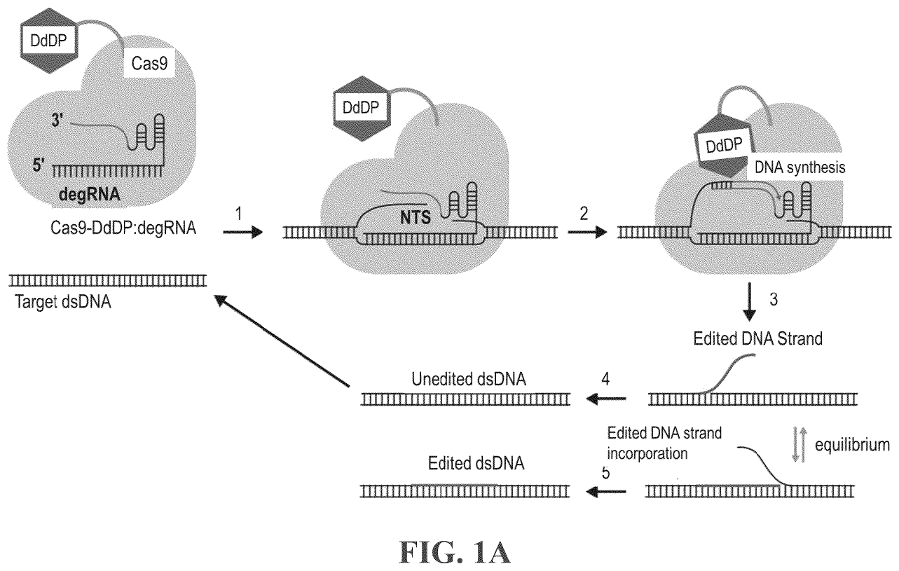

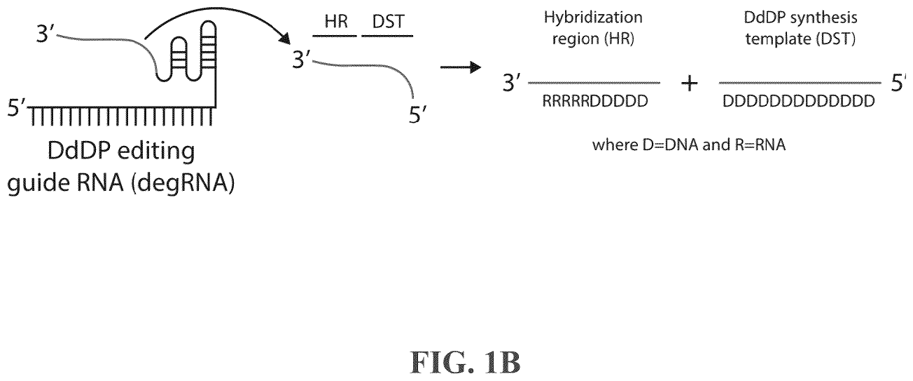

As shown in FIG. 1A, to construct a system for DNA synthesis, an engineered protein is made that includes a nickase and DNA-dependent DNA polymerase (DdDP) that targets a nucleic acid of interest using a guide polynucleotide. The guide polynucleotide can form a complex with the engineered protein to form a ribonucleoprotein (RNP). This RNP can target double-stranded DNA (dsDNA) in the presence of a protospacer adjacent motif (PAM) and high sequence homology between the guide polynucleotide spacer sequence and the dsDNA. Following engagement between the Cas9 nickase-DdDP:guide polynucleotide complex and dsDNA, the nickase performs a single-stranded break on the non-target strand (NTS, also referred to herein as the leading strand) allowing the release of a single-stranded DNA (Step 1). The hybridization region (HR) of the guide polynucleotide can then hybridize to this single-stranded DNA, presenting a substrate for DdDP to directly synthesize in genetic information encoded in the DNA-dependent DNA polymerase synthesis template (DST) of the guide polynucleotide (Step 2). Following the DNA synthesis step, the Cas9 nickase-DdDP:guide polynucleotide complex dissociates from the target dsDNA and this results in different conformations where the edited DNA strand is either unhybridized or hybridized to the dsDNA (Step 3). Subsequently, endogenous cellular enzymes for DNA repair incorporate either the original DNA strand or newly-synthesized edited DNA strand into the dsDNA, resulting in either an unedited dsDNA (Step 4) or edited dsDNA (Step 5), respectively.

The guide polynucleotide for the DNA synthesis system provided herein includes of two elements: a hybridization region (HR) and DNA-dependent DNA polymerase synthesis template (DST) (FIG. 1B). The hybridization region mediates hybridization with the non-target strand (NTS, also referred to herein as a leading strand) to present a primer structure for DdDP to perform DNA synthesis. The hybridization region comprises RNA (typically on the 3′ end) and DNA (typically on the 5′ end) at varying proportions to enhance DNA editing efficiency. The DST encodes the genetic information to be written into the target nucleic acid and serves as the synthesis template for DdDP during the editing event. The DST typically comprises deoxyribonucleobases to facilitate efficient DNA synthesis by DdDP. In some embodiments, the hybridization region comprises RNA only. In some embodiments, the DST comprises DNA only.

The systems provided herein include the following elements: (1) a guide polynucleotide; (2) a nuclease, a nickase, or a variant thereof; and a DNA-dependent DNA polymerase or a functional fragment thereof. A polynucleotide can be used that encodes the nuclease, nickase, or variant thereof, such as a DNA or an RNA. A polynucleotide can also be used that encodes the DNA-dependent DNA polymerase or a functional fragment thereof.

The systems provided herein are designed to synthesize a nucleic acid (e.g., DNA) with high fidelity and high processivity. The systems provided herein are useful in permitting precise, targeted insertion of polynucleotides into a target nucleic acid. In some embodiments, the systems provided herein permit targeted insertion of a polynucleotide that is at least 5, at least 10, at least 15, at least 20, at least 25, at least 30, at least 35, at least 40, at least 45, at least 50, at least 100, at least 200, at least 300, at least 400, at least 500, at least 600, at least 700, at least 800, at least 900, at least 1000, at least 10,000, at least 20,000, at least 30,000, at least 40,000, at least 50,000, at least 60,000, at least 70,000, at least 80,000, at least 90,000, or at least 100,000 or more. In some embodiments, the systems provided herein permit targeted insertion of a single stranded polynucleotide that is 10 kb or larger.

### Guide Polynucleotides

Provided herein are compositions and systems comprising a guide polynucleotide or a polynucleotide encoding the guide polynucleotide. A guide polynucleotide provided herein binds to a target nucleic acid (e.g., a DNA sequence) and a nuclease or a nickase provided herein to form a complex. In some embodiments, the complex facilitates cleavage of the target nucleic acid. A guide polynucleotide can also bind to a DNA polymerase via a hybridization region linked to a DNA-dependent DNA polymerase synthesis template (DST) region.

In some embodiments, the guide polynucleotide is a single guide sequence. In some embodiments, the guide polynucleotide comprises ribonucleosides and deoxyribonucleosides. In some embodiments, the guide polynucleotide comprises ribonucleotides and deoxyribonucleotides. In some embodiments, the guide polynucleotide comprises nucleobases. Examples of nucleobases include, but are not limited to, adenine (A), guanine (G), cytosine (C), thymine (T), and uracil (U). The nucleobase of a nucleotide can be independently selected from a purine, a pyrimidine, a purine or pyrimidine analog. In an embodiment, the nucleobase can include, for example, naturally-occurring and synthetic derivatives of a base. Guide polynucleotides provided herein can comprise non-naturally occurring sequences or engineered sequences.

In some embodiments, the guide polynucleotide comprises a modified nucleobase, a modified nucleotide, or a modified nucleoside. The modified nucleosides and modified nucleotides described herein, which can be incorporated into a target nucleic acid, can include a modified nucleobase. In some embodiments, the nucleobase or the nucleotide provided herein is chemically modified. Nucleobases and nucleotides provided herein can be modified or wholly replaced to provide modified nucleosides and modified nucleotides that can be incorporated into a target nucleic acid. In some embodiments, the nucleobases or nucleotides provided herein can be extended nucleic acids (also referred to as exNAs) which comprise an extra carbon incorporated 3′ of a phosphate. An exNA-phosphorothioate modification can be used to improve oligonucleotide stability and long-term efficacy.

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m C), N4-acetyl-cytidine (act), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k C), a-thio-cytidine, 2′-0-methyl-cytidine (Cm), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f 5Cm), N4,N4,2′-0-trimethyl-cytidine (m4 2Cm), 1-thio-cytidine, 2′-F-ara-cytidine, 2′-F-cytidine, 2′-OH-ara-cytidine, 2′-OMe-exNA-cytidine, and 2′-F-exNA-cytidine.

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (i A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m6 2A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-0-methyl-adenosine (Am), N6,2′-0-dimethyl-adenosine (m6Am), N6-Methyl-2′-deoxyadenosine, N6,N6,2′-0-trimethyl-adenosine (m6 2Am 1,2′-0-dimethyl-adenosine (i Am), 2′-0-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, N6-(19-amino-pentaoxanonadecyl)-adenosine, 2′-OMe-exNA-adenosine, and 2′-F-exNA-adenosine.

In some embodiments, the modified nucleobase is a modified guanine. Exemplary nucleobases and nucleosides having a modified guanine include without limitation inosine (I), 1-methyl-inosine (iVl), wyosine (imG), methylwyosine (mimG), 4-demethyl-wyosine (imG-14), isowyosine (imG2), wybutosine (yW), peroxywybutosine (o2yW), hydroxy wybuto sine (OHyW), undermodified hydroxy wybuto sine (OHyW*), 7-deaza-guanosine, queuosine (Q),epoxyqueuosine (oQ), galactosyl-queuosine (galQ), mannosyl-queuosine (manQ), 7-cyano-7-deaza-guanosine (preQo), 7-aminomethyl-7-deaza-guanosine (preQO, archaeosine (G+), 7-deaza-8-aza-guanosine, 6-thio-guanosine, 6-thio-7-deaza-guanosine, 6-thio-7-deaza-8-aza-guanosine, 7-methyl-guanosine (m G), 6-thio-7-methyl-guanosine, 7-methyl-inosine, 6-methoxy-guanosine, 1-methyl-guanosine (m′G), N2-methyl-guanosine (m 2 G), N2,N2-dimethyl-guanosine (m 2 2G), N2,7-dimethyl-guanosine (m 2,7G), N2, N2,7-dimethyl-guanosine (m 2,2,7G), 8-oxo-guanosine, 7-methyl-8-oxo-guanosine, 1-meth thio-guanosine, N2-methyl-6-thio-guanosine, N2,N2-dimethyl-6-thio-guanosine, a-thio-guanosine, 2′-0-methyl-guanosine (Gm), N2-methyl-2′-0-methyl-guanosine (m 2″Gm), N2,N2-dimethyl-2′-0-methyl-guano sine (m 2 2Gm), 1-methyl-2′-0-methyl-guanosine (m′Gm), N2,7-dimethyl-2′-0-methyl-guanosine (m″,7Gm), 2′-0-methyl-inosine (Im), 1,2′-0-dimethyl-inosine (m′lm), 06-phenyl-2′-deoxyinosine, 2′-0-ribosylguanosine (phosphate) (Gr(p)), 1-thio-guanosine, 06-methyl-guanosine, 06-Methyl-2′-deoxy guanosine, Z—F-ara-guanosine, 2′-F-guanosine, 2′-OMe-exNA-guanosine, and 2′-F-exNA-guanosine.

In some embodiments, the modified nucleobase is a modified uracil. Exemplary nucleobases and nucleosides having a modified uracil include without limitation pseudouridine (W), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m 3 U), 5-methoxy-uridine (mo 5 U), uridine 5-oxyacetic acid (cmo 5 U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm 5 se 2 U), 5-carbamoylmethyl-uridine (ncm 5 U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (xcm5U), 1-taurinomethyl-pseudouridine, 5-taurinomethyl-2-thio-uridine(Tm5s2U), 1-taurinomethyl-4-thio-pseudouridine, 5-methyl-uridine (m5U, i.e., having the nucleobase deoxythymine), 1-methyl-pseudouridine (i1′), 5-methyl-2-thio-uridine (m5s2U), 1-methyl-4-thio-pseudouridine (m xi/), 4-thio-1-methyl-pseudouridine, 3-methyl-pseudouridine (m W), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N 1-methyl-pseudouridine, 3-(3-amino-3-carboxypropyl)uridine (acp 3 U), 1-methyl-3-(3-amino-3-carboxypropyl)pseudouridine (acp 3 ψ), 5-(isopentenylaminomethyl)uridine (inm5U), 5-(isopentenylaminomethyl)-2-thio-uridine(inm5s2U), a-thio-uridine, 2′-0-methyl-uridine (Um), 5,2′-0-dimethyl-uridine (m5Um), 2′-0-methyl-pseudouridine (ψηι), 2-thio-2′-0-methyl-uridine (s2Um), 5-methoxycarbonylmethyl-2′-O-methyl-uridine (mem 5Um), 5-carbamoylmethyl-2′-0-methyl-uridine (ncm 5Um), 5-carboxymethylaminomethyl-2′-0-methyl-uridine (cmnm 5 Um), 3,2′-0-dimethyl-uridine (m 3 Um), 5-(isopentenylaminomethyl)-2′-0-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, hypoxanthine, 2′-OMe-exNA-uridine, and 2′-F-exNA-uridine.

In some embodiments, the modified nucleobase is a modified thymine. In some embodiments, the modified nucleoside is a modified thymidine. Non-limiting examples of modified thymine and thymidine include: 6-(azo)thymine, 3′-azido-3′-deoxythymidine, 2′,3′-didehydro-2′,3′-dideoxythymidine; 1-(2,3-dideoxy-beta-D-glyceropent-2-enofuranosyl)thymine, 3-(2-chloroethyl)thymidine, 3′-fluoro-3′-deoxythymidine, β-L-2′-deoxythymidine, thieno[3,4-d]-pyrimidine T-mimic deoxynucleoside, 1-(2-Deoxy-β-D-threo-pentofuranosyl)thymine, 5-ethynyl-2′-deoxyuridine, bromodeoxyuridine, tritiated thymidine, 5-chlorodeoxyuridine (CldU), 5-iododeoxyuridine (IdU), 2-thiothymidine triphosphate, 5-(α-tert-butylortho-bromobenzyloxy) methyl-2′-deoxyuridine, 5-(α-methylbenzyloxy)methyluracil, and 5-ethyldeoxyuridine.

Nucleic acids can be modified using various chemistries and modifications. In some embodiments, regular internucleosidic linkages between nucleotides can be altered by mono- or di-thioation of the phosphodiester bonds to yield phosphorothioate esters or phosphorodithioate esters, respectively. Other modifications of the internucleosidic linkages can include amidation or peptide linkers. A ribose sugar can be modified by substitution of the 2′-O moiety with a lower alkyl (C1-4, such as 2′-O-Me), alkenyl (C2-4), alkynyl (C2-4), methoxyethyl (2′-MOE), or other substituent. In some cases, substituents of the 2′ OH group can comprise a methyl, methoxyethyl or 3,3′-dimethylallyl group. In some cases, locked nucleic acid sequences (LNAs), comprising a 2′-4′ intramolecular bridge (such as a methylene bridge between the 2′ oxygen and 4′ carbon) linkage inside the ribose ring, can be applied. Purine nucleobases and/or pyrimidine nucleobases can be modified to alter their properties, for example by amination or deamination of the heterocyclic rings. Many of these modified nucleobases and their corresponding ribonucleosides are available from commercial suppliers. If desired, the guide polynucleotides can contain phosphoramidate, phosphorothioate, and/or methylphosphonate linkages. Several suitable methods can be used to produce nucleic acid molecules and nucleic acids containing modified nucleobases. For example, a guide nucleic acid that contains modified nucleotides can be prepared by transcribing (e.g., in vitro transcription) a DNA that encodes for a guide RNA using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction can contain nucleotides and modified nucleotides, and other components that support the activity of the selected polymerase, such as a suitable buffer, and suitable salts. The incorporation of nucleotide analogs into a guide polynucleotides may be engineered, for example, to alter the stability of such RNA/DNA molecules or to increase resistance against RNases.

In some embodiments, the guide polynucleotide comprises a secondary structure or a tertiary structure. In some embodiments, the secondary structure comprises a bulge, a stem, a stem loop, a loop, a tetraloop, a hairpin, a wobble base pair, a pseudoknot, a nexus, or a combination thereof. In some embodiments, the bulge, the stem loop, or the hairpin comprise an unpaired region of nucleotides within a nucleic acid duplex.

In some embodiments, the guide polynucleotides provided herein comprise a targeting region that has complementarity to a target nucleic acid. In some embodiments, the targeting region comprises RNA. In some embodiments, the targeting region has at least 80% complementarity to the target nucleic acid. In some embodiments, the targeting region has at least 85% complementarity to the target nucleic acid. In some embodiments, the targeting region has at least 90% complementarity to the target nucleic acid. In some embodiments, the targeting region has at least 95% complementarity to the target nucleic acid. In some embodiments, the targeting region has at least 99% complementarity to the target nucleic acid. In some embodiments, the targeting region has 100% complementarity to the target nucleic acid.

In some embodiments, the targeting region comprises at least 10 ribonucleotides, at least 15 ribonucleotides, at least 20 ribonucleotides, at least 25 ribonucleotides, at least 30 ribonucleotides, at least 35 ribonucleotides, at least 40 ribonucleotides, at least 45 ribonucleotides, at least 50 ribonucleotides or more. In some embodiments, the targeting region comprises at least about 10 up to 15 ribonucleotides for enhanced specificity. In some embodiments, the targeting region comprises at least about 20 to 30 ribonucleotides for enhanced structural stability and specificity.

In some embodiments, the targeting region hybridizes to a target nucleic acid. In some embodiments, a nuclease or a nickase cleaves the target nucleic acid (e.g., DNA) to generate a leading strand and a complementary strand (also referred to herein as a lagging strand). In some embodiments, the targeting region binds to at least a portion of the target nucleic acid which results in cleavage of the target nucleic acid by an engineered protein construct provided herein or a nickase. In some embodiments, the targeting region binds to a complementary strand. In some embodiments, the complementary strand comprises a protospacer adjacent motif (PAM). A PAM is a short nucleic acid sequence (usually 2-6 base pairs in length) that precedes the region targeted for cleavage by the nuclease or the nickase provided herein. In some embodiments, the targeting region binds to a target gene sequence that is within at least 20 nucleotides, at least 15 nucleotides, at least 10 nucleotides, or at least 5 nucleotides from a protospacer adjacent motif (PAM). The guide polynucleotide can bind upstream 5′ of a protospacer adjacent motif (PAM) sequence or downstream 3′ of a PAM sequence via the targeting region.

In some embodiments, the guide polynucleotides comprise a protein binding region. In some embodiments, the protein binding region comprises a secondary structure that binds to a protein. In some embodiments, the protein binding region binds to a nuclease, a nickase, an endonuclease, or an exonuclease. In some embodiments, the protein binding region binds to a Cas protein. The secondary structure can minimize the potential of the guide polynucleotide interfere with nuclease or nickase activity when the guide polynucleotide is in complex with an engineered protein provided herein. In some embodiments, the secondary structure comprises: a bulge, a stem, a loop, a hairpin, a wobble base pair, a pseudoknot, or a combination thereof.

In some embodiments, the guide polynucleotides comprise a DNA-dependent DNA polymerase synthesis template (DST) region. In some embodiments, the DST region is 5′ of the hybridization region. In some embodiments, the DST region comprises deoxyribonucleotides. In some embodiments, the DST region is within the protein-binding region of the guide polynucleotide. In some embodiments, the DST region comprises secondary or tertiary structure that enhances the stability of the guide polynucleotide. In some embodiments, the DST region has complementarity to the target nucleic acid. In some embodiments, the DST region comprises at least one mismatch nucleobase relative to the target nucleic acid. In some embodiments, the DST region comprises at least about 3 nucleotides up to 1,000,000 nucleotides in length. In some embodiments, the DST region comprises at least about 5 nucleotides up to 10,000 nucleotides. In some embodiments, the DST region comprises at least about 7 nucleotides up to 10,000 nucleotides.

The DST region of the guide polynucleotide provides the template for a DNA-dependent DNA polymerase to synthesize a DNA sequence that has complementarity to the target nucleic acid or a strand thereof and can further correct an aberration in a target sequence relative to the wild-type sequence. For example, a wild-type sequence can include a reference sequence, a nucleic acid sequence from a healthy subject, or a nucleic acid sequence from a healthy cell. Methods of obtaining a reference sequence for a polynucleotide can include sequencing or sequence alignment tools and databases such as NCBI BLAST or UniProt.

In some embodiments, the DST region comprises the reverse complement sequence of the target nucleic acid. In some embodiments, the DST region comprises the reverse complement sequence of the complementary strand of the nicked target nucleic acid. In some embodiments, the DST region comprises the reverse complement sequence of the leading strand of the nicked target nucleic acid.

In some embodiments, the DST region comprises the complement sequence of the target nucleic acid. In some embodiments, the DST region comprises the complement sequence of the leading strand of the nicked target nucleic acid. In some embodiments, the DST region comprises the complement sequence of the complementary strand of the nicked target nucleic acid.

In some embodiments, the DST region comprises a reverse complement sequence of a sequence encoding an intron or a variant thereof. In some embodiments, the DST region comprises a complement sequence of a sequence encoding an intron or a variant thereof. In some embodiments, the DST region comprises a reverse complement sequence of a sequence encoding an exon or a variant thereof. In some embodiments, the DST region comprises a complement sequence of a sequence encoding an exon or a variant thereof. In some embodiments, the DST region comprises a reverse complement sequence of a sequence encoding for an exon and an intron.

In some embodiments, the DST region comprises a complement sequence of a sequence encoding for an exon and an intron. In some embodiments, the DST region comprises a reverse complement sequence of a sequence encoding a non-coding element or a variant thereof. In some embodiments, the DST region comprises a complement sequence of a sequence encoding a non-coding element or a variant thereof. In some embodiments, the non-coding element is a promoter or enhancer. In some embodiments, the DST region comprises a reverse complement sequence of a sequence encoding a coding region of a polynucleotide or a variant thereof. In some embodiments, the DST region comprises a complement sequence of a sequence encoding a coding region of a polynucleotide or a variant thereof.

In some embodiments, the DST region comprises one or more alterations. In some embodiments, the alteration is a change in at least one nucleobase, nucleoside, or nucleotide of the DST region. In some embodiments, the DST region comprises a sequence comprising at least one nucleobase that is complementary to or is mismatched with a sequence encoding a splice acceptor site. In some embodiments, the alteration comprises a mismatch between the DST region and the complementary strand of the target DNA. In some embodiments, the DST region comprises an A/C mismatch, an A/T mismatch, an A/G mismatch, a T/C mismatch, a T/G mismatch, a T/A mismatch, a C/G mismatch, a C/A mismatch, a C/T mismatch, a G/C mismatch, a G/T mismatch, a G/A mismatch, or any combination thereof relative to the target nucleic acid or the complementary strand (also referred to as a lagging strand) provided herein.

In some embodiments, the guide polynucleotides further comprise a DNA-dependent DNA polymerase (DdDP)-recruiting region. In some embodiments, the DdDP-recruiting region comprises a free hydroxyl group at the 3′ end of the hybridization region. In some embodiments, the DdDP-recruiting region comprises a DNA polymerase recruitment protein linked to the guide polynucleotide. Non-limiting examples of DNA polymerase recruitment proteins include, proliferating cell nuclear antigen (PCNA), single-stranded DNA-binding protein (SSBP), tumor necrosis factor, alpha-induced protein (TNFAIP), polymerase delta-interacting protein (PolDIP), X-ray repair cross-complementing protein (XRCC), 5-Hydroxymethylcytosine Binding, ES Cell Specific (HMCES) protein, RADI, RAD9, and EtUS1.

In some embodiments, the guide polynucleotides provided herein comprise a hybridization region. In some embodiments, the hybridization region comprises a ribonucleotide, ribonucleotides, or an RNA region. In some embodiments, the hybridization region comprises a deoxyribonucleotide, deoxyribonucleotides, or a DNA region. In some embodiments, the hybridization region comprises ribonucleotides and deoxyribonucleotides. In some embodiments, the hybridization region comprises at least one ribonucleotide and at least one deoxyribonucleotide. In some embodiments, the hybridization region is at the 3′ end of the guide polynucleotide. In some embodiments, the hybridization region is within the protein binding region. In some embodiments, the hybridization region comprises secondary or tertiary structure that enhances the stability of the guide polynucleotide.

In some embodiments, the hybridization region forms an DNA-RNA (DR)-loop upon association with a target nucleic acid. The DR-loop provides structural framework and stability for the formation of the complex between the target nucleic acid, the guide polynucleotide, and the engineered protein provided herein. In some embodiments, the hybridization region forms an DR-loop upon association with a target nucleic acid and an engineered protein provided herein.

In some embodiments, the hybridization region forms a DNA (D)-loop upon association with a target nucleic acid. A D loop forms following nickase cleavage of the target DNA. The D-loop is a DNA structure where the two strands of a double-stranded DNA molecule are separated for a stretch and held apart by a third strand of DNA. In some embodiments, the third strand of DNA in the D-loop is the DST region or the hybridization region of the guide polynucleotide.

In some embodiments, the hybridization region forms an R-loop upon association with a target nucleic acid. In some embodiments, the hybridization region forms an RNA (R)-loop upon association with a target nucleic acid and an engineered protein provided herein. The R-loop can form following nickase cleavage of the target nucleic acid between the complementary strand the target DNA, the RNA portion of the hybridization region of the guide polynucleotide, and the leading strand of the target DNA.

In some embodiments, the hybridization region hybridizes to a leading strand of a DNA upon cleavage of the target nucleic acid by an engineered protein construct provided herein. In some embodiments, the ribonucleotide, the ribonucleotides, or the RNA region within the hybridization region hybridizes to the leading strand.

In some embodiments the hybridization region comprises a ratio of ribonucleic acids (RNA) to deoxyribonucleic acids (DNA) of 1:1 up to 20:1. In some embodiments, the hybridization region comprises a ratio of ribonucleic acids (RNA) to deoxyribonucleic acids (DNA) of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, or 19:18. In some embodiments, the hybridization region comprises at least about 5 nucleotides up to about 20 nucleotides.

In some embodiments, the guide polynucleotide comprises a hybridization region provided herein. In some embodiments, the hybridization region comprises one or more ribonucleotides or one or more deoxyribonucleotides. In some embodiments, the hybridization region comprises in a 5′ to 3′ direction: a DNA region and an RNA region. In some embodiments, the guide polynucleotide comprises a DNA-dependent DNA polymerase synthesis template (DST). The interaction between the guide polynucleotide provided herein and an engineered protein provided herein is illustrated in FIG. 1A. An exemplary guide polynucleotide structure is shown in FIG. 1B. Thus, in some embodiments, the guide polynucleotides provided herein comprise in a 5′ to 3′ direction: (i) a targeting region that has complementarity to a target nucleic acid; (ii) a protein binding region, wherein the protein binding region comprises a secondary structure that binds to the engineered protein construct comprising a nickase region; and (iii) a DNA-dependent DNA polymerase synthesis template (DST) region, wherein the DST has complementarity to the target nucleic acid and at least one alteration relative to the target nucleic acid; and (iv) a hybridization region, wherein the hybridization region comprises: a deoxyribonucleotide and a ribonucleotide, wherein the hybridization region has complementarity to the target nucleic acid. In some embodiments, upon introduction to a cell or a cell-free system, the compositions or systems provided herein synthesize a nucleic acid sequence that is incorporated into a target nucleic acid.

In some embodiments, the newly synthesized nucleic acid comprises a single-stranded DNA sequence that is substantially complementary to the complementary strand (that comprises a PAM sequence) and also comprises at least one mismatched nucleobase relative to the complementary strand.

In some embodiments, the guide polynucleotides comprise a sequence or a portion of a sequence in Table 5 or Table 6. In some embodiments, the guide polynucleotides comprise a sequence that is at least 75% identical, 80% identical, 85% identical, 90% identical, 95% identical, 99% identical, or 100% identical to any one of SEQ ID NOS: 45 to 57 or SEQ ID NOS: 187-233.

The guide polynucleotides provided herein can be produced, for example, by phosphoramidite chemical synthesis, in-vitro transcription (IVT) techniques, M13 bacteriophage methods, DNA isolation, RNA isolation, ligation, tagmentation, and combinations thereof. Furthermore, guide polynucleotides can be generated and expressed by transducing or transfecting cells with a plasmid DNA or a vector that comprises a polynucleotide encoding for an expression cassette.

In some embodiments, the guide polynucleotides comprise a 5′ cap. Free 5′ hydroxyl groups can be capped by acetylation. In some embodiments, the guide polynucleotides comprise a 3′ polyadenylated tail. In some embodiments, the guide polynucleotides comprise a polynucleotide linker or spacer regions.

The engineered proteins that interact with the guide polynucleotides provided herein can be used in the systems and compositions provided herein to synthesize a nucleic acid. Engineered proteins useful in the synthesis of nucleic acids and targeting cleavage of a particular target sequence are described in further detail below.

### Engineered Proteins

Provided herein are engineered proteins that specifically bind to a target nucleic acid using the guide polynucleotide to hybridize to the target sequence or polynucleotides encoding the engineered proteins provided herein. The engineered proteins also synthesize a nucleic acid sequence for incorporation into the target nucleic acid. The engineered proteins provided herein can be fused to one or more additional protein constructs for a particular application (e.g., DNA synthesis, cleavage of a nucleic acid). Multimerization of an engineered protein construct provided herein can be achieved by direct fusion with another engineered protein construct. In some embodiments, each protein construct is operably linked by a linker polypeptide.

Provided herein are systems, compositions, and engineered proteins that comprise a nuclease or a nickase region. A nuclease is an enzyme that cleaves a nucleic acid. For example, a nuclease can create a single or a double-stranded break in a target nucleic acid. Nucleases and nickases provided herein target specific nucleic acid sequences by binding to a guide polynucleotide that hybridizes to the target nucleic acid. Various types of nucleases and nickases can be used in the systems and compositions provided herein.

Provided herein are systems, compositions, and engineered proteins that comprise a nickase or a nickase region. In some embodiments, the engineered proteins comprise a nickase. In some embodiment the engineered proteins provided herein comprise: an engineered protein construct comprising a nickase region. A nickase is an enzyme that cuts one strand of a double-stranded DNA at a restriction site. In some embodiments, the nickase provided herein cut one strand of the DNA duplex to produce DNA molecules that are nicked.

Exemplary amino acid sequences for a nickase can include but are not limited to, for example, NCBI Gene ID: 1238121: NP_858382.1 [conjugal transfer nickase/helicase TraI (plasmid) [Shigella flexneri 2a str. 301]]

(SEQ ID NO: 58)
MKAGEESVAQVSGVREQAILTQAIRSELKTQGVLGHPEVTMTALSPVWLDSRSRYLRDMYRPG
MVMEQWNPETRSHDRYVTERVTAQSHSLTLRNAQGETQVVRISSLDSSWSLFRPEKMPVADGE
RLRVTGKIPGLRVSGGDRLQVASVSEDAMTVVVPGRAEPATLPVSDSPFTALKLENGWVETPGH
SVSDSATVFASVTQMAMDNATLNGLARSGRDVRLYSSLDETRTAEKLARHPSFTVVSEQIKARA
GETLLETAISLQKAGLHTPAQQAIHLALPVLESKNLAFSMVDLLTEAKSFAAEGTGFADLGGEIN
AQIKRGDLLYVDVAKGYGTGLLVSRASYEAEKSILRHILEGKEAVTPLMERVPGELMEKLTSGQ
RAATRMILETSDRFTVVQGYAGVGKTTQFRAVMSAVNMLPESERPRVVGLGPTHRAVGEMRSA
GVDAQTLASFLHDTQLQQRSGETPDFSNTLFLLDESSMVGNTDMARAYALIAAGGGRAVASGD
TDQLQAIAPGQPFRLQQTRSAADVVIMKEIVRQTPELREAVYSLINRDVERALSGLERVKPSQVP
RLEGAWAPEHSVTEFSHSQEAKLAEAQQKAMLKGEAFPDVPMTLYEAIVRDYTGRTPEAREQTL
IVTHLNEDRRVLNSMIHDAREKAGELGKVQVMVPVLNTANIRDGELRRLSTWENNPDALALVD
NVYHRIAGISKDDGLITLQDAEGNTRLISPREAVAEGVTLYTPDTIRVGTGDRIRFTKSDRERGYV
ANSVWTVTAVSGDSVTLSDGQQTRVIRPGQERAEQHIDLAYAITAHGAQGASETFAIALEGTEG
NRKLMAGFESAYVALSRMKQHVQVYTDNRQGWTDAINNAVQKGTAHDVFEPKPDREVMNAE
RLFSTARELRDVAAGRAVLRQAGLAGGDSPARFIAPGRKYPQPYVALPAFDRNGKSAGIWLNPL
TTDDGNGLRGFSGEGRVKGSGDAQFVALQGSRNGESLLADNMQDGVRIARDNPDSGVVVRIAG
EGRPWNPGAITGGRVWGDIPDNSVQPGAGNGEPVTAEVLAQRQAEEAIRRETERRADEIVRKMA
ENKPDLPDGKTEQAVREIAGQERDRAAITEREAALPESVLREPQRVREAVREVARENLLQERLQQ
MERDMVRDLQKEKTPGGD;
UniProt: Q8YS92 [DNA Nickase-Nostoc sp. (strain PCC 7120/SAG 25.82/UTEX
2576)]:
(SEQ ID NO: 59)
MVSTLDDTKRNAIAEKLADAKLLQELIIENQERFLRESTDNEISNRIRDFLEDDRKNLGIIETVIVQ
YGIQKEPRQTVREMVDQVRQLMQGSQLNFFEKVAQHELLKHKQVMSGLLVHKAAQKVGADVL
AAIGPLNTVNFENRAHQEQLKGILEILGVRELTGQDADQGIWGRVQDAIAAFSGAVGSAVTQGS
DKQDMNIQDVIRMDHNKVNILFTELQQSNDPQKIQEYFGQIYKDLTAHAEAEEEVLYPRVRSFY
GEGDTQELYDEQSEMKRLLEQIKAISPSAPEFKDRVRQLADIVMDHVRQEESTLFAAIRNNLSSE
QTEQWATEFKAAKSKIQQRLGGQATGAGV;
Streptococcus pyogenes Cas9 H840A nickase (bold/underlined
indicates amino acid substitution):
(SEQ ID NO: 60)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYD
EHHQDLTLLKALVRQQLPEKYKEIFFDQSKNGYAGYIDGGASQEEFYKFIKPILEKMDGTEELLV
KLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQFYK
VREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQLG
GD;
Staphylococcus aureus Cas9 N580A nickase (bold/underlined indicates amino
acid substitution):
(SEQ ID NO: 61)
MKRNYILGLDIGITSVGYGIIDYETRDVIDAGVRLFKEANVENNEGRRSKRGARRLKRRRRHRIQ
RVKKLLFDYNLLTDHSELSGINPYEARVKGLSQKLSEEEFSAALLHLAKRRGVHNVNEVEEDTG
NELSTKEQISRNSKALEEKYVAELQLERLKKDGEVRGSINRFKTSDYVKEAKQLLKVQKAYHQL
DQSFIDTYIDLLETRRTYYEGPGEGSPFGWKDIKEWYEMLMGHCTYFPEELRSVKYAYNADLYN
ALNDLNNLVITRDENEKLEYYEKFQIIENVFKQKKKPTLKQIAKEILVNEEDIKGYRVTSTGKPEF
TNLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIK
VINAIIKKYGLPNDIIIELAREKNSKDAQKMINEMQKRNRQTNERIEEIIRTTGKENAKYLIEKIKLH
DMQEGKCLYSLEAIPLEDLLNNPFNYEVDHIIPRSVSFDNSFNNKVLVKQEEASKKGNRTPFQYL
SSSDSKISYETFKKHILNLAKGKGRISKTKKEYLLEERDINRFSVQKDFINRNLVDTRYATRGLMN
LLRSYFRVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNRELINDT
LYSTRKDDKGNTLIVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNSRNKVVKLSLKPYRF
DVYLDNGVYKFVTVKNLDVIKKENYYEVNSKCYEEAKKLKKISNQAEFIASFYNNDLIKINGEL
YRVIGVNNDLLNRIEVNMIDITYREYLENMNDKRPPRIIKTIASKTQSIKKYSTDILGNLYEVKSKK
HPQIIKKG;
Francisella novicida Cas9 H969A nickase (bold/underlined indicates amino
acid substitution):
(SEQ ID NO: 62)
MNFKILPIAIDLGVKNTGVFSAFYQKGTSLERLDNKNGKVYELSKDSYTLLMNNRTARRHQRRG
IDRKQLVKRLFKLIWTEQLNLEWDKDTQQAISFLFNRRGFSFITDGYSPEYLNIVPEQVKAILMDIF
DDYNGEDDLDSYLKLATEQESKISEIYNKLMQKILEFKLMKLCTDIKDDKVSTKTLKEITSYEFEL
LADYLANYSESLKTQKFSYTDKQGNLKELSYYHHDKYNIQEFLKRHATINDRILDTLLTDDLDIW
NFNFEKFDFDKNEEKLQNQEDKDHIQAHLHHFVFAVNKIKSEMASGGRHRSQYFQEITNVLDEN
NHQEGYLKNFCENLHNKKYSNLSVKNLVNLIGNLSNLELKPLRKYFNDKIHAKADHWDEQKFT
ETYCHWILGEWRVGVKDQDKKDGAKYSYKDLCNELKQKVTKAGLVDFLLELDPCRTIPPYLDN
NNRKPPKCQSLILNPKFLDNQYPNWQQYLQELKKLQSIQNYLDSFETDLKVLKSSKDQPYFVEY
KSSNQQIASGQRDYKDLDARILQFIFDRVKASDELLLNEIYFQAKKLKQKASSELEKLESSKKLDE
VIANSQLSQILKSQHTNGIFEQGTFLHLVCKYYKQRQRARDSRLYIMPEYRYDKKLHKYNNTGR
FDDDNQLLTYCNHKPRQKRYQLLNDLAGVLQVSPNFLKDKIGSDDDLFISKWLVEHIRGFKKAC
EDSLKIQKDNRGLLNHKINIARNTKGKCEKEIFNLICKIEGSEDKKGNYKHGLAYELGVLLFGEPN
EASKPEFDRKIKKFNSIYSFAQIQQIAFAERKGNANTCAVCSADNAHRMQQIKITEPVEDNKDKII
LSAKAQRLPAIPTRIVDGAVKKMATILAKNIVDDNWQNIKQVLSAKHQLHIPIITESNAFEFEPAL
ADVKGKSLKDRRKKALERISPENIFKDKNNRIKEFAKGISAYSGANLTDGDFDGAKEELDAIIPRS
HKKYGTLNDEANLICVTRGDNKNKGNRIFCLRDLADNYKLKQFETTDDLEIEKKIADTIWDANK
KDFKFGNYRSFINLTPQEQKAFRHALFLADENPIKQAVIRAINNRNRTFVNGTQRYFAEVLANNI
YLRAKKENLNTDKISFDYFGIPTIGNGRGIAEIRQLYEKVDSDIQAYAKGDKPQASYSHLIDAMLA
FCIAADEHRNDGSIGLEIDKNYSLYPLDKNTGEVFTKDIFSQIKITDNEFSDKKLVRKKAIEGENTH
RQMTRDGIYAENYLPILIHKELNEVRKGYTWKNSEEIKIFKGKKYDIQQLNNLVYCLKFVDKPISI
DIQISTLEELRNILTTNNIAATAEYYYINLKTQKLHEYYIENYNTALGYKKYSKEMEFLRSLAYRS
ERVKIKSIDDVKQVLDKDSNFIIGKITLPFKKEWQRLYREWQNTTIKDDYEFLKSFFNVKSITKLH
KKVRKDFSLPISTNEGKFLVKRKTWDNNFIYQILNDSDSRADGTKPFIPAFDISKNEIVEAIIDSFTS
KNIFWLPKNIELQKVDNKNIFAIDTSKWFEVETPSDLRDIGIATIQYKIDNNSRPKVRVKLDYVIDD
DSKINYFMNHSLLKSRYPDKVLEILKQSTIIEFESSGFNKTIKEMLGMKLAGIYNETSNN,

a variant, or a fragment thereof. In some embodiments, a system or an engineered protein provided herein comprises a sequence that is at least 85% identical to any one of SEQ ID NOS: 58-62. In some embodiments, a system or an engineered protein provided herein comprises a sequence that is at least 90% identical to any one of SEQ ID NOS: 58-62. In some embodiments, a system or an engineered protein provided herein comprises a sequence that is at least 95% identical to any one of SEQ ID NOS: 58-62. In some embodiments, a system or an engineered protein provided herein comprises a sequence that is at least 99% identical to any one of SEQ ID NOS: 58-62. In some embodiments, a system or an engineered protein provided herein comprises any one of SEQ ID NOS: 58-62.

In some embodiments, the engineered proteins comprise a Cas protein or a fragment thereof. In some embodiments, the engineered proteins comprise a Cas protein or a fragment thereof that comprise an amino acid substitution or amino acid substitutions relative to a reference amino acid sequence for the Cas protein of the fragment thereof (e.g., the wild-type sequence). In some embodiments, the Cas is a catalytically dead Cas or a partially dead Cas (e.g., a nickase). In some embodiments, the catalytically dead Cas or the partially dead Cas is selected from the group consisting of catalytically dead or partially dead derivatives of Cas1, Cas1B, Cas2, Cas3, Cas4, Cas5, Cas6, Cas7, Cas8, Cas9, Cas10, Cas 11, Cas 12a (Cpf1), Cas 12b, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, c2c1, c2c3, Cas9HiFi, xCas9, CasX, CasY, CasRX, SpCas9-VQR, SpCas9-VRQR, SpCas9-VRER, SaCas9-KKH, SpCas9-NG, SpCas9-NRRH, SpCas9-NRTH, SpCas9-NRCH, iSpyMac, St1Cas9 LMD9-LMG18311, St1Cas9 LMD9-CNRZ1066, St1Cas9-KQKL, a variant, a fragment, a mutant, or a derivative thereof. In some embodiments, the Cas proteins or the mutant Cas proteins provided herein are a Type V Cas protein. In some embodiments, the Type V Cas protein comprises: a Cas12a, a Cas12b, a Cas12c, a Cas12d, a Cas12e, a Cas14, a Cas12g, a Cas12h, a Cas12i, a Cas12j, a Cas12k, a variant, a fragment, a mutant, or a derivative thereof. In some embodiments, the Cas protein is a chimeric Cas protein comprising domains from different Cas proteins provided herein. In some embodiments, the Cas protein is a Cas9 protein or a variant thereof. In some embodiments, the Cas protein is genetically modified. In some embodiments, the genetically modified Cas comprises a nickase.

In some embodiments, the Cas protein is derived from a bacterium. In some embodiments the bacterium is of the genus Streptococcus, Staphylococcus, Francisella, Lactococcus, Lactobacillus, Pseudomonas, Geobacillus, Clostridium, Streptomyces, Actinoplanes, Synechococcus, Corynebacterium, Haloferax, Haloarcula, Methanococcus, Neisseria, or Campylobacter.

In some embodiments, the Cas protein is derived from a bacterium, wherein the bacterium is Streptococcus pyogenes (e.g., SpCas9). In some embodiments, the Cas protein is an Streptococcus pyogenes Cas9 (spCas9), wherein the Streptococcus pyogenes Cas9 comprises a sequence that is at least 90% identical to SEQ ID NO: 92. In some embodiments, the Cas protein is an Streptococcus pyogenes Cas9, wherein the an Streptococcus pyogenes Cas9 comprises a sequence that is at least 95% identical to SEQ ID NO: 92. In some embodiments, the Cas protein is an Streptococcus pyogenes Cas9, wherein the an Streptococcus pyogenes Cas9 comprises a sequence that is at least 99% identical to SEQ ID NO: 92. In some embodiments, the Cas protein is an Streptococcus pyogenes Cas9, wherein the an Streptococcus pyogenes Cas9 comprises a sequence that is identical to SEQ ID NO: 92. In some embodiments, the Streptococcus pyogenes Cas9 comprises a mutation. In some embodiments, the Streptococcus pyogenes Cas9 comprises a mutation, wherein the mutation comprises one or more amino acid substitutions.

In some embodiments, the Cas protein is derived from a bacterium, wherein the bacterium is Streptococcus thermophilus (e.g., St1Cas9). In some embodiments, the Cas protein is a Streptococcus thermophilus Cas9 (St1Cas9), wherein the Streptococcus thermophilus Cas9 comprises a sequence that is at least 90% identical to SEQ ID NO: 97. In some embodiments, the Cas protein is a Streptococcus thermophilus Cas9, wherein the Streptococcus thermophilus Cas9 comprises a sequence that is at least 95% identical to SEQ ID NO: 97. In some embodiments, the Cas protein is a Streptococcus thermophilus Cas9, wherein the Streptococcus thermophilus Cas9 comprises a sequence that is at least 99% identical to SEQ ID NO: 97. In some embodiments, the Cas protein is a Streptococcus thermophilus Cas9, wherein the Streptococcus thermophilus Cas9 comprises a sequence that is identical to SEQ ID NO: 97. In some embodiments, the Streptococcus thermophilus Cas9 comprises a mutation. In some embodiments, the Streptococcus thermophilus Cas9 comprises a mutation, wherein the mutation comprises one or more amino acid substitutions.

In some embodiments, the engineered proteins provided herein comprise a nickase having a sequence that is at least 85%, at least 90%, at least 95%, at least 99%, or 10000 identical to a sequence listed in Table 1 below.

TABLE 1
Nickase Sequences

| SEQ | Cas | | |
|---|---|---|---|
| ID | Protein | | |
| NO: | Name | Sequence | Mutations |
| 92 | SpCas9 | MDKKYSIGLDIGTNSVGWAVITDEYKVPSKKFKVLGNTDRHSIKK | — |
| | | NLIGALLFDSGETAEATRLKRTARRRYTRRKNRICYLQEIFSNEMA | |
| | | KVDDSFFHRLEESFLVEEDKKHERHPIFGNIVDEVAYHEKYPTIYH | |
| | | LRKKLVDSTDKADLRLIYLALAHMIKFRGHFLIEGDLNPDNSDVD | |
| | | KLFIQLVQTYNQLFEENPINASGVDAKAILSARLSKSRRLENLIAQL | |
| | | PGEKKNGLFGNLIALSLGLTPNFKSNFDLAEDAKLQLSKDTYDDD | |
| | | LDNLLAQIGDQYADLFLAAKNLSDAILLSDILRVNTEITKAPLSAS | |
| | | MIKRYDEHHQDLTLLKALVRQQLPEKYKEIFFDQSKNGYAGYIDG | |
| | | GASQEEFYKFIKPILEKMDGTEELLVKLNREDLLRKQRTFDNGSIP | |
| | | HQIHLGELHAILRRQEDFYPFLKDNREKIEKILTFRIPYYVGPLARG | |
| | | NSRFAWMTRKSEETITPWNFEEVVDKGASAQSFIERMTNFDKNLP | |
| | | NEKVLPKHSLLYEYFTVYNELTKVKYVTEGMRKPAFLSGEQKKAI | |
| | | VDLLFKTNRKVTVKQLKEDYFKKIECFDSVEISGVEDRFNASLGT | |
| | | YHDLLKIIKDKDFLDNEENEDILEDIVLTLTLFEDREMIEERLKTYA | |
| | | HLFDDKVMKQLKRRRYTGWGRLSRKLINGIRDKQSGKTILDFLKS | |
| | | DGFANRNFMQLIHDDSLTFKEDIQKAQVSGQGDSLHEHIANLAGS | |
| | | PAIKKGILQTVKVVDELVKVMGRHKPENIVIEMARENQTTQKGQK | |
| | | NSRERMKRIEEGIKELGSQILKEHPVENTQLQNEKLYLYYLQNGR | |
| | | DMYVDQELDINRLSDYDVDHIVPQSFLKDDSIDNKVLTRSDKNRG | |
| | | KSDNVPSEEVVKKMKNYWRQLLNAKLITQRKFDNLTKAERGGLS | |
| | | ELDKAGFIKRQLVETRQITKHVAQILDSRMNTKYDENDKLIREVK | |
| | | VITLKSKLVSDFRKDFQFYKVREINNYHHAHDAYLNAVVGTALIK | |
| | | KYPKLESEFVYGDYKVYDVRKMIAKSEQEIGKATAKYFFYSNIMN | |
| | | FFKTEITLANGEIRKRPLIETNGETGEIVWDKGRDFATVRKVLSMP | |
| | | QVNIVKKTEVQTGGFSKESILPKRNSDKLIARKKDWDPKKYGGFD | |
| | | SPTVAYSVLVVAKVEKGKSKKLKSVKELLGITIMERSSFEKNPIDFL | |
| | | EAKGYKEVKKDLIIKLPKYSLFELENGRKRMLASAGELQKGNELA | |
| | | LPSKYVNFLYLASHYEKLKGSPEDNEQKQLFVEQHKHYLDEIIEQI | |
| | | SEFSKRVILADANLDKVLSAYNKHRDKPIREQAENIIHLFTLTNLG | |
| | | APAAFKYFDTTIDRKRYTSTKEVLDATLIHQSITGLYETRIDLSQLG | |
| | | GD | |
| 93 | SpCas9 | MDKKYSIGLDIGTNSVGWAVITDEYKVPSKKFKVLGNTDRHSIKK | R221K, |
| | | NLIGALLFDSGETAEATRLKRTARRRYTRRKNRICYLQEIFSNEMA | N394K, |
| | | KVDDSFFHRLEESFLVEEDKKHERHPIFGNIVDEVAYHEKYPTIYH | H840A |
| | | LRKKLVDSTDKADLRLIYLALAHMIKFRGHFLIEGDLNPDNSDVD | |
| | | KLFIQLVQTYNQLFEENPINASGVDAKAILSARLSKSRKLENLIAQ | |
| | | LPGEKKNGLFGNLIALSLGLTPNFKSNFDLAEDAKLQLSKDTYDD | |
| | | DLDNLLAQIGDQYADLFLAAKNLSDAILLSDILRVNTEITKAPLSAS | |
| | | MIKRYDEHHQDLTLLKALVRQQLPEKYKEIFFDQSKNGYAGYIDG | |
| | | GASQEEFYKFIKPILEKMDGTEELLVKLKREDLLRKQRTFDNGSIP | |
| | | HQIHLGELHAILRRQEDFYPFLKDNREKIEKILTFRIPYYVGPLARG | |
| | | NSRFAWMTRKSEETITPWNFEEVVDKGASAQSFIERMTNFDKNLP | |
| | | NEKVLPKHSLLYEYFTVYNELTKVKYVTEGMRKPAFLSGEQKKAI | |
| | | VDLLFKTNRKVTVKQLKEDYFKKIECFDSVEISGVEDRFNASLGT | |
| | | YHDLLKIIKDKDFLDNEENEDILEDIVLTLTLFEDREMIEERLKTYA | |
| | | HLFDDKVMKQLKRRRYTGWGRLSRKLINGIRDKQSGKTILDFLKS | |
| | | DGFANRNFMQLIHDDSLTFKEDIQKAQVSGQGDSLHEHIANLAGS | |
| | | PAIKKGILQTVKVVDELVKVMGRHKPENIVIEMARENQTTQKGQK | |
| | | NSRERMKRIEEGIKELGSQILKEHPVENTQLQNEKLYLYYLQNGR | |
| | | DMYVDQELDINRLSDYDVDAIVPQSFLKDDSIDNKVLTRSDKNRG | |
| | | KSDNVPSEEVVKKMKNYWRQLLNAKLITQRKFDNLTKAERGGLS | |
| | | ELDKAGFIKRQLVETRQITKHVAQILDSRMNTKYDENDKLIREVK | |
| | | VITLKSKLVSDFRKDFQFYKVREINNYHHAHDAYLNAVVGTALIK | |
| | | KYPKLESEFVYGDYKVYDVRKMIAKSEQEIGKATAKYFFYSNIMN | |
| | | FFKTEITLANGEIRKRPLIETNGETGEIVWDKGRDFATVRKVLSMP | |
| | | QVNIVKKTEVQTGGFSKESILPKRNSDKLIARKKDWDPKKYGGFD | |
| | | SPTVAYSVLVVAKVEKGKSKKLKSVKELLGITIMERSSFEKNPIDFL | |
| | | EAKGYKEVKKDLIIKLPKYSLFELENGRKRMLASAGELQKGNELA | |
| | | LPSKYVNFLYLASHYEKLKGSPEDNEQKQLFVEQHKHYLDEIIEQI | |
| | | SEFSKRVILADANLDKVLSAYNKHRDKPIREQAENIIHLFTLTNLG | |
| | | APAAFKYFDTTIDRKRYTSTKEVLDATLIHQSITGLYETRIDLSQLG | |
| | | GD | |
| 94 | SpCas9 | MDKKYSIGLDIGTNSVGWAVITDEYKVPSKKFKVLGNTDRHSIKK | R221K, |
| | | NLIGALLFDSGETAEATRLKRTARRRYTRRKNRICYLQEIFSNEMA | N394K, |
| | | KVDDSFFHRLEESFLVEEDKKHERHPIFGNIVDEVAYHEKYPTIYH | H840A, |
| | | LRKKLVDSTDKADLRLIYLALAHMIKFRGHFLIEGDLNPDNSDVD | D1135V, |
| | | KLFIQLVQTYNQLFEENPINASGVDAKAILSARLSKSRKLENLIAQL | G1218R, |
| | | PGEKKNGLFGNLIALSLGLTPNFKSNFDLAEDAKLQLSKDTYDDD | R1335Q, |
| | | LDNLLAQIGDQYADLFLAAKNLSDAILLSDILRVNTEITKAPLSAS | T1337R |
| | | MIKRYDEHHQDLTLLKALVRQQLPEKYKEIFFDQSKNGYAGYIDG | |
| | | GASQEEFYKFIKPILEKMDGTEELLVKLKREDLLRKQRTFDNGSIP | |
| | | HQIHLGELHAILRRQEDFYPFLKDNREKIEKILTFRIPYYVGPLARG | |
| | | NSRFAWMTRKSEETITPWNFEEVVDKGASAQSFIERMTNFDKNLP | |
| | | NEKVLPKHSLLYEYFTVYNELTKVKYVTEGMRKPAFLSGEQKKAI | |
| | | VDLLFKTNRKVTVKQLKEDYFKKIECFDSVEISGVEDRFNASLGT | |
| | | YHDLLKIIKDKDFLDNEENEDILEDIVLTLTLFEDREMIEERLKTYA | |
| | | HLFDDKVMKQLKRRRYTGWGRLSRKLINGIRDKQSGKTILDFLKS | |
| | | DGFANRNFMQLIHDDSLTFKEDIQKAQVSGQGDSLHEHIANLAGS | |
| | | PAIKKGILQTVKVVDELVKVMGRHKPENIVIEMARENQTTQKGQK | |
| | | NSRERMKRIEEGIKELGSQILKEHPVENTQLQNEKLYLYYLQNGR | |
| | | DMYVDQELDINRLSDYDVDAIVPQSFLKDDSIDNKVLTRSDKNRG | |
| | | KSDNVPSEEVVKKMKNYWRQLLNAKLITQRKFDNLTKAERGGLS | |
| | | ELDKAGFIKRQLVETRQITKHVAQILDSRMNTKYDENDKLIREVK | |
| | | VITLKSKLVSDFRKDFQFYKVREINNYHHAHDAYLNAVVGTALIK | |
| | | KYPKLESEFVYGDYKVYDVRKMIAKSEQEIGKATAKYFFYSNIMN | |
| | | FFKTEITLANGEIRKRPLIETNGETGEIVWDKGRDFATVRKVLSMP | |
| | | QVNIVKKTEVQTGGFSKESILPKRNSDKLIARKKDWDPKKYGGFV | |
| | | SPTVAYSVLVVAKVEKGKSKKLKSVKELLGITIMERSSFEKNPIDFL | |
| | | EAKGYKEVKKDLIIKLPKYSLFELENGRKRMLASARELQKGNELA | |
| | | LPSKYVNFLYLASHYEKLKGSPEDNEQKQLFVEQHKHYLDEIIEQI | |
| | | SEFSKRVILADANLDKVLSAYNKHRDKPIREQAENIIHLFTLTNLG | |
| | | APAAFKYFDTTIDRKQYRSTKEVLDATLIHQSITGLYETRIDLSQLG | |
| | | GD | |
| 95 | SpCas9 | MDKKYSIGLDIGTNSVGWAVITDEYKVPSKKFKVLGNTDRHSIKK | R221K, |
| | | NLIGALLFDSGETAEATRLKRTARRRYTRRKNRICYLQEIFSNEMA | N394K, |
| | | KVDDSFFHRLEESFLVEEDKKHERHPIFGNIVDEVAYHEKYPTIYH | H840A, |
| | | LRKKLVDSTDKADLRLIYLALAHMIKFRGHFLIEGDLNPDNSDVD | D1135L, |
| | | KLFIQLVQTYNQLFEENPINASGVDAKAILSARLSKSRKLENLIAQL | S1136W, |
| | | PGEKKNGLFGNLIALSLGLTPNFKSNFDLAEDAKLQLSKDTYDDD | G1218K, |
| | | LDNLLAQIGDQYADLFLAAKNLSDAILLSDILRVNTEITKAPLSAS | E1219Q, |
| | | MIKRYDEHHQDLTLLKALVRQQLPEKYKEIFFDQSKNGYAGYIDG | R1335Q, |
| | | GASQEEFYKFIKPILEKMDGTEELLVKLKREDLLRKQRTFDNGSIP | T1337R |
| | | HQIHLGELHAILRRQEDFYPFLKDNREKIEKILTFRIPYYVGPLARG | |
| | | NSRFAWMTRKSEETITPWNFEEVVDKGASAQSFIERMTNFDKNLP | |
| | | NEKVLPKHSLLYEYFTVYNELTKVKYVTEGMRKPAFLSGEQKKAI | |
| | | VDLLFKTNRKVTVKQLKEDYFKKIECFDSVEISGVEDRFNASLGT | |
| | | YHDLLKIIKDKDFLDNEENEDILEDIVLTLTLFEDREMIEERLKTYA | |
| | | HLFDDKVMKQLKRRRYTGWGRLSRKLINGIRDKQSGKTILDFLKS | |
| | | DGFANRNFMQLIHDDSLTFKEDIQKAQVSGQGDSLHEHIANLAGS | |
| | | PAIKKGILQTVKVVDELVKVMGRHKPENIVIEMARENQTTQKGQK | |
| | | NSRERMKRIEEGIKELGSQILKEHPVENTQLQNEKLYLYYLQNGR | |
| | | DMYVDQELDINRLSDYDVDAIVPQSFLKDDSIDNKVLTRSDKNRG | |
| | | KSDNVPSEEVVKKMKNYWRQLLNAKLITQRKFDNLTKAERGGLS | |
| | | ELDKAGFIKRQLVETRQITKHVAQILDSRMNTKYDENDKLIREVK | |
| | | VITLKSKLVSDFRKDFQFYKVREINNYHHAHDAYLNAVVGTALIK | |
| | | KYPKLESEFVYGDYKVYDVRKMIAKSEQEIGKATAKYFFYSNIMN | |
| | | FFKTEITLANGEIRKRPLIETNGETGEIVWDKGRDFATVRKVLSMP | |
| | | QVNIVKKTEVQTGGFSKESILPKRNSDKLIARKKDWDPKKYGGFL | |
| | | WPTVAYSVLVVAKVEKGKSKKLKSVKELLGITIMERSSFEKNPIDF | |
| | | LEAKGYKEVKKDLIIKLPKYSLFELENGRKRMLASAKQLQKGNEL | |
| | | ALPSKYVNFLYLASHYEKLKGSPEDNEQKQLFVEQHKHYLDEIIE | |
| | | QISEFSKRVILADANLDKVLSAYNKHRDKPIREQAENIIHLFTLTNL | |
| | | GAPAAFKYFDTTIDRKQYRSTKEVLDATLIHQSITGLYETRIDLSQL | |
| | | GGD | |
| 96 | SpCas9 | MDKKYSIGLDIGTNSVGWAVITDEYKVPSKKFKVLGNTDRHSIKK | A61R, |
| | | NLIGALLFDSGETAERTRLKRTARRRYTRRKNRICYLQEIFSNEMA | R221K, |
| | | KVDDSFFHRLEESFLVEEDKKHERHPIFGNIVDEVAYHEKYPTIYH | N394K, |
| | | LRKKLVDSTDKADLRLIYLALAHMIKFRGHFLIEGDLNPDNSDVD | H840A, |
| | | KLFIQLVQTYNQLFEENPINASGVDAKAILSARLSKSRKLENLIAQL | L1111R, |
| | | PGEKKNGLFGNLIALSLGLTPNFKSNFDLAEDAKLQLSKDTYDDD | D1135L, |
| | | LDNLLAQIGDQYADLFLAAKNLSDAILLSDILRVNTEITKAPLSAS | S1136W, |
| | | MIKRYDEHHQDLTLLKALVRQQLPEKYKEIFFDQSKNGYAGYIDG | G1218K, |
| | | GASQEEFYKFIKPILEKMDGTEELLVKLKREDLLRKQRTFDNGSIP | E1219Q, |
| | | HQIHLGELHAILRRQEDFYPFLKDNREKIEKILTFRIPYYVGPLARG | N1317R, |
| | | NSRFAWMTRKSEETITPWNFEEVVDKGASAQSFIERMTNFDKNLP | A1322R, |
| | | NEKVLPKHSLLYEYFTVYNELTKVKYVTEGMRKPAFLSGEQKKAI | R1333P, |
| | | VDLLFKTNRKVTVKQLKEDYFKKIECFDSVEISGVEDRFNASLGT | R1335Q, |
| | | YHDLLKIIKDKDFLDNEENEDILEDIVLTLTLFEDREMIEERLKTYA | T1337R |
| | | HLFDDKVMKQLKRRRYTGWGRLSRKLINGIRDKQSGKTILDFLKS | |
| | | DGFANRNFMQLIHDDSLTFKEDIQKAQVSGQGDSLHEHIANLAGS | |
| | | PAIKKGILQTVKVVDELVKVMGRHKPENIVIEMARENQTTQKGQK | |
| | | NSRERMKRIEEGIKELGSQILKEHPVENTQLQNEKLYLYYLQNGR | |
| | | DMYVDQELDINRLSDYDVDAIVPQSFLKDDSIDNKVLTRSDKNRG | |
| | | KSDNVPSEEVVKKMKNYWRQLLNAKLITQRKFDNLTKAERGGLS | |
| | | ELDKAGFIKRQLVETRQITKHVAQILDSRMNTKYDENDKLIREVK | |
| | | VITLKSKLVSDFRKDFQFYKVREINNYHHAHDAYLNAVVGTALIK | |
| | | KYPKLESEFVYGDYKVYDVRKMIAKSEQEIGKATAKYFFYSNIMN | |
| | | FFKTEITLANGEIRKRPLIETNGETGEIVWDKGRDFATVRKVLSMP | |
| | | QVNIVKKTEVQTGGFSKESIRPKRNSDKLIARKKDWDPKKYGGFL | |
| | | WPTVAYSVLVVAKVEKGKSKKLKSVKELLGITIMERSSFEKNPIDF | |
| | | LEAKGYKEVKKDLIIKLPKYSLFELENGRKRMLASAKELQKGNEL | |
| | | ALPSKYVNFLYLASHYEKLKGSPEDNEQKQLFVEQHKHYLDEIIE | |
| | | QISEFSKRVILADANLDKVLSAYNKHRDKPIREQAENIIHLFTLTRL | |
| | | GAPRAFKYFDTTIDPKQYRSTKEVLDATLIHQSITGLYETRIDLSQL | |
| | | GGD | |
| 97 | St1Cas9 | MSDLVLGLDIGIGSVGVGILNKVTGEIIHKNSRIFPAAQAENNLVRR | — |
| | | TNRQGRRLARRKKHRRVRLNRLFEESGLITDFTKISINLNPYQLRV | |
| | | KGLTDELSNEELFIALKNMVKHRGISYLDDASDDGNSSVGDYAQI | |
| | | VKENSKQLETKTPGQIQLERYQTYGQLRGDFTVEKDGKKHRLINV | |
| | | FPTSAYRSEALRILQTQQEFNPQITDEFINRYLEILTGKRKYYHGPG | |
| | | NEKSRTDYGRYRTSGETLDNIFGILIGKCTFYPDEFRAAKASYTAQ | |
| | | EFNLLNDLNNLTVPTETKKLSKEQKNQIINYVKNEKAMGPAKLFK | |
| | | YIAKLLSCDVADIKGYRIDKSGKAEIHTFEAYRKMKTLETLDIEQM | |
| | | DRETLDKLAYVLTLNTEREGIQEALEHEFADGSFSQKQVDELVQF | |
| | | RKANSSIFGKGWHNFSVKLMMELIPELYETSEEQMTILTRLGKQK | |
| | | TTSSSNKTKYIDEKLLTEEIYNPVVAKSVRQAIKIVNAAIKEYGDFD | |
| | | NIVIEMARETNEDDEKKAIQKIQKANKDEKDAAMLKAANQYNG | |
| | | KAELPHSVFHGHKQLATKIRLWHQQGERCLYTGKTISIHDLINNSN | |
| | | QFEVDHILPLSITFDDSLANKVLVYATANQEKGQRTPYQALDSMD | |
| | | DAWSFRELKAFVRESKTLSNKKKEYLLTEEDISKFDVRKKFIERNL | |
| | | VDTRYASRVVLNALQEHFRAHKIDTKVSVVRGQFTSQLRRHWGIE | |
| | | KTRDTYHHHAVDALIIAASSQLNLWKKQKNTLVSYSEDQLLDIET | |
| | | GELISDDEYKESVFKAPYQHFVDTLKSKEFEDSILFSYQVDSKFNR | |
| | | KISDATIYATRQAKVGKDKADETYVLGKIKDIYTQDGYDAFMKIY | |
| | | KKDKSKFLMYRHDPQTFEKVIEPILENYPNKQINEKGKEVPCNPFL | |
| | | KYKEEHGYIRKYSKKGNGPEIKSLKYYDSKLGNHIDITPKDSNNK | |
| | | VVLQSVSPWRADVYFNKTTGKYEILGLKYADLQFEKGTGTYKISQ | |
| | | EKYNDIKKKEGVDSDSEFKFTLYKNDLLLVKDTETKEQQLFRFLS | |
| | | RTMPKQKHYVELKPYDKQKFEGGEALIKVLGNVANSGQCKKGLG | |
| | | KSNISIYKVRTDVLGNQHIIKNEGDKPKLDF | |
| 98 | St1Cas9 | MSDLVLGLDIGIGSVGVGILNKVTGEIIHKNSRIFPAAQAENNLVRR | H599A |
| | | TNRQGRRLARRKKHRRVRLNRLFEESGLITDFTKISINLNPYQLRV | |
| | | KGLTDELSNEELFIALKNMVKHRGISYLDDASDDGNSSVGDYAQI | |
| | | VKENSKQLETKTPGQIQLERYQTYGQLRGDFTVEKDGKKHRLINV | |
| | | FPTSAYRSEALRILQTQQEFNPQITDEFINRYLEILTGKRKYYHGPG | |
| | | NEKSRTDYGRYRTSGETLDNIFGILIGKCTFYPDEFRAAKASYTAQ | |
| | | EFNLLNDLNNLTVPTETKKLSKEQKNQIINYVKNEKAMGPAKLFK | |
| | | YIAKLLSCDVADIKGYRIDKSGKAEIHTFEAYRKMKTLETLDIEQM | |
| | | DRETLDKLAYVLTLNTEREGIQEALEHEFADGSFSQKQVDELVQF | |
| | | RKANSSIFGKGWHNFSVKLMMELIPELYETSEEQMTILTRLGKQK | |
| | | TTSSSNKTKYIDEKLLTEEIYNPVVAKSVRQAIKIVNAAIKEYGDFD | |
| | | NIVIEMARETNEDDEKKAIQKIQKANKDEKDAAMLKAANQYNG | |
| | | KAELPHSVFHGHKQLATKIRLWHQQGERCLYTGKTISIHDLINNSN | |
| | | QFEVDAILPLSITFDDSLANKVLVYATANQEKGQRTPYQALDSMD | |
| | | DAWSFRELKAFVRESKTLSNKKKEYLLTEEDISKFDVRKKFIERNL | |
| | | VDTRYASRVVLNALQEHFRAHKIDTKVSVVRGQFTSQLRRHWGIE | |
| | | KTRDTYHHHAVDALIIAASSQLNLWKKQKNTLVSYSEDQLLDIET | |
| | | GELISDDEYKESVFKAPYQHFVDTLKSKEFEDSILFSYQVDSKFNR | |
| | | KISDATIYATRQAKVGKDKADETYVLGKIKDIYTQDGYDAFMKIY | |
| | | KKDKSKFLMYRHDPQTFEKVIEPILENYPNKQINEKGKEVPCNPFL | |
| | | KYKEEHGYIRKYSKKGNGPEIKSLKYYDSKLGNHIDITPKDSNNK | |
| | | VVLQSVSPWRADVYFNKTTGKYEILGLKYADLQFEKGTGTYKISQ | |
| | | EKYNDIKKKEGVDSDSEFKFTLYKNDLLLVKDTETKEQQLFRFLS | |
| | | RTMPKQKHYVELKPYDKQKFEGGEALIKVLGNVANSGQCKKGLG | |
| | | KSNISIYKVRTDVLGNQHIIKNEGDKPKLDF | |

In some embodiments, the nuclease or the nickase region of the protein construct provided herein binds to a guide polynucleotide provided herein. In some embodiments, the nuclease or the nickase region of the protein construct binds to the protein binding region of the guide polynucleotide provided herein. In some embodiments, the nuclease or the nickase region of the protein construct binds to a target nucleic acid to form a complex. In some embodiments, the complex further comprises a portion of the guide polynucleotide sequence. For example, the protein binding region and the targeting region of the guide polynucleotide form a complex between the nuclease or the nickase region of an engineered protein provided herein and the target nucleic acid via the targeting sequence.

Binding of the nuclease, the nickase, or the fragments thereof is mediated by full or partial complementarity of the targeting region of the guide polynucleotide provided herein. In some embodiments, the engineered protein construct (e.g., the nickase or the nuclease) binds to a PAM sequence. Enzymes and proteins from different bacterial species can recognize different sequence motifs or PAMs. In some embodiments, the engineered protein construct binds to the guide polynucleotide that binds to the target nucleic acid within 5 nucleobases of a PAM sequence, 10 nucleobases of a PAM sequence, 15 nucleobases of a PAM sequence, or 20 nucleobases of a PAM sequence.

Upon binding to a target nucleic acid via the guide sequence, the nucleases and nickases provided herein specifically cut the target nucleic acid (e.g., DNA) at the distal end or the proximal end of the PAM. In some embodiments, the nuclease or nickase region of the protein construct generates a single-stranded break in the target nucleic acid. In some embodiments, the nuclease or the nickase region generates a double-stranded break in the target nucleic acid. In some embodiments, the nucleases and the nickases provided herein produce staggered ends when cutting the target nucleic acid that enables higher integration rates of synthesized DNA, improving gene editing efficiency. In some embodiments, the nucleases and the nickases provided herein cut the leading strand that will facilitate DNA synthesis upon hybridization with the hybridization region (HR) and DNA synthesis templated from the DST region of the guide polynucleotide. In some embodiments, the nucleases and the nickases provided herein cut the complementary strand. In some embodiments, an additional guide polynucleotide is introduced to cut the complementary strand. An additional guide polynucleotide can improve DNA editing efficiency and targeting of the engineered protein provided herein.

Provided herein are systems, compositions, and engineered proteins that comprise a DNA-dependent DNA polymerase (DdDP) or a fragment thereof. In some embodiments, the engineered proteins comprise a DdDP or a DdDP region. In some embodiments, the DdDP region is operably linked to the nuclease region or the nickase region provided herein. In some embodiments, the DNA polymerase is a bacterial, eukaryotic, insect or plant DNA polymerase. In some embodiments, the DNA polymerase is a eukaryotic DNA polymerase and is selected from the group consisting of: DNA polymerase α, β, γ, δ, and ε, or a corresponding DNA polymerase thereof. In some embodiments, the DNA polymerase is a bacterial DNA polymerase and is selected from the group consisting of DNA polymerase I, II, and III, or a corresponding DNA polymerase thereof. In some embodiments, the DdDP, the DdDP region, or the functional fragment thereof comprises: a T7 DNA polymerase, a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol μ, a Pol κ, a Pol ι, a Pol η, a Pol IV, a Pol V, a terminal deoxynucleotidyl transferase, or any combination thereof. In some embodiments, the DdDP or the function fragment thereof comprises a Klenow fragment of a DNA Pol I. In some embodiments, the DdDP is not a reverse transcriptase enzyme. In some embodiments, the DdDP is not error prone. In some embodiments, the DdDP is a high-fidelity DdDP. In some embodiments, the DdDP or the functional fragment thereof comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof.

Exemplary amino acid sequences for a DdDP region provided herein can include but are not limited to:

a phi29 DNA polymerase:
(SEQ ID NO: 63)
MKHMPRKMYSCDFETTTKVEDCRVWAYGYMNIEDHSEYKIGNSLDEFMAWVLKVQADLYFH
NLKFDGAFIINWLERNGFKWSADGLPNTYNTIISRMGQWYMIDICLGYKGKRKIHTVIYDSLKKL
PFPVKKIAKDFKLTVLKGDIDYHKERPVGYKITPEEYAYIKNDIQIIAEALLIQFKQGLDRMTAGS
DSLKGFKDIITTKKFKKVFPTLSLGLDKEVRYAYRGGFTWLNDRFKEKEIGEGMVFDVNSLYPA
QMYSRLLPYGEPIVFEGKYVWDEDYPLHIQHIRCEFELKEGYIPTIQIKRSRFYKGNEYLKSSGGEI
ADLWLSNVDLELMKEHYDLYNVEYISGLKFKATTGLFKDFIDKWTYIKTTSEGAIKQLAKLMLN
SLYGKFASNPDVTGKVPYLKENGALGFRLGEEETKDPVYTPMGVFITAWARYTTITAAQACYDR
IIYCDTDSIHLTGTEIPDVIKDIVDPKKLGYWAHESTFKRAKYLRQKTYIQDIYMKEVDGKLVEGS
PDDYTDIKFSVKCAGMTDKIKKEVTFENFKVGFSRKMKPKPVQVPGGVVLVDDTFTIK;
a Ba71V DNA polymerase:
(SEQ ID NO: 64)
MLTLIQGKKIVNHLRSRLAFEYNGQLIKILSKNIVAVGSLRREEKMLNDVDLLIIVPEKKLLKHVL
PNIRIKGLSFSVKVCGERKCVLFIEWEKKTYQLDLFTALAEEKPYAIFHFTGPVSYLIRIRAALKKK
NYKLNQYGLFKNQTLVPLKITTEKELIKELGFTYRIPKKRL;
a human DNA polymerase lambda:
(SEQ ID NO: 65)
MDPRGILKAFPKRQKIHADASSKVLAKIPRREEGEEAEEWLSSLRAHVVRTGIGRARAELFEKQI
VQHGGQLCPAQGPGVTHIVVDEGMDYERALRLLRLPQLPPGAQLVKSAWLSLCLQERRLVDVA
GFSIFIPSRYLDHPQPSKAEQDASIPPGTHEALLQTALSPPPPPTRPVSPPQKAKEAPNTQAQPISDD
EASDGEETQVSAADLEALISGHYPTSLEGDCEPSPAPAVLDKWVCAQPSSQKATNHNLHITEKLE
VLAKAYSVQGDKWRALGYAKAINALKSFHKPVTSYQEACSIPGIGKRMAEKIIEILESGHLRKLD
HISESVPVLELFSNIWGAGTKTAQMWYQQGFRSLEDIRSQASLTTQQAIGLKHYSDFLERMPREE
ATEIEQTVQKAAQAFNSGLLCVACGSYRRGKATCGDVDVLITHPDGRSHRGIFSRLLDSLRQEGF
LTDDLVSQEENGQQQKYLGVCRLPGPGRRHRRLDIIVVPYSEFACALLYFTGSAHFNRSMRALA
KTKGMSLSEHALSTAVVRNTHGCKVGPGRVLPTPTEKDVFRLLGLPYREPAERDW;
a human DNA polymerase beta
(SEQ ID NO: 66)
MSKRKAPQETLNGGITDMLTELANFEKNVSQAIHKYNAYRKAASVIAKYPHKIKSGAEAKKLPG
VGTKIAEKIDEFLATGKLRKLEKIRQDDTSSSINFLTRVSGIGPSAARKFVDEGIKTLEDLRKNEDK
LNHHQRIGLKYFGDFEKRIPREEMLQMQDIVLNEVKKVDSEYIATVCGSFRRGAESSGDMDVLL
THPSFTSESTKQPKLLHQVVEQLQKVHFITDTLSKGETKFMGVCQLPSKNDEKEYPHRRIDIRLIP
KDQYYCGVLYFTGSDIFNKNMRAHALEKGFTINEYTIRPLGVTGVAGEPLPVDSEKDIFDYIQWK
YREPKDRSE;
a Bsu DNA polymerase I
(SEQ ID NO: 67)
MKNKLVLIDGNSVAYRAFFALPLLHNDKGIHTNAVYGFTMMLNKILAEEQPTHILVAFDAGKTT
FRHETFQDYKGGRQQTPPELSEQFPLLRELLKAYRIPAYELDHYEADDIIGTMAARAEREGFAVK
VISGDRDLTQLASPQVTVEITKKGITDIESYTPETVVEKYGLTPEQIVDLKGLMGDKSDNIPGVPGI
GEKTAVKLLKQFGTVENVLASIDEIKGEKLKENLRQYRDLALLSKQLAAICRDAPVELTLDDIVY
KGEDREKVVALFQELGFQSFLDKMAVQTDEGEKPLAGMDFAIADSVTDEMLADKAALVVEVV
GDNYHHAPIVGIALANERGRFFLRPETALADPKFLAWLGDETKKKTMFDSKRAAVALKWKGIEL
RGVVFDLLLAAYLLDPAQAAGDVAAVAKMHQYEAVRSDEAVYGKGAKRTVPDEPTLAEHLVR
KAAAIWALEEPLMDELRRNEQDRLLTELEQPLAGILANMEFTGVKVDTKRLEQMGAELTEQLQ
AVERRIYELAGQEFNINSPKQLGTVLFDKLQLPVLKKTKTGYSTSADVLEKLAPHHEIVEHILHYR
QLGKLQSTYIEGLLKVVHPVTGKVHTMFNQALTQTGRLSSVEPNLQNIPIRLEEGRKIRQAFVPSE
PDWLIFAADYSQIELRVLAHIAEDDNLIEAFRRGLDIHTKTAMDIFHVSEEDVTANMRRQAKAVN
FGIVYGISDYGLAQNLNITRKEAAEFIERYFASFPGVKQYMDNIVQEAKQKGYVTTLLHRRRYLP
DITSRNFNVRSFAERTAMNTPIQGSAADIIKKAMIDLSVRLREERLQARLLLQVHDELILEAPKEEI
ERLCRLVPEVMEQAVTLRVPLKVDYHYGPTWYDAK;
an E. coli DNA polymerase I
(SEQ ID NO: 68)
MVQIPQNPLILVDGSSYLYRAYHAFPPLTNSAGEPTGAMYGVLNMLRSLIMQYKPTHAAVVFDA
KGKTFRDELFEHYKSHRPPMPDDLRAQIEPLHAMVKAMGLPLLAVSGVEADDVIGTLAREAEKA
GRPVLISTGDKDMAQLVTPNITLINTMTNTILGPEEVVNKYGVPPELIIDFLALMGDSSDNIPGVPG
VGEKTAQALLQGLGGLDTLYAEPEKIAGLSFRGAKTMAAKLEQNKEVAYLSYQLATIKTDVELE
LTCEQLEVQQPAAEELLGLFKKYEFKRWTADVEAGKWLQAKGAKPAAKPQETSVADEAPEVTA
TVISYDNYVTILDEETLKAWIAKLEKAPVFAFDTETDSLDNISANLVGLSFAIEPGVAAYIPVAHD
YLDAPDQISRERALELLKPLLEDEKALKVGQNLKYDRGILANYGIELRGIAFDTMLESYILNSVAG
RHDMDSLAERWLKHKTITFEEIAGKGKNQLTFNQIALEEAGRYAAEDADVTLQLHLKMWPDLQ
KHKGPLNVFENIEMPLVPVLSRIERNGVKIDPKVLHNHSEELTLRLAELEKKAHEIAGEEFNLSST
KQLQTILFEKQGIKPLKKTPGGAPSTSEEVLEELALDYPLPKVILEYRGLAKLKSTYTDKLPLMINP
KTGRVHTSYHQAVTATGRLSSTDPNLQNIPVRNEEGRRIRQAFIAPEDYVIVSADYSQIELRIMAH
LSRDKGLLTAFAEGKDIHRATAAEVFGLPLETVTSEQRRSAKAINFGLIYGMSAFGLARQLNIPRK
EAQKYMDLYFERYPGVLEYMERTRAQAKEQGYVETLDGRRLYLPDIKSSNGARRAAAERAAIN
APMQGTAADIIKRAMIAVDAWLQAEQPRVRMIMQVHDELVFEVHKDDVDAVAKQIHQLMENC
TRLDVPLLVEVGSGENWDQAH;
a Klenow fragment
(SEQ ID NO: 69)
MVISYDNYVTILDEETLKAWIAKLEKAPVFAFDTETDSLDNISANLVGLSFAIEPGVAAYIPVAHD
YLDAPDQISRERALELLKPLLEDEKALKVGQNLKYDRGILANYGIELRGIAFDTMLESYILNSVAG
RHDMDSLAERWLKHKTITFEEIAGKGKNQLTFNQIALEEAGRYAAEDADVTLQLHLKMWPDLQ
KHKGPLNVFENIEMPLVPVLSRIERNGVKIDPKVLHNHSEELTLRLAELEKKAHEIAGEEFNLSST
KQLQTILFEKQGIKPLKKTPGGAPSTSEEVLEELALDYPLPKVILEYRGLAKLKSTYTDKLPLMINP
KTGRVHTSYHQAVTATGRLSSTDPNLQNIPVRNEEGRRIRQAFIAPEDYVIVSADYSQIELRIMAH
LSRDKGLLTAFAEGKDIHRATAAEVFGLPLETVTSEQRRSAKAINFGLIYGMSAFGLARQLNIPRK
EAQKYMDLYFERYPGVLEYMERTRAQAKEQGYVETLDGRRLYLPDIKSSNGARRAAAERAAIN
APMQGTAADIIKRAMIAVDAWLQAEQPRVRMIMQVHDELVFEVHKDDVDAVAKQIHQLMENC
TRLDVPLLVEVGSGENWDQAH;
a T3 DNA polymerase
(SEQ ID NO: 70)
MLVSDIEANNLLEKVTKFHCGVIYDYRDGEYHSYRPGDFGAYLDALEAEVKRGGLIVFHNGHK
YDVPALTKLAKLQLNREFHLPRENCIDTLVLSRLIHSNLKDTDMGLLRSGKLPGKRFGSHALEA
WGYRLGEMKGEYKDDFKRMLEEQGEEYVDGMEWWNFNEEMMDYNVQDVVVTKALLEKLLS
DKHYFPPEIDFTDVGYTTFWSESLEAVDVEHRAAWLLAKQERNGFPFDTKAIEELYVELAARRSE
LLRNLTETFGSWYQPKGGTEMFCHPRTGKPLPKYPRIKIPKVGGIFKKPKNKAQREGREPCELDT
REYVAGAPYTPVEHVVFNPSSRDHIQKKLQEAGWVPTKFTDKGAPVVDDEVLEGVRVDDPEKQ
AAIDLIKEYLMIQKRIGQSAEGDKAWLRYVAEDGKIHGSVNPNGAVTGRATHAFPNLAQIPGVR
SPYGEQCRAAFGAEHHLDGITGKPWVQAGIDASGLELRCLAHFMARFDNGEYAHEILNGDIHTK
NQMAAELPTRDNAKTFIYGFLYGAGDEKIGQIVGAGKERGKELKKKFLENTPAIAALRESIQQTL
VESSQWVAGEQQVKWKRRWIKGLDGRKVHVRSPHAALNTLLQSAGALICKLWIIKTEEMLVEK
GLKHGWDGDFAYMAWIHDEIQVACRTEEIAKTVIEVAQEAMRWVGEHWNFRCLLDTEGKMGA
NWKECH;
a T4 DNA polymerase
(SEQ ID NO: 71)
MKEFYISIETVGNNIVERYIDENGKERTREVEYLPTMFRHCKEESKYKDIYGKNCAPQKFPSMKD
ARDWMKRMEDIGLEALGMNDFKLAYISDTYGSEIVYDRKFVRVANCDIEVTGDKFPDPMKAEY
EIDAITHYDSIDDRFYVFDLLNSMYGSVSKWDAKLAAKLDCEGGDEVPQEILDRVIYMPFDNER
DMLMEYINLWEQKRPAIFTGWNIEGFDVPYIMNRVKMILGERSMKRFSPIGRVKSKLIQNMYGS
KEIYSIDGVSILDYLDLYKKFAFTNLPSFSLESVAQHETKKGKLPYDGPINKLRETNHQRYISYNII
DVESVQAIDKIRGFIDLVLSMSYYAKMPFSGVMSPIKTWDAIIFNSLKGEHKVIPQQGSHVKQSFP
GAFVFEPKPIARRYIMSFDLTSLYPSIIRQVNISPETIRGQFKVHPIHEYIAGTAPKPSDEYSCSPNG
WMYDKHQEGIIPKEIAKVFFQRKDWKKKMFAEEMNAEAIKKIIMKGAGSCSTKPEVERYVKFSD
DFLNELSNYTESVLNSLIEECEKAATLANTNQLNRKILINSLYGALGNIHFRYYDLRNATAITIFGQ
VGIQWIARKINEYLNKVCGTNDEDFIAAGDTDSVYVCVDKVIEKVGLDRFKEQNDLVEFMNQFG
KKKMEPMIDVAYRELCDYMNNREHLMHMDREAISCPPLGSKGVGGFWKAKKRYALNVYDME
DKRFAEPHLKIMGMETQQSSTPKAVQEALEESIRRILQEGEESVQEYYKNFEKEYRQLDYKVIAE
VKTANDIAKYDDKGWPGFKCPFHIRGVLTYRRAVSGLGVAPILDGNKVMVLPLREGNPFGDKCI
AWPSGTELPKEIRSDVLSWIDHSTLFQKSFVKPLAGMCESAGMDYEEKASLDFLFG;
a T5 DNA polymerase
(SEQ ID NO: 72)
MKIAVVDKALNNTRYDKHFQLYGEEVDVFHMCNEKLSGRLLKKHITIGTPENPFDPNDYDFVIL
VGAEPFLYFAGKKGIGDYTGKRVEYNGYANWIASISPAQLHFKPEMKPVFDATVENIHDIINGRE
KIAKAGDYRPITDPDEAEEYIKMVYNMVIGPVAFDSETSALYCRDGYLLGVSISHQEYQGVYIDS
DCLTEVAVYYLQKILDSENHTIVFHNLKFDMHFYKYHLGLTFDKAHKERRLHDTMLQHYVLDE
RRGTHGLKSLAMKYTDMGDYDFELDKFKDDYCKAHKIKKEDFTYDLIPFDIMWPYAAKDTDAT
IRLHNFFLPKIEKNEKLCSLYYDVLMPGCVFLQRVEDRGVPISIDRLKEAQYQLTHNLNKAREKL
YTYPEVKQLEQDQNEAFNPNSVKQLRVLLFDYVGLTPTGKLTDTGADSTDAEALNELATQHPIA
KTLLEIRKLTKLISTYVEKILLSIDADGCIRTGFHEHMTTSGRLSSSGKLNLQQLPRDESIIKGCVVA
PPGYRVIAWDLTTAEVYYAAVLSGDRNMQQVFINMRNEPDKYPDFHSNIAHMVFKLQCEPRDV
KKLFPALRQAAKAITFGILYGSGPAKVAHSVNEALLEQAAKTGEPFVECTVADAKEYIETYFGQF
PQLKRWIDKCHDQIKNHGFIYSHFGRKRRLHNIHSEDRGVQGEEIRSGFNAIIQSASSDSLLLGAV
DADNEIISLGLEQEMKIVMLVHDSVVAIVREDLIDQYNEILIRNIQKDRGISIPGCPIGIDSDSEAGG
SRDYSCGKMKKQHPSIACIDDDEYTRYVKGVLLDAEFEYKKLAAMDKEHPDHSKYKDDKFIAV
CKDLDNVKRILGA;
a T7 DNA polymerase
(SEQ ID NO: 73)
MIVSDIEANALLESVTKFHCGVIYDYSTAEYVSYRPSDFGAYLDALEAEVARGGLIVFHNGHKYD
VPALTKLAKLQLNREFHLPRENCIDTLVLSRLIHSNLKDTDMGLLRSGKLPGKRFGSHALEAWG
YRLGEMKGEYKDDFKRMLEEQGEEYVDGMEWWNFNEEMMDYNVQDVVVTKALLEKLLSDK
HYFPPEIDFTDVGYTTFWSESLEAVDIEHRAAWLLAKQERNGFPFDTKAIEELYVELAARRSELL
RKLTETFGSWYQPKGGTEMFCHPRTGKPLPKYPRIKTPKVGGIFKKPKNKAQREGREPCELDTRE
YVAGAPYTPVEHVVFNPSSRDHIQKKLQEAGWVPTKYTDKGAPVVDDEVLEGVRVDDPEKQA
AIDLIKEYLMIQKRIGQSAEGDKAWLRYVAEDGKIHGSVNPNGAVTGRATHAFPNLAQIPGVRSP
YGEQCRAAFGAEHHLDGITGKPWVQAGIDASGLELRCLAHFMARFDNGEYAHEILNGDIHTKN
QIAAELPTRDNAKTFIYGFLYGAGDEKIGQIVGAGKERGKELKKKFLENTPAIAALRESIQQTLVE
SSQWVAGEQQVKWKRRWIKGLDGRKVHVRSPHAALNTLLQSAGALICKLWIIKTEEMLVEKGL
KHGWDGDFAYMAWVHDEIQVGCRTEEIAQVVIETAQEAMRWVGDHWNFRCLLDTEGKMGPN
WAICH;
a Bst DNA polymerase
(SEQ ID NO: 74)
MKKKLVLIDGNSVAYRAFFALPLLHNDKGIHTNAVYGFTMMLNKILAEEQPTHLLVAFDAGKTT
FRHETFQEYKGGRQQTPPELSEQFPLLRELLKAYRIPAYELDHYEADDIIGTLAARAEQEGFEVKII
SGDRDLTQLASRHVTVDITKKGITDIEPYTPETVREKYGLTPEQIVDLKGLMGDKSDNIPGVPGIG
EKTAVKLLKQFGTVENVLASIDEVKGEKLKENLRQHRDLALLSKQLASICRDAPVELSLDDIVYE
GQDREKVIALFKELGFQSFLEKMAAPAAEGEKPLEEMEFAIVDVITEEMLADKAALVVEVMEEN
YHDAPIVGIALVNEHGRFFMRPETALADSQFLAWLADETKKKSMFDAKRAVVALKWKGIELRG
VAFDLLLAAYLLNPAQDAGDIAAVAKMKQYEAVRSDEAVYGKGVKRSLPDEQTLAEHLVRKA
AAIWALEQPFMDDLRNNEQDQLLTKLEQPLAAILAEMEFTGVNVDTKRLEQMGSELAEQLRAIE
QRIYELAGQEFNINSPKQLGVILFEKLQLPVLKKTKTGYSTSADVLEKLAPHHEIVENILHYRQLG
KLQSTYIEGLLKVVRPDTGKVHTMFNQALTQTGRLSSAEPNLQNIPIRLEEGRKIRQAFVPSEPDW
LIFAADYSQIELRVLAHIADDDNLIEAFQRDLDIHTKTAMDIFHVSEEEVTANMRRQAKAVNFGI
VYGISDYGLAQNLNITRKEAAEFIERYFASFPGVKQYMENIVQEAKQKGYVTTLLHRRRYLPDIT
SRNFNVRSFAERTAMNTPIQGSAADIIKKAMIDLAARLKEEQLQARLLLQVHDELILEAPKEEIER
LCELVPEVMEQAVTLRVPLKVDYHYGPTWYDAK;
a human DNA polymerase alpha
(SEQ ID NO: 75)
MSASAQQLAEELQIFGLDCEEALIEKLVELCVQYGQNEEGMVGELIAFCTSTHKVGLTSEILNSFE
HEFLSKRLSKARHSTCKDSGHAGARDIVSIQELIEVEEEEEILLNSYTTPSKGSQKRAISTPETPLTK
RSVSTRSPHQLLSPSSFSPSATPSQKYNSRSNRGEVVTSFGLAQGVSWSGRGGAGNISLKVLGCPE
ALTGSYKSMFQKLPDIREVLTCKIEELGSELKEHYKIEAFTPLLAPAQEPVTLLGQIGCDSNGKLN
NKSVILEGDREHSSGAQIPVDLSELKEYSLFPGQVVIMEGINTTGRKLVATKLYEGVPLPFYQPTE
EDADFEQSMVLVACGPYTTSDSITYDPLLDLIAVINHDRPDVCILFGPFLESKHEQVENCLLTSPFE
DIFKQCLRTIIEGTRSSGSHLVFVPSLRDVHHEPVYPQPPFSYSDLSREDKKQVQFVSEPCSLSING
VIFGLTSTDLLFHLGAEEISSSSGTSDRFSRILKHILTQRSYYPLYPPQEDMAIDYESFYVYAQLPVT
PDVLIIPSELRYFVKDVLGCVCVNPGRLTKGQVGGTFARLYLRRPAADGAERQSPCIAVQVVRI;
a human alpha herpesvirus 1 DNA polymerase
(SEQ ID NO: 76)
MFSGGGGPLSPGGKSAARAASGFFAPAGPRGAGRGPPPCLRQNFYNPYLAPVGTQQKPTGPTQR
HTYYSECDEFRFIAPRVLDEDAPPEKRAGVHDGHLKRAPKVYCGGDERDVLRVGSGGFWPRRS
RLWGGVDHAPAGFNPTVTVFHVYDILENVEHAYGMRAAQFHARFMDAITPTGTVITLLGLTPEG
HRVAVHVYGTRQYFYMNKEEVDRHLQCRAPRDLCERMAAALRESPGASFRGISADHFEAEVVE
RTDVYYYETRPALFYRVYVRSGRVLSYLCDNFCPAIKKYEGGVDATTRFILDNPGFVTFGWYRL
KPGRNNTLAQPRAPMAFGTSSDVEFNCTADNLAIEGGMSDLPAYKLMCFDIECKAGGEDELAFP
VAGHPEDLVIQISCLLYDLSTTALEHVLLFSLGSCDLPESHLNELAARGLPTPVVLEFDSEFEMLL
AFMTLVKQYGPEFVTGYNIINFDWPFLLAKLTDIYKVPLDGYGRMNGRGVFRVWDIGQSHFQKR
SKIKVNGMVNIDMYGIITDKIKLSSYKLNAVAEAVLKDKKKDLSYRDIPAYYAAGPAQRGVIGE
YCIQDSLLVGQLFFKFLPHLELSAVARLAGINITRTIYDGQQIRVFTCLLRLADQKGFILPDTQGRF
RGAGGEAPKRPAAAREDEERPEEEGEDEDEREEGGGEREPEGARETAGRHVGYQGARVHDPTS
GFHVNPVVGFDFASLYPSIIQAHNLCFSTLSLRADAVAHLEAGKDYLEIEVGGRRLFFVKAHVRE
SLLSILLRDWLAMRKQIRSRIPQSSPEEAVLLDKQQAAIKVVCNSVYGFTGVQHGLLPCLHVAAT
VTTIGREMLLATREYVHARWAAFEQLLADFPEAADMRAPGPYSMRIIYGDTDSIFVLCRGLTAA
GLTAMGDKMASHISRALFLPPIKLECEKTFTKLLLIAKKKYIGVIYGGKMLIKGVDLVRKNNCAFI
NRTSRALVDLLFYDDTVSGAAAALAERPAEEWLARPLPEGLQAFGAVLVDAHRRITDPERDIQD
FVLTAELSRHPRAYTNKRLAHLTVYYKLMARRAQVPSIKDRIPYVIVAQTREVEETVARLAALRE
LDAAAPGDEPAPPAALPSPAKRPRETPSHADPPGGASKPRKLLVSELAEDPAYAIAHGVALNTDY
YFSHLLGAACVTFKALFGNNAKITESLLKRFIPEVWHPPDDVAARLRAAGFGAVGAGATAEETR
RMLHRAFDTLA, a variant, or a fragment thereof.

In some embodiments, a DNA-dependent DNA polymerase provided herein comprise a phi29 DNA-dependent DNA polymerase. In some embodiments, the Phi29 DNA polymerase comprises a mutation, wherein the mutation comprises an amino acid substitution of an amino acid residue of position: 8, 12, 51, 66, 97, 137, 197, 221, 369, 372, 375, 377, 378, 497, 512, or 526 as compared to SEQ ID NO: 63. In some embodiments, a DNA-dependent DNA polymerase provided herein comprise a sequence listed in Table 2.

TABLE 2
Phi29 DNA polymerase sequences.

| SEQ ID | | |
|---|---|---|
| NO: | Sequence | Mutations |
| 99 | MKHMPRKMYSCAFETTTKVEDCRVWAYGYMNIEDHSEYKIGNSL | D12A, D66A |
| | DEFMAWVLKVQADLYFHNLKFAGAFIINWLERNGFKWSADGLPN | |
| | TYNTIISRMGQWYMIDICLGYKGKRKIHTVIYDSLKKLPFPVKKIA | |
| | KDFKLTVLKGDIDYHKERPVGYKITPEEYAYIKNDIQIIAEALLIQFK | |
| | QGLDRMTAGSDSLKGFKDIITTKKFKKVFPTLSLGLDKEVRYAYRG | |
| | GFTWLNDRFKEKEIGEGMVFDVNSLYPAQMYSRLLPYGEPIVFEGK | |
| | YVWDEDYPLHIQHIRCEFELKEGYIPTIQIKRSRFYKGNEYLKSSGG | |
| | EIADLWLSNVDLELMKEHYDLYNVEYISGLKFKATTGLFKDFIDKW | |
| | TYIKTTSEGAIKQLAKLMLNSLYGKFASNPDVTGKVPYLKENGALG | |
| | FRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYM | |
| | KEVDGKLVEGSPDDYTDIKFSVKCAGMTDKIKKEVTFENFKVGFS | |
| | RKMKPKPVQVPGGVVLVDDTFTIK | |
| 100 | MKHMPRKRYSCAFETTTKVEDCRVWAYGYMNIEDHSEYKIGNSLD | M8R, D12A, |
| | EFMAWALKVQADLYFHNLKFAGAFIINWLERNGFKWSADGLPNTY | V51A, D66A, |
| | NTIISRTGQWYMIDICLGYKGKRKIHTVIYDSLKKLPFPVKKIAKDF | M97T, |
| | KLTVLKGDIDYHKERPVGYKITPEEYAYIKNDIQIIAEALLIQFKQGL | G197D, |
| | DRMTAGSDSLKDFKDIITTKKFKKVFPTLSLGLDKKVRYAYRGGFT | E221K |
| | WLNDRFKEKEIGEGMVFDVNSLYPAQMYSRLLPYGEPIVFEGKYV | |
| | WDEDYPLHIQHIRCEFELKEGYIPTIQIKRSRFYKGNEYLKSSGGEIA | |
| | DLWLSNVDLELMKEHYDLYNVEYISGLKFKATTGLFKDFIDKWTYI | |
| | KTTSEGAIKQLAKLMLNSLYGKFASNPDVTGKVPYLKENGALGFR | |
| | LGEEETKDPVYTPMGVFITAWARYTTITAAQACYDRIIYCDTDSIHL | |
| | TGTEIPDVIKDIVDPKKLGYWAHESTFKRAKYLRQKTYIQDIYMKE | |
| | VDGKLVEGSPDDYTDIKFSVKCAGMTDKIKKEVTFENFKVGFSRK | |
| | MKPKPVQVPGGVVLVDDTFTIK | |
| 101 | MKHMPRKRYSCAFETTTKVEDCRVWAYGYMNIEDHSEYKIGNSLD | M8R, D12A, |
| | EFMAWALKVQADLYFHNLKFAGAFIINWLERNGFKWSADGLPNTY | V51A, D66A, |
| | NTIISRTGQWYMIDICLGYKGKRKIHTVIYDSLKKLPFPVKKIAKDF | M97T, |
| | KLTVLKGDIDYHKERPVGYKITPEEYAYIKNDIQIIAEALLIQFKQGL | G197D, |
| | DRMTAGSDSLKDFKDIITTKKFKKVFPTLSLGLDKKVRYAYRGGFT | E221K, |
| | WLNDRFKEKEIGEGMVFDVNSLYPAQMYSRLLPYGEPIVFEGKYV | Q497P, |
| | WDEDYPLHIQHIRCEFELKEGYIPTIQIKRSRFYKGNEYLKSSGGEIA | K512E, F526L |
| | DLWLSNVDLELMKEHYDLYNVEYISGLKFKATTGLFKDFIDKWTYI | |
| | KTTSEGAIKQLAKLMLNSLYGKFASNPDVTGKVPYLKENGALGFR | |
| | LGEEETKDPVYTPMGVFITAWARYTTITAAPACYDRIIYCDTDSIHLT | |
| | GTEIPDVIKDIVDPKKLGYWAHESTFKRAKYLRQKTYIQDIYMKEV | |
| | DGELVEGSPDDYTDIKLSVKCAGMTDKIKKEVTFENFKVGFSRKM | |
| | KPKPVQVPGGVVLVDDTFTIK | |
| 102 | MKHMPRKRYSCAFETTTKVEDCRVWAYGYMNIEDHSEYKIGNSLD | M8R, D12A, |
| | EFMAWALKVQADLYFHNLKFAGAFIINWLERNGFKWSADGLPNTY | V51A, D66A, |
| | NTIISRTGQWYMIDICLGYKGKRKIHTVIYDSLKKLPFPVKKIAKDC | M97T, F137C, |
| | KLTVLKGDIDYHKERPVGYKITPEEYAYIKNDIQIIAEALLIQFKQGL | G197D, |
| | DRMTAIITTKKFKKVFPTLSLGLDKKVRYAYRGGFTWLNDRFKEKE | E221K |
| | IGEGMVFDVNSLYPAQMYSRLLPYGEPIVFEGKYVWDEDYPLHIQ | A377C, |
| | HIRCEFELKEGYIPTIQIKRSRFYKGNEYLKSSGGEIADLWLSNVDL | Q497P, |
| | ELMKEHYDLYNVEYISGLKFKATTGLFKDFIDKWTYIKTTSEGCIK | K512E, F526L |
| | QLAKLMLNSLYGKFASNPDVTGKVPYLKENGALGFRLGEEETKDP | |
| | VYTPMGVFITAWARYTTITAAQACYDRIIYCDTDSIHLTGTEIPDVIK | |
| | DIVDPKKLGYWAHESTFKRAKYLRPKTYIQDIYMKEVDGELVEGS | |
| | PDDYTDIKLSVKCAGMTDKIKKEVTFENFKVGFSRKMKPKPVQVP | |
| | GGVVLVDDTFTIK | |
| 103 | MKHMPRKRYSCDFETTTKVEDCRVWAYGYMNIEDHSEYKIGNSLD | M8R, V51A, |
| | EFMAWALKVQADLYFHNLKFDGAFIINWLERNGFKWSADGLPNT | M97T, F137C, |
| | YNTIISRTGQWYMIDICLGYKGKRKIHTVIYDSLKKLPFPVKKIAKD | G197D, |
| | CKLTVLKGDIDYHKERPVGYKITPEEYAYIKNDIQIIAEALLIQFKQG | E221K, |
| | LDRMTAGSDSLKDFKDIITTKKFKKVFPTLSLGLDKKVRYAYRGGF | A377C, |
| | TWLNDRFKEKEIGEGMVFDVNSLYPAQMYSRLLPYGEPIVFEGKY | Q497P, |
| | VWDEDYPLHIQHIRCEFELKEGYIPTIQIKRSRFYKGNEYLKSSGGEI | K512E, F526L |
| | ADLWLSNVDLELMKEHYDLYNVEYISGLKFKATTGLFKDFIDKWT | |
| | YIKTTSEGCIKQLAKLMLNSLYGKFASNPDVTGKVPYLKENGALGF | |
| | RLGEEETKDPVYTPMGVFITAWARYTTITAAQACYDRIIYCDTDSIH | |
| | LTGTEIPDVIKDIVDPKKLGYWAHESTFKRAKYLRPKTYIQDIYMK | |
| | EVDGELVEGSPDDYTDIKLSVKCAGMTDKIKKEVTFENFKVGFSR | |
| | KMKPKPVQVPGGVVLVDDTFTIK | |
| 104 | MKHMPRKMYSCAFETTTKVEDCRVWAYGYMNIEDHSEYKIGNSL | D12A, D66A, |
| | DEFMAWVLKVQADLYFHNLKFAGAFIINWLERNGFKWSADGLPN | G197D, |
| | TYNTIISRMGQWYMIDICLGYKGKRKIHTVIYDSLKKLPFPVKKIA | Y369E, |
| | KDFKLTVLKGDIDYHKERPVGYKITPEEYAYIKNDIQIIAEALLIQFK | T372N, |
| | QGLDRMTAGSDSLKDFKDIITTKKFKKVFPTLSLGLDKEVRYAYRG | E375D, I378R |
| | GFTWLNDRFKEKEIGEGMVFDVNSLYPAQMYSRLLPYGEPIVFEGK | |
| | YVWDEDYPLHIQHIRCEFELKEGYIPTIQIKRSRFYKGNEYLKSSGG | |
| | EIADLWLSNVDLELMKEHYDLYNVEYISGLKFKATTGLFKDFIDKW | |
| | TEIKNTSDGARKQLAKLMLNSLYGKFASNPDVTGKVPYLKENGAL | |
| | GFRLGEEETKDPVYTPMGVFITAWARYTTITAAQACYDRIIYCDTDS | |
| | IHLTGTEIPDVIKDIVDPKKLGYWAHESTFKRAKYLRQKTYIQDIYM | |
| | KEVDGKLVEGSPDDYTDIKFSVKCAGMTDKIKKEVTFENFKVGFS | |
| | RKMKPKPVQVPGGVVLVDDTFTIK | |
| 105 | MKHMPRKRYSCAFETTTKVEDCRVWAYGYMNIEDHSEYKIGNSLD | M8R, D12A, |
| | EFMAWALKVQADLYFHNLKFAGAFIINWLERNGFKWSADGLPNTY | V51A, D66A, |
| | NTIISRTGQWYMIDICLGYKGKRKIHTVIYDSLKKLPFPVKKIAKDF | M97T, |
| | KLTVLKGDIDYHKERPVGYKITPEEYAYIKNDIQIIAEALLIQFKQGL | G197D, |
| | DRMTAGSDSLKDFKDIITTKKFKKVFPTLSLGLDKKVRYAYRGGFT | E221K, |
| | WLNDRFKEKEIGEGMVFDVNSLYPAQMYSRLLPYGEPIVFEGKYV | Y369E, |
| | WDEDYPLHIQHIRCEFELKEGYIPTIQIKRSRFYKGNEYLKSSGGEIA | T372N, |
| | DLWLSNVDLELMKEHYDLYNVEYISGLKFKATTGLFKDFIDKWTEI | E375D, I378R, |
| | KNTSDGARKQLAKLMLNSLYGKFASNPDVTGKVPYLKENGALGF | Q497P, |
| | RLGEEETKDPVYTPMGVFITAWARYTTITAAQACYDRIIYCDTDSIH | K512E, F526L |
| | LTGTEIPDVIKDIVDPKKLGYWAHESTFKRAKYLRPKTYIQDIYMK | |
| | EVDGELVEGSPDDYTDIKLSVKCAGMTDKIKKEVTFENFKVGFSR | |
| | KMKPKPVQVPGGVVLVDDTFTIK | |
| 106 | MKHMPRKRYSCAFETTTKVEDCRVWAYGYMNIEDHSEYKIGNSLD | M8R, D12A, |
| | EFMAWALKVQADLYFHNLKFAGAFIINWLERNGFKWSADGLPNTY | V51A, D66A, |
| | NTIISRTGQWYMIDICLGYKGKRKIHTVIYDSLKKLPFPVKKIAKDC | M97T, F137C, |
| | KLTVLKGDIDYHKERPVGYKITPEEYAYIKNDIQIIAEALLIQFKQGL | G197D, |
| | DRMTAGSDSLKDFKDIITTKKFKKVFPTLSLGLDKKVRYAYRGGFT | E221K, |
| | WLNDRFKEKEIGEGMVFDVNSLYPAQMYSRLLPYGEPIVFEGKYV | A377C, |
| | WDEDYPLHIQHIRCEFELKEGYIPTIQIKRSRFYKGNEYLKSSGGEIA | Y369E, |
| | DLWLSNVDLELMKEHYDLYNVEYISGLKFKATTGLFKDFIDKWTEI | T372N, |
| | KNTSDGARKQLAKLMLNSLYGKFASNPDVTGKVPYLKENGALGF | E375D, I378R, |
| | RLGEEETKDPVYTPMGVFITAWARYTTITAAQACYDRIIYCDTDSIH | Q497P, |
| | LTGTEIPDVIKDIVDPKKLGYWAHESTFKRAKYLRPKTYIQDIYMK | K512E, F526L |
| | EVDGELVEGSPDDYTDIKLSVKCAGMTDKIKKEVTFENFKVGFSR | |
| | KMKPKPVQVPGGVVLVDDTFTIK | |

In some embodiments, a system or an engineered protein provided herein comprises an amino acid sequence that is at least 85% identical to any one of SEQ ID NOS: 63-76, 99-106. In some embodiments, a system or an engineered protein provided herein comprises an amino acid sequence that is at least 90% identical to any one of SEQ ID NOS: 63-76, 99-106. In some embodiments, a system or an engineered protein provided herein comprises an amino acid sequence that is at least 95% identical to any one of SEQ ID NOS: 63-76, 99-106. In some embodiments, a system or an engineered protein provided herein comprises an amino acid sequence that is at least 99% identical to any one of SEQ ID NOS: 63-76, 99-106. In some embodiments, a system or an engineered protein provided herein comprises any one of SEQ ID NOS: 63-76, 99-106.

In some embodiments, a DNA-dependent DNA polymerase provided herein comprises a modified T3 DNA-dependent DNA polymerase. In some embodiments, the modified T3 DNA-dependent DNA polymerase comprises a mutation, wherein the mutation is an amino acid substitution at position 5 or position 7 as compared to SEQ ID NO: 70. In some embodiments, the modified T3 DNA-dependent DNA polymerase comprises a mutation, wherein the mutation is an amino acid substitution at position 5 and position 7 as compared to SEQ ID NO: 70. In some embodiments, the modified T3 DNA-dependent DNA polymerase comprises a mutation, wherein the mutation comprises a D5A amino acid substitution. In some embodiments, the modified T3 DNA-dependent DNA polymerase comprises a mutation, wherein the mutation comprises a E7A amino acid substitution. In some embodiments, the modified T3 DNA-dependent polymerase comprises.

(SEQ ID NO: 107)
MLVSAIAANNLLEKVTKFHCGVIYDYRDGEYHSYRPGDFGAYLDALEAEVKRGGLIVFHNGHK
YDVPALTKLAKLQLNREFHLPRENCIDTLVLSRLIHSNLKDTDMGLLRSGKLPGKRFGSHALEA
WGYRLGEMKGEYKDDFKRMLEEQGEEYVDGMEWWNFNEEMMDYNVQDVVVTKALLEKLLS
DKHYFPPEIDFTDVGYTTFWSESLEAVDVEHRAAWLLAKQERNGFPFDTKAIEELYVELAARRSE
LLRNLTETFGSWYQPKGGTEMFCHPRTGKPLPKYPRIKIPKVGGIFKKPKNKAQREGREPCELDT
REYVAGAPYTPVEHVVFNPSSRDHIQKKLQEAGWVPTKFTDKGAPVVDDEVLEGVRVDDPEKQ
AAIDLIKEYLMIQKRIGQSAEGDKAWLRYVAEDGKIHGSVNPNGAVTGRATHAFPNLAQILRCL
AHFMARFDNGEYAHEILNGDIHTKNQMAAELPTRDNAKTFIYGFLYGAGDEKIGQIVGAGKERG
KELKKKFLENTPAIAALRESIQQTLVESSQWVAGEQQVKWKRRWIKGLDGRKVHVRSPHAALN
TLLQSAGALICKLWIIKTEEMLVEKGLKHGWDGDFAYMAWIHDEIQVACRTEEIAKTVIEVAQE
AMRWVGEHWNFRCLLDTEGKMGANWKECH

or a variant thereof. Modifications in SEQ ID: 107 relative to a T3 DNA-dependent DNA polymerase comprising SEQ ID NO: 70 are provided in bold/underlined font above.

In some embodiments, a DNA-dependent DNA polymerase provided herein comprises a modified T4 DNA-dependent DNA polymerase. In some embodiments, the modified T4 DNA-dependent DNA polymerase comprises a mutation, wherein the mutation is an amino acid substitution at position 112 or 114 as compared to SEQ ID NO: 71. In some embodiments, the modified T4 DNA-dependent DNA polymerase comprises a mutation, wherein the mutation is an amino acid substitution at position 112 and 114 as compared to SEQ ID NO: 71. In some embodiments, the modified T4 DNA-dependent DNA polymerase comprises a mutation, wherein the mutation comprises a D112A amino acid substitution. In some embodiments, the modified T4 DNA-dependent DNA polymerase comprises a mutation, wherein the mutation comprises a E114A amino acid substitution. In some embodiments, the modified T4 DNA-dependent polymerase comprises:

(SEQ ID NO: 108)
MKEFYISIETVGNNIVERYIDENGKERTREVEYLPTMFRHCKEESKYKDIYGKNCAPQKFMKDAR
DWMKRMEDIGLEALGMNDFKLAYISDTYGSEIVYDRKFVRVANCAIAVTGDKFPDPMKAEYEI
DAITHYDSIDDRFYVFDLLNSMYGSVSKWDAKLAAKLDCEGGDEVPQEILDRVIYMPFDNERDM
LMEYINLWEQKRPAIFTGWNIEGFDVPYIMNRVKMILGERSMKRFSPIGRVKSKLIQNMYGSKEI
YSIDGVSILDYLDLYKKFAFTNLPSFSLESVAQHETKKGKLPYDGPINKLRETNHQRYISYNIIDVE
SVQAIDKIRGFIDLVLSMSYYAKMPFSGVMSPIKTWDAIIFNSLKGEHKVIPQQGSHVKQSFPGAF
VFEPKPIARRYIMSFDLTSLYPSIIRQVNISPETIRGQFKVHPIHEYIAGTAPKPSDEYSCSPNGWMY
DKHQEGIIPKEIAKVFFQRKDWKKKMFAEEMNAEAIKKIIMKGAGSCSTKPEVERYVKFSDDFLN
ELSNYTESVLNSLIEECEKAATLANTNQLNRKILINSLYGALGNIHFRYYDLRNATAITIFGQVGIQ
WIARKINEYLNKVCGTNDEDFIAAGDTDSVYVCVDKVIEKVGLDRFKEQNDLVEFMNQFGKKK
MEPMIDVAYRELCDYMNNREHLMHMDREAISCPPLGSKGVGGFWKAKKRYALNVYDMEDKR
FAEPHLKIMGMETQQSSTPKAVQEALEESIRRILQEGEESVQEYYKNFEKEYRQLDYKVIAEVKT
ANDIAKYDDKGWPGFKCPFHIRGVLTYRRAVSGLGVAPILDGNKVMVLPLREGNPFGDKCIAWP
SGTELPKEIRSDVLSWIDHSTLFQKSFVKPLAGMCESAGMDYEEKASLDFLFG

or a variant thereof. Modifications in SEQ ID: 108 relative to a T4 DNA-dependent DNA polymerase comprising SEQ ID NO: 71 are provided in bold/underlined font above.
In some embodiments, a DNA-dependent DNA polymerase provided herein comprises a modified T5 DNA-dependent DNA polymerase. In some embodiments, the modified T5 DNA-dependent DNA polymerase comprises a mutation, wherein the mutation is an amino acid substitution at position 164 or 166 as compared to SEQ ID NO: 72. In some embodiments, the modified T5 DNA-dependent DNA polymerase comprises a mutation, wherein the mutation is an amino acid substitution at position 164 and 166 as compared to SEQ ID NO: 72. In some embodiments, the modified T5 DNA-dependent DNA polymerase comprises a mutation, wherein the mutation comprises a D164A amino acid substitution. In some embodiments, the modified T5 DNA-dependent DNA polymerase comprises a mutation, wherein the mutation comprises a E166A amino acid substitution. In some embodiments, the modified T5 DNA-dependent polymerase comprises:

(SEQ ID NO: 109)
MKIAVVDKALNNTRYDKHFQLYGEEVDVFHMCNEKLSGRLLKKHITIGTPENPFDPNDYDFVIL
VGAEPFLYFAGKKGIGDYTGKRVEYNGYANWIASISPAQLHFKPEMKPVFDATVENIHDIINGRE
KIAKAGDYRPITDPDEAEEYIKMVYNMVIGPVAFASATSALYCRDGYLLGVSISHQEYQGVYIDS
DCLTEVAVYYLQKILDSENHTIVFHNLKFDMHFYKYHLGLTFDKAHKERRLHDTMLQHYVLDE
RRGTHGLKSLAMKYTDMGDYDFELDKFKDDYCKAHKIKKEDFTYDLIPFDIMWPYAAKDTDAT
IRLHNFFLPKIEKNEKLCSLYYDVLMPGCVFLQRVEDRGVPISIDRLKEAQYQLTHNLNKAREKL
YTYPEVKQLEQDQNEAFNPNSVKQLRVLLFDYVGLTPTGKLTDTGADSTDAEALNELATQHPIA
KTLLEIRKLTKLISTYVEKILLSIDADGCIRTGFHEHMTTSGRLSSSGKLNLQQLPRDESIIKGCVVA
PPGYRVIAWDLTTAEVYYAAVLSGDRNMQQVFINMRNEPDKYPDFHSNIAHMVFKLQCEPRDV
KKLFPALRQAAKAITFGILYGSGPAKVAHSVNEALLEQAAKTGEPFVECTVADAKEYIETYFGQF
PQLKRWIDKCHDQIKNHGFIYSHFGRKRRLHNIHSEDRGVQGEEIRSGFNAIIQSASSDSLLLGAV
DADNEIISLGLEQEMKIVMLVHDSVVAIVREDLIDQYNEILIRNIQKDRGISIPGCPIGIDSDSEAGG
SRDYSCGKMKKQHPSIACIDDDEYTRYVKGVLLDAEFEYKKLAAMDKEHPDHSKYKDDKFIAV
CKDLDNVKRILGA

or a variant thereof. Modifications in SEQ ID: 109 relative to a T5 DNA-dependent DNA polymerase comprising SEQ ID NO: 72 are provided in bold/underlined font above.

In some embodiments, a DNA-dependent DNA polymerase provided herein comprises a modified T7 DNA-dependent DNA polymerase. In some embodiments, the modified T7 DNA-dependent DNA polymerase comprises a mutation, wherein the mutation is an amino acid substitution at position 5 or position 7 as compared to SEQ ID NO: 73. In some embodiments, the modified T7 DNA-dependent DNA polymerase comprises a mutation, wherein the mutation is an amino acid substitution at position 5 and position 7 as compared to SEQ ID NO: 73. In some embodiments, the modified T7 DNA-dependent DNA polymerase comprises a mutation, wherein the mutation comprises a D5A amino acid substitution. In some embodiments, the modified T7 DNA-dependent DNA polymerase comprises a mutation, wherein the mutation comprises a E7A amino acid substitution. In some embodiments, the modified T7 DNA-dependent polymerase comprises:

(SEQ ID NO: 110)
MIVSAIAEANALLESVTKFHCGVIYDYSTAEYVSYRPSDFGAYLDALEAEVARGGLIVFHNGHKY
DVPALTKLAKLQLNREFHLPRENCIDTLVLSRLIHSNLKDTDMGLLRSGKLPGKRFGSHALEAW
GYRLGEMKGEYKDDFKRMLEEQGEEYVDGMEWWNFNEEMMDYNVQDVVVTKALLEKLLSD
KHYFPPEIDFTDVGYTTFWSESLEAVDIEHRAAWLLAKQERNGFPFDTKAIEELYVELAARRSEL
LRKLTETFGSWYQPKGGTEMFCHPRTGKPLPKYPRIKTPKVGGIFKKPKNKAQREGREPCELDTR
EYVAGAPYTPVEHVVFNPSSRDHIQKKLQEAGWVPTKYTDKGAPVVDDEVLEGVRVDDPEKQA
AIDLIKEYLMIQKRIGQSAEGDKAWLRYVAEDGKIHGSVNPNGAVTGRATHAFPNLAQIPGVRSP
YGEQCRAAFGAEHHLDGITGKPWVQAGIDASGLELRCLAHFMARFDNGEYAHEILNGDIHTKN
QIAAELPTRDNAKTFIYGFLYGAGDEKIGQIVGAGKERGKELKKKFLENTPAIAALRESIQQTLVE
SSQWVAGEQQVKWKRRWIKGLDGRKVHVRSPHAALNTLLQSAGALICKLWIIKTEEMLVEKGL
KHGWDGDFAYMAWVHDEIQVGCRTEEIAQVVIETAQEAMRWVGDHWNFRCLLDTEGKMGPN
WAICH

or a variant thereof. Modifications in SEQ ID: 110 relative to a T7 DNA-dependent DNA polymerase comprising SEQ ID NO: 73 are provided in bold/underlined font above.

In some embodiments, a DNA-dependent DNA polymerase provided herein comprises a modified Klenow fragment, wherein the modified Klenow fragment comprises an amino acid substitution at position 33 or position 35 as compared to SEQ ID NO: 69. In some embodiments, the modified Klenow fragment comprises an amino acid substitution at position 33 and position 35 as compared to SEQ ID NO: 69. In some embodiments, the modified Klenow fragment comprises a D33A amino acid substitution as compared to SEQ ID NO: 69. In some embodiments, the modified Klenow fragment comprises a E35A amino acid substitution as compared to SEQ ID NO: 69. In some embodiments, the modified Klenow fragment comprises:

(SEQ ID NO: 264)
DMVISYDNYVTILDEETLKAWIAKLEKAPVFAFATATDSLDNISANLVGLSFAIEPGVAAYIPVAH
DYLDAPDQISRERALELLKPLLEDEKALKVGQNLKYDRGILANYGIELRGIAFDTMLESYILNSVA
GRHDMDSLAERWLKHKTITFEEIAGKGKNQLTFNQIALEEAGRYAAEDADVTLQLHLKMWPDL
QKHKGPLNVFENIEMPLVPVLSRIERNGVKIDPKVLHNHSEELTLRLAELEKKAHEIAGEEFNLSS
TKQLQTILFEKQGIKPLKKTPGGAPSTSEEVLEELALDYPLPKVILEYRGLAKLKSTYTDKLPLMI
NPKTGRVHTSYHQAVTATGRLSSTDPNLQNIPVRNEEGRRIRQAFIAPEDYVIVSADYSQIELRIM
AHLSRDKGLLTAFAEGKDIHRATAAEVFGLPLETVTSEQRRSAKAINFGLIYGMSAFGLARQLNIP
RKEAQKYMDLYFERYPGVLEYMERTRAQAKEQGYVETLDGRRLYLPDIKSSNGARRAAAERAA
INAPMQGTAADIIKRAMIAVDAWLQAEQPRVRMIMQVHDELVFEVHKDDVDAVAKQIHQLME
NCTRLDVPLLVEVGSGENWDQAH.

Modifications in SEQ ID: 264 relative to a Klenow fragment DNA-dependent DNA polymerase comprising SEQ ID NO: 69 are provided in bold/underlined text above.

In some embodiments, the DdDP, the DdDP region, or the DdDP fragment binds to the DNA-dependent DNA polymerase synthesis template (DST) region of the guide polynucleotide of the guide polynucleotide. In some embodiments, the DdDP, the DdDP region, or the DdDP fragment binds to the DST region of the guide polynucleotide. In some embodiments, the DdDP, the DdDP region, or the DdDP fragment binds to the hybridization region of the guide polynucleotide of the guide polynucleotide. In some embodiments, the DdDP, the DdDP region, or the DdDP fragment binds to the hybridization region of the guide polynucleotide. In some embodiments, the DdDP does not bind to the target nucleic acid. In some embodiments, the DdDP synthesizes a new DNA strand that has complementarity to the target nucleic acid. The new DNA strand can replace at least a portion of a target sequence in a double-stranded target nucleic acid, thereby editing the double-stranded target nucleic acid. In some embodiments, the new DNA strand restores gene expression of a protein encoded by the target nucleic acid.

In some embodiments, an engineered protein provided herein further comprises one or more additional protein or protein constructs. In some embodiments, the one or more additional protein or protein construct comprises: a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof. In some embodiments, the compositions and systems provided herein comprise an additional engineered protein. In some embodiments, the additional engineered protein comprises: a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof. In some embodiments, the engineered protein provided herein is in complex with a single-stranded polynucleotide that facilitates targeting of the nickase and/or the DNA polymerase provided herein.

In some embodiments, an engineered protein provided herein comprises a protein construct that modulates transcription. In some embodiments, an engineered protein provided herein comprises a transcriptional repressor protein construct. In some embodiments, an engineered protein provided herein comprises a zinc finger protein construct. In some embodiments, the zinc finger protein construct comprises a Krippel-associated box (KRAB) protein or a functional fragment thereof. In some embodiments, the engineered protein further comprises a KRAB domain that binds to a transcriptional corepressor protein. In some embodiments, an engineered protein provided herein comprises a SUMO protein construct.

In some embodiments, an engineered protein provided herein comprises a transcriptional activator protein construct. A transcriptional activator protein construct recruits transcription factors from a host cell to the target nucleic acid for regulation of target nucleic acid expression. Exemplary transcriptional activators include VP64, VP16, VP160, VP48, VP96, p65, Rta, VPR, hsf1, and p300. In some embodiments, an engineered protein provided herein comprises one or more VP16 protein construct. In some embodiments, an engineered protein provided herein comprises one or more VP64 protein construct. In some embodiments, an engineered protein provided herein comprises one or more VPR protein construct. In some embodiments, an engineered protein provided herein comprises one or more SunTag protein construct. In some embodiments, an engineered protein provided herein comprises VP64, p65, and HSF1 (SunTag-p65-heat-shock factor 1 or SPH). In some embodiments, an engineered protein provided herein comprises a CREB-binding protein (CPB). CBP can be used to recruit transcriptional machinery and function as a histone acetyltransferase (HAT) that alters chromatin structure.

In some embodiments, an engineered protein provided herein further comprises an aptamer.

In some embodiments, an engineered protein provided herein comprises a self-cleaving protein sequence. Non-limiting examples of self-cleaving protein sequences include: E2A, P2A and T2A. In some embodiments, an engineered protein provided herein further comprises an antibiotic resistance protein construct or an antibiotic resistance selectable marker. Non-limiting examples of antibiotic resistance proteins and selectable markers include: aminoglycoside acetyltransferase, rifampin ADP-ribosyltransferase, dihydrofolate reductase, multidrug and toxic compound extrusion transporters, antibiotic resistance ATP-binding cassette family F (ARE ABC-F) proteins (e.g., MsrE, Erm, Vga, Lsa, Sal, OptrA), β-lactamase, blasticidin-S deaminase, penicillin-binding proteins (PBPs), and puromycin-N-acetyltransferase.

In some embodiments, an engineered protein provided herein comprises a base editor. In some embodiments, the base editor is selected from the group consisting of: an adenine base editor, an adenosine base editor, a cytidine deaminase, a cytosine to guanine base editor, a deaminase dimer. In some embodiments, the cytidine deaminase is an activation-induced deaminase (AID), an APOBEC deaminase, APOBEC3G, APOBEC1, cytidine deaminase 1 (CDA1), a functional fragment, or a derivative thereof. In some embodiments, the adenosine base editor is ecTadA, saTadA, a functional fragment, or a derivative thereof. In some embodiments, the engineered proteins provided herein further comprise a uracil-DNA glycosylase, a uracil-DNA glycosylase inhibitor, a functional fragment, or a derivative thereof.

In some embodiments, a system, a composition, or an engineered protein provided herein further comprises a nuclear localization sequence (NLS). An NLS targets a protein to the nucleus of a cell, localizing an engineered protein provided herein in close proximity to a target nucleic acid within the nucleus of a cell. In some embodiments, a system, a composition, or an engineered protein comprises more than one nuclear localization sequence (NLS). In some embodiments, the NLS is from a Simian Vacuolating Virus 40 (SV40). In some embodiments, the NLS comprises a monopartite SV40 NLS. In some embodiments, the NLS comprises a bipartite SV40 NLS. In some embodiments, the NLS comprises PKKKRKV (SEQ ID NO: 77) or KRTADGSEFEPKKKRKV (SEQ ID NO: 78). In some embodiments, the NLS comprises a nucleoplasmin sequence. In some embodiments, the nucleoplasmin sequence comprises: KRPAATKKAGQAKKKK (SEQ ID NO: 79).

In some embodiments, a system, a composition, or an engineered protein provided herein further comprises a linker. A linker is a molecular entity that can directly or indirectly connect at two parts of a composition, e.g., from the first protein construct to the second protein construct and the second protein construct to a third protein construct, and so on. Linkers can be configured according to a specific need, e.g., stability or length between two amino acid sequences. In some embodiments, linkers can be configured to allow multimerization of the nuclease or nickase with a DdDP provided herein (e.g., to from a di-, tri-, tetra-, penta-, or higher multimeric complex) while retaining biological activity (e.g., cleavage of a target nucleic acid or set of target nucleic acids).

In some embodiments, the engineered proteins provided herein comprise a linker listed in Table 3.

TABLE 3
Linker Sequences.

| | Sequence (where ₙ = a |
|---|---|
| SEQ ID NO: | number between 1 and 1000) |
| SEQ ID NO: 80 | (GGS)ₙ |
| SEQ ID NO: 81 | (GGGGS)ₙ |
| SEQ ID NO: 82 | (EAAAK)ₙ |
| SEQ ID NO: 83 | MSRPDPA |
| SEQ ID NO: 84 | MKIIEQLPSA |
| SEQ ID NO: 85 | VRHKLKRVGS |
| SEQ ID NO: 86 | SIVAQLSRPDPA |
| SEQ ID NO: 87 | GHGTGSTGSGSS |
| SEQ ID NO: 88 | GSAGSAAGSGEF |
| SEQ ID NO: 89 | VPFLLEPDNINGKTC |
| SEQ ID NO: 90 | SGSETPGTSESATPES |
| SEQ ID NO: 91 | SGGSSGGSSGSETPGTSESATPESSGGSSG |
| | GSS |

In some embodiments, linkers are configured to facilitate expression and purification of the engineered proteins provided herein. In some embodiments, an engineered protein provided herein comprises a cleavable linker or a non-cleavable linker between the different protein constructs or domains of the engineered protein. For example, a linker can be a polypeptide linker, such as a linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids long. Generally, the number of additional elements in the engineered protein will depend on a number of factors, including, e.g., size or molecule weight of the protein for delivery to a cell, or translocation of the protein desired in a cell type of interest.

Provided herein are engineered proteins and polynucleotides encoding for engineered proteins, wherein the engineered proteins comprise: (a) an engineered protein construct comprising a nickase region or a nuclease region; and (b) a DNA-dependent DNA polymerase (DdDP) or a functional fragment thereof. In some embodiments, the engineered protein further comprises a linker between the nickase region or the nuclease region; and the DdDP or the functional fragment thereof. In some embodiments, the linker comprises an amino acid sequence that is at least 95% identical to any one of SEQ ID NOS: 80-91. In some embodiments, the linker comprises an amino acid sequence that is at least 99% identical to any one of SEQ ID NOS: 80-91. In some embodiments, the linker comprises any one of SEQ ID NOS: 80-91.

### Combination Compositions

Provided herein are compositions comprising: (a) a polynucleotide a DNA-dependent DNA polymerase, optionally wherein the polynucleotide encodes for a phi29 DNA-dependent DNA polymerase or a variant thereof; and (b) a polynucleotide encoding a nickase provided herein. In some embodiments, the DNA-dependent DNA polymerase and the nickase are linked together as a fusion protein by a linker provided herein. In some embodiments, the DNA-dependent DNA polymerase and the nickase are encoded by a single polynucleotide. In some embodiments, the DNA-dependent DNA polymerase and the nickase are encoded by a plurality of polynucleotides. In some embodiments, the nickase comprises a Type II Cas protein. In some embodiments, the Type II Cas protein comprises: a Cas9, a Cas1, a Cas2, or a Csn2. In some embodiments, the Cas9 protein comprises a Streptococcus pyogenes Cas9 protein, a Streptococcus pyogenes Cas9 nickase, or a variant thereof. In some embodiments, the Cas9 protein comprises a Streptococcus thermophilus Cas9 protein, a Streptococcus thermophilus Cas9 nickase, or a variant thereof. In some embodiments, the phi29 DNA-dependent DNA polymerase comprises a sequence that is at least 90% identical to SEQ ID NO: 63. In some embodiments, the phi29 DNA-dependent DNA polymerase variant comprises a mutation, wherein the mutation comprises an amino acid substitution of an amino acid residue of position: 8, 12, 51, 66, 97, 137, 197, 221, 369, 372, 375, 377, 378, 497, 512, or 526 as compared to SEQ ID NO: 63. In some embodiments, the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue. In some embodiments, the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is an arginine (Arg, R), an alanine (Ala, A), a glutamic acid (Glu, E), a proline (Pro, P), a threonine (Thr, T), an aspartic acid (Asp, D), a cysteine (Cys, C), a leucine (Leu, L) or a lysine (Lys, K). In some embodiments, the amino acid substitution comprises: M8R, D12A, V51A, D66A, M97T, F137C, G197D, E221K, Y369E, T372N, E375D, A377C, I378R, Q497P, K512E, F526L, or any combination thereof. In some embodiments, the phi29 DNA-dependent DNA polymerase variant comprises any one of SEQ ID NO: 99-SEQ ID NO: 106.

In some embodiments, the phi29 DNA-dependent DNA polymerase variant comprises SEQ ID NO: 102. In some embodiments, the phi29 DNA-dependent DNA polymerase variant comprises SEQ ID NO: 104. In some embodiments, the phi29 DNA-dependent DNA polymerase variant comprises SEQ ID NO: 105. In some embodiments, the composition further comprises a guide polynucleotide provided herein. In some embodiments, the composition further comprises a target polynucleotide provided herein. In some embodiments, the composition further comprises a cell, a nucleus, or a target DNA sequence.

Provided herein are compositions comprising: (a) a polynucleotide encoding a DNA-dependent DNA polymerase or a variant thereof; and (b) a polynucleotide encoding a nickase, optionally wherein the polynucleotide encodes for a Streptococcus pyogenes Cas9 nickase or a variant thereof. In some embodiments, the DNA-dependent polymerase or the variant thereof and the nickase are linked by a linker provided herein as a fusion protein. In some embodiments, the DNA-dependent DNA polymerase and the nickase are encoded by a single polynucleotide. In some embodiments, the DNA-dependent DNA polymerase and the nickase are encoded by a plurality of polynucleotides. In some embodiments, the DNA-dependent polymerase comprises a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol ρ, a Pol κ, a Pol ι, a Pol η, a Pol IV, a Pol V, a terminal deoxynucleotidyl transferase, or a combination thereof. In some embodiments, the DNA-dependent polymerase comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof. In some embodiments, the Streptococcus pyogenes Cas9 nickase variant comprises an amino acid substitution at position: 61, 221, 394, 840, 1111, 1135, 1136, 1137, 1218, 1219, 1317, 1322, 1333, 1335, or 1337 as compared to SEQ ID NO: 92. In some embodiments, the Streptococcus pyogenes Cas9 nickase variant comprises an amino acid substitution, wherein the amino acid substitution comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue. In some embodiments, the Streptococcus pyogenes Cas9 nickase variant comprises an amino acid substitution comprises an amino acid substitution at position 840. In some embodiments, the Streptococcus pyogenes Cas9 nickase variant comprises an amino acid substitution comprises an amino acid substitution at position 221, 394, and/or 840. In some embodiments, the amino acid substitution comprises H840A. In some embodiments, the amino acid substitution comprises: R221K, N394K, H840A, and/or any combination thereof. In some embodiments, the Streptococcus pyogenes Cas9 nickase variant comprises amino acid substitutions at positions 221, 394, 840, 1135, 1218, 1335, and/or 1337. In some embodiments, the Streptococcus pyogenes Cas9 nickase variant comprises amino acid substitutions selected from: R221K, N394K, H840A, D1135V, G1218R, R1335Q, and T1337R. In some embodiments, the Streptococcus pyogenes Cas9 nickase variant comprises amino acid substitutions at positions 221, 394, 840, 1135, 1136, 1218, 1219, 1335, and/or 1337. In some embodiments, the Streptococcus pyogenes Cas9 nickase variant comprises amino acid substitutions selected from: R221K, N394K, H840A, D1135L, S1136W, G1218K, E1219Q, R1335Q, and T1337R. In some embodiments, the Streptococcus pyogenes Cas9 nickase variant comprises amino acid substitutions at positions 61, 221, 394, 840, 1111, 1135, 1136, 1218, 1219, 1317, 1322, 1333, 1335, and/or 1337. In some embodiments, the Streptococcus pyogenes Cas9 nickase variant comprises amino acid selected from: A61R, R221K, N394K, H840A, L1111R, D1135L, S1136W, G1218K, E1219Q, N1317R, A1322R, R1333P, R1335Q, and T1337R. In some embodiments, the Streptococcus pyogenes Cas9 nickase variant comprises a sequence selected from any one of SEQ ID NO: 93 to SEQ ID NO: 96. In some embodiments, the Streptococcus pyogenes Cas9 nickase variant comprises SEQ ID NO: 93. In some embodiments, the composition further comprises a guide polynucleotide provided herein. In some embodiments, the composition further comprises a target polynucleotide provided herein. In some embodiments, the composition further comprises a cell, a nucleus, or a target DNA sequence.

Provided herein are compositions comprising: (a) a polynucleotide encoding a DNA-dependent DNA polymerase or a variant thereof; and (b) a polynucleotide encoding a nickase, optionally wherein the polynucleotide encodes for a Streptococcus thermophilus Cas9 nickase or a variant thereof. In some embodiments, the DNA-dependent DNA polymerase and the nickase are encoded by a single polynucleotide. In some embodiments, the DNA-dependent DNA polymerase and the nickase are encoded by a plurality of polynucleotides. In some embodiments, the DNA-dependent polymerase or the variant thereof and the nickase are linked by a linker provided herein as a fusion protein. In some embodiments, the DNA-dependent polymerase comprises a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol μ, a Pol κ, a Pol ι, a Pol η, a Pol IV, a Pol V, a terminal deoxynucleotidyl transferase, or a combination thereof. In some embodiments, the DNA-dependent polymerase comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof. In some embodiments, the Streptococcus thermophilus Cas9 nickase variant comprises an amino acid substitution at position 599 as compared to SEQ ID NO: 97. In some embodiments, the Streptococcus thermophilus Cas9 nickase variant comprises an amino acid substitution that comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue. In some embodiments, the Streptococcus thermophilus Cas9 nickase variant comprises an amino acid substitution of H599A. In some embodiments, the Streptococcus thermophilus Cas9 nickase comprises SEQ ID NO: 97. In some embodiments, the Streptococcus thermophilus Cas9 nickase variant comprises SEQ ID NO: 98. In some embodiments, the composition further comprises a guide polynucleotide provided herein. In some embodiments, the composition further comprises a target polynucleotide provided herein. In some embodiments, the composition further comprises a cell, a nucleus, or a target DNA sequence.

Provided herein are engineered fusion proteins comprising: (a) a phi29 DNA-dependent DNA polymerase or a variant thereof, and (b) a nickase or a variant thereof. In some embodiments, the engineered fusion protein comprises a phi29 DNA-dependent DNA polymerase having a sequence that is at least 90% identical to SEQ ID NO: 63. In some embodiments, the engineered fusion protein comprises a phi29 DNA-dependent DNA polymerase having a sequence that is at least 95% identical to SEQ ID NO: 63. In some embodiments, the engineered fusion protein comprises a phi29 DNA-dependent DNA polymerase having a sequence that is at least 99% identical to SEQ ID NO: 63. In some embodiments, the engineered fusion protein comprises a phi29 DNA-dependent DNA polymerase having the sequence of SEQ ID NO: 63. In some embodiments, the engineered fusion protein comprises the phi29 DNA-dependent DNA polymerase variant, wherein the phi29 DNA-dependent DNA polymerase variant comprises a sequence that is at least 90% identical to any one of SEQ ID NO: 99-SEQ ID NO: 106. In some embodiments, the engineered fusion protein comprises the phi29 DNA-dependent DNA polymerase variant, wherein the phi29 DNA-dependent DNA polymerase variant comprises a sequence that is at least 95% identical to any one of SEQ ID NO: 99-SEQ ID NO: 106. In some embodiments, the engineered fusion protein comprises the phi29 DNA-dependent DNA polymerase variant, wherein the phi29 DNA-dependent DNA polymerase variant comprises a sequence that is at least 99% identical to any one of SEQ ID NO: 99-SEQ ID NO: 106. In some embodiments, wherein the engineered fusion protein comprises the phi29 DNA-dependent DNA polymerase variant, wherein the phi29 DNA-dependent DNA polymerase variant comprises any one of SEQ ID NO: 99-SEQ ID NO: 106. In some embodiments, the phi29 DNA-dependent DNA polymerase variant comprises SEQ ID NO: 102, SEQ ID NO: 103, or SEQ ID NO: 104. In some embodiments, the engineered fusion protein comprises a Cas protein or a mutant Cas protein. In some embodiments, the fusion protein a Type V Cas protein. In some embodiments, the engineered fusion protein comprises a Cas12a, a Cas12b, a Cas12c, a Cas12d, a Cas12e, a Cas14, a Cas12g, a Cas12h, a Cas12i, a Cas12j, or a Cas12k. In some embodiments, the engineered fusion protein comprises a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Csc1, a Csc2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a SpCas9-VQR, a SpCas9-VRQR, a SpCas9-VRER, a SaCas9-KKH, a SpCas9-NG, a SpCas9-NRRH, a SpCas9-NRTH, a SpCas9-NRCH, a iSpyMac, a St1Cas9 LMD9-LMG18311, a St1Cas9 LMD9-CNRZ1066, a St1Cas9-KQKL, a variant, or any combination thereof. In some embodiments, the engineered fusion protein comprises a Type II Cas protein. In some embodiments, the engineered fusion protein comprises a Cas9, an SpCas9, an St1Cas9, a Cas1, a Cas2, or a Csn2. In some embodiments, the engineered fusion protein comprises a nickase having SEQ ID NO: 93. In some embodiments, the engineered fusion protein further comprises a linker, a nuclear localization sequence (NLS), or an additional protein construct. In some embodiments, the engineered fusion protein further comprises a cell-targeting moiety, a receptor-targeting moiety, a regulatory element, a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof.

Provided herein are engineered fusion proteins comprising a sequence having at least 90% identity to any one of SEQ ID NO: 250, SEQ ID NO: 252, SEQ ID NO: 255, or SEQ ID NO: 256. Provided herein are engineered fusion proteins comprising a sequence having at least 95% identity to any one of SEQ ID NO: 250, SEQ ID NO: 252, SEQ ID NO: 255, or SEQ ID NO: 256. Provided herein are engineered fusion proteins comprising a sequence having at least 96% identity to any one of SEQ ID NO: 250, SEQ ID NO: 252, SEQ ID NO: 255, or SEQ ID NO: 256. Provided herein are engineered fusion proteins comprising a sequence having at least 97% identity to any one of SEQ ID NO: 250, SEQ ID NO: 252, SEQ ID NO: 255, or SEQ ID NO: 256. Provided herein are engineered fusion proteins comprising a sequence having at least 98% identity to any one of SEQ ID NO: 250, SEQ ID NO: 252, SEQ ID NO: 255, or SEQ ID NO: 256. Provided herein are engineered fusion proteins comprising a sequence having at least 99% identity to any one of SEQ ID NO: 250, SEQ ID NO: 252, SEQ ID NO: 255, or SEQ ID NO: 256. Provided herein are engineered fusion proteins comprising a sequence having at least 95% identity to any one of SEQ ID NO: 250, SEQ ID NO: 252, SEQ ID NO: 255, or SEQ ID NO: 256, wherein the engineered fusion proteins comprise at least one, at least two, at least three, at least four, at least five, at least six, or at least seven amino acid substitutions in the DNA-dependent DNA polymerase region. Provided herein are engineered fusion proteins comprising a sequence having at least 95% identity to any one of SEQ ID NO: 250, SEQ ID NO: 252, SEQ ID NO: 255, or SEQ ID NO: 256, wherein the engineered fusion proteins comprise at least one, at least two, at least three, at least four, at least five, at least six, or at least seven amino acid substitutions in the nuclease or the nickase region. Provided herein are engineered fusion proteins comprising an amino acid sequence of any one of SEQ ID NO: 250, SEQ ID NO: 252, SEQ ID NO: 255, or SEQ ID NO: 256.

Provided herein are nucleic acids and polynucleotides encoding any one of the engineered fusion proteins provided herein. In some embodiments, the polynucleotide encoding the engineered fusion protein comprises DNA. In some embodiments, the polynucleotide encoding the engineered fusion protein comprises RNA. Provided herein are vectors comprising any nucleic acid or polynucleotide provided herein or a plurality of nucleic acids or polynucleotides as provided herein. In some embodiments, the nucleic acids, vectors, or viral vectors provided herein further comprise a guide polynucleotide provided herein. In some embodiments, the nucleic acids, vectors, or viral vectors provided herein further comprise a DNA encoding for a guide polynucleotide provided herein.

The engineered proteins, fusion proteins, DNA-dependent DNA polymerases, nucleases, and nickases provided herein can comprise one or more modifications or mutations in the amino acid sequence. The modification can comprise a substitution, a deletion, or an insertion of one or more amino acids in a protein sequence by modifying the nucleic acid encoding the protein. For example, up to 1, 2, 3, 4, 5, 6, 7, 8, 9, 10, 11, 12, 13, 14, 15, 20, 25, 30, 40, 50, or more nucleotides/amino acids in a polynucleotide (cDNA, gene) or a polypeptide sequence can be substituted, deleted, and/or inserted. A mutation can affect the coding sequence of a gene or its regulatory sequence. A mutation can also affect the structure of the genomic sequence or the structure/stability of the encoded mRNA and/or the translated polypeptide structure and function. Methods of modifying a nucleic acid sequence encoding an engineered protein provided herein can include, for example, direct mutagenesis or random mutagenesis.

In some embodiments, a nuclease or a nickase provided herein comprises one or more amino acid substitution. In some embodiments, a nuclease or a nickase provided herein comprises two or more amino acid substitutions. In some embodiments, a nuclease or a nickase provided herein comprises three or more amino acid substitutions. In some embodiments, a DNA-dependent DNA polymerase provided herein comprises one or more amino acid substitution. In some embodiments, a DNA-dependent DNA polymerase provided herein comprises two or more amino acid substitutions. In some embodiments, a DNA-dependent DNA polymerase provided herein comprises three or more amino acid substitutions.

### Target Nucleic Acids

A guide polynucleotide provided herein can comprise a degree of complementarity to a target polynucleotide sequence of interest or a strand thereof. A targeting region of a guide polynucleotide provided herein can comprise at least partial sequence complementarity to a target polynucleotide. The targeting sequence may have a degree of sequence complementarity to the target nucleic acid that is sufficient for the guide polynucleotide to hybridize with the target polynucleotide. In some cases, the targeting sequence comprises 95%, 96%, 97%, 98%, 99%, or 100% sequence complementarity to the target polynucleotide. Sequence complementarity can be determined by using alignment methods known in the art, for instance alignment of the sequences can be conducted using publicly available software such as BLAST, Align, ClustalW2. In some embodiments, a target nucleic acid provided herein comprises a gene or a polynucleotide comprising DNA. In some embodiments, the gene or the polynucleotide comprises a mammalian gene or polynucleotide. In some embodiments, the gene or the polynucleotide comprises a human gene or polynucleotide. In some embodiments, a target nucleic acid provided herein comprises a DNA. In some embodiments, a target nucleic acid provided herein comprises a single-stranded DNA. In some embodiments, a target nucleic acid provided herein comprises double-stranded DNA In some embodiments, a target nucleic acid provided herein comprises RNA. In some embodiments, a target nucleic acid provided herein comprises a single-stranded RNA. In some embodiments, a target nucleic acid provided herein comprises double-stranded RNA.

In some embodiments, the target nucleic acid or a complementary strand provided herein comprises a protospacer-adjacent motif (“PAM”), wherein the PAM is a short T-rich sequence. In some embodiments, cleavage of a target nucleic acid by the engineered protein provided herein occurs downstream or 3′ from the PAM sequence. In some embodiments, cleavage of a target nucleic acid occurs upstream or 5′ from the PAM sequence. In some embodiments, the PAM is an NGG PAM sequence. In some embodiments, the PAM comprises NGAN, NGNG, NGAG, NGCG, wherein N is A, G, C or T. In some embodiments, the PAM is a T-rich PAM. In some embodiments, the PAM has the nucleotide sequence (T)XN, wherein the X is the number of thymines (e.g., 1-10), and N is A, G, C or T. In certain embodiments, X is equal to 2, and thus, the PAM is TTN. In some embodiments, X is 3, and thus, the PAM is TTTN. In some embodiments, a nuclease or a nickase provided herein recognizes the sequence motif TTTN and directs cleavage of a target nucleic acid sequence 1-24 bp (e.g., 1, 2, 3, 4, 5, 6, 7, 8, 9, 10, 11, 12, 13, 14, 15, 16, 17, 18, 19, 20, 21, 22, 23, or 24) downstream from that sequence.

Without limitation, a target nucleic acid sequence can be from any cell or organism. Determining the appropriate sequence for the guide polynucleotide to bind to a target nucleic acid provided herein will depend on the sequence of the desired target and structure of the nuclease or the nickase provided herein. Methods of designing the targeting region of a guide polynucleotide for gene editing are known in the art and include, e.g., using software and databases such as Breaking-Cas, Cas-OFFinder, CRISPR-DT, CHOPCHOP, CCTOP, CRISPick, or CRISPOR.

In some embodiments, the target nucleic acid comprises a double-stranded DNA (dsDNA). In the case of dsDNA, the polynucleotide and the nickase region of the engineered protein provided herein binds to the target nucleic acid and generates a single-strand break in the dsDNA. The cleavage can occur on the bottom strand or the top strand of the double stranded DNA. Cleavage of the dsDNA generates a DNA replication fork and a leading strand and a complementary strand (also called a lagging strand) form. The guide polynucleotide binds to both strands of the target DNA via different regions of the guide polynucleotide. For example, the hybridization region binds to the leading strand of the DNA following cleavage of the dsDNA by a nickase or a nickase region of an engineered polynucleotide. The targeting region of the guide polynucleotide comprises RNA and binds to the complementary strand that comprises a PAM sequence. Once the hybridization region is bound to the leading strand, the DdDP of the engineered protein synthesizes a new strand using the DST region as a template. The DdDP does not bind to the complementary strand comprising the PAM sequence. The DdDP synthesizes DNA that are complementary to the DST region. Once, the DdDP has completed DNA synthesis of the new strand, the guide polynucleotide dissociates from the complementary strand of the nicked target nucleic acid and the new strand is incorporated into the target nucleic acid. In some cases, a DNA bulge forms where the DNA edit is included in the newly synthesized DNA as the sequence differs from the complementary target DNA strand. Additional DNA mismatch repair proteins recognize the bulge and can promote the correction of an aberrant sequence in the target nucleic acid (relative to a wild-type reference sequence) and modify the target nucleic acid. In some embodiments, a break in the target nucleic acid may be repaired by homology directed repair (HDR) proteins or non-homologous end joining (NHEJ) repair proteins. In some embodiments, the HDR proteins and/or the NHEJ proteins are endogenous to a cell. In some embodiments, the HDR proteins and/or the NHEJ proteins are introduced to a cell or a cell-free system. In some cases, when the nickase cleaves the bottom strand of DNA, for example, the complementary strand, away from the edit, the newly synthesized DNA edit can integrate into the genome.

### (2) Delivery Vehicles and Vectors

Provided herein are compositions comprising a guide polynucleotide provided herein and a delivery vehicle. Provided herein are compositions comprising an engineered protein provided herein and a delivery vehicle. Provided herein are systems provided herein and one or more delivery vehicles. The compositions and cells provided herein can be delivered to a target cell, tissue, organ, or subject by any suitable means.

The engineered proteins, guide polynucleotides, and any polynucleotide encoding for the engineered proteins or the guide polynucleotides provided herein can be admixed with a delivery vehicle that permits delivery of the system to the target nucleic acid sequence within a cell, tissue, or subject. Polynucleotides and sets of polynucleotides that encode the engineered protein and/or the guide polynucleotide provided herein comprises DNA, RNA, or both DNA and RNA. In some embodiments, an RNA encodes for the engineered protein provided herein or the guide polynucleotides provided herein. For example, RNA delivery of proteins can improve expression of the engineered proteins in human cells. In some embodiments, the polynucleotides provided herein comprises a self-replicating RNA or a viral RNA.

In some embodiments, the delivery vehicle comprises a vector, a lipid, a nanoparticle, a plasmid, a virus, a liposome, an extracellular vesicle, or a combination thereof. Additional non-limiting examples of delivery vehicles include an emulsion, a suspension, a liposome, a micelle, an exosome, an endosome, a virus, a vector, a particle, a nanoparticle, a polymer, microcapsules, recombinant cells, cell culture medium, blood, or serum. Specific types of delivery vehicles that can be used in a composition provided herein are further described below.

In some embodiments, the delivery vehicle is a liposome. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 pm. Sonication of MLVs results in the formation of small unilamellar vesicles (SUVs) with diameters in the range of 200 to 500 angstroms containing an aqueous solution in the core. Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 pm) should be designed using polymers able to be degraded in vivo. Biodegradable polyalkyl-cyanoacrylate nanoparticles can also be used as a delivery vehicle.

In some embodiments, the delivery vehicle is a phospholipid. Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a tightly packed, ordered structure, known as the gel state, to a loosely packed, less-ordered structure, known as the fluid state. This occurs at a characteristic phase-transition temperature and results in an increase in permeability to ions, sugars and drugs.

In some embodiments, the delivery vehicle is a nanoparticle. Nanoparticle carriers that specifically target a tissue provided herein may also be used as a pharmaceutically acceptable carrier. In some embodiments, the nanoparticle is a gold nanoparticle, a platinum nanoparticle, an iron-oxide nanoparticle, a lipid nanoparticle, a selenium nanoparticle, a tumor-targeting glycol chitosan nanoparticle (CNP), a cathepsin B sensitive nanoparticle, a hyaluronic acid nanoparticle, a paramagnetic nanoparticle, or a polymeric nanoparticle. In some embodiments, the delivery vehicle is a lipid nanoparticle.

Provided herein are compositions comprising a lipid nanoparticle comprising: a system provided herein, a set of polynucleotides encoding the system provided herein, a vector provided herein, an engineered protein provided herein, a guide polynucleotide or a portion thereof, or any composition provided herein. In some embodiments, the lipid nanoparticle is a solid lipid nanoparticle (SLN) or a nanostructured lipid carrier (NLC).

The lipid nanoparticles provided herein can comprise cationic lipids, ionizable lipids, a mixture of lipids, zwitterionic lipids, and/or phospholipids. In some embodiments, the lipid nanoparticle comprises a cationic lipid selected from the group consisting of 1,2-di-O-octadecenyl-3-trimethylammonium-propane (DOTMA), 1,2-dioleoy]-sn-glycero-3-phosphoethanolamine (DOPE), 1,2-dioleoyl-3-trimethylammonium-propane (DOTAP), Dimethyldioctadecylammonium bromide (DDAB), and Ethylphosphatidylcholine (ePC). In some embodiments, the lipid nanoparticle comprises an ionizable lipid selected from the group consisting of: 2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol and N4-cholesteryl-spermine (GL67), 9Z,12Z-octadecadienoic acid, 3-[4,4-bis(octyloxy)-1-oxobutoxy]-2-[[[[3-(diethylamino)propoxy]carbonyl]oxy]methyl]propyl ester (LPO1), heptadecan-9-yl 8-[2-hydroxyethyl-(6-oxo-6-undecoxyhexyl)amino]octanoate (SM-102), [(4-Hydroxybutyl)azanediyl]di(hexane-6,1-diyl) bis(2-hexyldecanoate) (ALC-0315), bis(2-butyloctyl) 10-(N-(3-(dimethylamino)propyl)nonanamido)nonadecanedioate (Lipid A9), 5-(dimethylamino)-pentanoic acid, (6Z)-1,2-di-(4Z)-4-decen-1-yl-6-dodecen-1-yl ester (Lipid CL1), or 7-[(2-Hydroxyethyl)[8-(nonyloxy)-8-oxooctyl]amino]heptyl 2-octyldecanoate (Lipid 5). In some embodiments, the lipid nanoparticle comprises a phosphatidylcholine. In some embodiments, the lipid nanoparticle comprises cholesterol or a cholesterol analog. In some embodiments, the lipid nanoparticle comprises a cholesterol analog selected from the group consisting of β-sitosterol, Vitamin D3, Vitamin D2, calcipotriol, stigmasterol, betulin, lupeol, ursolic acid, oleanolic acid, stigmastanol, campesterol, fucosterol, brassicasterol, and ergosterol.

In some embodiments, the lipid nanoparticle comprises polyethylene glycol (PEG). In some embodiments, the PEG is 1,2-dimyristoyl-rac-glycero-3-methoxypolyethylene glycol-2000 (PEG₂₀₀₀-DMG) or 1,2-distearoyl-rac-glycero-3-methoxypolyethylene glycol-2000 (PEG₂₀₀₀-DSG). In some embodiments, the lipid nanoparticle comprises N-acetylgalactosamine (GalNAc). In some embodiments, the GalNAc is 1,2-distearoyl-sn-glycero-3-phosphoethanolamine-N-[tris-GalNAc-GABA-(polyethylene glycol)-2000](Tri-GalNAc-PEG2000-DSPE).

In some embodiments, the composition comprising the lipid nanoparticle is in the form of a emulsion. In some embodiments, the composition comprising the lipid nanoparticle is in the form of a liquid. In some embodiments, the composition comprising the lipid nanoparticle is in the form of a gel. In some embodiments, the composition comprising the lipid nanoparticle is in the form of a solid. In some embodiments, the system provided herein, the set of polynucleotides encoding the system provided herein, the vector provided herein, the engineered protein provided herein, or the guide polynucleotide or a portion thereof are encapsulated by the lipid nanoparticle. In some embodiments, the system provided herein, the set of polynucleotides encoding the system provided herein, the vector provided herein, the engineered protein provided herein, or the guide polynucleotide or a portion thereof are in complex with the lipid nanoparticle.

The compositions provided herein can be delivered to a cell system using vectors, for example containing polynucleotide sequences encoding a system, a guide polynucleotide, an engineered protein, or a composition provided herein. In some embodiments, a system as described herein can be delivered absent a viral vector. Any vector systems can be used including, but not limited to, plasmid vectors, viral vectors, and oncolytic viral vectors. Furthermore, any of these vectors can comprise one or more transcription factor, transgene, or molecular tag.

In some embodiments, the vectors provided herein are viral vectors. Exemplary viral vectors include, but are not limited to, lentiviral vectors, retroviral vectors, adeno-associated viral vectors (AAV), adenoviral vectors, herpes simplex viral vectors, alphaviral vectors, flaviviral vectors, rhabdoviral vectors, measles viral vectors, Newcastle disease viral vectors, poxviral vectors, picornaviral vectors, and oncolytic viral vectors.

In some embodiments, the viral vector comprises an AAV. AAVs can have one or more of the AAV wild-type genes deleted in whole or part, e.g., the rep and/or cap genes, but retain functional flanking ITR sequences. Functional ITR sequences are necessary for the rescue, replication, and packaging of the AAV virion. The ITRs need not be the wild-type nucleotide sequences, and may be altered, e.g., by the insertion, deletion or substitution of nucleotides, so long as the sequences provide for functional rescue, replication and packaging. A recombinant AAV vector (rAAV) comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. In some embodiments, the AAV or the rAAV provided herein comprises a serotype of AAV1, AAV2, AAV3, AAV4, AAV5, AAV6, AAV7, AAV8, AAV9, AAV10, AAV11, AAV12, AAV13, AAVrh10, or any combination thereof. In some embodiments, the delivery vehicle comprises a hybrid AAV-lipid nanoparticle delivery system.

In some embodiments, the viral vector is a lentiviral vector. In some embodiments, the lentiviral vector is selected from the group consisting of: a human immunodeficiency virus 1 (HIV-1); a human immunodeficiency virus 2 (HIV-2), a visna-maedi virus (VMV) virus; a caprine arthritis-encephalitis virus (CAEV); an equine infectious anemia virus (EIAV); a feline immunodeficiency virus (FIV); a bovine immune deficiency virus (BIV); and a simian immunodeficiency virus (SIV), fragments, derivatives, or variants thereof.

Conventional viral and non-viral based gene transfer methods can be used to introduce polynucleotides encoding for a composition, system, guide polynucleotide, or an engineered protein provided herein to cells (e.g., mammalian cells) and target tissues. Exemplary non-viral vector delivery systems can include DNA plasmids, naked nucleic acid, and nucleic acids complexed with a delivery vehicle such as a liposome or poloxamer. Viral vector delivery systems can also include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. Additional exemplary nucleic acid delivery systems include those provided by AMAXA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. Lipofection reagents are sold commercially (e.g., TRANSFECTAM® and LIPOFECTIN®).

Delivery of the compositions and systems provided herein can be to cells (ex vivo administration) or target tissues (in vivo administration). Additional methods of delivery include the use of packaging the polynucleotides to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

Vectors including viral and non-viral vectors containing nucleic acids encoding a nucleic editing system provided herein can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. More than one route can be used to administer a particular composition.

In some embodiments, a composition, protein, guide polynucleotide, or a system provided herein can be shuttled to a cellular nucleus. For example, a vector can contain a nuclear localization sequence (NLS). A vector or any composition provided herein can also be shuttled by a protein or protein complex. In some embodiments, a composition or a system provided herein can be introduced to a cell or a target tissue by a minicircle vector.

In some embodiments, a vector or a polynucleotide provided herein can be pre-complexed with an engineered protein provided herein prior to electroporation into a cell. An engineered protein that can be used for shuttling can be a nickase or a catalytically dead Cas protein. A nuclease that can be used for shuttling can be a nuclease-competent protein. In some embodiments, an engineered protein herein can be pre-mixed with a guide polynucleotide provided herein an any additional elements (e.g., transgenes or other engineered proteins).

A cell can be transfected with a mutant or chimeric adeno-associated viral vector encoding a system or a composition provided herein. For example, an AAV vector concentration can be from 0.5 nanograms to 50 micrograms.

A system or a composition provided herein can also be introduced to a cell via electroporation techniques. The amount of polynucleotides that can be introduced into the cell by electroporation can be varied to optimize transfection efficiency and/or cell viability. In some embodiments, less than about 100 picograms of nucleic acid can be added to each cell sample (e.g., one or more cells being electroporated). In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid can be added to each cell sample (e.g., one or more cells being electroporated). For example, 1 microgram of dsDNA can be added to each cell sample for electroporation. In some embodiments, the amount of nucleic acid (e.g., dsDNA) required for optimal transfection efficiency and/or cell viability can be specific to the cell type. In some embodiments, the amount of nucleic acid (e.g., dsDNA) used for each sample can directly correspond to the transfection efficiency and/or cell viability. The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Viral particles, such as AAV, can be used to deliver a viral vector comprising a gene of interest or a transgene into a cell ex vivo or in vivo. In some embodiments, a mutated or chimeric adeno-associated viral vector as disclosed herein can be measured as pfu (plaque forming units). In some embodiments, the pfu of recombinant virus or mutated or chimeric adeno-associated viral vector of the compositions and methods of the disclosure can be about 10⁸ to about 5×10¹⁰ pfu. In some embodiments, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, a mutated or chimeric adeno-associated viral vector of the disclosure can be measured as vector genomes. In some embodiments, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some embodiments, a mutated or chimeric adeno-associated viral vector of the disclosure can be measured using multiplicity of infection (MOI). In some embodiments, MOI can refer to the ratio, or multiple of vector or viral genomes to the cells to which the nucleic can be delivered. In some embodiments, MOI can refer to the ratio, or multiple of vector or viral genomes to the cells to which the nucleic can be delivered. In some embodiments, the MOI can be 1×10⁶ GC/mL. In some embodiments, the MOI can be 1×10⁵ GC/mL to 1×10⁷ GC/mL. In some embodiments, the MOI can be 1×10⁴ GC/mL to 1×10⁸ GC/mL. In some embodiments, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some embodiments, a mutated or chimeric adeno-associated viruses of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI.

In some aspects, a non-viral vector or nucleic acid can be delivered without the use of a mutated or chimeric adeno-associated viral vector and can be measured according to the quantity of nucleic acid. Generally, any suitable amount of nucleic acid can be used with the compositions and methods of this disclosure. In some embodiments, nucleic acid can be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some embodiments, nucleic acid can be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 pg, 100 μg, 200 pg, 300 μg, 400 pg, 500 μg, 600 pg, 700 μg, 800 pg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

Proteins, vectors, plasmids, compositions, systems, engineered proteins, and guide polynucleotides provided herein can be delivered by any suitable method, including transfection, electroporation, liposome delivery, membrane fusion techniques, high velocity DNA-coated pellets, viral infection and protoplast fusion. The methods used to construct any embodiment of the compositions provided herein include genetic engineering, recombinant engineering, and synthetic techniques.

An engineered protein, a guide polynucleotide, or a polynucleotide encoding a composition provided herein can be delivered to a cell by electroporation. Electroporation using, for example, the NEON® Transfection System (ThermoFisher Scientific) or the AMAXA® Nucleofector (AMAXA® Biosystems) can also be used for delivery of nucleic acids and proteins into a cell. For example, an engineered protein provided herein can be purified and complexed with a suitable guide polynucleotide for delivery into a cell. Electroporation parameters can be adjusted to optimize delivery efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some embodiments, the electroporation pulse voltage, the electroporation pulse width, number of pulses, cell density, and tip type can be adjusted to optimize transfection efficiency and/or cell viability.

### (3) Cells and Cell-Free Systems

Provided herein are cells comprising a system, a guide polynucleotide, a composition, or an engineered protein provided herein. In some embodiments, a polynucleotide encoding for an engineered protein provided herein and a polynucleotide encoding for a guide polynucleotide provided herein are administered to a cell or a population of cells.

The compositions, polynucleotides, engineered proteins, guide polynucleotides and systems provided herein can be delivered to any suitable cell. In some embodiments, compositions, polynucleotides, engineered proteins, guide polynucleotides and systems provided herein modulate a gene, a protein, and/or a functional phenotype of a cell.

Suitable cells can include but are not limited to eukaryotic and prokaryotic cells and/or cell lines. A suitable cell can be, for example, a human primary cell. A primary cell can be taken directly from living tissue (i.e., biopsy material) and established for growth in vitro, that have undergone very few population doublings and are therefore more representative of the main functional components and characteristics of tissues from which they are derived from, in comparison to continuous tumorigenic or artificially immortalized cell lines. A primary cell can be acquired from a variety of sources such as an organ, vasculature, buffy coat, whole blood, apheresis, plasma, bone marrow, tumor, cell-bank, cryopreservation bank, or a blood sample. A primary cell can be a stem cell.

Suitable cells that can contacted with a composition, a polynucleotide, an engineered protein, a guide polynucleotide, or a system provided herein include but are not limited to: epithelial cells, fibroblast cells, neural cells, keratinocytes, hematopoietic cells, melanocytes, chondrocytes, leukocytes, lymphocytes (B, NK, and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, Leydig cells, peritubular cells, Sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, dopaminergic cells, embryonic stem cells, fibroblasts and fetal fibroblasts. Further, the one or more cells can be, for example, pancreatic islet cells and/or cell clusters or the like, including, but not limited to pancreatic α cells, pancreatic β cells, pancreatic δ cells, pancreatic F cells (e.g., PP cells), or pancreatic ε cells.

Suitable cells also include stem cells such as, by way of example, embryonic stem cells, induced pluripotent stem cells, hematopoietic stem cells, neuronal stem cells and mesenchymal stem cells. Suitable cells can comprise any number of primary cells, such as human cells, non-human cells, and/or mouse cells. Suitable cells can be progenitor cells. Suitable cells can be derived from the subject to be treated (e.g., a subject with a disease, a subject in need of treatment, or a subject that is immunocompromised). Suitable cells can be derived from a human donor.

In some embodiments, the cell is genetically modified by the methods, systems, and compositions provided herein. Cells provided herein can be administered to a subject in need thereof, e.g., a subject with a disease or a condition in need of treatment.

A method of attaining suitable cells, such as human primary cells, can comprise selecting cells. In some embodiments, a cell can comprise a marker that can be selected for the cell. For example, such marker can comprise GFP, a resistance gene (for example, a gene conferring antibiotic resistance), a cell surface marker, an endogenous tag. Cells can be selected using any endogenous marker. Suitable cells can be selected using any technology. Such technology can comprise flow cytometry and/or magnetic columns. The selected cells can also be expanded to large numbers.

Delivery vehicles for in vivo and ex vivo use can include pharmaceutically acceptable carriers. Pharmaceutically acceptable carriers are determined in part by the particular composition being administered (e.g., a polynucleotide, a protein, a vector, or a cell), as well as by the particular method used to administer the composition.

Provided herein are cell-free systems comprising a system, a composition, a guide polynucleotide, or an engineered protein provided herein. A cell-free system comprises components sufficient for a template-directed synthetic reaction and in some cases, retain DdDP and nickase bioactivity when stored under room temperature for a period of time. In some embodiments, the cell-free system comprises a set of reagents capable of providing for or supporting a biosynthetic reaction (e.g., DNA replication, transcription, translation, or a combination of reactions) in vitro in the absence of cells. Cell-free systems can be prepared using enzymes, coenzymes, and other subcellular components either isolated or purified from eukaryotic or prokaryotic cells, including recombinant cells, or prepared as extracts or fractions of such cells. A cell-free system can be derived from a variety of sources, including, but not limited to, eukaryotic and prokaryotic cells, such as bacteria including, but not limited to, E. coli, thermophilic bacteria and the like, wheat germ, rabbit reticulocytes, mouse L cells, Ehrlich's ascitic cancer cells, HeLa cells, CHO cells and budding yeast and the like. In some embodiments, the cell-free system is lyophilized. In some embodiments, the cell-free system further comprises a scaffold.

### (4) Pharmaceutical Compositions, Dosing, and Administration

Provided herein are pharmaceutical compositions comprising: a composition, a guide polynucleotide, an engineered protein, or a polynucleotide provided herein; and a pharmaceutically acceptable diluent, carrier, or excipient. Provided herein is a pharmaceutical composition comprising a vector provided herein; and a pharmaceutically acceptable diluent, carrier, or excipient.

In some embodiments, compositions provided herein (e.g., a nanoparticle comprising a system or a composition provided herein) are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions and systems provided herein may be formulated in dosage unit form for ease of administration and uniformity of dosage. A unit dosage form is a physically discrete unit of a composition provided herein appropriate for a subject to be treated. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

Provided herein are pharmaceutical compositions for administering a composition or a system to a subject in need thereof (e.g., as a treatment of a disease). In some embodiments, pharmaceutical compositions provided herein are in a form that allows for compositions provided herein to be administered to a subject. In some embodiments, the pharmaceutical composition is formulated for intratumoral delivery. In some embodiments, administration of a pharmaceutical composition provided herein is local administration or systemic administration. In some embodiments, a pharmaceutical composition provided herein is formulated for administration/for use in administration via an intratumoral, subcutaneous, intradermal, intramuscular, inhalation, intravenous, intraperitoneal, or intracranial route. In some embodiments, the administering is every 1, 2, 4, 6, 8, 12, 24, 36, or 48 hours. In some embodiments, the administering is at least about 5 hours, at least about 10 hours, at least about 12 hours, at least about 15 hours, at least about 20 hours, at least about 24 hours (1 day), at least about 48 hours (2 days), at least about 72 hours (3 days), at least about 96 hours (4 days), at least about 120 hours (5 days), at least about 144 hours (6 days), at least about 168 hours (7 days), at least about 336 hours (14 days), at least about 504 hours (21 days), at least about 672 hours (28 days), up to 744 hours (31 days). In some embodiments, the administering is every 744 hours (e.g., once per month) or once per year (365 days).

Vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, T cells, bone marrow aspirates, or tissue biopsy), followed by reimplantation of the cells into a subject, usually after selection for cells which have incorporated the vector. Prior to or after selection, the cells can be expanded.

Provided herein are cells expressing a system or a composition provided herein. In some embodiments, the cell is contacted in vitro or ex vivo with a nucleic acid encoding for a composition or a system provided herein. In some embodiments, the cell is contacted in vitro or ex vivo with a vector encoding for an engineered protein provided herein, a system provided herein, or a composition provided herein.

### (5) Scaffolds and Systems

Provided herein are scaffolds, wherein the scaffolds comprise any composition, system, or guide polynucleotide provided herein. The scaffolds provided herein can be useful in the detection of newly synthesized nucleic acids or identifying a target nucleic acid provided herein. In some embodiments, the compositions provided herein are immobilized to the scaffold. In some embodiments, the scaffold comprises a surface. In some embodiments, the surface comprises a solid, a semi-solid, or a gel surface. In some embodiments, the scaffold comprises a reaction chip, a paper, a quartz microfiber, mixed esters of cellulose, a porous aluminum oxide, a patterned surface, a tube, a well, or a matrix. In some embodiments, the scaffold comprises a patterned surface suitable for immobilization of molecules in an ordered pattern. In some embodiments, a patterned surface refers to an arrangement of different regions in or on an exposed layer of a scaffold. In some embodiments, the scaffold comprises an array of wells or depressions in a surface. The composition and geometry of the scaffold can vary with its use. In some embodiments, the scaffold is a planar structure such as a slide, chip, microchip and/or array. As such, the surface of the scaffold can be in the form of a planar layer. In some embodiments, the scaffold comprises one or more surfaces of a flowcell. A flowcell is a type of chamber comprising a solid surface across which one or more fluid reagents can be flowed. In some embodiments, the scaffold or its surface is non-planar, such as the inner or outer surface of a tube or vessel. In some embodiments, the scaffold comprise microspheres or beads. Microspheres, beads, or particles can be made of various material including, but not limited to, plastics, ceramics, glass, and polystyrene. In some embodiments, the microspheres are magnetic microspheres or beads. Alternatively or additionally, the beads may be porous. The bead sizes range from nanometers, e.g., about 100 nm, to millimeters, e.g., about 1 mm.

In some embodiments, the scaffold comprises a set of engineered proteins, a set of polynucleotides provided herein, a set of systems provided herein, or any combination thereof. Provided herein are systems further a scaffold, wherein the scaffold comprises a surface. In some embodiments, the scaffold further comprises a cell-free system provided herein. In some embodiments, the systems and scaffolds provided herein further comprise reagents for nucleic acid amplification. In some embodiments, the systems and scaffolds provided herein further comprise reagents for DNA replication. In some embodiments, the systems provided herein further comprise: (a) a scaffold provided herein; (b) a reporter molecule; and (c) a detector. In some embodiments, when a target nucleic acid forms a complex with the scaffold, a guide polynucleotide provided herein, or an engineered protein provided herein, the reporter molecule produces a detectable signal that is detected by the detector. In some embodiments, the reporter molecule is selected from the group consisting of a fluorophore, a dye, a polypeptide, an antibody, a nucleic acid, and any combination thereof. In some embodiments, the detectable signal is a calorimetric signal, a potentiometric signal, an amperometric signal, an optical signal, or a piezo-electric signal. A reporter molecule can be used to identify a cell comprising a new nucleic acid synthesized by the engineered protein provided herein. The reporter molecule can also be used for example, cell sorting, nucleic acid isolation, nucleic acid sequencing, or immunochemistry techniques.

### (6) Kits

Provided herein are kits, wherein the kits comprise: a system, a composition, a polynucleotide, a vector, a guide polynucleotide, an engineered protein, or an engineered RNA encoding the engineered protein provided herein; and packaging and materials therefor. In some embodiments, the kit further comprises a scaffold provided herein. In some embodiments, the kit further comprises a cell-free system. In some embodiments, the kit further comprises a population of cells. In some embodiments, the cells are stored in a cryopreservation medium. In some embodiments, the cryopreservation medium comprises: dimethyl sulfoxide (DMSO). In some embodiments, the cryopreservation medium comprises a buffer, an isotonic agent or an apoptosis inhibitor. Non-limiting examples of buffer elements include: citrate, phosphate, succinate, tartrate, fumarate, gluconate, oxalate, lactate, acetate, histidine and tris. Non-limiting examples of isotonic agents include, for example, citrate, phosphate, succinate, tartrate, fumarate, gluconate, oxalate, lactate, acetate, histidine, and tris. Additional isotonic agents include sodium chloride, potassium chloride, boric acid, sodium borate, mannitol, glycerin, propylene glycol, polyethylene glycol, maltose, sucrose, erythritol, arabitol, xylitol, sorbitol trehalose, and glucose. The apoptosis inhibitor can include, for example, a Rho associated kinase (ROCK) inhibitor, catalase, and zVAD-fmk. In some embodiments, the kits comprise reagents. In some embodiments, the reagents comprise saccharides and saccharide derivatives (e.g., sodium carboxymethyl cellulose and cellulose acetate), detergents, glycols, polyols, esters, buffering agents, alginic acid, and organic solvents.

In some embodiments, a formulation of a composition described herein is prepared in a single container for administration to a cell, a cell-free system, or a subject. In some embodiments, a formulation of a composition provided herein is prepared two containers for administration, separating the guide polynucleotide or polynucleotide encoding the guide polynucleotide and/or the polynucleotide encoding the engineered protein provided herein. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some embodiments, the containers are RNase free.

Provided herein are kits comprising: a first container comprising: a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises: (1) an DST region that has complementarity to a target nucleic acid and at least one alteration relative to the target nucleic acid; and (2) a hybridization region, wherein the hybridization region comprises: a deoxyribonucleotide and a ribonucleotide, and a second container comprising: an engineered protein or a polynucleotide encoding for the engineered protein, wherein the engineered protein comprises: a nickase operably linked to a DNA-dependent DNA polymerase. In some embodiments, a kit provided herein further comprises reagents for nucleic acid amplification, transcription, translation, or nucleic acid isolation. In some embodiments, a kit provided herein further comprises a reporter molecule provided herein.

### (7) Methods of Determining Gene Editing Efficiency

Provided herein are methods of determining gene editing efficiency and selectivity of an engineered protein provided herein for a target nucleic acid sequence. In some embodiments, the target nucleic acid is cleaved by a nuclease or a nickase provided herein upon binding of the guide polynucleotide to the target nucleic acid sequence. In some embodiments, the target nucleic acid is a DNA. In some embodiments, the nickase region of the engineered protein provided herein cleaves the DNA resulting in a single-stranded break. In some embodiments, the nickase region of the engineered protein provided herein cleaves the target DNA via a staggered DNA double-stranded break. In some embodiments, the staggered DNA double-stranded break generates a 5′ overhang. The 5′ overhang can facilitate binding of the hybridization region of the guide polynucleotide and promote DNA replication by the DdDP of the engineered protein. In some embodiments, the 5′ overhang is at least 3 base pairs (bps) or more, at least 4 bps or more, at least 5 bps or more, or at least 6 bps or more. In some embodiments, the 5′ overhang is about 4 bps to 5 bps in length. The ability of a nuclease or the nickase to recognize a PAM sequence can be determined by an in vitro selection assay.

The activity of a system provided herein may be assayed using a cell expressing a reporter protein or containing a reporter gene. For example, a reporter gene may be engineered to contain an obstruction, such as a stop codon, a frameshift mutation, a spacer, a linker, or a transcriptional terminator; the system may then be used to remove the obstruction and the resultant functional reporter protein may be detected. Similarly, a reporter gene can be introduced into the target nucleic acid for detection of the target. In some embodiments, the reporter gene may be designed such that a specific sequence modification is required to restore functionality of the reporter protein. In other embodiments, the reporter gene may be designed such that any insertion or deletion which results in a frame shift of one or two bases in the target nucleic acid may be sufficient to restore functionality of the reporter protein. Examples of reporter proteins encoded by a reporter gene include colorimetric enzymes, metabolic enzymes, fluorescent proteins, enzymes and transporters associated with antibiotic resistance, and luminescent enzymes. Examples of such reporter proteins include β-galactosidase, Chloramphenicol acetyltransferase, Green fluorescent protein, Red fluorescent protein, and Firefly and Renilla luciferase. Different detection methods may be used for different reporter proteins. For example, the reporter protein may affect cell viability, cell growth, fluorescence, luminescence, or expression of a detectable product. In some embodiments, the reporter protein may be detected using a colorimetric assay. In some embodiments, the reporter protein may be a fluorescent protein, and DNA editing may be assayed by measuring the degree of fluorescence in treated cells, or the number of treated cells with at least a threshold level of fluorescence. In some embodiments, transcript levels of a reporter gene may be assessed. In other embodiments, a reporter gene may be assessed by sequencing.

Integration of the new nucleic acid synthesized by DdDP into the target nucleic acid can be measured using any technique, e.g., integration can be measured by denaturing urea polyacrylamide gel electrophoresis, PAGE gel electrophoresis, flow cytometry, a surveyor nuclease assay, tracking of indels by decomposition (TIDE), junction PCR, droplet digital PCR or any combination thereof. In other embodiments, transgene integration can be measured by PCR. A TIDE analysis can also be performed on engineered cells. Ex vivo cell transfection can also be used for diagnostics, research, or for gene therapy (e.g., via re-infusion of the transfected cells into the host organism). In some embodiments, cells are isolated from the subject organism, transfected with a nucleic acid (e.g., gene or cDNA), and re-infused back into the subject organism (e.g., subject).

The amount of genetically modified cells that can be necessary to be therapeutically effective in a subject can vary depending on the viability of the cells, and the efficiency with which the cells have been genetically modified (e.g., the efficiency with which a transgene has been integrated into one or more cells). In some embodiments, the product (e.g., multiplication) of the viability of cells post genetic modification and the efficiency of integration of a transgene can correspond to the therapeutic aliquot of cells available for administration to a subject. In some embodiments, an increase in the viability of cells post-genetic modification can correspond to a decrease in the amount of cells that are necessary for administration to be therapeutically effective in a subject. In some embodiments, an increase in the efficiency with which a transgene has been integrated into one or more cells can correspond to a decrease in the amount of cells that are necessary for administration to be therapeutically effective in a subject. In some embodiments, determining an amount of cells that are necessary to be therapeutically effective can comprise determining a function corresponding to a change in the viability of cells over time. In some embodiments, determining an amount of cells that are necessary to be therapeutically effective can comprise determining a function corresponding to a change in the efficiency with which a transgene can be integrated into one or more cells with respect to time dependent variables (e.g., cell culture time, electroporation time, cell stimulation time).

Non-homologous end joining (NHEJ) and homology-directed repair (HDR) can be quantified using a variety of methods. For example, a percent of NHEJ, HDR, or a combination of both can be determined by co-delivering the gene editing molecules, for example a guide polynucleotide and an engineered protein provided herein, with a donor nucleic acid template that encodes a promoter-less tag or marker (e.g., GFP) into cells. After a duration of time (e.g., 72-96 hours), flow cytometry can be performed to quantify the total cell number (NTotal), tag-positive cell number and tag/GFP-negative cell number. Among the tag-negative cells, next-generation sequencing can be performed to identify cells without mutations and with mutations. HDR efficiency and NHEJ efficiency can be calculated from the assay.

Additional assays for determining gene editing efficiency of a system or composition provided herein can include but is not limited to: RT-PCR, nucleic acid sequencing, T7 endonuclease 1 (T7E1) mismatch detection assays, tracking of indels by decomposition (TIDE) assays, and indel detection by amplicon analysis (IDAA) assays. The indel pattern that is induced at the target site of a programmable nuclease or nickase may also be determined by PCR-amplifying the respective region and subsequent next generation sequencing. To obtain a quantitative single-cell view of gene editing efficiency, cell surface markers can be targeted and the loss of signal that occurs as a consequence of indel formation by flow cytometry can be quantified. Single-cell sequencing can also be employed to directly assess the gene-editing efficiency.

### (8) Applications

Provided herein are methods of modifying a target nucleic acid or generating an alteration in a target nucleic acid or a gene in a cell. In some embodiments, the methods comprise, administering to a cell, a tissue, or a subject the system provided herein, the composition provided herein, or a cell provided herein wherein the administering generates an alteration in a target nucleic acid. In some embodiments, the number of alterations in the target nucleic acid is at least 1 alteration. In some embodiments, the percentage of target nucleic acid molecule alteration is at least 0.05%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. In some embodiments, the method of modifying the target nucleic acid further comprises mutation of a target nucleic acid sequence. In some embodiments, the alteration or the modification of the target nucleic acid comprises: an insertion, a deletion, a substitution, a change in copy number, a point mutation, a frameshift mutation, a missense mutation, a nonsense mutation, a mutation in a stop codon, an epigenetic mark, or any combination thereof. In some embodiments, the alteration is made in the target nucleic acid to form an edited nucleic acid. In some embodiments, the edited nucleic acid restores expression of a wild-type protein that is encoded by the gene relative to a comparable cell or population of cells that were not contacted with the system or the composition.

Provided herein are methods of ex vivo modifying a cell. In some embodiments, the methods comprise: contacting a cell with a composition provided herein under conditions that permit nuclease or nickase cleavage of a target nucleic acid molecule, thereby modifying said cell. Provided herein are methods of ex vivo modifying a cell. In some embodiments, the methods comprise: contacting a cell with a composition provided herein under conditions that permit DNA synthesis of new nucleic acid sequence for incorporation into the target nucleic acid, thereby modifying said target nucleic acid and the cell.

Further provided herein are methods of ex vivo modifying a cell, the method comprising: contacting a cell with a ribonucleoprotein (RNP) complex, wherein the RNP complex comprises: (i) an engineered protein provided herein; and (ii) a polynucleotide that binds to a target nucleic acid, wherein upon contacting the cell with the RNP complex, the engineered protein cleaves a target nucleic acid molecule and synthesizes a new nucleic acid, thereby modifying said cell. In some embodiments, the cell is an immune cell or a stem cell. In some embodiments, the immune cell is a leukocyte, a lymphocyte, a natural killer cell, a dendritic cell, a macrophage, a myeloid cell, a T-cell, a B cell, a stem cell, an induced-pluripotent derived cell, a cancer cell, or an endothelial cell. In some embodiments, the stem cell is an embryonic stem cell, an induced-pluripotent stem cell (iPSC), or an adult stem cell.

Methods of modifying a target nucleic acid provided herein can be used for agricultural applications, for example, the generation of improved plant species; biomedical applications; drug screening; and therapeutic treatments.

Provided herein are methods of synthesizing a nucleic acid, the method comprising: contacting a cell or a cell-free system with: (a) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region has complementarity to a target nucleic acid and at least one alteration relative to the target nucleic acid; and (iv) a hybridization region (HR), wherein the hybridization region comprises: a deoxyribonucleotide and a ribonucleotide; and (b) an engineered protein or a polynucleotide encoding the engineered protein, wherein the engineered protein comprises: (i) a nickase region; and (ii) a DdDP region; wherein: the guide polynucleotide forms a complex with the engineered protein via the protein binding region, the targeting sequence hybridizes with the complementary strand, the nickase region of the engineered protein generates a single strand break in the target nucleic acid to generate a leading strand, the hybridization region forms a complex with the leading strand, and wherein the DdDP region of the engineered protein associates with the DST region of the guide polynucleotide, thereby synthesizing a new nucleic acid. In some embodiments, the targeting region dissociates from the complementary strand. In some embodiments, the new nucleic acid is incorporated into the target nucleic acid by hybridizing to the complementary strand. In some embodiments, DNA repair proteins facilitate the incorporation of the new nucleic acid into the target nucleic acid and edits a nucleobase of the target nucleic acid.

Provided herein are methods of detecting a newly synthesized nucleic acid in a test sample, the method comprising: (a) immobilizing a guide polynucleotide onto a scaffold provided herein; and (b) contacting the guide polynucleotide with: (i) a test sample; and (ii) an engineered protein provided herein. In some embodiments the test sample comprises a target nucleic acid capable of binding to the guide polynucleotide. In some embodiments, a complex is formed between the guide polynucleotide, an engineered protein provided herein, and the target nucleic acid. In some embodiments, upon formation of the complex the engineered protein (e.g., the nickase region) cleaves the target nucleic acid. In some embodiments, the method further comprises detecting a signal indicating cleavage of the target nucleic acid molecule or detecting anew nucleic acid strand comprising a reporter molecule, thereby detecting a target nucleic acid in the sample.

In some embodiments, prior to the detecting step, the method further comprises, amplifying the target nucleic acid. In some embodiments, the amplifying comprises polymerase chain reaction (PCR), nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), multiple displacement amplification (MDA), rolling circle amplification (RCA), improved multiple displacement amplification (EVIDA), 1 simple method amplifying RNA targets (SMART), single primer isothermal amplification (SPIA), ligase chain reaction (LCR), transcription mediated amplification (TMA), ramification amplification method (RAM), or any combination thereof. In some embodiments, the methods further comprise performing an endonuclease mismatch detection assay, an immunoassay, gel electrophoresis, a plasmid interference assay, nucleic acid sequencing, or any combination thereof. In some embodiments, the detecting comprises calorimetric detection, potentiometric detection, amperometric detection, optical detection, piezo-electric detection, or any combination thereof.

Provided herein are methods of treating a disease or a condition in a subject in need thereof. In some embodiments, the subject has, is suspected of having, or is diagnosed with a disease or a condition. In some embodiments, the methods comprise administering to a subject a system, composition, vector, pharmaceutical composition, or polynucleotide, provided herein. In some embodiments, the administering is local or systemic. In some embodiments, the administering is intranasal administration, subcutaneous administration, intravenous administration, inhalation, intramuscular administration, intratumoral administration, peritumoral administration, intrathecal administration, vaginal administration, or intradermal administration. In some embodiments, the method further comprises administering to the subject a therapeutic agent.

Provided is the use of the compositions and systems provided herein in the manufacture of a medicament. Also provided is the use of the compositions described herein in the manufacture of a medicament for therapeutic and/or prophylactic treatment of a disease or condition described herein. In some embodiments, a disease or a condition is caused by a mutated disease-associated gene. In some embodiments, a disease-associated gene is any gene associated with an increase in the risk of having or developing a disease. In some embodiments, a disease-associated gene is any gene or polynucleotide which is yielding transcription or translation products at an abnormal level or in an abnormal form in cells derived from a disease-affected tissues compared with tissues or cells of a non-disease control. In some embodiments, a disease-associated gene is a gene that becomes expressed at an abnormally high level. In some embodiments, a disease-associated gene is a gene that becomes expressed at an abnormally low level, where the altered expression correlates with the occurrence and/or progression of the disease. In some embodiments, a disease-associated gene is a gene possessing mutation(s) or genetic variation that is responsible or is in linkage disequilibrium with a gene(s) that is responsible for the etiology of a disease. The transcribed or translated products may be known or unknown, and may be at a normal or abnormal level.

In some embodiments, a disease or a condition is caused by mutations associated with DNA repeat instability and neurological disorders, Specific aspects of tandem repeat sequences have been found to be responsible for more than twenty human diseases. The system may be harnessed to correct these defects of genomic instability. In some embodiments, the disease or the condition is a neurological disease, a neurodegenerative disease, a cardiovascular disease, a cancer, a respiratory disease, diabetes, obesity or complications associated with obesity, an eye disease, loss of hearing, blindness, a birth defect, a cardiac arrythmia disorder, a cancer, epilepsy, asthma, allergies, multiple sclerosis, muscular dystrophy, Huntington's disease, amyotrophic lateral sclerosis (ALS), a metabolic disease, an autoimmune disease, a developmental disorder, a learning disorder, a kidney disease, a liver disease, a gastrointestinal disease, or a rare genetic disease or disorder. In some embodiments, the disease or the condition is age-related macular degeneration, a schizophrenic disorder, trinucleotide repeat disorder, or Fragile X Syndrome. In some embodiments, the disease or the condition is a Secretase Related Disorder. In some embodiments, the disease or the condition is a Prion-related disorder. In some embodiments, the disease or the condition is ALS. In some embodiments, the disease or the condition is a drug addiction. In some embodiments, the disease or the condition is Autism. In some embodiments, the disease or the condition is Alzheimer's Disease. In some embodiments, the disease or the condition is inflammation. In some embodiments, the disease or the condition is Parkinson's Disease. Further examples of diseases and conditions treatable with the systems and compositions provided herein include but are not limited to: Aieardi-Goutieres Syndrome; Alexander Disease; Allan-Herndon-Dudiey Syndrome; POLG-Related Disorders; Alpha-Mannosidosis (Type II and III); Alstrom Syndrome, Angelman: Syndrome, Ataxia-Telangiectasia: Neuronal Ceroid-Lipofuscinoses: Beta-rrhalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich's Ataxia [FRDA]; Fryns Syndrome; Fucosidosis; Fukuyana Congenital Muscular Dystrophy; Galactosialidosis; Gaucher Disease; Organic Acidemias; Hemophagocytic Lymphohistiocytosis; Progeria Syndrome; Mucolipidosis II; Infantile Free Sialic Acid Storage Disease; PLA2C6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome: LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy: Arvisulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders: COL 1 A1/2-Related Osteogenesis Imperfecta; Mitochondrial DNA Deletion Syndromes; PLP1-Related Disorders; Perry Syndrome; Phelan-hMcDernild Syndrome; Glycogen Storage Disease Type 11 (Pomnpe Disease) (Infantile); MAPT-Related Disorders; MECP2-Related Disorders; Rhi zomelic Chondrodysplasia Punctata Type 1; Roberts Syndrome; Sandhoff Disease; Sehindler Disease-Type 1; Adenosine Deammase Deficiency; Smith-Lenmli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type 1; Congenital Muscular Dystrophy; Wolf-Hirschhorn Syndrome; Lysosomal Acid Lipase Deficiency; and Xeroderma Pigmentosum. In some embodiments, the subject is a mammal. In some embodiments, the subject is a human.

### Exemplary Embodiments

Provided herein are compositions, wherein the compositions comprise: (a) a polynucleotide encoding a phi29 DNA-dependent DNA polymerase or a variant thereof; (b) a polynucleotide encoding a nickase; and (c) a guide polynucleotide comprising: (i) a targeting region that has complementarity to a target nucleic acid; (ii) a protein binding region, wherein the protein binding region comprises a secondary structure that binds to the nickase; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides. Further provided herein are compositions, wherein the nickase comprises a Type II Cas protein. Further provided herein are compositions, wherein the Type II Cas protein comprises: a Cas9, a Cas1, a Cas2, or a Csn2. Further provided herein are compositions, wherein the Cas9 protein comprises a Streptococcus pyogenes Cas9 protein or a variant thereof. Further provided herein are compositions, wherein the Cas9 protein comprises a Streptococcus thermophilus Cas9 protein or a variant thereof. Further provided herein are compositions, wherein the phi29 DNA-dependent DNA polymerase comprises a sequence that is at least 90% identical to SEQ ID NO: 63. Further provided herein are compositions, wherein the phi29 DNA-dependent DNA polymerase comprises a sequence that is at least 95% identical to SEQ ID NO: 63. Further provided herein are compositions, wherein the phi29 DNA-dependent DNA polymerase comprises a sequence that is at least 99% identical to SEQ ID NO: 63. Further provided herein are compositions, wherein the phi29 DNA-dependent DNA polymerase comprises SEQ ID NO: 63. Further provided herein are compositions, wherein the phi29 DNA-dependent DNA polymerase variant comprises a mutation, wherein the mutation comprises an amino acid substitution of an amino acid residue of position: 8, 12, 51, 66, 97, 137, 197, 221, 369, 372, 375, 377, 378, 497, 512, or 526 as compared to SEQ ID NO: 63. Further provided herein are compositions, wherein the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue as compared to SEQ ID NO: 63. Further provided herein are compositions, wherein the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is an arginine (Arg, R), an alanine (Ala, A), a glutamic acid (Glu, E), a proline (Pro, P), a threonine (Thr, T), an aspartic acid (Asp, D), a cysteine (Cys, C), a leucine (Leu, L) or a lysine (Lys, K) as compared to SEQ ID NO: 63. Further provided herein are compositions, wherein the amino acid substitution comprises: M8R, D12A, V51A, D66A, M97T, F137C, G197D, E221K, Y369E, T372N, E375D, A377C, I378R, Q497P, K512E, F526L, or any combination thereof as compared to SEQ ID NO: 63. Further provided herein are compositions, wherein the phi29 DNA-dependent DNA polymerase variant comprises any one of SEQ ID NO: 99-SEQ ID NO: 106. Further provided herein are compositions, wherein the phi29 DNA-dependent DNA polymerase variant comprises SEQ ID NO: 102. Further provided herein are compositions, wherein the phi29 DNA-dependent DNA polymerase variant comprises SEQ ID NO: 103. Further provided herein are compositions, wherein the phi29 DNA-dependent DNA polymerase variant comprises SEQ ID NO: 104. Further provided herein are compositions, wherein the polynucleotide encoding the phi29 DNA-dependent DNA polymerase or a variant thereof is linked to the polynucleotide encoding the nickase. Further provided herein are compositions, wherein the hybridization region comprises at least about 5 nucleotides up to 20 nucleotides. Further provided herein are compositions, wherein the hybridization region comprises a ratio of ribonucleic acids (RNAs) to deoxyribonucleic acids (DNAs) of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. Further provided herein are compositions, wherein the secondary structure of the protein binding region of the guide polynucleotide comprises: a bulge, a stem, a loop, a hairpin, a wobble base pair, a pseudoknot, or a combination thereof. Further provided herein are compositions, wherein the DST region comprises at least about 5 nucleotides up to 10,000 nucleotides.

Provided herein are compositions, wherein the compositions comprise: (a) an engineered fusion protein comprising: (i) a phi29 DNA-dependent DNA polymerase or a variant thereof, and (ii) a nickase, wherein the phi29 DNA-dependent DNA polymerase or a variant thereof is linked to the nickase, (b) a guide polynucleotide comprising: (i) a targeting region that has complementarity to a target nucleic acid; (ii) a protein binding region, wherein the protein binding region comprises a secondary structure that binds to the nickase; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and (iv) a hybridization region, wherein the hybridization region comprises: deoxyribonucleotides and ribonucleotides.

Provided herein are compositions, wherein the compositions comprise: (a) a polynucleotide encoding a DNA-dependent DNA polymerase or a variant thereof, (b) a polynucleotide encoding a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92, (c) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides. Further provided herein are compositions, wherein the DNA-dependent polymerase comprises a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol μ, a Pol κ, a Pol ι, a Pol η, a Pol IV, a Pol V, a terminal deoxynucleotidyl transferase, or a combination thereof. Further provided herein are compositions, wherein the DNA-dependent polymerase comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof. Further provided herein are compositions, wherein the Streptococcus pyogenes Cas9 nickase variant comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue as compared to SEQ ID NO: 92. Further provided herein are compositions, wherein the Streptococcus pyogenes Cas9 nickase variant comprises an amino acid substitution comprises an amino acid substitution at position 840. Further provided herein are compositions, wherein the Streptococcus pyogenes Cas9 nickase variant comprises an amino acid substitution comprises an amino acid substitution at position 221, 394, and 840. Further provided herein are compositions, wherein the amino acid substitution comprises H840A as compared to SEQ ID NO: 92. Further provided herein are compositions, wherein the amino acid substitution comprises: R221K, N394K, H840A, or any combination thereof as compared to SEQ ID NO: 92. Further provided herein are compositions, wherein the Streptococcus pyogenes Cas9 nickase variant comprises amino acid substitutions at positions 221, 394, 840, 1135, 1218, 1335, or 1337 as compared to SEQ ID NO: 92. Further provided herein are compositions, wherein the Streptococcus pyogenes Cas9 nickase variant comprises amino acid substitutions selected from: R221K, N394K, H840A, D1135V, G1218R, R1335Q, and T1337R as compared to SEQ ID NO: 92. Further provided herein are compositions, wherein the Streptococcus pyogenes Cas9 nickase variant comprises amino acid substitutions at positions 221, 394, 840, 1135, 1136, 1218, 1219, 1335, or 1337 as compared to SEQ ID NO: 92. Further provided herein are compositions, wherein the Streptococcus pyogenes Cas9 nickase variant comprises amino acid substitutions selected from: R221K, N394K, H840A, D1135L, S1136W, G1218K, E1219Q, R1335Q, and T1337R as compared to SEQ ID NO: 92. Further provided herein are compositions, wherein the Streptococcus pyogenes Cas9 nickase variant comprises amino acid substitutions at any one of positions 61, 221, 394, 840, 1111, 1135, 1136, 1218, 1219, 1317, 1322, 1333, 1335, or 1337 as compared to SEQ ID NO: 92. Further provided herein are compositions, wherein the Streptococcus pyogenes Cas9 nickase variant comprises amino acid substitutions selected from: A61R, R221K, N394K, H840A, L1111R, D1135L, S1136W, G1218K, E1219Q, N1317R, A1322R, R1333P, R1335Q, and T1337R as compared to SEQ ID NO: 92. Further provided herein are compositions, wherein the Streptococcus pyogenes Cas9 nickase variant comprises any one of SEQ ID NO: 93 to SEQ ID NO: 96. Further provided herein are compositions, wherein the polynucleotide encoding the DNA-dependent DNA polymerase or a variant thereof is linked to the polynucleotide encoding the Streptococcus pyogenes Cas9 or the variant thereof. Further provided herein are compositions, wherein the hybridization region comprises at least about 5 nucleotides up to 20 nucleotides. Further provided herein are compositions, wherein the hybridization region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. Further provided herein are compositions, wherein the secondary structure of the protein binding region of the guide polynucleotide comprises: a bulge, a stem, a loop, a hairpin, a wobble base pair, a pseudoknot, or a combination thereof. Further provided herein are compositions, herein the DST region comprises at least about 5 nucleotides up to 10,000 nucleotides.

Provided herein are compositions, wherein the compositions comprise: (a) an engineered fusion protein comprising: (i) a DNA-dependent DNA polymerase or a variant thereof; (ii) a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92, (b) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and (iv) a hybridization region, wherein the hybridization region comprises: deoxyribonucleotides and ribonucleotides.

Provided herein are compositions, wherein the compositions comprise: (a) a polynucleotide encoding a DNA-dependent DNA polymerase or a variant thereof, (b) a polynucleotide encoding a Streptococcus thermophilus Cas9 nickase or a variant thereof, wherein the variant comprises an amino acid substitution at position 599 as compared to SEQ ID NO: 97, (c) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides. Further provided herein are compositions, wherein the DNA-dependent polymerase comprises a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol μ, a Pol κ, a Pol ι, a Pol η, a Pol IV, a Pol V, a terminal deoxynucleotidyl transferase, or a combination thereof. Further provided herein are compositions, wherein the DNA-dependent polymerase comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof. Further provided herein are compositions, wherein the Streptococcus thermophilus Cas9 nickase variant comprises an amino acid substitution comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue as compared to SEQ ID NO: 97. Further provided herein are compositions, wherein the Streptococcus thermophilus Cas9 nickase variant comprises an amino acid substitution of H599A as compared to SEQ ID NO: 97. Further provided herein are compositions, wherein the Streptococcus thermophilus Cas9 nickase variant comprises SEQ ID NO: 98. Further provided herein are compositions, wherein the polynucleotide encoding the DNA-dependent DNA polymerase or the variant thereof is linked to the polynucleotide encoding the Streptococcus thermophilus Cas9 or the variant thereof. Further provided herein are compositions, wherein the hybridization region comprises at least about 5 nucleotides up to 20 nucleotides. Further provided herein are compositions, herein the hybridization region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. Further provided herein are compositions, wherein the secondary structure of the protein binding region of the guide polynucleotide comprises: a bulge, a stem, a loop, a hairpin, a wobble base pair, a pseudoknot, or a combination thereof. Further provided herein are compositions, wherein the DST region comprises at least about 5 nucleotides up to 10,000 nucleotides.

Provided herein are compositions, wherein the compositions comprise: (a) an engineered fusion protein comprising: (i) a DNA-dependent DNA polymerase or a variant thereof, and (ii) a Streptococcus thermophilus Cas9 nickase or a variant thereof, wherein the variant comprises an amino acid substitution at position 599 as compared to SEQ ID NO: 97, (b) a guide polynucleotide comprising: (i) a targeting region that has complementarity to a target nucleic acid; (ii) a protein binding region, wherein the protein binding region comprises a secondary structure that binds to a protein; and (iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof; and (iv) a hybridization region, wherein the hybridization region comprises: deoxyribonucleotides and ribonucleotides.

Provided herein are compositions, wherein the compositions comprise: an engineered fusion protein comprising: (a) a phi29 DNA-dependent DNA polymerase or a variant thereof; and (b) a nickase or a variant thereof. Further provided herein are compositions, wherein the engineered fusion protein comprises a phi29 DNA-dependent DNA polymerase having a sequence that is at least 90% identical to SEQ ID NO: 63. Further provided herein are compositions, wherein the engineered fusion protein comprises a phi29 DNA-dependent DNA polymerase having a sequence that is at least 95% identical to SEQ ID NO: 63. Further provided herein are compositions, wherein the engineered fusion protein comprises a phi29 DNA-dependent DNA polymerase having a sequence that is at least 99% identical to SEQ ID NO: 63. Further provided herein are compositions, wherein the engineered fusion protein comprises a phi29 DNA-dependent DNA polymerase having a sequence of SEQ ID NO: 63. Further provided herein are compositions, wherein the engineered fusion protein comprises the phi29 DNA-dependent DNA polymerase variant, wherein the phi29 DNA-dependent DNA polymerase variant comprises a sequence that is at least 90% identical to any one of SEQ ID NO: 99-SEQ ID NO: 106. Further provided herein are compositions, wherein the engineered fusion protein comprises the phi29 DNA-dependent DNA polymerase variant, wherein the phi29 DNA-dependent DNA polymerase variant comprises a sequence that is at least 95% identical to any one of SEQ ID NO: 99-SEQ ID NO: 106. Further provided herein are compositions, wherein the engineered fusion protein comprises the phi29 DNA-dependent DNA polymerase variant, wherein the phi29 DNA-dependent DNA polymerase variant comprises a sequence that is at least 99% identical to any one of SEQ ID NO: 99-SEQ ID NO: 106. Further provided herein are compositions, wherein the engineered fusion protein comprises the phi29 DNA-dependent DNA polymerase variant, wherein the phi29 DNA-dependent DNA polymerase variant comprises any one of SEQ ID NO: 99-SEQ ID NO: 106. Further provided herein are compositions, wherein the engineered fusion protein comprises the phi29 DNA-dependent DNA polymerase variant, wherein the phi29 DNA-dependent DNA polymerase variant comprises SEQ ID NO: 102, SEQ ID NO: 103, or SEQ ID NO: 104. Further provided herein are compositions, wherein the nickase comprises a Cas protein or a mutant Cas protein. Further provided herein are compositions, wherein the Cas protein or the mutant Cas protein is a Type V Cas protein. Further provided herein are compositions, wherein the Type V Cas protein comprises: a Cas12a, a Cas12b, a Cas12c, a Cas12d, a Cas12e, a Cas14, a Cas12g, a Cas12h, a Cas12i, a Cas12j, or a Cas12k. Further provided herein are compositions, wherein the Cas protein or the mutant Cas protein comprises: a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a SpCas9-VQR, a SpCas9-VRQR, a SpCas9-VRER, a SaCas9-KKH, a SpCas9-NG, a SpCas9-NRRH, a SpCas9-NRTH, a SpCas9-NRCH, a iSpyMac, a St1Cas9 LMD9-LMG18311, a St1Cas9 LMD9-CNRZ1066, a St1Cas9-KQKL, a variant, or any combination thereof. Further provided herein are compositions, wherein the Cas protein or the mutant Cas protein is a Type II Cas protein. Further provided herein are compositions, wherein the Type II Cas protein comprises: a Cas9, an spCas9, an St1Cas9, a CasI, a Cas2, or a Csn2. Further provided herein are compositions, wherein the nickase or the variant thereof comprises a Streptococcus thermophilus Cas9 nickase or a variant thereof. Further provided herein are compositions, wherein the nickase or the variant thereof comprises a Streptococcus pyogenes Cas9 nickase or a variant thereof. Further provided herein are compositions, wherein the engineered fusion protein comprises a sequence that is at least 90% identical to any one of SEQ ID NO: 234 to SEQ ID NO: 263. Further provided herein are compositions, wherein the engineered fusion protein comprises a sequence that is at least 95% identical to any one of SEQ ID NO: 234 to SEQ ID NO: 263. Further provided herein are compositions, wherein the engineered fusion protein comprises a sequence that is at least 99% identical to any one of SEQ ID NO: 234 to SEQ ID NO: 263. Further provided herein are compositions, wherein the engineered fusion protein comprises any one of SEQ ID NO: 234 to SEQ ID NO: 263. Further provided herein are compositions, wherein the engineered fusion protein comprises SEQ ID NO: 250. Further provided herein are compositions, wherein the engineered fusion protein comprises SEQ ID NO: 252. Further provided herein are compositions, wherein the engineered fusion protein comprises SEQ ID NO: 255. Further provided herein are compositions, wherein the engineered fusion protein comprises SEQ ID NO: 256. Further provided herein are compositions, further comprising a linker, a nuclear localization sequence (NLS), an additional protein construct, or any combination thereof. Further provided herein are compositions, wherein the additional protein construct comprises a cell-targeting moiety, a receptor-targeting moiety, a regulatory element, a nuclease, an acetylase, an acetyltransferase, an ATPase, an Argonaute protein, a base editor, a Cas polypeptide, a catalytically dead Cas polypeptide, a deacetylase, a deaminase, a decapping protein, an endonuclease, an exonuclease, a helicase, a ligase, a meganuclease, a methylase, a methyltransferase, a nickase, a polymerase, a protease, a recombinase, a restriction enzyme, a ribonucleoprotein (RNP), a self-cleaving protein sequence, a splicing factor, a transcriptional activator, a transcription activator-like effector nuclease (TALEN), a transcriptional repressor, a transposase, a zinc finger, or any combination thereof.

Provided herein are compositions, wherein the compositions comprise: an engineered fusion protein comprising: (a) a phi29 DNA-dependent DNA polymerase or a variant thereof; and (b) a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92.

Provided herein are compositions, wherein the compositions comprise: an engineered fusion protein comprising: (a) a phi29 DNA-dependent DNA polymerase or a variant thereof; and (b) a Streptococcus thermophilus Cas9 nickase or a variant thereof, wherein the variant comprises an amino acid substitution at position 599 as compared to SEQ ID NO: 97.

Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region has complementarity to the target nucleic acid, wherein upon introduction to a cell or a cell-free system, the system synthesizes a nucleic acid sequence that is incorporated into the target nucleic acid. Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: one or more ribonucleotides or one or more deoxyribonucleotides, wherein the hybridization region has complementarity to the target nucleic acid, wherein upon introduction to a cell or a cell-free system, the system synthesizes a nucleic acid sequence that is incorporated into the target nucleic acid. Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: one or more deoxyribonucleotides, wherein the hybridization region has complementarity to the target nucleic acid, wherein upon introduction to a cell or a cell-free system, the system synthesizes a nucleic acid sequence that is incorporated into the target nucleic acid. Provided herein are systems for synthesizing a nucleic acid sequence, wherein the systems comprise: (a) an engineered protein construct comprising a nickase region; (b) a DNA-dependent DNA polymerase (DdDP) or a functional fragment thereof; and (c) a guide polynucleotide, wherein the guide polynucleotide comprises: (i) a targeting region that has complementarity to a target nucleic acid; (ii) a protein binding region, wherein the protein binding region comprises a secondary structure that binds to the engineered protein construct comprising a nickase region; (iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and (iv) a hybridization region, wherein the hybridization region comprises: a deoxyribonucleotide and a ribonucleotide, wherein the hybridization region has complementarity to the target nucleic acid, wherein upon introduction to a cell or a cell-free system, the system synthesizes a nucleic acid sequence that is incorporated into the target nucleic acid. Further provided herein are systems, wherein the hybridization region forms a DNA-RNA-(DR) loop upon association with the target nucleic acid and the DdDP. Further provided herein are systems, wherein the hybridization region comprises a ratio of ribonucleic acids (RNAs) to deoxyribonucleic acids (DNAs) of: 1:1 up to 20:1. Further provided herein are systems, wherein the hybridization region comprises a ratio of ribonucleic acids (RNAs) to deoxyribonucleic acids (DNAs) of: 1 up to 20. Further provided herein are systems, wherein the hybridization region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, or 19:18. Further provided herein are systems, wherein the hybridization region hybridizes to a leading strand upon cleavage of the target nucleic acid by the engineered protein construct. Further provided herein are systems, wherein the ribonucleotides and/or deoxyribonucleotides of the hybridization region hybridize to the leading strand. Further provided herein are systems, wherein the DST region comprises at least about 5 nucleotides up to 10,000 nucleotides. Further provided herein are systems, wherein the DST region comprises at least about 7 nucleotides up to 1,000 nucleotides. Further provided herein are systems, wherein the hybridization region comprises at least about 5 nucleotides up to 20 nucleotides. Further provided herein are systems, wherein the DST region comprises a reverse complement sequence of a non-coding polynucleotide sequence or a variant thereof. Further provided herein are systems, wherein the DST region comprises a reverse complement sequence of a sequence encoding a coding region of a polynucleotide sequence or a variant thereof. Further provided herein are systems, wherein the DST region comprises a reverse complement sequence of a sequence encoding for an exon or an intron. Further provided herein are systems, wherein the DST region comprises a complement sequence of a sequence encoding a non-coding polynucleotide sequence or a variant thereof. Further provided herein are systems, wherein the DST region comprises a complement sequence of a sequence encoding a coding region of a polynucleotide sequence or a variant thereof. Further provided herein are systems, wherein the DST region comprises a complement sequence of a sequence encoding for an exon or an intron. Further provided herein are systems, wherein the DST region comprises a sequence comprising at least one nucleobase that is complementary to or mismatched with a sequence encoding a splice acceptor site. Further provided herein are systems, wherein the targeting region hybridizes to a complementary strand of the target nucleic acid and the nickase region cleaves the target nucleic acid. Further provided herein are systems, wherein the targeting region hybridizes to the complementary strand of the target nucleic acids within at least about 5 nucleobases of a PAM sequence, at least about 10 nucleobases of a PAM sequence, at least about 15 nucleobases of a PAM sequence, or at least about 20 nucleobases of a PAM sequence. Further provided herein are systems, wherein the engineered protein construct comprises a Cas protein or a mutant Cas protein. Further provided herein are systems, wherein the Cas protein or the mutant Cas protein is a Type V Cas protein. Further provided herein are systems, wherein the Type V Cas protein comprises: a Cas12a, a Cas12b, a Cas12c, a Cas12d, a Cas12e, a Cas14, a Cas12g, a Cas12h, a Cas12i, a Cas12j, or a Cas12k. Further provided herein are systems, wherein the Cas protein or the mutant Cas protein comprises: a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a SpCas9-VQR, a SpCas9-VRQR, a SpCas9-VRER, a SaCas9-KKH, a SpCas9-NG, a SpCas9-NRRH, a SpCas9-NRTH, a SpCas9-NRCH, a iSpyMac, a St1Cas9 LMD9-LMG18311, a St1Cas9 LMD9-CNRZ1066, a St1Cas9-KQKL, a variant, or any combination thereof. Further provided herein are systems, wherein the Cas protein or the mutant Cas protein is a Type II Cas protein. Further provided herein are systems, wherein the Type II Cas protein comprises: a Cas9, a CasI, a Cas2, or a Csn2. Further provided herein are systems, wherein the engineered protein construct cleaves the target nucleic acid 5′ upstream of a protospacer adjacent motif (PAM) sequence. Further provided herein are systems, wherein the engineered protein construct cleaves the target nucleic acid to generate two single strands of DNA or two single strands of an RNA duplex. Further provided herein are systems, wherein the engineered protein construct generates a single-stranded break in the target nucleic acid. Further provided herein are systems, wherein the DdDP or the functional fragment thereof comprises a Pol I, a Pol γ, a Pol θ, a Pol ν, a Pol II, a Pol B, a Pol ζ, a Pol α, a Pol δ, a Pol ε, a Pol III, a PolD, a Pol β, a Pol σ, a Pol λ, a Pol μ, a Pol κ, a Pol ι, a Pol η, a Pol IV, a Pol V, a terminal deoxynucleotidyl transferase, or a combination thereof. Further provided herein are systems, wherein the DdDP or the functional fragment thereof comprises a Klenow fragment of a E. coli DNA Pol I, a phi29 DNA Pol, a Ba71V DNA Pol, a human DNA Pol λ, a human DNA Pol β, a Bsu DNA Pol I, a T3 DNA Pol, a T4 DNA Pol, a T5 DNA Pol, a T7 DNA Pol, a Bst DNA Pol, a human DNA Pol α, a human alpha herpesvirus DNA Pol, a Phi X 174 DNA Pol, a Herpes simplex virus (HSV) DNA Pol, a Hepatitis B virus (HBV) DNA Pol, a Epstein-Barr virus (EBV) DNA Pol, or any combination thereof. Further provided herein are systems, wherein the DdDP binds to a DdDP recruiting region of the DNA-dependent DNA polymerase synthesis template (DST) region of the guide polynucleotide. Further provided herein are systems, wherein the DdDP does not bind to the target nucleic acid. Further provided herein are systems, wherein the DdDP synthesizes a new DNA strand that has complementarity to the target nucleic acid. Further provided herein are systems, wherein the new DNA strand has at least 50% up to 99.99% complementarity to the target nucleic acid. Further provided herein are systems, wherein the new DNA strand comprises a mismatched nucleobase relative to the target nucleic acid. Further provided herein are systems, wherein the secondary structure of the protein binding region of the guide polynucleotide comprises: a bulge, a stem, a loop, a hairpin, a wobble base pair, a pseudoknot, or a combination thereof. Further provided herein are systems, wherein the target nucleic acid comprises DNA. Further provided herein are systems, wherein the target nucleic acid comprises RNA. Further provided herein are systems, wherein the systems further comprise an additional protein construct, a linker, an NLS, or any combination thereof. Further provided herein are systems, wherein the protein construct and the DdDP are linked together by a polypeptide linker.

Provided herein are compositions, wherein the compositions comprise: a system provided herein, an engineered protein construct provided herein, a DNA-dependent DNA polymerase provided herein or a functional fragment thereof, or a guide polynucleotide provided herein.

Provided herein are compositions, wherein the compositions comprise: the systems provided herein; and a delivery vehicle.

Provided herein are polynucleotides, wherein the polynucleotides encode for a composition provided herein, a system provided herein, an engineered protein provided herein, or a guide polynucleotide provided herein. Further provided herein are polynucleotides, wherein the polynucleotides comprise DNA. Further provided herein are polynucleotides, wherein the polynucleotides comprise RNA. Further provided herein are polynucleotides, wherein the polynucleotides comprise DNA, RNA, or both DNA and RNA.

Provided herein are sets of polynucleotides, wherein the sets of polynucleotides encode for a system provided herein, an engineered protein provided herein, or a guide polynucleotide provided herein.

Provided herein are nanoparticles, wherein the nanoparticles comprise: the polynucleotides provided herein, the sets of polynucleotides provided herein, the systems provided herein, the compositions provided herein, the cells provided herein, the vectors provided herein or any portion thereof. Further provided herein are nanoparticles, wherein the nanoparticles are lipid nanoparticles.

Provided herein are vectors, wherein the vectors comprise: the polynucleotides provided herein.

Provided herein are cells comprising: a system provided herein, a polynucleotide provided herein, a vector provided herein, a composition provided herein, an engineered protein provided herein, or a guide polynucleotide provided herein.

Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region; and an engineered protein or a polynucleotide encoding the engineered protein, wherein the engineered protein comprises a nickase region; and a DNA-dependent DNA polymerase (DdDP) region. Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises one or more ribonucleotides. Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof, and at least one alteration relative to the target nucleic acid or at least one nucleic acid strand thereof, and (d) a hybridization region, wherein the hybridization region comprises a deoxyribonucleotide and a ribonucleotide. Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof, and (d) a hybridization region, wherein the hybridization region comprises one or more ribonucleotides or one or more deoxyribonucleotides; and an engineered protein or a polynucleotide encoding the engineered protein, wherein the engineered protein comprises a nickase region; and a DNA-dependent DNA polymerase (DdDP) region. Provided herein are compositions, wherein the compositions comprise: a guide polynucleotide or a polynucleotide encoding the guide polynucleotide, wherein the guide polynucleotide comprises: (a) a targeting region that has complementarity to a target nucleic acid; (b) a protein binding region, wherein the protein binding region comprises a secondary structure that binds to a protein; and (c) a DNA-dependent DNA polymerase synthesis template (DST), wherein the DNA-dependent DNA polymerase synthesis template (DST) region comprises: a sequence that has complementarity to the target nucleic acid or at least one nucleic acid strand thereof; and at least one alteration relative to the target nucleic acid or at least one nucleic acid strand thereof; and (d) a hybridization region, wherein the hybridization region comprises a deoxyribonucleotide and a ribonucleotide; and an engineered protein or a polynucleotide encoding the engineered protein, wherein the engineered protein comprises a nickase region; and a DNA-dependent DNA polymerase (DdDP) region. Further provided herein are compositions, wherein the target nucleic acid comprises a dsDNA or a double-stranded RNA duplex that is cleaved by a nickase. Further provided herein are compositions, wherein the protein binding region binds to a nickase or an endonuclease. Further provided herein are compositions, wherein the secondary structure comprises a bulge, a stem, a loop, a hairpin, a wobble base pair, a pseudoknot, or a combination thereof. Further provided herein are compositions, wherein the DST region comprises at least about 5 nucleotides up to 10,000 nucleotides. Further provided herein are compositions, wherein the DST region comprises at least about 7 nucleotides up to 1,000 nucleotides. Further provided herein are compositions, wherein the DST region comprises at least one alteration that is a mismatched nucleobase. Further provided herein are compositions, wherein the mismatched nucleobase comprises an A/C mismatch, a A/T mismatch, an A/G mismatch, and T/C mismatch, a T/G mismatch, a T/A mismatch, a C/G mismatch, a C/A mismatch, a C/T mismatch, a G/C mismatch, a G/T mismatch, a G/A mismatch, or a combination thereof relative to the target nucleic acid sequence. Further provided herein are compositions, wherein the DST region comprises a reverse complement sequence of a non-coding region of a polynucleotide or a variant thereof. Further provided herein are compositions, wherein the DST region comprises a reverse complement sequence of a sequence encoding a coding region of a polynucleotide sequence or a variant thereof. Further provided herein are compositions, wherein the DST region comprises a reverse complement sequence of a sequence encoding for an exon or an intron. Further provided herein are compositions, wherein the DST region comprises a sequence comprising at least one nucleobase that is complementary to or mismatched with a sequence encoding a splice acceptor site. Further provided herein are compositions, wherein the DST region comprises a complement sequence of a non-coding region of a polynucleotide or a variant thereof. Further provided herein are compositions, wherein the DST region comprises a complement sequence of a sequence encoding a coding region of a polynucleotide sequence or a variant thereof. Further provided herein are compositions, wherein the DST region comprises a complement sequence of a sequence encoding for an exon or an intron. Further provided herein are compositions, wherein the hybridization region forms a DNA-RNA loop upon association with the target nucleic acid and a DdDP. Further provided herein are compositions, wherein the hybridization region comprises a ration of RNAs to DNA of 1 up to 20. Further provided herein are compositions, wherein the hybridization region comprises a ration of RNAs to DNA of 1:1 up to 20:1. Further provided herein are compositions, wherein the hybridization region comprises a ratio of ribonucleic acids (RNAs) to deoxyribonucleic acids (DNAs) of: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 2:15, 2:17, 2:19, 3:1, 3:2, 3:4, 3:5, 3:7, 3:8, 3:10, 3:11, 3:13, 3:14, 3:15, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 6:1, 6:5, 6:7, 6:9, 6:11, 6:13, 6:15, 7:1, 7:2, 7:3, 7:4, 7:5, 7:6, 7:8, 7:9, 7:10, 7:11, 7:12, 7:13, 7:15, 8:1, 8:3, 8:5, 8:7, 8:9, 8:11, 8:13, 8:15 9:1, 9:2, 9:4, 9:5, 9:7, 9:8, 9:10, 9:11, 9:13, 9:15, 9:17, 9:19, 9:20, 10:1, 10:3, 10:7, 10:9, 10:11, 10:13, 10:15, 10:17, 10:19, 11:1, 11:2, 11:3, 11:4, 11:5, 11:6, 11:7, 11:8, 11:9, 11:10, 11:12, 11:13, 11:15, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, or 20:1. Further provided herein are compositions, wherein the hybridization region hybridizes to the target nucleic acid upon cleavage of the target nucleic acid by a nickase, wherein the nickase generates a leading strand and a complementary strand. Further provided herein are compositions, wherein the ribonucleotides hybridize to the leading strand. Further provided herein are compositions, wherein the hybridization region comprises at least about 5 nucleotides up to 10,000 nucleotides. Further provided herein are compositions, wherein the hybridization region comprises at least about 7 nucleotides up to 1,000 nucleotides. Further provided herein are compositions, wherein the hybridization region comprises at least about 5 nucleotides up to 20 nucleotides. Further provided herein are compositions, wherein the hybridization region comprises a reverse complement sequence of a sequence encoding an intron or a variant thereof. Further provided herein are compositions, wherein the hybridization region comprises a reverse complement sequence of a sequence encoding an exon or a variant thereof. Further provided herein are compositions, wherein the hybridization region comprises a reverse complement sequence of a sequence encoding an exon and an intron. Further provided herein are compositions, wherein the hybridization region comprises a sequence encoding for a splice acceptor site or a nucleotide complementary to the splice acceptor site. Further provided herein are compositions, wherein the hybridization region comprises a complement sequence of a sequence encoding an intron or a variant thereof. Further provided herein are compositions, wherein the hybridization region comprises a complement sequence of a sequence encoding an exon or a variant thereof. Further provided herein are compositions, wherein the hybridization region comprises a complement sequence of a sequence encoding an exon and an intron. Further provided herein are compositions, wherein the hybridization region comprises a mismatched nucleobase relative to a splice acceptor site in a sequence of the target nucleic acid. Further provided herein are compositions, wherein the ribonucleotides are on the 3′ end of the hybridization region. Further provided herein are compositions, wherein the hybridization region comprises 1 ribonucleotide, 2 ribonucleotides, 3 ribonucleotides, 4 ribonucleotides, 5 ribonucleotides, or up to 10 ribonucleotides. Further provided herein are compositions, wherein the hybridization region is at least about 5 nucleotides up to 10 nucleotides in length. Further provided herein are compositions, wherein the compositions further comprise an engineered protein or a polypeptide encoding the engineered protein. Further provided herein are compositions, wherein the engineered protein comprises: (a) a nickase region; and (b) a DdDP region. Further provided herein are compositions, wherein the DdDP region binds to the DNA-dependent DNA polymerase synthesis template (DST) region of the guide polynucleotide. Further provided herein are compositions, wherein the DdDP region synthesizes a new DNA strand using the DST region of the guide polynucleotide. Further provided herein are compositions, wherein the nickase region comprises a mutant Cas protein that generates a single-stranded break in a target nucleic acid. Further provided herein are compositions, wherein the Cas protein comprises: a Cas1, a Cas1B, a Cas2, a Cas3, a Cas4, a Cas5, a Cas6, a Cas7, a Cas8, a Cas9, a Cas10, a Cas11, a Cas12, a Cas13, a Cas14, a Csy1, a Csy2, a Csy3, a Cse1, a Cse2, a Csc1, a Csc2, a Csa5, a Csn2, a Csm2, a Csm3, a Csm4, a Csm5, a Csm6, a Cmr1, a Cmr3, a Cmr4, a Cmr5, a Cmr6, a Csb1, a Csb2, a Csb3, a Csx17, a Csx14, a Csx10, a Csx16, a CsaX, a Csx3, a Csx1, a Csx1S, a Csf1, a Csf2, a CsO, a Csf4, a c2c1, a c2c3, a Cas9HiFi, an xCas9, a CasX, a CasY, a CasRX, a SpCas9-VQR, a SpCas9-VRQR, a SpCas9-VRER, a SaCas9-KKH, a SpCas9-NG, a SpCas9-NRRH, a SpCas9-NRTH, a SpCas9-NRCH, a iSpyMac, a St1Cas9 LMD9-LMG18311, a St1Cas9 LMD9-CNRZ1066, a St1Cas9-KQKL, a variant, or any combination thereof. Further provided herein are compositions, wherein the compositions further comprise a delivery vehicle. Further provided herein are compositions, wherein the delivery vehicle comprises a vector, a lipid, a nanoparticle, a plasmid, a virus, a liposome, an extracellular vesicle, an emulsion, a peptide, a carbohydrate, a polymer, chitosan, polyethyleneimine (PEI), Poly (lactide-co-glycolide) (PLGA), Poly-L-lysine (PLL), or a combination thereof.

Provided herein are methods of synthesizing a nucleic acid, wherein the methods comprise: contacting a cell or a cell-free system with: a system provided herein, a polynucleotide provided herein, a vector provided herein, a composition provided herein, an engineered protein provided herein, or a guide polynucleotide provided herein.

Provided herein are methods of synthesizing a nucleic acid, wherein the methods comprise: contacting a cell or a cell-free system with: (a) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration relative to the target nucleic acid; (iv) a hybridization region, wherein the hybridization region comprises one or more ribonucleotides; (b) an engineered protein or a polynucleotide encoding the engineered protein, wherein the engineered protein comprises: (i) a nickase region; and (ii) a DdDP region; wherein: the guide polynucleotide forms a complex with the engineered protein via the protein binding region, the targeting sequence forms a complex with a complementary strand of the target nucleic acid, the nickase region of the engineered protein generates a single strand break in the target nucleic acid to generate a leading strand, the hybridization region forms a complex with the leading strand, and wherein the guide polynucleotide associates with the DdDP region of the engineered protein, thereby synthesizing a nucleic acid. Provided herein are methods of synthesizing a nucleic acid, wherein the methods comprise: contacting a cell or a cell-free system with: (a) a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration relative to the target nucleic acid; (iv) a hybridization region, wherein the hybridization region comprises a deoxyribonucleotide and a ribonucleotide; (b) an engineered protein or a polynucleotide encoding the engineered protein, wherein the engineered protein comprises: (i) a nickase region; and (ii) a DdDP region; wherein: the guide polynucleotide forms a complex with the engineered protein via the protein binding region, the targeting sequence forms a complex with a complementary strand of the target nucleic acid, the nickase region of the engineered protein generates a single strand break in the target nucleic acid to generate a leading strand, the hybridization region forms a complex with the leading strand, and wherein the guide polynucleotide associates with the DdDP region of the engineered protein, thereby synthesizing a nucleic acid. Further provided herein are methods, wherein the targeting strand dissociates from the complementary strand, wherein the complementary strand comprises a PAM sequence. Further provided herein are methods, wherein the new nucleic acid is incorporated into the target nucleic acid by hybridizing to the complementary strand. Further provided herein are methods, wherein the incorporation of the new nucleic acid recruits a DNA repair protein to the target nucleic acid that edits a nucleobase of the target nucleic acid.

Provided herein are methods, wherein the methods comprise: administering to a cell, a tissue, or a subject a system provided herein, a composition provided herein, a vector provided herein, a polynucleotide provided herein, or a cell provided herein, wherein the administering generates an alteration in a target nucleic acid. Further provided herein are methods, wherein the alteration comprises: an insertion, a deletion, a substitution, a change in copy number, a point mutation, a frameshift mutation, a missense mutation, a nonsense mutation, a mutation in a stop codon, an epigenetic mark, or any combination thereof. Further provided herein are methods, wherein the administering is local or systemic. Further provided herein are methods, wherein the administering is intranasal administration, subcutaneous administration, intravenous administration, inhalation, intramuscular administration, intratumoral administration, peritumoral administration, intrathecal administration, vaginal administration, or intradermal administration. Further provided herein are methods, wherein the subject is a mammal. Further provided herein are methods, wherein the subject has, is suspected of having, or is diagnosed with a disease or a condition. Further provided herein are methods, wherein the disease or the condition comprises a genetic disease or condition. Further provided herein are methods, wherein the methods further comprise administering to the subject a therapeutic agent.

Provided herein are methods, wherein the methods comprise: administering to a cell, a tissue, or a subject a system provided herein, a composition provided herein, a vector provided herein, a polynucleotide provided herein, thereby generating an alteration in a gene of the cell. Further provided herein are methods, wherein the contacting is performed in vitro, in vivo, or ex vivo. Further provided herein are methods, wherein the alteration in the gene comprises: an insertion, a deletion, a substitution, a change in copy number, a point mutation, a frameshift mutation, a missense mutation, a nonsense mutation, a mutation in a stop codon, an epigenetic mark, or any combination thereof. Further provided herein are methods, wherein the alteration in the gene restores expression of a wild-type protein that is encoded by the gene relative to a comparable cell or population of cells that were not contacted with the system or the composition. Further provided herein are methods, wherein the cell comprises a eukaryotic cell or a prokaryotic cell. Further provided herein are methods, wherein the cell comprises a mammalian cell. Further provided herein are methods, wherein the population of cells comprises a population of human leukocytes, a population of stem cells, or a population of bacteria.

Provided herein are populations of cells made by the methods provided herein.

Provided herein are kits, wherein the kits comprise: a system provided herein, a composition provided herein, a vector provided herein, a polynucleotide provided herein, or a cell provided herein, packaging and materials therefor.

Provided herein are kits, wherein the kits comprise: a first container comprising: a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration nucleobase relative to the target nucleic acid; and (iv) a hybridization region, wherein the hybridization region comprises: a deoxyribonucleotide and a ribonucleotide, and a second container comprising: an engineered protein comprising a nickase operably linked to a DNA-dependent DNA polymerase. Provided herein are kits, wherein the kits comprise: a first container comprising: a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease or a nickase; and (iii) a DNA-dependent DNA polymerase synthesis template (DST) region, wherein the DST region comprises a sequence that has complementarity to a target nucleic acid and at least one alteration nucleobase relative to the target nucleic acid; and (iv) a hybridization region, wherein the hybridization region comprises: one or more ribonucleotides, and a second container comprising: an engineered protein comprising a nickase operably linked to a DNA-dependent DNA polymerase.

Provided herein are scaffolds, wherein the scaffold comprise: a system provided herein or a composition provided herein; and a surface, wherein the system or the composition are immobilized to the surface.

### EXAMPLES

### Example 1: Editing a Double-Stranded Target with the DNA-Dependent DNA Polymerase Editing System

A DNA editing system comprising Cas9 H840A nickase, DdDP and gRNA was made as described herein and the editing function of the system was evaluated with different double-stranded DNA targets (double-stranded DNA substrate). In particular, double-stranded DNA substrates, gRNA, and the RNP formed by Cas9 H840A nickase, DdDP and gRNA were generated and assessed as described below.

Double-stranded DNA substrate generation: 5′-FAM-labeled double-stranded DNA (dsDNA) were generated by annealing two oligos, Oligo 1 and Oligo 2, at a 1:1 ratio. In summary, the oligos were resuspended in nuclease-free water to 100 μM concentration. Subsequently, 10 μL of 5′-FAM-labeled oligo (Oligo 1) and 10 μL of Oligo 2 oligo were mixed with 10 μL of NEBuffer r3.1 (10×) and 70 μL of nuclease-free water in PCR tubes and incubated at 95 degrees Celsius for 5 mins and slowly cooled down at room temperature. The annealed FAM-labelled dsDNA was made at a concentration of 10 μM, or 10 picomoles/μL. Oligo sequences are listed in Table 3.

In vitro guide RNA generation: In summary, a PCR was performed (Q5® Hot Start High-Fidelity 2× Master Mix, NEB) using oligos (Oligo 3, Oligo 4, Oligo 5 and Oligo 6) to generate double-stranded DNA template containing the T7 promoter for in vitro transcription. DNA from the PCR reaction was purified using AMPure XP® Beads (Beckman Coulter) and the concentration was measured using NanoDrop® (Thermo Fisher Scientific, Waltham, MA). 75 ng of DNA template was used for in vitro transcription reaction using HiScribe® T7 Quick High Yield RNA Synthesis Kit (NEB). The gRNA product was purified using the Monarch® RNA Cleanup Kit (NEB) and the concentration was measured using the NanoDrop® (Thermo Fisher Scientific). Oligo sequences are listed in Table 4. In Table 4, A, G, C, T are deoxyribonucleotides (DNA) and rA, rG, rC, rU are ribonucleotides (RNA).

TABLE 4
Oligonucleotides.

| SEQ | | |
|---|---|---|
| ID | | |
| NO: | Name | Sequence (Listed from 5′ to 3′) |
| 1 | Oligo 1 | /5′6- |
| | | FAM/GATCACTTAGAGCAATCGGCCCAGACTGAGCACGTGATGGCAGAGTACT |
| | | AGGAT |
| 2 | Oligo 2 | ATCCTAGTACTCTGCCATCACGTGCTCAGTCTGGGCCGATTGCTCTAAGTGATC |
| 3 | Oligo 3 | GGATCCTAATACGACTCACTATAGGCCCAGACTGAGCACGTGAGTTTTAGAGC |
| | | TAGAA |
| 4 | Oligo 4 | GCACCGACTCGGTGCCACTTTTTCAAGTTGATAACGGACTAGCCTTATTTTAAC |
| | | TTGCTATTTCTAGCTCTAAAAC |
| 5 | Oligo 5 | GGATCCTAATACGACTCACTATAG |
| 6 | Oligo 6 | AAAAGCACCGACTCGG |
| 7 | Oligo 7 | AAAAGAGCACGAGATTGCAGAGTACTGCACCGACTCGG |
| 8 | Oligo 8 | AAAAACTGAGCACGAGATTGCAGAGTACTGCACCGACTCGG |
| 9 | Oligo 9 | AAAAGAGCACGAGATTGCAGAGTACTAGATGACGTAGCACCGACTCGG |
| 10 | Oligo | AAAAACTGAGCACGAGATTGCAGAGTACTAGATGACGTAGCACCGACTCGG |
| | 10 | |
| 11 | Oligo | /5Phos/AGATTGCAGAGTACT |
| | 11 | |
| 12 | Oligo | /5Phos/AGATTGCAGAGTACTAGATGACGTA |
| | 12 | |
| 13 | Oligo | /5Phos/AGATTGCAGAGTACTAGATGACGTACGCAG |
| | 13 | |
| 14 | Oligo | /5Phos/AGATTGCAGAGTACTAGATGACGTACGCAGACCAAGAACCGCAAGATG |
| | 14 | CG |
| 15 | Oligo | /5Phos/AGATTGCAGAGTACTAGATGACGTACGCAGACCAAGAACCGCAAGATG |
| | 15 | CGACGGTGTACAAGTAATTGTC |
| 16 | Oligo | /5Phos/AGATTGCAGAGTACTAGATGACGTACGCAGACCAAGAACCGCAAGATG |
| | 16 | CGACGGTGTACAAGTAATTGTCAACAGACCATCGTGTTTTCA |
| 17 | Oligo | AGTACTCTGCAATCTCGTGCATTT |
| | 17 | |
| 18 | Oligo | AGTACTCTGCAATCTCGTGCTTTTT |
| | 18 | |
| 19 | Oligo | AGTACTCTGCAATCTCGTGCTCTTTT |
| | 19 | |
| 20 | Oligo | AGTACTCTGCAATCTCGTGCTCATTTT |
| | 20 | |
| 21 | Oligo | AGTACTCTGCAATCTCGTGCTCAGATTT |
| | 21 | |
| 22 | Oligo | AGTACTCTGCAATCTCGTGCTCAGTTTTT |
| | 22 | |
| 23 | Oligo | CTGCGTACGTCATCTAGTACTCTGCAATCTCGTGCATTT |
| | 23 | |
| 24 | Oligo | CGCATCTTGCGGTTCTTGGTCTGCGTACGTCATCTAGTACTCTGCAATCTCGTG |
| | 24 | CATTT |
| 25 | Oligo | CTGCGTACGTCATCTAGTACTCTGCAATCTCGTGCTCTTTT |
| | 25 | |
| 26 | Oligo | CGCATCTTGCGGTTCTTGGTCTGCGTACGTCATCTAGTACTCTGCAATCTCGTG |
| | 26 | CTCTTT |
| 27 | Oligo | AGTACTCTGCAATCTCGTGCrUrCTTTT |
| | 27 | |
| 28 | Oligo | AGTACTCTGCAATCTCGTGrCrUrCTTTT |
| | 28 | |
| 29 | Oligo | AGTACTCTGCAATCTCGTrGrCrUrCTTTT |
| | 29 | |
| 30 | Oligo | AGTACTCTGCAATCTCGrUrGrCrUrCTTTT |
| | 30 | |
| 31 | Oligo | AGTACTCTGCAATCTCrGrUrGrCrUrCTTTT |
| | 31 | |
| 32 | Oligo | /56-FAM/GATCACTTAGAGCAATCGGCCCAGACTGAGCACG |
| | 32 | |
| 33 | Oligo | TGATGGCAGAGTACTAGGAT |
| | 33 | |
| 34 | Oligo | CTGCGTACGTCATCTAGTACTCTGCAATCTCGTGCTCATTTT |
| | 34 | |
| 35 | Oligo | CTGCGTACGTCATCTAGTACTCTGCAATCTCGTGCTCAGATTT |
| | 35 | |
| 36 | Oligo | CTGCGTACGTCATCTAGTACTCTGCAATCTCGTGCTCAGTTTTT |
| | 36 | |
| 37 | Oligo | CTGCGTACGTCATCTAGTACTCTGCAATCTCGTGCTCAGTCATTT |
| | 37 | |
| 38 | Oligo | CTGCGTACGTCATCTAGTACTCTGCAATCTCGTGCTCAGTCTTTTT |
| | 38 | |
| 39 | Oligo | CTGCGTACGTCATCTAGTACTCTGCAATCTCGTGCTCAGTCTGTTTT |
| | 39 | |
| 40 | Oligo | /5Phos/GGATCGCAGAGGAAA |
| | 40 | |
| 41 | Oligo | /5Phos/GGATCGCAGAGGAAAGGAAGCCCTGCTTCC |
| | 41 | |
| 42 | Oligo | AGTACTCTGCAATCTrCrGrUrGrCrUrCTTTT |
| | 42 | |
| 43 | Oligo | AGTACTCTGCAATCTCGTGCTICTTTT |
| | 43 | |
| 44 | Oligo | AGTACTCTGCAATCTCGTGCTCTTTT |
| | 44 | |
| 113 | oRVB_ | ACACTCTTTCCCTACACGACGCTCTTCCGATCTAACGGATGGATGTGGCGCAG |
| | 1 | GTAG |
| 114 | oRVB_ | ACACTCTTTCCCTACACGACGCTCTTCCGATCTAAGTGATGGATGTGGCGCAG |
| | 2 | GTAG |
| 115 | oRVB_ | ACACTCTTTCCCTACACGACGCTCTTCCGATCTAGCTGATGGATGTGGCGCAG |
| | 3 | GTAG |
| 116 | oRVB_ | ACACTCTTTCCCTACACGACGCTCTTCCGATCTATAGGATGGATGTGGCGCAG |
| | 4 | GTAG |
| 117 | oRVB_ | ACACTCTTTCCCTACACGACGCTCTTCCGATCTCGGAGATGGATGTGGCGCAG |
| | 5 | GTAG |
| 118 | oRVB_ | ACACTCTTTCCCTACACGACGCTCTTCCGATCTCTTGGATGGATGTGGCGCAG |
| | 6 | GTAG |
| 119 | oRVB_ | ACACTCTTTCCCTACACGACGCTCTTCCGATCTGAACGATGGATGTGGCGCAG |
| | 7 | GTAG |
| 120 | oRVB_ | ACACTCTTTCCCTACACGACGCTCTTCCGATCTGAGTGATGGATGTGGCGCAG |
| | 8 | GTAG |
| 121 | oRVB_ | ACACTCTTTCCCTACACGACGCTCTTCCGATCTGCAGGATGGATGTGGCGCAG |
| | 9 | GTAG |
| 122 | oRVB_ | ACACTCTTTCCCTACACGACGCTCTTCCGATCTGTAAGATGGATGTGGCGCAG |
| | 10 | GTAG |
| 123 | oRVB_ | ACACTCTTTCCCTACACGACGCTCTTCCGATCTTACAGATGGATGTGGCGCAG |
| | 11 | GTAG |
| 124 | oRVB_ | ACACTCTTTCCCTACACGACGCTCTTCCGATCTTCTAGATGGATGTGGCGCAG |
| | 12 | GTAG |
| 125 | oRVB_ | GACTGGAGTTCAGACGTGTGCTCTTCCGATCTACAGAGGCGTATCATTTCGCG |
| | 13 | GAT |
| 126 | oRVB_ | GACTGGAGTTCAGACGTGTGCTCTTCCGATCTAGAAAGGCGTATCATTTCGCG |
| | 14 | GAT |
| 127 | oRVB_ | GACTGGAGTTCAGACGTGTGCTCTTCCGATCTCTAAAGGCGTATCATTTCGCG |
| | 15 | GAT |
| 128 | oRVB_ | GACTGGAGTTCAGACGTGTGCTCTTCCGATCTCTGCAGGCGTATCATTTCGCG |
| | 16 | GAT |
| 129 | oRVB_ | GACTGGAGTTCAGACGTGTGCTCTTCCGATCTGCGGAGGCGTATCATTTCGCG |
| | 17 | GAT |
| 130 | oRVB_ | GACTGGAGTTCAGACGTGTGCTCTTCCGATCTGGATAGGCGTATCATTTCGCG |
| | 18 | GAT |
| 131 | oRVB_ | GACTGGAGTTCAGACGTGTGCTCTTCCGATCTTCAAAGGCGTATCATTTCGCG |
| | 19 | GAT |
| 132 | oRVB_ | GACTGGAGTTCAGACGTGTGCTCTTCCGATCTTTCCAGGCGTATCATTTCGCG |
| | 20 | GAT |
| 133 | oRVB_ | ACACTCTTTCCCTACACGACGCTCTTCCGATCTAACGGCATGGATGAGAGAAG |
| | 21 | CCTGGAG |
| 134 | oRVB_ | ACACTCTTTCCCTACACGACGCTCTTCCGATCTAAGTGCATGGATGAGAGAAG |
| | 22 | CCTGGAG |
| 135 | oRVB_ | ACACTCTTTCCCTACACGACGCTCTTCCGATCTAGCTGCATGGATGAGAGAAG |
| | 23 | CCTGGAG |
| 136 | oRVB_ | ACACTCTTTCCCTACACGACGCTCTTCCGATCTATAGGCATGGATGAGAGAAG |
| | 24 | CCTGGAG |
| 137 | oRVB_ | ACACTCTTTCCCTACACGACGCTCTTCCGATCTCGGAGCATGGATGAGAGAAG |
| | 25 | CCTGGAG |
| 138 | oRVB_ | ACACTCTTTCCCTACACGACGCTCTTCCGATCTCTTGGCATGGATGAGAGAAG |
| | 26 | CCTGGAG |
| 139 | oRVB_ | ACACTCTTTCCCTACACGACGCTCTTCCGATCTGAACGCATGGATGAGAGAAG |
| | 27 | CCTGGAG |
| 140 | oRVB_ | ACACTCTTTCCCTACACGACGCTCTTCCGATCTGAGTGCATGGATGAGAGAAG |
| | 28 | CCTGGAG |
| 141 | oRVB_ | ACACTCTTTCCCTACACGACGCTCTTCCGATCTGCAGGCATGGATGAGAGAAG |
| | 29 | CCTGGAG |
| 142 | oRVB_ | ACACTCTTTCCCTACACGACGCTCTTCCGATCTGTAAGCATGGATGAGAGAAG |
| | 30 | CCTGGAG |
| 143 | oRVB_ | ACACTCTTTCCCTACACGACGCTCTTCCGATCTTACAGCATGGATGAGAGAAG |
| | 31 | CCTGGAG |
| 144 | oRVB_ | ACACTCTTTCCCTACACGACGCTCTTCCGATCTTCTAGCATGGATGAGAGAAG |
| | 32 | CCTGGAG |
| 145 | oRVB_ | GACTGGAGTTCAGACGTGTGCTCTTCCGATCTACAGCCCAGCCAAACTTGTCA |
| | 33 | ACC |
| 146 | oRVB_ | GACTGGAGTTCAGACGTGTGCTCTTCCGATCTAGAACCCAGCCAAACTTGTCA |
| | 34 | ACC |
| 147 | oRVB_ | GACTGGAGTTCAGACGTGTGCTCTTCCGATCTCTAACCCAGCCAAACTTGTCA |
| | 35 | ACC |
| 148 | oRVB_ | GACTGGAGTTCAGACGTGTGCTCTTCCGATCTCTGCCCCAGCCAAACTTGTCA |
| | 36 | ACC |
| 149 | oRVB_ | GACTGGAGTTCAGACGTGTGCTCTTCCGATCTGCGGCCCAGCCAAACTTGTC |
| | 37 | AACC |
| 150 | oRVB_ | GACTGGAGTTCAGACGTGTGCTCTTCCGATCTGGATCCCAGCCAAACTTGTCA |
| | 38 | ACC |
| 151 | oRVB_ | GACTGGAGTTCAGACGTGTGCTCTTCCGATCTTCAACCCAGCCAAACTTGTCA |
| | 39 | ACC |
| 152 | oRVB_ | GACTGGAGTTCAGACGTGTGCTCTTCCGATCTTTCCCCCAGCCAAACTTGTCA |
| | 40 | ACC |
| 153 | oRVB_ | ACACTCTTTCCCTACACGACGCTCTTCCGATCTTGCTGAACATGCGGAAGCGG |
| | 41 | |
| 154 | oRVB_ | GACTGGAGTTCAGACGTGTGCTCTTCCGATCTTTTCCAGCCTTGGGCATAGTC |
| | 42 | AGG |
| 155 | oRVB_ | ACACTCTTTCCCTACACGACGCTCTTCCGATCTTTCACAGCCAACGACTCCGG |
| | 43 | C |
| 156 | oRVB_ | GACTGGAGTTCAGACGTGTGCTCTTCCGATCTGCATATGAGGTGAAAACACTG |
| | 44 | CTTTAGTAAAAATGG |
| 157 | oRVB_ | ACACTCTTTCCCTACACGACGCTCTTCCGATCTAGGTCCCCTGGGAGGGGC |
| | 45 | |
| 158 | oRVB_ | GACTGGAGTTCAGACGTGTGCTCTTCCGATCTAGCCTGCTGACGCTGCCTT |
| | 46 | |
| 159 | oRVB_ | ACACTCTTTCCCTACACGACGCTCTTCCGATCTTCCGCTAAAGGCTGCTCTCTC |
| | 47 | AAC |
| 160 | oRVB_ | GACTGGAGTTCAGACGTGTGCTCTTCCGATCTTGTGACTGATGCTGTGGCAGG |
| | 48 | T |
| 161 | oRVB_ | ACACTCTTTCCCTACACGACGCTCTTCCGATCTCCATTATCTAATGGACGACCC |
| | 49 | CAGGG |
| 162 | oRVB_ | GACTGGAGTTCAGACGTGTGCTCTTCCGATCTACGTACAGCTGCCCATCCTTC |
| | 50 | C |
| 163 | oRVB_ | ACACTCTTTCCCTACACGACGCTCTTCCGATCTTGAAGCCAACCCACACAGTA |
| | 51 | TACCTATAGT |
| 164 | oRVB_ | GACTGGAGTTCAGACGTGTGCTCTTCCGATCTGAAGCATTTTCGATTCCGTGA |
| | 52 | CTTGGAAG |
| 165 | oRVB_ | ACACTCTTTCCCTACACGACGCTCTTCCGATCTCTGAACACTCCTCAAACGGT |
| | 53 | CCCC |
| 166 | oRVB_ | GACTGGAGTTCAGACGTGTGCTCTTCCGATCTCTTTCTCAAGGGGCTGCTGTG |
| | 54 | AGG |
| 167 | oRVB_ | ACACTCTTTCCCTACACGACGCTCTTCCGATCTAACGGGGGAGACTTGGTATT |
| | 55 | TTGTTCA |
| 168 | oRVB_ | ACACTCTTTCCCTACACGACGCTCTTCCGATCTAAGTGGGGAGACTTGGTATTT |
| | 56 | TGTTCA |
| 169 | oRVB_ | ACACTCTTTCCCTACACGACGCTCTTCCGATCTAGCTGGGGAGACTTGGTATTT |
| | 57 | TGTTCA |
| 170 | oRVB_ | ACACTCTTTCCCTACACGACGCTCTTCCGATCTATAGGGGGAGACTTGGTATTT |
| | 58 | TGTTCA |
| 171 | oRVB_ | ACACTCTTTCCCTACACGACGCTCTTCCGATCTCGGAGGGGAGACTTGGTATT |
| | 59 | TTGTTCA |
| 172 | oRVB_ | ACACTCTTTCCCTACACGACGCTCTTCCGATCTCTTGGGGGAGACTTGGTATTT |
| | 60 | TGTTCA |
| 173 | oRVB_ | ACACTCTTTCCCTACACGACGCTCTTCCGATCTGAACGGGGAGACTTGGTATT |
| | 61 | TTGTTCA |
| 174 | oRVB_ | ACACTCTTTCCCTACACGACGCTCTTCCGATCTGAGTGGGGAGACTTGGTATT |
| | 62 | TTGTTCA |
| 175 | oRVB_ | ACACTCTTTCCCTACACGACGCTCTTCCGATCTGCAGGGGGAGACTTGGTATT |
| | 63 | TTGTTCA |
| 176 | oRVB_ | ACACTCTTTCCCTACACGACGCTCTTCCGATCTGTAAGGGGAGACTTGGTATTT |
| | 64 | TGTTCA |
| 177 | oRVB_ | ACACTCTTTCCCTACACGACGCTCTTCCGATCTTACAGGGGAGACTTGGTATTT |
| | 65 | TGTTCA |
| 178 | oRVB_ | ACACTCTTTCCCTACACGACGCTCTTCCGATCTTCTAGGGGAGACTTGGTATTT |
| | 66 | TGTTCA |
| 179 | oRVB_ | GACTGGAGTTCAGACGTGTGCTCTTCCGATCTACAGGGAGTGAGCGCTTCCTG |
| | 67 | |
| 180 | oRVB_ | GACTGGAGTTCAGACGTGTGCTCTTCCGATCTAGAAGGAGTGAGCGCTTCCT |
| | 68 | G |
| 181 | oRVB_ | GACTGGAGTTCAGACGTGTGCTCTTCCGATCTCTAAGGAGTGAGCGCTTCCTG |
| | 69 | |
| 182 | oRVB_ | GACTGGAGTTCAGACGTGTGCTCTTCCGATCTCTGCGGAGTGAGCGCTTCCTG |
| | 70 | |
| 183 | oRVB_ | GACTGGAGTTCAGACGTGTGCTCTTCCGATCTGCGGGGAGTGAGCGCTTCCT |
| | 71 | G |
| 184 | oRVB_ | GACTGGAGTTCAGACGTGTGCTCTTCCGATCTGGATGGAGTGAGCGCTTCCTG |
| | 72 | |
| 185 | oRVB_ | GACTGGAGTTCAGACGTGTGCTCTTCCGATCTTCAAGGAGTGAGCGCTTCCTG |
| | 73 | |
| 186 | oRVB_ | GACTGGAGTTCAGACGTGTGCTCTTCCGATCTTTCCGGAGTGAGCGCTTCCTG |
| | 74 | |

Synthetic guide RNA (Syn-gRNA) generation: Synthetic guide polynucleotides were generated as listed in Table 5 or Table 6 by chemical synthesis. Guide polynucleotides were HPLC purified and the mass of individual gRNAs were verified by mass-spectrometry (LC-MS). In Table 5, A, C, G, U are ribonucleotides (RNA) and dA, dC, dG, dT are deoxyribonucleotides (DNA).

TABLE 5
Synthetic guide polynucleotides.

| SEQ ID NO: | Name | Sequence |
|---|---|---|
| 45 | Guide 1 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | dTdGdCU |
| 46 | Guide 2 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | dTdGdCdTU |
| 47 | Guide 3 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | dTdGdCdTdCU |
| 48 | Guide 4 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | dTdGdCUCU |
| 49 | Guide 5 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTaTdTdCdCdTdCdTdGdCdGdAdTdCdCdCdG |
| | | dTdGCUCU |
| 50 | Guide 6 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | dTGCUCU |
| 51 | Guide 7 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCdG |
| | | UGCUCU |
| 52 | Guide 8 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCdCGU |
| | | GCUCU |
| 53 | Guide 9 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdTdTdCdCdTdCdTdGdCdGdAdTdCdCCGU |
| | | GCUCU |
| 54 | Guide 10 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdGdGdAdAdGdCdAdGdGdGdCdTdTdCdCdTd |
| | | TdTdCdCdTdCdTaGdCdGdAdTdCdCCGUGCUCU |
| 55 | Guide 11 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCUUUCCUCUGCGAUCCCGUGCUCU |
| 56 | Guide 12 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCdTdCdTdGdCdGdAdTdCdCCGUGCUCUUUU |
| 57 | Guide 13 | GGCCCAGACUGAGCACGUGAGUUUUAGAGCUAGAAAUAGCA |
| | | AGUUAAAAUAAGGCUAGUCCGUUAUCAACUUGAAAAAGUGG |
| | | CACCGAGUCGGUGCUCUGCGdAdTdCdCdCdGdTdGCUCUUUU |

In Table 6, A, C, G, U are ribonucleotides (RNA) and dA, dC, dG, dT are deoxyribonucleotides (DNA), +A, +T, +C, +G are LNA, * signifies a Phosphorothioate (PS) bond, mA, mU, mC, mG correspond to 2′-O-Methyl RNA, and exNA-mU corresponds to 2′-OMe-exNA-uridine.

TABLE 6
Guide RNA Constructs.

| SEQ ID | | |
|---|---|---|
| NO: | Name | Sequence |
| 187 | gRVB_1 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdCdAdTdCdCdCdGdTGCUCAGUCUG |
| | | *mU*mU*mU |
| 188 | gRVB_2 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdCdAdTdCdCdCdGUGCUCAGUCUG* |
| | | mU*mU*mU |
| 189 | gRVB_3 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdCdAdTdCdCdCGUGCUCAGUCUG* |
| | | mU*mU*mU |
| 190 | gRVB_4 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGdCUGCAGAAG |
| | | *mG*mG*mA |
| 191 | gRVB_5 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGdCUGCAGA*m |
| | | A*mG*mG |
| 192 | gRVB_6 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGdCUGCA*mG* |
| | | mA*mA |
| 193 | gRVB_7 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdGdAdTdCdAdCdGdTdGdCUCA*mG* |
| | | mU*mC |
| 194 | gRVB_8 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdGdAdTdCdAdCdGUGCUCA*mG*mU |
| | | *mC |
| 195 | gRVB_9 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdTdTdCdCdTdCdTdGdCdGdAdTdCdAdCGUGCUCA*mG*mU* |
| | | mC |
| 196 | gRVB_10 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGdCdTdGdCAGA |
| | | *mA*mG*mG |
| 197 | gRVB_11 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGdCdTGCAGA*m |
| | | A*mG*mG |
| 198 | gRVB_12 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGCUGCAGA*mA |
| | | *mG*mG |
| 199 | gRVB_13 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdAdTdGdCUGCAGA*m |
| | | A*mG*mG |
| 200 | gRVB_14 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdCdTdGdCUGCAGA*m |
| | | A*mG*mG |
| 201 | gRVB_15 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdTdTdGdCUGCAGA*m |
| | | A*mG*mG |
| 202 | gRVB_16 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdGdCdGdCUGCAGA*m |
| | | A*mG*mG |
| 203 | gRVB_17 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdGdAdGdCUGCAGA*m |
| | | A*mG*mG |
| 204 | gRVB_18 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdCdAdGdGdGdGdCUGCAGA*m |
| | | A*mG*mG |
| 205 | gRVB_19 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdCdTdGdCdCdAdTdCdAdAdAdGdCGUGCUCA*mG*mU*mC |
| 206 | gRVB_20 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCdTdGdGdAdGdGdAdAdGdCdAdGdGdGdCdTdTdCdCdTdTdTdCd |
| | | CdTdCdTdGdCdCdAdCGUGCUCA*mG*mU*mC |
| 207 | gRVB_21 | mG*mU*mC*AACCAGUAUCCCGGUGCGUUUUAGAGCUAGAAAUAGCAA |
| | | GUUAAAAUAAGGCUAGUCCGUUAUCAACUUGAAAAAGUGGCACCGAG |
| | | UCGGUGCmU*mU*mU*U |
| 208 | gRVB_22 | mG*mG*mG*GUCCCAGGUGCUGACGUGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 209 | gRVB_23 | mA*mA*mA*UAAGGUAAAGAGAGGGAGUUUUAGAGCUAGAAAUAGCA |
| | | AGUUAAAAUAAGGCUAGUCCGUUAUCAACUUGAAAAAGUGGCACCGA |
| | | GUCGGUGCdTdCdCdCdTdGdGdGdTdCdCdCdTdGdCdCdAdGdGdTdCdCdCd |
| | | CUCUCUUUACCU*mU*mA*mU |
| 210 | gRVB_24 | mG*mC*mA*UAGUCAGGGACUCUCGUGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 211 | gRVB_25 | mA*mG*mU*CCCUCAUUCCUUGGGAUGUUUUAGAGCUAGAAAUAGCA |
| | | AGUUAAAAUAAGGCUAGUCCGUUAUCAACUUGAAAAAGUGGCACCGA |
| | | GUCGGUGCdTdCdCdAdCdCdAdCdGdGdCdTdGdTdCdAdCdAdAdAdTdCdCC |
| | | AAGGAAUG*mA*mG*mG |
| 212 | gRVB_26 | mG*mU*mA*UUCACAGCCAACGACUCGUUUUAGAGCUAGAAAUAGCAA |
| | | GUUAAAAUAAGGCUAGUCCGUUAUCAACUUGAAAAAGUGGCACCGAG |
| | | UCGGUGCmU*mU*mU*U |
| 213 | gRVB_27 | mG*mU*mU*UCGGCGCCAUAAGGUGGGUUUUAGAGCUAGAAAUAGCA |
| | | AGUUAAAAUAAGGCUAGUCCGUUAUCAACUUGAAAAAGUGGCACCGA |
| | | GUCGGUGCdGdCdAdAdAdTdTdGdTdCdAdGdAdCdGdAdCdAdAdCdCdAdC |
| | | CUUAUGG*mC*mG*mC |
| 214 | gRVB_28 | mC*mG*mC*UGCCUUGCCCUCCCAGUGUUUUAGAGCUAGAAAUAGCAA |
| | | GUUAAAAUAAGGCUAGUCCGUUAUCAACUUGAAAAAGUGGCACCGAG |
| | | UCGGUGCmU*mU*mU*U |
| 215 | gRVB_29 | mG*mC*mC*CAGAGCUGGGGCCCCCAGUUUUAGAGCUAGAAAUAGCAA |
| | | GUUAAAAUAAGGCUAGUCCGUUAUCAACUUGAAAAAGUGGCACCGAG |
| | | UCGGUGCdGdCdTdCdAdCdCdTdTdCdCdTdGdCdAdGdTdCdGdTdGdGdGGG |
| | | CCC*mC*mA*mG |
| 216 | gRVB_30 | mG*mA*mG*GAACGAUUUAAGUCUUGGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 217 | gRVB_31 | mA*mA*mA*GAGCAUGAUCACAUGCUGUUUUAGAGCUAGAAAUAGCA |
| | | AGUUAAAAUAAGGCUAGUCCGUUAUCAACUUGAAAAAGUGGCACCGA |
| | | GUCGGUGCdAdAdAdTdAdTdGdGdCdGdTdCdAdAdGdCdAUGUGAUCAUG* |
| | | mC*mU*mC |
| 218 | gRVB_32 | mU*mU*mA*UCUAAUGGACGACCCCAGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 219 | gRVB_33 | mA*mC*mU*GAGGGAUGUCUGAAGGCGUUUUAGAGCUAGAAAUAGCA |
| | | AGUUAAAAUAAGGCUAGUCCGUUAUCAACUUGAAAAAGUGGCACCGA |
| | | GUCGGUGCdGdCdCdAdGdTdAdTdCdCdCdTdGdGdCdCdTUCAGACAUC*m |
| | | C*mC*mU |
| 220 | gRVB_34 | mG*mC*mA*UUUUCGAUUCCGUGACUGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 221 | gRVB_35 | mG*mG*mA*GUGAGGGAAACGGCCCCGUUUUAGAGCUAGAAAUAGCA |
| | | AGUUAAAAUAAGGCUAGUCCGUUAUCAACUUGAAAAAGUGGCACCGA |
| | | GUCGGUGCdGdCdTdGdGdCdCdCdTdGdGdGdGdAdTdGCCGUUU*mC*mC* |
| | | mC |
| 222 | gRVB_36 | mA*mG*mC*UGCUCACUUGAGCCUCUGUUUUAGAGCUAGAAAUAGCAA |
| | | GUUAAAAUAAGGCUAGUCCGUUAUCAACUUGAAAAAGUGGCACCGAG |
| | | UCGGUGCmU*mU*mU*U |
| 223 | gRVB_37 | mG*mU*mG*CUGACCAUCGACGAGAAGUUUUAGAGCUAGAAAUAGCA |
| | | AGUUAAAAUAAGGCUAGUCCGUUAUCAACUUGAAAAAGUGGCACCGA |
| | | GUCGGUGCdAdGdTdCdCdGdTdTdTdCdTdTGUCGAUG*mG*mU*mC |
| 224 | gRVB_38 | mG*mA*mC*CUCGGGGGGGAUAGACAGUUUUAGAGCUAGAAAUAGCA |
| | | AGUUAAAAUAAGGCUAGUCCGUUAUCAACUUGAAAAAGUGGCACCGA |
| | | GUCGGUGCmU*mU*mU*U |
| 225 | gRVB_39 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGmCmUmGmCm |
| | | AmGmAmAmGmG*mU*mU*mUU |
| 226 | gRVB_40 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGC+GdG+AdA+AdA+GdC+GdA+TdC+GdTdGdAdTdGmCmUmGmCm |
| | | AmGmAmAmGmG*mU*mU*mUU |
| 227 | gRVB_41 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdG+GdA+AdA+AdG+CdG+AdT+CdG+TdG+AdT+GmCmUmGmC |
| | | mAmGmAmAmGmG*mU*mU*mUU |
| 228 | gRVB_42 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdG*dG*dA*dA*dA*dA*dG*dC*dG*dA*dT*dC*dG*dT*dG*dA*dT |
| | | *dGmCmUmGmCmAmGmAmAmGmG*mU*mU*mUU |
| 229 | gRVB_43 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdG*dAdA*dAdA*dGdC*dGdA*dTdC*dGdT*dGdA*dTdGmCmU |
| | | mGmCmAmGmAmAmGmG*mU*mU*mUU |
| 230 | gRVB_44 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdG+G*dA+A*dA+A*dG+C*dG+A*dT+C*dG+T*dG+A*dTdGmCm |
| | | UmGmCmAmGmAmAmGmG*mU*mU*mUU |
| 231 | gRVB_45 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCdGdGdAdAdAdAdGdCdGdAdTdCdGdTdGdAdTdGCUGCAGAAGG |
| | | *mU*mU*exNA-mU*exNA-mU |
| 232 | gRVB_46 | mG*mG*mC*CCAGACUGAGCACGUGAGUUUUAGAGCUAGAAAUAGCA |
| | | AGUUAAAAUAAGGCUAGUCCGUUAUCAACUUGAAAAAGUGGCACCGA |
| | | GUCGGUGCUCUGCCAUCAAAGCGUGCUCA*mG*mU*mC |
| 233 | gRVB_47 | mG*mG*mA*AUCCCUUCUGCAGCACCGUUUUAGAGCUAGAAAUAGCAA |
| | | GUUAAAAUAAGGCUAGUCCGUUAUCAACUUGAAAAAGUGGCACCGAG |
| | | UCGGUGCGGAAAAGCGAUCGUGAUGCUGCAGA*mA*mG*mG |

In vitro DNA nicking, DNA editing by DNA polymerase I, and analysis: First, CRISPR ribonucleoprotein (RNP) were formulated by combining 61 picomoles S.p. Cas9 H840A Nickase with 80 picomoles of guide RNA and incubated at room temperature for 10 mins to allow RNP complexation. Cas9 Nickase and DNA polymerase activities were performed in a total of 7 μL volume containing 1 μL of FAM dsDNA (10 picomoles), 100 picomoles oligo for trans DNA editing (for cis DNA editing, the DNA template was incorporated into the Syn-gRNA sequence), 0.7 μL of 10× NEBuffer 2, 0.5 μL of DNA polymerase, 2 μL of Cas9 Nickase-gRNA RNP and remaining volume of nuclease-free water to make a final volume of 7 μL. The reaction was incubated at 37 degrees C. for 1 hour and samples were treated with 0.5 μL of proteinase K solution (20 mg/mL, Qiagen) and incubated at 56 degrees Celsius for 30 min. Samples were heat inactivated at 95 degrees Celsius for 10 min, reaction products were combined with Gel Loading Buffer II (2×, ThermoFisher Scientific) and denatured at 95° C. for 5 min, and separated by denaturing polyacrylamide gel (15% TBE-urea, 60 degrees Celsius, 150V) for 1 hour. DNA products were visualized by FAM fluorescence signal using a Life Technologies Gel Imaging system.

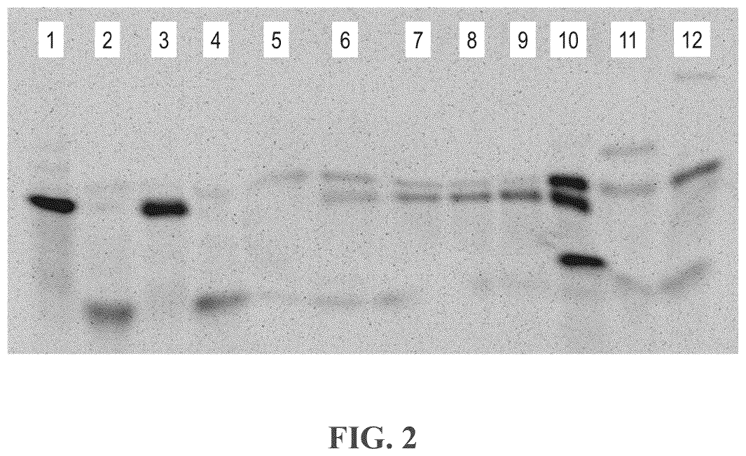

The editing products of targeted DNA editing by DNA-dependent DNA polymerase with the DNA-dependent DNA polymerase synthesis template (DST) and hybridization region (HR) were analyzed by denaturing urea polyacrylamide gel electrophoresis as shown in FIG. 2. The target edited products from DNA editing with DNA-dependent DNA polymerase synthesis template (DST) and hybridization region (HR) in trans with various double-stranded DNA substrates, SEQ ID NO: 17, SEQ ID NO: 18, SEQ ID NO: 19, SEQ ID NO: 20, SEQ ID NO: 21, and SEQ ID NO: 22, were shown in lanes 3-8 of FIG. 2, showing the DNA editing system successfully inserted 15 nucleotides into the DNA substrates. The DNA editing system also showed successful insertion of larger DNA fragments. As shown in lanes 10 and 11 of FIG. 2, the DNA editing system inserted 30 nucleotides and 50 nucleotides into the DNA substrates.

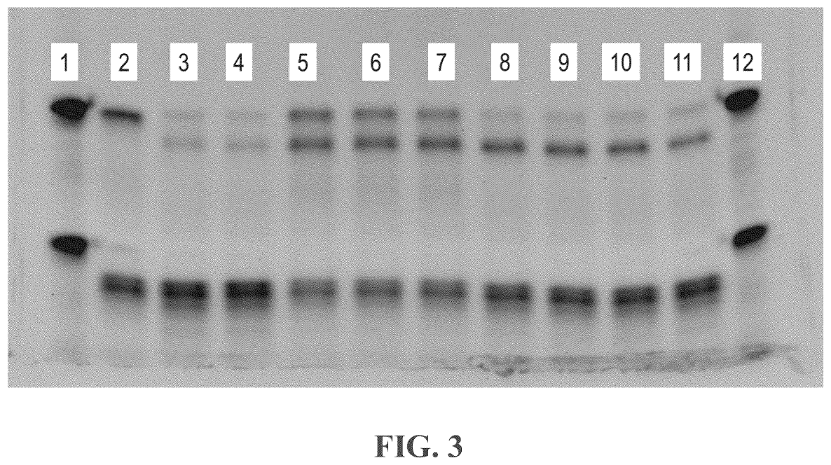

The effects on editing products of targeted DNA editing by DNA-dependent DNA polymerase with different synthetic single gRNAs were analyzed by denaturing urea polyacrylamide gel electrophoresis as shown in FIG. 3. In particular, the DNA-dependent DNA polymerase synthesis template comprises primarily all DNA bases, while the hybridization region comprises either deoxyribonucleotides (SEQ ID NOs: 45-47), ribonucleotides bases, or both RNA and DNA (SEQ ID NOs: 48-53). The hybridization region in Guide 1, Guide 2, and Guide 3 have the following architecture: 5′ DDDDD 3′, 5′ DDDDDD 3′, and 5′ DDDDDDD 3′, respectively (D represents DNA and R represents RNA). The hybridization region in Guide 4, Guide 5, and Guide 6, Guide 7, Guide 8, and Guide 9 have the following architecture: 5′ DDDDDRR 3′, 5′ DDDDRRR 3′, 5′ DDDRRRR 3′, 5′ DDRRRRR 3′, 5′ DRRRRRR 3′ and 5′ RRRRRRR 3′, respectively.

The results show that chimeric guide polynucleotides that include both DNA and RNA enhance DNA synthesis and editing of the target nucleic acid relative to non-chimeric RNA and DNA guides. Furthermore, increasing the length of the hybridization region increases the editing efficiency. In particular, a minimum length of 7 for the hybridization region nucleotides is recommended (lanes 5-7 as compared to lanes 3-4 in FIG. 3) for efficient editing. Additionally, increasing the amount of RNA in the hybridization region from 2 to 7 reduces the amount of non-nicked products, suggesting increasing the amount of RNA in the hybridization region of the guide polynucleotide increases the editing efficiency.

### Example 2: Plasmid Cloning and Editor Constructs

A backbone plasmid containing T7 RNA polymerase promoter, 5′ UTR sequence, and 3′ UTR sequence was constructed to clone various editor constructs. Gene fragments containing various Cas enzymes and DNA-dependent DNA polymerases (DdDP) were assembled utilizing ligation-based cloning or Gibson assembly into the backbone plasmid to serve as a template for in vitro transcription. All plasmids were midi or maxi prepped with the Qiagen Midi/Maxi Plus kits.

The amino acid sequences of 30 full-length DdDP and Nickase fusion proteins that were generated are provided in Table 7 below. The type of nickase is indicated in column 2 followed by any modifications relative to the reference sequence. For SpCas9, the reference sequence is SEQ ID NO: 92. For St1Cas9, the reference sequence is SEQ ID NO: 97. The type of DNA-dependent DNA polymerase is indicated in column 3 followed by any modifications relative to the reference sequence. The reference sequence for phi29 DNA polymerase is SEQ ID NO: 63.

TABLE 7
Gene Editing System Fusion Protein Sequences.

| | Cas- | DdDP- | |
|---|---|---|---|
| SEQ ID NO: | Substitutions | Substitutions | Editor Sequence |
| 234 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | | D12A, D66A | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSKHMPRK |
| | | | MYSCAFETTTKVEDCRVWAYGYMNIEDHSEYKIGNSLDEFMAWVLKVQADLYFHNLKFAGAFIINWLERNGFKWSADGLPN |
| | | | TYNTIISRMGQWYMIDICLGYKGKRKIHTVIYDSLKKLPFPVKKIAKDFKLTVLKGDIDYHKERPVGYKITPEEYAYIKNDIQIIA |
| | | | EALLIQFKQGLDRMTAGSDSLKGFKDIITTKKFKKVFPTLSLGLDKEVRYAYRGGFTWLNDRFKEKEIGEGMVFDVNSLYPAQ |
| | | | MYSRLLPYGEPIVFEGKYVWDEDYPLHIQHIRCEFELKEGYIPTIQIKRSRFYKGNEYLKSSGGEIADLWLSNVDLELMKEHYD |
| | | | LYNVEYISGLKFKATTGLFKDFIDKWTYIKTTSEGAIKQLAKLMLNSLYGKFASNPDVTGKVPYLKENGALGFRLGEEETKDP |
| | | | VYTPMGVFITAWARYTTITAAQACYDRIIYCDTDSIHLTGTEIPDVIKDIVDPKKLGYWAHESTFKRAKYLRQKTYIQDIYMKE |
| | | | VDGKLVEGSPDDYTDIKFSVKCAGMTDKIKKEVTFENFKVGFSRKMKPKPVQVPGGVVLVDDTFTIKSGGSKRTADGSEFEPK |
| | | | KKRKV |
| 235 | SpCas9 | Ba71V DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase | RTARRRYTRRKNRICYLQEIFSNEMAKVDDSFFHRLEESFLVEEDKKHERHPIFGNIVDEVAYHEKYPTIYHLRKKLVDSTDKA |
| | | | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSLTLIQGK |
| | | | KIVNHLRSRLAFEYNGQLIKILSKNIVAVGSLRREEKMLNDVDLLIIVPEKKLLKHVLPNIRIKGLSFSVKVCGERKCVLFIEWEK |
| | | | KTYQLDLFTALAEEKPYAIFHFTGPVSYLIRIRAALKKKNYKLNQYGLFKNQTLVPLKITTEKELIKELGFTYRIPKKRLSGGSK |
| | | | RTADGSEFEPKKKRKV |
| 236 | SpCas9 | Human DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase | RTARRRYTRRKNRICYLQEIFSNEMAKVDDSFFHRLEESFLVEEDKKHERHPIFGNIVDEVAYHEKYPTIYHLRKKLVDSTDKA |
| | | lambda | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSDPRGILK |
| | | | AFPKRQKIHADASSKVLAKIPRREEGEEAEEWLSSLRAHVVRTGIGRARAELFEKQIVQHGGQLCPAQGPGVTHIVVDEGMDY |
| | | | ERALRLLRLPQLPPGAQLVKSAWLSLCLQERRLVDVAGFSIFIPSRYLDHPQPSKAEQDASIPPGTHEALLQTALSPPPPPTRPVS |
| | | | PPQKAKEAPNTQAQPISDDEASDGEETQVSAADLEALISGHYPTSLEGDCEPSPAPAVLDKWVCAQPSSQKATNHNLHITEKLE |
| | | | VLAKAYSVQGDKWRALGYAKAINALKSFHKPVTSYQEACSIPGIGKRMAEKIIEILESGHLRKLDHISESVPVLELFSNIWGAGT |
| | | | KTAQMWYQQGFRSLEDIRSQASLTTQQAIGLKHYSDFLERMPREEATEIEQTVQKAAQAFNSGLLCVACGSYRRGKATCGDV |
| | | | DVLITHPDGRSHRGIFSRLLDSLRQEGFLTDDLVSQEENGQQQKYLGVCRLPGPGRRHRRLDIIVVPYSEFACALLYFTGSAHFN |
| | | | RSMRALAKTKGMSLSEHALSTAVVRNTHGCKVGPGRVLPTPTEKDVFRLLGLPYREPAERDWSGGSKRTADGSEFEPKKKRK |
| | | | V |
| 237 | SpCas9 | Human DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase | RTARRRYTRRKNRICYLQEIFSNEMAKVDDSFFHRLEESFLVEEDKKHERHPIFGNIVDEVAYHEKYPTIYHLRKKLVDSTDKA |
| | | beta | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSSKRKAP |
| | | | QETLNGGITDMLTELANFEKNVSQAIHKYNAYRKAASVIAKYPHKIKSGAEAKKLPGVGTKIAEKIDEFLATGKLRKLEKIRQD |
| | | | DTSSSINFLTRVSGIGPSAARKFVDEGIKTLEDLRKNEDKLNHHQRIGLKYFGDFEKRIPREEMLQMQDIVLNEVKKVDSEYIAT |
| | | | VCGSFRRGAESSGDMDVLLTHPSFTSESTKQPKLLHQVVEQLQKVHFITDTLSKGETKFMGVCQLPSKNDEKEYPHRRIDIRLIP |
| | | | KDQYYCGVLYFTGSDIFNKNMRAHALEKGFTINEYTIRPLGVTGVAGEPLPVDSEKDIFDYIQWKYREPKDRSESGGSKRTAD |
| | | | GSEFEPKKKRKV |
| 238 | SpCas9 | Bsu DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase I | RTARRRYTRRKNRICYLQEIFSNEMAKVDDSFFHRLEESFLVEEDKKHERHPIFGNIVDEVAYHEKYPTIYHLRKKLVDSTDKA |
| | | | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSKNKLVL |
| | | | IDGNSVAYRAFFALPLLHNDKGIHTNAVYGFTMMLNKILAEEQPTHILVAFDAGKTTFRHETFQDYKGGRQQTPPELSEQFPLL |
| | | | RELLKAYRIPAYELDHYEADDIIGTMAARAEREGFAVKVISGDRDLTQLASPQVTVEITKKGITDIESYTPETVVEKYGLTPEQI |
| | | | VDLKGLMGDKSDNIPGVPGIGEKTAVKLLKQFGTVENVLASIDEIKGEKLKENLRQYRDLALLSKQLAAICRDAPVELTLDDIV |
| | | | YKGEDREKVVALFQELGFQSFLDKMAVQTDEGEKPLAGMDFAIADSVTDEMLADKAALVVEVVGDNYHHAPIVGIALANER |
| | | | GRFFLRPETALADPKFLAWLGDETKKKTMFDSKRAAVALKWKGIELRGVVFDLLLAAYLLDPAQAAGDVAAVAKMHQYEA |
| | | | VRSDEAVYGKGAKRTVPDEPTLAEHLVRKAAAIWALEEPLMDELRRNEQDRLLTELEQPLAGILANMEFTGVKVDTKRLEQ |
| | | | MGAELTEQLQAVERRIYELAGQEFNINSPKQLGTVLFDKLQLPVLKKTKTGYSTSADVLEKLAPHHEIVEHILHYRQLGKLQST |
| | | | YIEGLLKVVHPVTGKVHTMFNQALTQTGRLSSVEPNLQNIPIRLEEGRKIRQAFVPSEPDWLIFAADYSQIELRVLAHIAEDDNL |
| | | | IEAFRRGLDIHTKTAMDIFHVSEEDVTANMRRQAKAVNFGIVYGISDYGLAQNLNITRKEAAEFIERYFASFPGVKQYMDNIVQ |
| | | | EAKQKGYVTTLLHRRRYLPDITSRNFNVRSFAERTAMNTPIQGSAADIIKKAMIDLSVRLREERLQARLLLQVHDELILEAPKEE |
| | | | IERLCRLVPEVMEQAVTLRVPLKVDYHYGPTWYDAKSGGSKRTADGSEFEPKKKRKV |
| 239 | SpCas9 | Klenow | MKRTADGSEFESPKKKRKVDKKYSIGLDIGTNSVGWAVITDEYKVPSKKFKVLGNTDRHSIKKNLIGALLFDSGETAEATRLK |
| | H840A | fragment - | RTARRRYTRRKNRICYLQEIFSNEMAKVDDSFFHRLEESFLVEEDKKHERHPIFGNIVDEVAYHEKYPTIYHLRKKLVDSTDKA |
| | | D33A, E35A | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSVISYDN |
| | | | YVTILDEETLKAWIAKLEKAPVFAFATATDSLDNISANLVGLSFAIEPGVAAYIPVAHDYLDAPDQISRERALELLKPLLEDEKA |
| | | | LKVGQNLKYDRGILANYGIELRGIAFDTMLESYILNSVAGRHDMDSLAERWLKHKTITFEEIAGKGKNQLTFNQIALEEAGRY |
| | | | AAEDADVTLQLHLKMWPDLQKHKGPLNVFENIEMPLVPVLSRIERNGVKIDPKVLHNHSEELTLRLAELEKKAHEIAGEEFNL |
| | | | SSTKQLQTILFEKQGIKPLKKTPGGAPSTSEEVLEELALDYPLPKVILEYRGLAKLKSTYTDKLPLMINPKTGRVHTSYHQAVTA |
| | | | TGRLSSTDPNLQNIPVRNEEGRRIRQAFIAPEDYVIVSADYSQIELRIMAHLSRDKGLLTAFAEGKDIHRATAAEVFGLPLETVTS |
| | | | EQRRSAKAINFGLIYGMSAFGLARQLNIPRKEAQKYMDLYFERYPGVLEYMERTRAQAKEQGYVETLDGRRLYLPDIKSSNG |
| | | | ARRAAAERAAINAPMQGTAADIIKRAMIAVDAWLQAEQPRVRMIMQVHDELVFEVHKDDVDAVAKQIHQLMENCTRLDVP |
| | | | LLVEVGSGENWDQAHSGGSKRTADGSEFEPKKKRKV |
| 240 | SpCas9 | T3 DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | | D5A, E7A | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSLVSAIAA |
| | | | NNLLEKVTKFHCGVIYDYRDGEYHSYRPGDFGAYLDALEAEVKRGGLIVFHNGHKYDVPALTKLAKLQLNREFHLPRENCID |
| | | | TLVLSRLIHSNLKDTDMGLLRSGKLPGKRFGSHALEAWGYRLGEMKGEYKDDFKRMLEEQGEEYVDGMEWWNFNEEMMD |
| | | | YNVQDVVVTKALLEKLLSDKHYFPPEIDFTDVGYTTFWSESLEAVDVEHRAAWLLAKQERNGFPFDTKAIEELYVELAARRSE |
| | | | LLRNLTETFGSWYQPKGGTEMFCHPRTGKPLPKYPRIKIPKVGGIFKKPKNKAQREGREPCELDTREYVAGAPYTPVEHVVFN |
| | | | PSSRDHIQKKLQEAGWVPTKFTDKGAPVVDDEVLEGVRVDDPEKQAAIDLIKEYLMIQKRIGQSAEGDKAWLRYVAEDGKIH |
| | | | GSVNPNGAVTGRATHAFPNLAQIPGVRSPYGEQCRAAFGAEHHLDGITGKPWVQAGIDASGLELRCLAHFMARFDNGEYAHE |
| | | | ILNGDIHTKNQMAAELPTRDNAKTFIYGFLYGAGDEKIGQIVGAGKERGKELKKKFLENTPAIAALRESIQQTLVESSQWVAGE |
| | | | QQVKWKRRWIKGLDGRKVHVRSPHAALNTLLQSAGALICKLWIIKTEEMLVEKGLKHGWDGDFAYMAWIHDEIQVACRTEE |
| | | | IAKTVIEVAQEAMRWVGEHWNFRCLLDTEGKMGANWKECHSGGSKRTADGSEFEPKKKRKV |
| 241 | SpCas9 | T4 DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | | D112A,E114A | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSKEFYISI |
| | | | ETVGNNIVERYIDENGKERTREVEYLPTMFRHCKEESKYKDIYGKNCAPQKFPSMKDARDWMKRMEDIGLEALGMNDFKLA |
| | | | YISDTYGSEIVYDRKFVRVANCAIAVTGDKFPDPMKAEYEIDAITHYDSIDDRFYVFDLLNSMYGSVSKWDAKLAAKLDCEGG |
| | | | DEVPQEILDRVIYMPFDNERDMLMEYINLWEQKRPAIFTGWNIEGFDVPYIMNRVKMILGERSMKRFSPIGRVKSKLIQNMYG |
| | | | SKEIYSIDGVSILDYLDLYKKFAFTNLPSFSLESVAQHETKKGKLPYDGPINKLRETNHQRYISYNIIDVESVQAIDKIRGFIDLVL |
| | | | SMSYYAKMPFSGVMSPIKTWDAIIFNSLKGEHKVIPQQGSHVKQSFPGAFVFEPKPIARRYIMSFDLTSLYPSIIRQVNISPETIRG |
| | | | QFKVHPIHEYIAGTAPKPSDEYSCSPNGWMYDKHQEGIIPKEIAKVFFQRKDWKKKMFAEEMNAEAIKKIIMKGAGSCSTKPE |
| | | | VERYVKFSDDFLNELSNYTESVLNSLIEECEKAATLANTNQLNRKILINSLYGALGNIHFRYYDLRNATAITIFGQVGIQWIARKI |
| | | | NEYLNKVCGTNDEDFIAAGDTDSVYVCVDKVIEKVGLDRFKEQNDLVEFMNQFGKKKMEPMIDVAYRELCDYMNNREHLM |
| | | | HMDREAISCPPLGSKGVGGFWKAKKRYALNVYDMEDKRFAEPHLKIMGMETQQSSTPKAVQEALEESIRRILQEGEESVQEY |
| | | | YKNFEKEYRQLDYKVIAEVKTANDIAKYDDKGWPGFKCPFHIRGVLTYRRAVSGLGVAPILDGNKVMVLPLREGNPFGDKCI |
| | | | AWPSGTELPKEIRSDVLSWIDHSTLFQKSFVKPLAGMCESAGMDYEEKASLDFLFGSGGSKRTADGSEFEPKKKRKV |
| 242 | SpCas9 | T5 DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | | D164A, | DLRLIYLALAHMIKFRGHFLIEGDLNPDNSDVDKLFIQLVQTYNQLFEENPINASGVDAKAILSARLSKSRRLENLIAQLPGEKK |
| | | E166A | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSKIAVVD |
| | | | KALNNTRYDKHFQLYGEEVDVFHMCNEKLSGRLLKKHITIGTPENPFDPNDYDFVILVGAEPFLYFAGKKGIGDYTGKRVEYN |
| | | | GYANWIASISPAQLHFKPEMKPVFDATVENIHDIINGREKIAKAGDYRPITDPDEAEEYIKMVYNMVIGPVAFASATSALYCRD |
| | | | GYLLGVSISHQEYQGVYIDSDCLTEVAVYYLQKILDSENHTIVFHNLKFDMHFYKYHLGLTFDKAHKERRLHDTMLQHYVLD |
| | | | ERRGTHGLKSLAMKYTDMGDYDFELDKFKDDYCKAHKIKKEDFTYDLIPFDIMWPYAAKDTDATIRLHNFFLPKIEKNEKLCS |
| | | | LYYDVLMPGCVFLQRVEDRGVPISIDRLKEAQYQLTHNLNKAREKLYTYPEVKQLEQDQNEAFNPNSVKQLRVLLFDYVGLT |
| | | | PTGKLTDTGADSTDAEALNELATQHPIAKTLLEIRKLTKLISTYVEKILLSIDADGCIRTGFHEHMTTSGRLSSSGKLNLQQLPRD |
| | | | ESIIKGCVVAPPGYRVIAWDLTTAEVYYAAVLSGDRNMQQVFINMRNEPDKYPDFHSNIAHMVFKLQCEPRDVKKLFPALRQ |
| | | | AAKAITFGILYGSGPAKVAHSVNEALLEQAAKTGEPFVECTVADAKEYIETYFGQFPQLKRWIDKCHDQIKNHGFIYSHFGRKR |
| | | | RLHNIHSEDRGVQGEEIRSGFNAIIQSASSDSLLLGAVDADNEIISLGLEQEMKIVMLVHDSVVAIVREDLIDQYNEILIRNIQKD |
| | | | RGISIPGCPIGIDSDSEAGGSRDYSCGKMKKQHPSIACIDDDEYTRYVKGVLLDAEFEYKKLAAMDKEHPDHSKYKDDKFIAVC |
| | | | KDLDNVKRILGASGGSKRTADGSEFEPKKKRKV |
| 243 | SpCas9 | T7 DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | | D5A, E7A | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSIVSAIAA |
| | | | NALLESVTKFHCGVIYDYSTAEYVSYRPSDFGAYLDALEAEVARGGLIVFHNGHKYDVPALTKLAKLQLNREFHLPRENCIDT |
| | | | LVLSRLIHSNLKDTDMGLLRSGKLPGKRFGSHALEAWGYRLGEMKGEYKDDFKRMLEEQGEEYVDGMEWWNFNEEMMDY |
| | | | NVQDVVVTKALLEKLLSDKHYFPPEIDFTDVGYTTFWSESLEAVDIEHRAAWLLAKQERNGFPFDTKAIEELYVELAARRSEL |
| | | | LRKLTETFGSWYQPKGGTEMFCHPRTGKPLPKYPRIKTPKVGGIFKKPKNKAQREGREPCELDTREYVAGAPYTPVEHVVFNP |
| | | | SSRDHIQKKLQEAGWVPTKYTDKGAPVVDDEVLEGVRVDDPEKQAAIDLIKEYLMIQKRIGQSAEGDKAWLRYVAEDGKIHG |
| | | | SVNPNGAVTGRATHAFPNLAQIPGVRSPYGEQCRAAFGAEHHLDGITGKPWVQAGIDASGLELRCLAHFMARFDNGEYAHEI |
| | | | LNGDIHTKNQIAAELPTRDNAKTFIYGFLYGAGDEKIGQIVGAGKERGKELKKKFLENTPAIAALRESIQQTLVESSQWVAGEQ |
| | | | QVKWKRRWIKGLDGRKVHVRSPHAALNTLLQSAGALICKLWIIKTEEMLVEKGLKHGWDGDFAYMAWVHDEIQVGCRTEEI |
| | | | AQVVIETAQEAMRWVGDHWNFRCLLDTEGKMGPNWAICHSGGSKRTADGSEFEPKKKRKV |
| 244 | SpCas9 | Bst DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase | RTARRRYTRRKNRICYLQEIFSNEMAKVDDSFFHRLEESFLVEEDKKHERHPIFGNIVDEVAYHEKYPTIYHLRKKLVDSTDKA |
| | | | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSKKKLVL |
| | | | IDGNSVAYRAFFALPLLHNDKGIHTNAVYGFTMMLNKILAEEQPTHLLVAFDAGKTTFRHETFQEYKGGRQQTPPELSEQFPL |
| | | | LRELLKAYRIPAYELDHYEADDIIGTLAARAEQEGFEVKIISGDRDLTQLASRHVTVDITKKGITDIEPYTPETVREKYGLTPEQI |
| | | | VDLKGLMGDKSDNIPGVPGIGEKTAVKLLKQFGTVENVLASIDEVKGEKLKENLRQHRDLALLSKQLASICRDAPVELSLDDI |
| | | | VYEGQDREKVIALFKELGFQSFLEKMAAPAAEGEKPLEEMEFAIVDVITEEMLADKAALVVEVMEENYHDAPIVGIALVNEHG |
| | | | RFFMRPETALADSQFLAWLADETKKKSMFDAKRAVVALKWKGIELRGVAFDLLLAAYLLNPAQDAGDIAAVAKMKQYEAV |
| | | | RSDEAVYGKGVKRSLPDEQTLAEHLVRKAAAIWALEQPFMDDLRNNEQDQLLTKLEQPLAAILAEMEFTGVNVDTKRLEQM |
| | | | GSELAEQLRAIEQRIYELAGQEFNINSPKQLGVILFEKLQLPVLKKTKTGYSTSADVLEKLAPHHEIVENILHYRQLGKLQSTYIE |
| | | | GLLKVVRPDTGKVHTMFNQALTQTGRLSSAEPNLQNIPIRLEEGRKIRQAFVPSEPDWLIFAADYSQIELRVLAHIADDDNLIEA |
| | | | FQRDLDIHTKTAMDIFHVSEEEVTANMRRQAKAVNFGIVYGISDYGLAQNLNITRKEAAEFIERYFASFPGVKQYMENIVQEA |
| | | | KQKGYVTTLLHRRRYLPDITSRNFNVRSFAERTAMNTPIQGSAADIIKKAMIDLAARLKEEQLQARLLLQVHDELILEAPKEEIE |
| | | | RLCELVPEVMEQAVTLRVPLKVDYHYGPTWYDAKSGGSKRTADGSEFEPKKKRKV |
| 245 | SpCas9 | Human DNA | MKRTADGSEFESPKKKRKVDKKYSIGLDIGTNSVGWAVITDEYKVPSKKFKVLGNTDRHSIKKNLIGALLFDSGETAEATRLK |
| | H840A | polymerase | RTARRRYTRRKNRICYLQEIFSNEMAKVDDSFFHRLEESFLVEEDKKHERHPIFGNIVDEVAYHEKYPTIYHLRKKLVDSTDKA |
| | | alpha | DLRLIYLALAHMIKFRGHFLIEGDLNPDNSDVDKLFIQLVQTYNQLFEENPINASGVDAKAILSARLSKSRRLENLIAQLPGEKK |
| | | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSSASAQQ |
| | | | LAEELQIFGLDCEEALIEKLVELCVQYGQNEEGMVGELIAFCTSTHKVGLTSEILNSFEHEFLSKRLSKARHSTCKDSGHAGAR |
| | | | DIVSIQELIEVEEEEEILLNSYTTPSKGSQKRAISTPETPLTKRSVSTRSPHQLLSPSSFSPSATPSQKYNSRSNRGEVVTSFGLAQG |
| | | | VSWSGRGGAGNISLKVLGCPEALTGSYKSMFQKLPDIREVLTCKIEELGSELKEHYKIEAFTPLLAPAQEPVTLLGQIGCDSNGK |
| | | | LNNKSVILEGDREHSSGAQIPVDLSELKEYSLFPGQVVIMEGINTTGRKLVATKLYEGVPLPFYQPTEEDADFEQSMVLVACGP |
| | | | YTTSDSITYDPLLDLIAVINHDRPDVCILFGPFLESKHEQVENCLLTSPFEDIFKQCLRTIIEGTRSSGSHLVFVPSLRDVHHEPVYP |
| | | | QPPFSYSDLSREDKKQVQFVSEPCSLSINGVIFGLTSTDLLFHLGAEEISSSSGTSDRFSRILKHILTQRSYYPLYPPQEDMAIDYES |
| | | | FYVYAQLPVTPDVLIIPSELRYFVKDVLGCVCVNPGRLTKGQVGGTFARLYLRRPAADGAERQSPCIAVQVVRISGGSKRTAD |
| | | | GSEFEPKKKRKV |
| 246 | SpCas9 | Human alpha | MKRTADGSEFESPKKKRKVDKKYSIGLDIGTNSVGWAVITDEYKVPSKKFKVLGNTDRHSIKKNLIGALLFDSGETAEATRLK |
| | H840A | herpesvirus 1 | RTARRRYTRRKNRICYLQEIFSNEMAKVDDSFFHRLEESFLVEEDKKHERHPIFGNIVDEVAYHEKYPTIYHLRKKLVDSTDKA |
| | | DNA | DLRLIYLALAHMIKFRGHFLIEGDLNPDNSDVDKLFIQLVQTYNQLFEENPINASGVDAKAILSARLSKSRRLENLIAQLPGEKK |
| | | polymerase | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLNREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSFSGGGG |
| | | | PLSPGGKSAARAASGFFAPAGPRGAGRGPPPCLRQNFYNPYLAPVGTQQKPTGPTQRHTYYSECDEFRFIAPRVLDEDAPPEKR |
| | | | AGVHDGHLKRAPKVYCGGDERDVLRVGSGGFWPRRSRLWGGVDHAPAGFNPTVTVFHVYDILENVEHAYGMRAAQFHARF |
| | | | MDAITPTGTVITLLGLTPEGHRVAVHVYGTRQYFYMNKEEVDRHLQCRAPRDLCERMAAALRESPGASFRGISADHFEAEVV |
| | | | ERTDVYYYETRPALFYRVYVRSGRVLSYLCDNFCPAIKKYEGGVDATTRFILDNPGFVTFGWYRLKPGRNNTLAQPRAPMAF |
| | | | GTSSDVEFNCTADNLAIEGGMSDLPAYKLMCFDIECKAGGEDELAFPVAGHPEDLVIQISCLLYDLSTTALEHVLLFSLGSCDLP |
| | | | ESHLNELAARGLPTPVVLEFDSEFEMLLAFMTLVKQYGPEFVTGYNIINFDWPFLLAKLTDIYKVPLDGYGRMNGRGVFRVW |
| | | | DIGQSHFQKRSKIKVNGMVNIDMYGIITDKIKLSSYKLNAVAEAVLKDKKKDLSYRDIPAYYAAGPAQRGVIGEYCIQDSLLV |
| | | | GQLFFKFLPHLELSAVARLAGINITRTIYDGQQIRVFTCLLRLADQKGFILPDTQGRFRGAGGEAPKRPAAAREDEERPEEEGED |
| | | | EDEREEGGGEREPEGARETAGRHVGYQGARVHDPTSGFHVNPVVGFDFASLYPSIIQAHNLCFSTLSLRADAVAHLEAGKDYL |
| | | | EIEVGGRRLFFVKAHVRESLLSILLRDWLAMRKQIRSRIPQSSPEEAVLLDKQQAAIKVVCNSVYGFTGVQHGLLPCLHVAATV |
| | | | TTIGREMLLATREYVHARWAAFEQLLADFPEAADMRAPGPYSMRIIYGDTDSIFVLCRGLTAAGLTAMGDKMASHISRALFLP |
| | | | PIKLECEKTFTKLLLIAKKKYIGVIYGGKMLIKGVDLVRKNNCAFINRTSRALVDLLFYDDTVSGAAAALAERPAEEWLARPLP |
| | | | EGLQAFGAVLVDAHRRITDPERDIQDFVLTAELSRHPRAYTNKRLAHLTVYYKLMARRAQVPSIKDRIPYVIVAQTREVEETV |
| | | | ARLAALRELDAAAPGDEPAPPAALPSPAKRPRETPSHADPPGGASKPRKLLVSELAEDPAYAIAHGVALNTDYYFSHLLGAAC |
| | | | VTFKALFGNNAKITESLLKRFIPEVWHPPDDVAARLRAAGFGAVGAGATAEETRRMLHRAFDTLASGGSKRTADGSEFEPKK |
| | | | KRKV |
| 247 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase- | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | D12A, D66A | DLRLIYLALAHMIKFRGHFLIEGDLNPDNSDVDKLFIQLVQTYNQLFEENPINASGVDAKAILSARLSKSRKLENLIAQLPGEKK |
| | H840A | | NGLFGNLIALSLGLTPNFKSNFDLAEDAKLQLSKDTYDDDLDNLLAQIGDQYADLFLAAKNLSDAILLSDILRVNTEITKAPLSA |
| | | | SMIKRYDEHHQDLTLLKALVRQQLPEKYKEIFFDQSKNGYAGYIDGGASQEEFYKFIKPILEKMDGTEELLVKLKREDLLRKQ |
| | | | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKMYSCDFETTTKVEDCRVWAYGYMNIEDHSEYKIGNSLDEFMAWVLKVQADLYFHNLKFDGAFIINWLERNGFKWSADG |
| | | | LPNTYNTIISRMGQWYMIDICLGYKGKRKIHTVIYDSLKKLPFPVKKIAKDFKLTVLKGDIDYHKERPVGYKITPEEYAYIKNDI |
| | | | QIIAEALLIQFKQGLDRMTAGSDSLKGFKDIITTKKFKKVFPTLSLGLDKEVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGAIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRQKTYIQDIY |
| | | | MKEVDGKLVEGSPDDYTDIKFSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 248 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, G197D, | SMIKRYDEHHQDLTLLKALVRQQLPEKYKEIFFDQSKNGYAGYIDGGASQEEFYKFIKPILEKMDGTEELLVKLKREDLLRKQ |
| | | E221K | RTFDNGSIPHQIHLGELHAILRRQEDFYPFLKDNREKIEKILTFRIPYYVGPLARGNSRFAWMTRKSEETITPWNFEEVVDKGAS |
| | | | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDFKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGAIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRQKTYIQDIY |
| | | | MKEVDGKLVEGSPDDYTDIKFSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 249 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase- | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, G197D, | SMIKRYDEHHQDLTLLKALVRQQLPEKYKEIFFDQSKNGYAGYIDGGASQEEFYKFIKPILEKMDGTEELLVKLKREDLLRKQ |
| | | E221K, Q497P, | RTFDNGSIPHQIHLGELHAILRRQEDFYPFLKDNREKIEKILTFRIPYYVGPLARGNSRFAWMTRKSEETITPWNFEEVVDKGAS |
| | | K512E, F526L | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDFKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGAIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 250 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K. | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 251 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKGTGSGAWKEWL |
| | | | ERKVGEGRARRLIEYFGSAGEVGKLVENAEVSKLLEVPGIGDEAVARKVPGSGGSKRTADGSEFESPKKKRKVGSGPAAKRV |
| | | | KLD |
| 252 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, V51A, | DLRLIYLALAHMIKFRGHFLIEGDLNPDNSDVDKLFIQLVQTYNQLFEENPINASGVDAKAILSARLSKSRKLENLIAQLPGEKK |
| | H840A | M97T, F137C, | NGLFGNLIALSLGLTPNFKSNFDLAEDAKLQLSKDTYDDDLDNLLAQIGDQYADLFLAAKNLSDAILLSDILRVNTEITKAPLSA |
| | | G197D, | SMIKRYDEHHQDLTLLKALVRQQLPEKYKEIFFDQSKNGYAGYIDGGASQEEFYKFIKPILEKMDGTEELLVKLKREDLLRKQ |
| | | E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, | AQSFIERMTNFDKNLPNEKVLPKHSLLYEYFTVYNELTKVKYVTEGMRKPAFLSGEQKKAIVDLLFKTNRKVTVKQLKEDYF |
| | | Q497P, | KKIECFDSVEISGVEDRFNASLGTYHDLLKIIKDKDFLDNEENEDILEDIVLTLTLFEDREMIEERLKTYAHLFDDKVMKQLKRR |
| | | K512E, F526L | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCDFETTTKVEDCRVWAYGYMNIEDHSEYKIGNSLDEFMAWALKVQADLYFHNLKFDGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 253 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSSGGSSGSET |
| | | | PGTSESATPESSGGSSGGSMAENGDNEKMAALEAKICHQIEYYFGDFNLPRDKFLKEQIKLDEGWVPLEIMIKFNRLNRLTTDF |
| | | | NVIVEALSKSKAELMEISEDKTKIRRSPSKPLPEVTDEYKNDVKNRSVYIKGFPTDATLDDIKEWLEDKGQVLNIQMRRTLHKA |
| | | | FKGSIFVVFDSIESAKKFVETPGQKYKETDLLILFKDDYFAKKNESGGSKRTADGSEFESPKKKRKVGSGPAAKRVKLD |
| 254 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, V51A, | DLRLIYLALAHMIKFRGHFLIEGDLNPDNSDVDKLFIQLVQTYNQLFEENPINASGVDAKAILSARLSKSRKLENLIAQLPGEKK |
| | H840A | M97T, F137C, | NGLFGNLIALSLGLTPNFKSNFDLAEDAKLQLSKDTYDDDLDNLLAQIGDQYADLFLAAKNLSDAILLSDILRVNTEITKAPLSA |
| | | G197D | SMIKRYDEHHQDLTLLKALVRQQLPEKYKEIFFDQSKNGYAGYIDGGASQEEFYKFIKPILEKMDGTEELLVKLKREDLLRKQ |
| | | E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, | AQSFIERMTNFDKNLPNEKVLPKHSLLYEYFTVYNELTKVKYVTEGMRKPAFLSGEQKKAIVDLLFKTNRKVTVKQLKEDYF |
| | | Q497P, | KKIECFDSVEISGVEDRFNASLGTYHDLLKIIKDKDFLDNEENEDILEDIVLTLTLFEDREMIEERLKTYAHLFDDKVMKQLKRR |
| | | K512E, F526L | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCDFETTTKVEDCRVWAYGYMNIEDHSEYKIGNSLDEFMAWALKVQADLYFHNLKFDGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSSGGSSGSET |
| | | | PGTSESATPESSGGSSGGSMAENGDNEKMAALEAKICHQIEYYFGDFNLPRDKFLKEQIKLDEGWVPLEIMIKFNRLNRLTTDF |
| | | | NVIVEALSKSKAELMEISEDKTKIRRSPSKPLPEVTDEYKNDVKNRSVYIKGFPTDATLDDIKEWLEDKGQVLNIQMRRTLHKA |
| | | | FKGSIFVVFDSIESAKKFVETPGQKYKETDLLILFKDDYFAKKNESGGSKRTADGSEFESPKKKRKVGSGPAAKRVKLD |
| 255 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | D12A, D66A, | DLRLIYLALAHMIKFRGHFLIEGDLNPDNSDVDKLFIQLVQTYNQLFEENPINASGVDAKAILSARLSKSRKLENLIAQLPGEKK |
| | H840A | G197D, | NGLFGNLIALSLGLTPNFKSNFDLAEDAKLQLSKDTYDDDLDNLLAQIGDQYADLFLAAKNLSDAILLSDILRVNTEITKAPLSA |
| | | Y369E, | SMIKRYDEHHQDLTLLKALVRQQLPEKYKEIFFDQSKNGYAGYIDGGASQEEFYKFIKPILEKMDGTEELLVKLKREDLLRKQ |
| | | T372N, | RTFDNGSIPHQIHLGELHAILRRQEDFYPFLKDNREKIEKILTFRIPYYVGPLARGNSRFAWMTRKSEETITPWNFEEVVDKGAS |
| | | E375D, 1378R | AQSFIERMTNFDKNLPNEKVLPKHSLLYEYFTVYNELTKVKYVTEGMRKPAFLSGEQKKAIVDLLFKTNRKVTVKQLKEDYF |
| | | | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKMYSCAFETTTKVEDCRVWAYGYMNIEDHSEYKIGNSLDEFMAWVLKVQADLYFHNLKFAGAFIINWLERNGFKWSADG |
| | | | LPNTYNTIISRMGQWYMIDICLGYKGKRKIHTVIYDSLKKLPFPVKKIAKDFKLTVLKGDIDYHKERPVGYKITPEEYAYIKNDI |
| | | | QIIAEALLIQFKQGLDRMTAGSDSLKDFKDIITTKKFKKVFPTLSLGLDKEVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTEIKNTSDGARKQLAKLMLNSLYGKFASNPDVTGKVPYLKENGALGFRLGEEE |
| | | | TKDPVYTPMGVFITAWARYTTITAAQACYDRIIYCDTDSIHLTGTEIPDVIKDIVDPKKLGYWAHESTFKRAKYLRQKTYIQDIY |
| | | | MKEVDGKLVEGSPDDYTDIKFSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 256 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, G197D, | SMIKRYDEHHQDLTLLKALVRQQLPEKYKEIFFDQSKNGYAGYIDGGASQEEFYKFIKPILEKMDGTEELLVKLKREDLLRKQ |
| | | E221K, Y369E, | RTFDNGSIPHQIHLGELHAILRRQEDFYPFLKDNREKIEKILTFRIPYYVGPLARGNSRFAWMTRKSEETITPWNFEEVVDKGAS |
| | | T372N, E375D, | AQSFIERMTNFDKNLPNEKVLPKHSLLYEYFTVYNELTKVKYVTEGMRKPAFLSGEQKKAIVDLLFKTNRKVTVKQLKEDYF |
| | | I378R, Q497P, | KKIECFDSVEISGVEDRFNASLGTYHDLLKIIKDKDFLDNEENEDILEDIVLTLTLFEDREMIEERLKTYAHLFDDKVMKQLKRR |
| | | K512E, F526L | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDFKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTEIKNTSDGARKQLAKLMLNSLYGKFASNPDVTGKVPYLKENGALGFRLGEEE |
| | | | TKDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 257 | SpCas9 | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | | A377C, Y369E, | AQSFIERMTNFDKNLPNEKVLPKHSLLYEYFTVYNELTKVKYVTEGMRKPAFLSGEQKKAIVDLLFKTNRKVTVKQLKEDYF |
| | | T372N, E375D, | KKIECFDSVEISGVEDRFNASLGTYHDLLKIIKDKDFLDNEENEDILEDIVLTLTLFEDREMIEERLKTYAHLFDDKVMKQLKRR |
| | | I378R, Q497P, | RYTGWGRLSRKLINGIRDKQSGKTILDFLKSDGFANRNFMQLIHDDSLTFKEDIQKAQVSGQGDSLHEHIANLAGSPAIKKGIL |
| | | K512E, F526L | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTEIKNTSDGCRKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 258 | VRQR | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | SpCas9 - | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | R221K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | N394K, H840A, | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | D1135V, | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | G1218R, | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | R1335Q, T1337R | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFVSPTVAYSVLVVAKVEK |
| | | | GKSKKLKSVKELLGITIMERSSFEKNPIDFLEAKGYKEVKKDLIIKLPKYSLFELENGRKRMLASARELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKQYRSTKEVLDATLIHQSITGLYETRIDLSQLGGDSGGSSGGSKRTADGSEFESPKKKRKVSGGSSGGSKHM |
| | | | PRKRYSCAFETTTKVEDCRVWAYGYMNIEDHSEYKIGNSLDEFMAWALKVQADLYFHNLKFAGAFIINWLERNGFKWSADG |
| | | | LPNTYNTIISRTGQWYMIDICLGYKGKRKIHTVIYDSLKKLPFPVKKIAKDCKLTVLKGDIDYHKERPVGYKITPEEYAYIKNDI |
| | | | QIIAEALLIQFKQGLDRMTAGSDSLKDFKDIITTKKFKKVFPTLSLGLDKKVRYAYRGGFTWLNDRFKEKEIGEGMVFDVNSLY |
| | | | PAQMYSRLLPYGEPIVFEGKYVWDEDYPLHIQHIRCEFELKEGYIPTIQIKRSRFYKGNEYLKSSGGEIADLWLSNVDLELMKE |
| | | | HYDLYNVEYISGLKFKATTGLFKDFIDKWTYIKTTSEGCIKQLAKLMLNSLYGKFASNPDVTGKVPYLKENGALGFRLGEEET |
| | | | KDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 259 | SpGSpCas9 - | phi29 DNA | MKRTADGSEFESPKKKRKVDKKYSIGLDIGTNSVGWAVITDEYKVPSKKFKVLGNTDRHSIKKNLIGALLFDSGETAEATRLK |
| | R221K, | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | N394K, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | H840A, | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | D1135L, | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | S1136W, | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | G1218K, | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | E1219Q, | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | R1335Q, | | RYTGWGRLSRKLINGIRDKQSGKTILDFLKSDGFANRNFMQLIHDDSLTFKEDIQKAQVSGQGDSLHEHIANLAGSPAIKKGIL |
| | T1337R | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFLWPTVAYSVLVVAKVE |
| | | | KGKSKKLKSVKELLGITIMERSSFEKNPIDFLEAKGYKEVKKDLIIKLPKYSLFELENGRKRMLASAKQLQKGNELALPSKYVN |
| | | | FLYLASHYEKLKGSPEDNEQKQLFVEQHKHYLDEIIEQISEFSKRVILADANLDKVLSAYNKHRDKPIREQAENIIHLFTLTNLG |
| | | | APAAFKYFDTTIDRKQYRSTKEVLDATLIHQSITGLYETRIDLSQLGGDSGGSSGGSKRTADGSEFESPKKKRKVSGGSSGGSK |
| | | | HMPRKRYSCAFETTTKVEDCRVWAYGYMNIEDHSEYKIGNSLDEFMAWALKVQADLYFHNLKFAGAFIINWLERNGFKWSA |
| | | | DGLPNTYNTIISRTGQWYMIDICLGYKGKRKIHTVIYDSLKKLPFPVKKIAKDCKLTVLKGDIDYHKERPVGYKITPEEYAYIKN |
| | | | DIQIIAEALLIQFKQGLDRMTAGSDSLKDFKDIITTKKFKKVFPTLSLGLDKKVRYAYRGGFTWLNDRFKEKEIGEGMVFDVNS |
| | | | LYPAQMYSRLLPYGEPIVFEGKYVWDEDYPLHIQHIRCEFELKEGYIPTIQIKRSRFYKGNEYLKSSGGEIADLWLSNVDLELM |
| | | | KEHYDLYNVEYISGLKFKATTGLFKDFIDKWTYIKTTSEGCIKQLAKLMLNSLYGKFASNPDVTGKVPYLKENGALGFRLGEE |
| | | | ETKDPVYTPMGVFITAWARYTTITAAQACYDRIIYCDTDSIHLTGTEIPDVIKDIVDPKKLGYWAHESTFKRAKYLRPKTYIQDI |
| | | | YMKEVDGELVEGSPDDYTDIKLSVKCAGMTDKIKKEVTFENFKVGFSRKMKPKPVQVPGGVVLVDDTFTIKSGGSKRTADGS |
| | | | EFESPKKKRKVGSGPAAKRVKLD |
| 260 | SpRY | phi29 DNA | MKRTADGSEFESPKKKRKVDKKYSIGLDIGTNSVGWAVITDEYKVPSKKFKVLGNTDRHSIKKNLIGALLFDSGETAERTRLK |
| | SpCas9 - | polymerase - | RTARRRYTRRKNRICYLQEIFSNEMAKVDDSFFHRLEESFLVEEDKKHERHPIFGNIVDEVAYHEKYPTIYHLRKKLVDSTDKA |
| | A61R, | M8R, D12A, | DLRLIYLALAHMIKFRGHFLIEGDLNPDNSDVDKLFIQLVQTYNQLFEENPINASGVDAKAILSARLSKSRKLENLIAQLPGEKK |
| | R221K, | V51A, D66A, | NGLFGNLIALSLGLTPNFKSNFDLAEDAKLQLSKDTYDDDLDNLLAQIGDQYADLFLAAKNLSDAILLSDILRVNTEITKAPLSA |
| | N394K, | M97T, F137C, | SMIKRYDEHHQDLTLLKALVRQQLPEKYKEIFFDQSKNGYAGYIDGGASQEEFYKFIKPILEKMDGTEELLVKLKREDLLRKQ |
| | H840A, | G197D, E221K, | RTFDNGSIPHQIHLGELHAILRRQEDFYPFLKDNREKIEKILTFRIPYYVGPLARGNSRFAWMTRKSEETITPWNFEEVVDKGAS |
| | L1111R, | A377C, Q497P, | AQSFIERMTNFDKNLPNEKVLPKHSLLYEYFTVYNELTKVKYVTEGMRKPAFLSGEQKKAIVDLLFKTNRKVTVKQLKEDYF |
| | D1135L, | K512E, F526L | KKIECFDSVEISGVEDRFNASLGTYHDLLKIIKDKDFLDNEENEDILEDIVLTLTLFEDREMIEERLKTYAHLFDDKVMKQLKRR |
| | S1136W, | | RYTGWGRLSRKLINGIRDKQSGKTILDFLKSDGFANRNFMQLIHDDSLTFKEDIQKAQVSGQGDSLHEHIANLAGSPAIKKGIL |
| | G1218K, | | QTVKVVDELVKVMGRHKPENIVIEMARENQTTQKGQKNSRERMKRIEEGIKELGSQILKEHPVENTQLQNEKLYLYYLQNGR |
| | E1219Q, | | DMYVDQELDINRLSDYDVDAIVPQSFLKDDSIDNKVLTRSDKNRGKSDNVPSEEVVKKMKNYWRQLLNAKLITQRKFDNLTK |
| | N1317R, | | AERGGLSELDKAGFIKRQLVETRQITKHVAQILDSRMNTKYDENDKLIREVKVITLKSKLVSDFRKDFQFYKVREINNYHHAH |
| | A1322R, | | DAYLNAVVGTALIKKYPKLESEFVYGDYKVYDVRKMIAKSEQEIGKATAKYFFYSNIMNFFKTEITLANGEIRKRPLIETNGET |
| | R1333P, | | GEIVWDKGRDFATVRKVLSMPQVNIVKKTEVQTGGFSKESIRPKRNSDKLIARKKDWDPKKYGGFLWPTVAYSVLVVAKVE |
| | R1335Q, | | KGKSKKLKSVKELLGITIMERSSFEKNPIDFLEAKGYKEVKKDLIIKLPKYSLFELENGRKRMLASAKQLQKGNELALPSKYVN |
| | T1337R | | FLYLASHYEKLKGSPEDNEQKQLFVEQHKHYLDEIIEQISEFSKRVILADANLDKVLSAYNKHRDKPIREQAENIIHLFTLTRLG |
| | | | APRAFKYFDTTIDPKQYRSTKEVLDATLIHQSITGLYETRIDLSQLGGDSGGSSGGSKRTADGSEFESPKKKRKVSGGSSGGSKH |
| | | | MPRKRYSCAFETTTKVEDCRVWAYGYMNIEDHSEYKIGNSLDEFMAWALKVQADLYFHNLKFAGAFIINWLERNGFKWSAD |
| | | | GLPNTYNTIISRTGQWYMIDICLGYKGKRKIHTVIYDSLKKLPFPVKKIAKDCKLTVLKGDIDYHKERPVGYKITPEEYAYIKND |
| | | | IQIIAEALLIQFKQGLDRMTAGSDSLKDFKDIITTKKFKKVFPTLSLGLDKKVRYAYRGGFTWLNDRFKEKEIGEGMVFDVNSL |
| | | | YPAQMYSRLLPYGEPIVFEGKYVWDEDYPLHIQHIRCEFELKEGYIPTIQIKRSRFYKGNEYLKSSGGEIADLWLSNVDLELMK |
| | | | EHYDLYNVEYISGLKFKATTGLFKDFIDKWTYIKTTSEGCIKQLAKLMLNSLYGKFASNPDVTGKVPYLKENGALGFRLGEEE |
| | | | TKDPVYTPMGVFITAWARYTTITAAQACYDRIIYCDTDSIHLTGTEIPDVIKDIVDPKKLGYWAHESTFKRAKYLRPKTYIQDIY |
| | | | MKEVDGELVEGSPDDYTDIKLSVKCAGMTDKIKKEVTFENFKVGFSRKMKPKPVQVPGGVVLVDDTFTIKSGGSKRTADGSE |
| | | | FESPKKKRKVGSGPAAKRVKLD |
| 261 | St1Cas9 | phi29 DNA | MKRTADGSEFESPKKKRKVSDLVLGLDIGIGSVGVGILNKVTGEIIHKNSRIFPAAQAENNLVRRTNRQGRRLARRKKHRRVRL |
| | | polymerase - | NRLFEESGLITDFTKISINLNPYQLRVKGLTDELSNEELFIALKNMVKHRGISYLDDASDDGNSSVGDYAQIVKENSKQLETKTP |
| | | M8R, D12A, | GQIQLERYQTYGQLRGDFTVEKDGKKHRLINVFPTSAYRSEALRILQTQQEFNPQITDEFINRYLEILTGKRKYYHGPGNEKSRT |
| | | V51A, D66A, | DYGRYRTSGETLDNIFGILIGKCTFYPDEFRAAKASYTAQEFNLLNDLNNLTVPTETKKLSKEQKNQIINYVKNEKAMGPAKLF |
| | | M97T, F137C, | KYIAKLLSCDVADIKGYRIDKSGKAEIHTFEAYRKMKTLETLDIEQMDRETLDKLAYVLTLNTEREGIQEALEHEFADGSFSQK |
| | | G197D, E221K, | QVDELVQFRKANSSIFGKGWHNFSVKLMMELIPELYETSEEQMTILTRLGKQKTTSSSNKTKYIDEKLLTEEIYNPVVAKSVRQ |
| | | A377C, Q497P, | AIKIVNAAIKEYGDFDNIVIEMARETNEDDEKKAIQKIQKANKDEKDAAMLKAANQYNGKAELPHSVFHGHKQLATKIRLWH |
| | | K512E, F526L | QQGERCLYTGKTISIHDLINNSNQFEVDHILPLSITFDDSLANKVLVYATANQEKGQRTPYQALDSMDDAWSFRELKAFVRESK |
| | | | TLSNKKKEYLLTEEDISKFDVRKKFIERNLVDTRYASRVVLNALQEHFRAHKIDTKVSVVRGQFTSQLRRHWGIEKTRDTYHH |
| | | | HAVDALIIAASSQLNLWKKQKNTLVSYSEDQLLDIETGELISDDEYKESVFKAPYQHFVDTLKSKEFEDSILFSYQVDSKFNRKI |
| | | | SDATIYATRQAKVGKDKADETYVLGKIKDIYTQDGYDAFMKIYKKDKSKFLMYRHDPQTFEKVIEPILENYPNKQINEKGKEV |
| | | | PCNPFLKYKEEHGYIRKYSKKGNGPEIKSLKYYDSKLGNHIDITPKDSNNKVVLQSVSPWRADVYFNKTTGKYEILGLKYADL |
| | | | QFEKGTGTYKISQEKYNDIKKKEGVDSDSEFKFTLYKNDLLLVKDTETKEQQLFRFLSRTMPKQKHYVELKPYDKQKFEGGE |
| | | | ALIKVLGNVANSGQCKKGLGKSNISIYKVRTDVLGNQHIIKNEGDKPKLDFSGGSSGGSKRTADGSEFESPKKKRKVSGGSSGG |
| | | | SKHMPRKRYSCAFETTTKVEDCRVWAYGYMNIEDHSEYKIGNSLDEFMAWALKVQADLYFHNLKFAGAFIINWLERNGFKW |
| | | | SADGLPNTYNTIISRTGQWYMIDICLGYKGKRKIHTVIYDSLKKLPFPVKKIAKDCKLTVLKGDIDYHKERPVGYKITPEEYAYI |
| | | | KNDIQIIAEALLIQFKQGLDRMTAGSDSLKDFKDIITTKKFKKVFPTLSLGLDKKVRYAYRGGFTWLNDRFKEKEIGEGMVFDV |
| | | | NSLYPAQMYSRLLPYGEPIVFEGKYVWDEDYPLHIQHIRCEFELKEGYIPTIQIKRSRFYKGNEYLKSSGGEIADLWLSNVDLEL |
| | | | MKEHYDLYNVEYISGLKFKATTGLFKDFIDKWTYIKTTSEGCIKQLAKLMLNSLYGKFASNPDVTGKVPYLKENGALGFRLGE |
| | | | EETKDPVYTPMGVFITAWARYTTITAAQACYDRIIYCDTDSIHLTGTEIPDVIKDIVDPKKLGYWAHESTFKRAKYLRPKTYIQD |
| | | | IYMKEVDGELVEGSPDDYTDIKLSVKCAGMTDKIKKEVTFENFKVGFSRKMKPKPVQVPGGVVLVDDTFTIKSGGSKRTADG |
| | | | SEFESPKKKRKVGSGPAAKRVKLD |
| 262 | St1Cas9- | phi29 DNA | MKRTADGSEFESPKKKRKVSDLVLGLDIGIGSVGVGILNKVTGEIIHKNSRIFPAAQAENNLVRRTNRQGRRLARRKKHRRVRL |
| | H599A | polymerase - | NRLFEESGLITDFTKISINLNPYQLRVKGLTDELSNEELFIALKNMVKHRGISYLDDASDDGNSSVGDYAQIVKENSKQLETKTP |
| | | M8R, D12A, | GQIQLERYQTYGQLRGDFTVEKDGKKHRLINVFPTSAYRSEALRILQTQQEFNPQITDEFINRYLEILTGKRKYYHGPGNEKSRT |
| | | V51A, D66A, | DYGRYRTSGETLDNIFGILIGKCTFYPDEFRAAKASYTAQEFNLLNDLNNLTVPTETKKLSKEQKNQIINYVKNEKAMGPAKLF |
| | | M97T, F137C, | KYIAKLLSCDVADIKGYRIDKSGKAEIHTFEAYRKMKTLETLDIEQMDRETLDKLAYVLTLNTEREGIQEALEHEFADGSFSQK |
| | | G197D, E221K, | QVDELVQFRKANSSIFGKGWHNFSVKLMMELIPELYETSEEQMTILTRLGKQKTTSSSNKTKYIDEKLLTEEIYNPVVAKSVRQ |
| | | A377C, Q497P, | AIKIVNAAIKEYGDFDNIVIEMARETNEDDEKKAIQKIQKANKDEKDAAMLKAANQYNGKAELPHSVFHGHKQLATKIRLWH |
| | | K512E, F526L | QQGERCLYTGKTISIHDLINNSNQFEVDAILPLSITFDDSLANKVLVYATANQEKGQRTPYQALDSMDDAWSFRELKAFVRESK |
| | | | TLSNKKKEYLLTEEDISKFDVRKKFIERNLVDTRYASRVVLNALQEHFRAHKIDTKVSVVRGQFTSQLRRHWGIEKTRDTYHH |
| | | | HAVDALIIAASSQLNLWKKQKNTLVSYSEDQLLDIETGELISDDEYKESVFKAPYQHFVDTLKSKEFEDSILFSYQVDSKFNRKI |
| | | | SDATIYATRQAKVGKDKADETYVLGKIKDIYTQDGYDAFMKIYKKDKSKFLMYRHDPQTFEKVIEPILENYPNKQINEKGKEV |
| | | | PCNPFLKYKEEHGYIRKYSKKGNGPEIKSLKYYDSKLGNHIDITPKDSNNKVVLQSVSPWRADVYFNKTTGKYEILGLKYADL |
| | | | QFEKGTGTYKISQEKYNDIKKKEGVDSDSEFKFTLYKNDLLLVKDTETKEQQLFRFLSRTMPKQKHYVELKPYDKQKFEGGE |
| | | | ALIKVLGNVANSGQCKKGLGKSNISIYKVRTDVLGNQHIIKNEGDKPKLDFSGGSSGGSKRTADGSEFESPKKKRKVSGGSSGG |
| | | | SKHMPRKRYSCAFETTTKVEDCRVWAYGYMNIEDHSEYKIGNSLDEFMAWALKVQADLYFHNLKFAGAFIINWLERNGFKW |
| | | | SADGLPNTYNTIISRTGQWYMIDICLGYKGKRKIHTVIYDSLKKLPFPVKKIAKDCKLTVLKGDIDYHKERPVGYKITPEEYAYI |
| | | | KNDIQIIAEALLIQFKQGLDRMTAGSDSLKDFKDIITTKKFKKVFPTLSLGLDKKVRYAYRGGFTWLNDRFKEKEIGEGMVFDV |
| | | | NSLYPAQMYSRLLPYGEPIVFEGKYVWDEDYPLHIQHIRCEFELKEGYIPTIQIKRSRFYKGNEYLKSSGGEIADLWLSNVDLEL |
| | | | MKEHYDLYNVEYISGLKFKATTGLFKDFIDKWTYIKTTSEGCIKQLAKLMLNSLYGKFASNPDVTGKVPYLKENGALGFRLGE |
| | | | EETKDPVYTPMGVFITAWARYTTITAAQACYDRIIYCDTDSIHLTGTEIPDVIKDIVDPKKLGYWAHESTFKRAKYLRPKTYIQD |
| | | | IYMKEVDGELVEGSPDDYTDIKLSVKCAGMTDKIKKEVTFENFKVGFSRKMKPKPVQVPGGVVLVDDTFTIKSGGSKRTADG |
| | | | SEFESPKKKRKVGSGPAAKRVKLD |
| 263 | SpCas9 - | MMLV | MKRTADGSEFESPKKKRKVDKKYSIGLDIGTNSVGWAVITDEYKVPSKKFKVLGNTDRHSIKKNLIGALLFDSGETAEATRLK |
| | H840A | reverse | RTARRRYTRRKNRICYLQEIFSNEMAKVDDSFFHRLEESFLVEEDKKHERHPIFGNIVDEVAYHEKYPTIYHLRKKLVDSTDKA |
| | | transcriptase - | DLRLIYLALAHMIKFRGHFLIEGDLNPDNSDVDKLFIQLVQTYNQLFEENPINASGVDAKAILSARLSKSRRLENLIAQLPGEKK |
| | | D201N, | NGLFGNLIALSLGLTPNFKSNFDLAEDAKLQLSKDTYDDDLDNLLAQIGDQYADLFLAAKNLSDAILLSDILRVNTEITKAPLSA |
| | | T307K, | SMIKRYDEHHQDLTLLKALVRQQLPEKYKEIFFDQSKNGYAGYIDGGASQEEFYKFIKPILEKMDGTEELLVKLNREDLLRKQ |
| | | W314F, | RTFDNGSIPHQIHLGELHAILRRQEDFYPFLKDNREKIEKILTFRIPYYVGPLARGNSRFAWMTRKSEETITPWNFEEVVDKGAS |
| | | T331P, | AQSFIERMTNFDKNLPNEKVLPKHSLLYEYFTVYNELTKVKYVTEGMRKPAFLSGEQKKAIVDLLFKTNRKVTVKQLKEDYF |
| | | L604W | KKIECFDSVEISGVEDRFNASLGTYHDLLKIIKDKDFLDNEENEDILEDIVLTLTLFEDREMIEERLKTYAHLFDDKVMKQLKRR |
| | | | RYTGWGRLSRKLINGIRDKQSGKTILDFLKSDGFANRNFMQLIHDDSLTFKEDIQKAQVSGQGDSLHEHIANLAGSPAIKKGIL |
| | | | QTVKVVDELVKVMGRHKPENIVIEMARENQTTQKGQKNSRERMKRIEEGIKELGSQILKEHPVENTQLQNEKLYLYYLQNGR |
| | | | DMYVDQELDINRLSDYDVDAIVPQSFLKDDSIDNKVLTRSDKNRGKSDNVPSEEVVKKMKNYWRQLLNAKLITQRKFDNLTK |
| | | | AERGGLSELDKAGFIKRQLVETRQITKHVAQILDSRMNTKYDENDKLIREVKVITLKSKLVSDFRKDFQFYKVREINNYHHAH |
| | | | DAYLNAVVGTALIKKYPKLESEFVYGDYKVYDVRKMIAKSEQEIGKATAKYFFYSNIMNFFKTEITLANGEIRKRPLIETNGET |
| | | | GEIVWDKGRDFATVRKVLSMPQVNIVKKTEVQTGGFSKESILPKRNSDKLIARKKDWDPKKYGGFDSPTVAYSVLVVAKVEK |
| | | | GKSKKLKSVKELLGITIMERSSFEKNPIDFLEAKGYKEVKKDLIIKLPKYSLFELENGRKRMLASAGELQKGNELALPSKYVNFL |
| | | | YLASHYEKLKGSPEDNEQKQLFVEQHKHYLDEIIEQISEFSKRVILADANLDKVLSAYNKHRDKPIREQAENIIHLFTLTNLGAP |
| | | | AAFKYFDTTIDRKRYTSTKEVLDATLIHQSITGLYETRIDLSQLGGDSGGSSGGSSGSETPGTSESATPESSGGSSGGSSTLNIEDE |
| | | | YRLHETSKEPDVSLGSTWLSDFPQAWAETGGMGLAVRQAPLIIPLKATSTPVSIKQYPMSQEARLGIKPHIQRLLDQGILVPCQS |
| | | | PWNTPLLPVKKPGTNDYRPVQDLREVNKRVEDIHPTVPNPYNLLSGLPPSHQWYTVLDLKDAFFCLRLHPTSQPLFAFEWRDP |
| | | | EMGISGQLTWTRLPQGFKNSPTLFNEALHRDLADFRIQHPDLILLQYVDDLLLAATSELDCQQGTRALLQTLGNLGYRASAKK |
| | | | AQICQKQVKYLGYLLKEGQRWLTEARKETVMGQPTPKTPRQLREFLGKAGFCRLFIPGFAEMAAPLYPLTKPGTLFNWGPDQ |
| | | | QKAYQEIKQALLTAPALGLPDLTKPFELFVDEKQGYAKGVLTQKLGPWRRPVAYLSKKLDPVAAGWPPCLRMVAAIAVLTK |
| | | | DAGKLTMGQPLVILAPHAVEALVKQPPDRWLSNARMTHYQALLLDTDRVQFGPVVALNPATLLPLPEEGLQHNCLDILAEAH |
| | | | GTRPDLTDQPLPDADHTWYTDGSSLLQEGQRKAGAAVTTETEVIWAKALPAGTSAQRAELIALTQALKMAEGKKLNVYTDS |
| | | | RYAFATAHIHGEIYRRRGWLTSEGKEIKNKDEILALLKALFLPKRLSIIHCPGHQKGHSAEARGNRMADQAARKAAITETPDTS |
| | | | TLLIENSSPSGGSKRTADGSEFEPKKKRKV |

### Example 3: HEK293T Culture and Electroporation

A frozen vial of HEK293T (ATCC CRL-3216) cell line was thawed at 37° C. and cultured in Dulbecco's Modified Eagle's Medium supplemented with 10% (v/v) fetal bovine serum (FBS) at 37° C. with 5% CO2. Cells were grown to 90% confluency before passaging. Cells were passaged 3 times before any experiments. Before electroporation, 48-well plates were coated with poly-D-lysine for 1 hour and subsequently washed 3 times with PBS. After PBS wash, 400 μL of complete media without any antibiotic was added to each well and incubated at 37° C. with 5% CO2 before use. All electroporations were performed using the Neon N×T electroporation system. For electroporation, 1 μg of purified mRNA, 100 pmol of degRNA, 33 pmol of nicking sgRNA and 100,000 cells were mixed in Buffer R and electroporated with 10-μl Neon tips. Electroporation settings of 1150V, 20 ms, and 2 pulses were used for all electroporations. Following electroporation, cells were added to a single well of a 48-well plate containing cell culture media and incubated at 37° C. with 5% CO₂. Genomic DNA was extracted 48 hours post-electroporation for NGS analysis.

### Example 4: Primary Human Hepatocyte (PHH) Culture and Transfection

For mRNA transfection in the 96-well format, 50,000 cryopreserved primary human hepatocytes were seeded in each well on a collagen I-coated plate. Leaving the newly seeded hepatocytes undisturbed for at least 4-6 hours, the plating medium was replaced with incubation medium after thawing and plating. The incubation medium was changed 24 hours after plating and followed by immediate dosing of each well with a 10 μL complex containing 180 ng of mRNA, 1.5 pmol of degRNA, 0.5 pmol of ngRNA, and 0.3 μL of Lipofectamine MessengerMAX reagent, added dropwise to the plate followed by a gentle swirl. The cell culture plate was incubated at 37° C. with 5% CO2 and media was replaced every 24 hours. Genomic DNA was extracted 72 hours post-transfection for NGS analysis.

### Example 5: Primary Human Hepatocyte (PHH) Culture and Electroporation

For electroporation, cells were thawed in thawing and plating medium. For each electroporation, 1 μg of mRNA, 100 pmoles of degRNA, and 33 pmol of ngRNA were mixed with 100,000 cells and electroporated using 4D-Nucleofactor (Lonza) instrument with program name, DS-150. After electroporation, cells were plated in a single well of a 96-well plate pre-coated with Collagen I and incubated at 37° C. with 5% CO2. Plating medium was changed 6 hours post electroporation. Genomic DNA was extracted 72 hours post-electroporation for NGS analysis.

### Example 6: In Vitro Transcribed (IVT) mRNA

Plasmids containing the gene of interest were completely digested and linearized by BsmBI (New England Biolabs) before use in the in vitro transcription reaction (IVT). The IVT reactions were performed using the NEB HiScribe T7 High Yield RNA Synthesis kit (New England Biolabs). In summary, the IVT reactions were performed at 37° C. with the addition of CleanCap Reagent AG (Trilink Biotechnologies) with a 100% replacement of UTP by N1-methylpseudo-UTP (Trilink Biotechnologies). The IVT reactions were terminated after 2 hours. Following IVT, each reaction was incubated with DNase I (New England Biolabs) for 15 minutes. Afterwards, the RNA was purified using the Monarch RNA Cleanup kit (New England Biolabs).

### Example 7: Next Generation Sequencing (NGS) Library Prep and Analysis

PCR primers (see Table 4) containing Illumina-compatible adapter sequences were used to amplify specific genomic regions of interest. Following standard PCR protocol using Q5 Hot Start High-Fidelity 2× Master Mix (New England Biolabs), the resulting PCR products were cleaned up with 0.7× Ampure XP Beads (Beckman Coulter). Purified PCR products were sequenced. Amplicon sequencing data was analyzed with CRISPResso. Editing efficiencies were calculated from the alleles frequency table file based on the number of sequencing reads containing the desired edit of interest relative to the total number of sequencing reads within a given sample.

### Example 8: Targeted Gene Editing in HEK293 Cells with DNA-Dependent DNA Polymerase and Nickase Engineered Proteins

mRNA encoding the DdDP editors in Table 7 (SEQ ID NO: 234-SEQ ID NO: 246) were in vitro transcribed according to the methods in Example 6 and synthetic guide RNAs in Table 5 (SEQ ID NO: 187-SEQ ID NO: 192) were manufactured by chemical synthesis. The mRNA and synthetic guide RNAs were delivered to HEK293T cells via electroporation as described in Example 3.

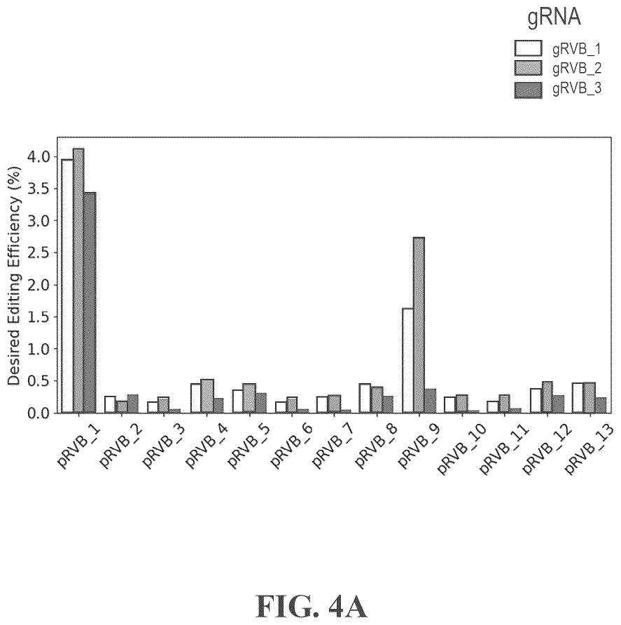

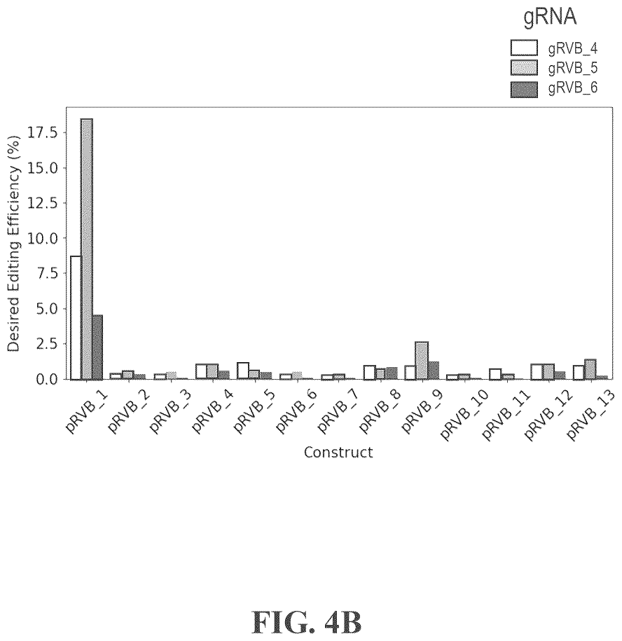

DNA-dependent DNA polymerase constructs (pRVB_1-13; SEQ ID NO: 234-SEQ ID NO: 246) were screened for precision genome editing activity in HEK293T cells at genomic target sites, HEK site 3 (FIG. 4A) and FANCF site 1 (FIG. 4B) across three guide RNA constructs for each target site as shown in Table 8 below and as indicated in FIGS. 4A-4B.

TABLE 8
Guide RNAs and Target Editing Site.

| Target Site | Guide RNA Name | SEQ ID NO: |
|---|---|---|
| HEK site 3 | gRVB_1 | SEQ ID NO: 187 |
| HEK site 3 | gRVB_2 | SEQ ID NO: 188 |
| HEK site 3 | gRVB_3 | SEQ ID NO: 189 |
| FANCF site 1 | gRVB_4 | SEQ ID NO: 190 |
| FANCF site 1 | gRVB_5 | SEQ ID NO: 191 |
| FANCF site 1 | gRVB_6 | SEQ ID NO: 192 |

The engineered DNA-dependent DNA polymerase (DdDP) genome editors mediated precise genome editing in human cells at the HEK Site 3 and FANCF Site 1.

To improve precision editing efficiency, unique combinations of mutations were introduced to enhance phi29 DNA-dependent DNA polymerase properties such as processivity, fidelity, stability, salt-tolerance, and dNTP affinity as summarized in Table 9 below. Engineered phi29 DNA-dependent DNA polymerase editor proteins were generated as indicated in Table 9, column 2 that correspond to the constructs in Table 7.

TABLE 9
Phi29 DNA polymerase modifications and properties.

| Phi 29 | | |
|---|---|---|
| DNA Pol | | |
| SEQ ID | Modifications in the DdDP | Engineered Protein Construct |
| NO: | relative to SEQ ID NO: 63 | Names and Sequences |
| 99 | D12A, D66A | pRVB_1 (SEQ ID NO: 234) |
| | | pRVB_14 (SEQ ID NO: 247) |
| 100 | M8R, D12A, V51A, D66A, | pRVB_15 (SEQ ID NO: 248) |
| | M97T, G197D, E221K | |
| 101 | M8R, D12A, V51A, D66A, | pRVB_16 (SEQ ID NO: 249) |
| | M97T, G197D, E221K, | |
| | Q497P, K512E, F526L | |
| 102 | M8R, D12A, V51A, D66A, | pRVB_17 (SEQ ID NO: 250) |
| | M97T, F137C, G197D, | pRVB_18 (SEQ ID NO: 251) |
| | E221K, A377C, Q497P, | pRVB_20 (SEQ ID NO: 253) |
| | K512E, F526L | pRVB_21 (SEQ ID NO: 254) |
| | | pRVB_25 (SEQ ID NO: 258) |
| | | pRVB_26 (SEQ ID NO: 259) |
| | | pRVB_27 (SEQ ID NO: 260) |
| | | pRVB_28 (SEQ ID NO: 261) |
| | | pRVB_29 (SEQ ID NO: 262) |
| 103 | M8R, V51A, M97T, F137C, | pRVB_19 (SEQ ID NO: 252) |
| | G197D, E221K, A377C, | |
| | Q497P, K512E, F526L | |
| 104 | D12A, D66A, G197D, | pRVB_22 (SEQ ID NO: 255) |
| | Y369E, T372N, E375D, | |
| | I378R | |
| 105 | M8R, D12A, V51A, D66A, | pRVB_23 (SEQ ID NO: 256) |
| | M97T, G197D, E221K, | |
| | Y369E, T372N, E375D, | |
| | I378R, Q497P, K512E, | |
| | F526L | |
| 106 | M8R, D12A, V51A, D66A, | pRVB_24 (SEQ ID NO: 257) |
| | M97T, F137C, G197D, | |
| | E221K, A377C, Y369E, | |
| | T372N, E375D, I378R, | |
| | Q497P, K512E, F526L | |

Additional synthetic guide RNAs were engineered by varying the composition of ribonucleotides (RNA) and deoxyribonucleotides (DNA) in the hybridization region (HR) to improve precision editing efficiency of the DNA-dependent DNA polymerase gene editing constructs as shown in Table 10.

TABLE 10
RNA Guides.

| SEQ ID NO: | Guide Name | Target Site |
|---|---|---|
| 205 | gRVB_19 | HEK Site 3 |
| 207 | gRVB_21 | HEK Site 3 |
| 198 | gRVB_12 | FANCF Site 1 |
| 208 | gRVB_22 | FANCF Site 1 |
| 216 | gRVB_30 | HEK site 3 |
| 232 | gRVB_46 | HEK site 3 |
| 233 | gRVB_47 | FANCF Site 1 |
| 208 | gRVB_22 | FANCF Site 1 |

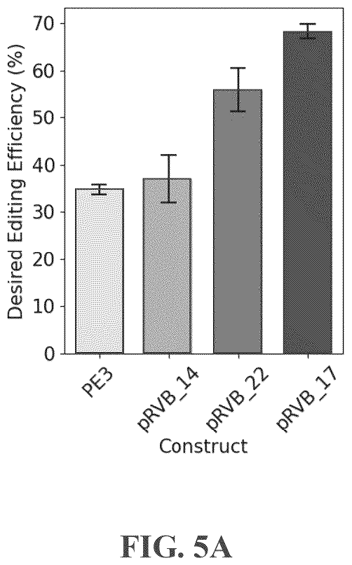

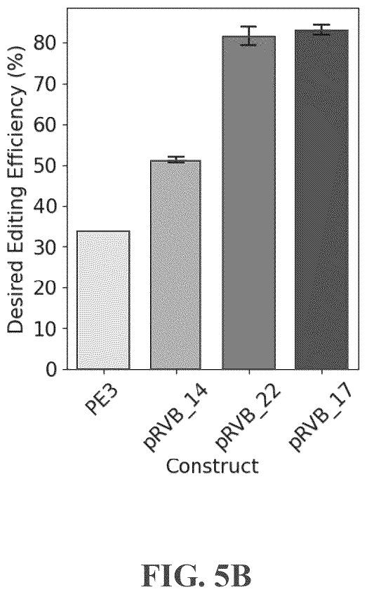

Multiple Phi29 DNA polymerase variants exhibited enhanced precision genome editing activity in HEK293T cells at genomic target sites HEK site 3 (FIG. 5A; pRVB_14 (SEQ ID NO: 247), pRVB_22 (SEQ ID NO: 255), pRVB_17 (SEQ ID NO: 250), gRVB_19 (SEQ ID NO: 205), gRVB_21 (SEQ ID NO: 207)) and FANCF site 1 (FIG. 51B; pRVB_14 (SEQ ID NO: 247), pRVB_22 (SEQ ID NO: 255), pRVB_17 (SEQ ID NO: 250), gRVB_12 (SEQ ID NO: 198), gRVB_22 (SEQ ID NO: 208)) compared to PE3 (HEK site 3: pRVB_30 (SEQ ID NO: 263), gRVB_46 (SEQ ID NO: 222), gRVB_21 (SEQ ID NO: 207); FANCF site 1: pRVB_30 (SEQ ID NO: 263), gRVB_47 (SEQ ID NO: 223), gRVB_22 (SEQ ID NO: 208)). PE3 (also referred to as pRVB_30, comprising a modified Murine leukemia virus (MMLV) reverse transcriptase in place of the DdDP region of the fusion protein. The MMLV reverse transcriptase was engineered to comprise amino acid substitutions D200N, T306K, W313F, T330P, and L603W as compared to SEQ ID NO: 111.

MMLV reverse transcriptase:
(SEQ ID NO: 111)
MTLNIEDEYRLHETSKEPDVSLGSTWLSDFPQAWAETGGMGLAVRQAPLIIPLKATSTPVSIKQYP
MSQEARLGIKPHIQRLLDQGILVPCQSPWNTPLLPVKKPGTNDYRPVQDLREVNKRVEDIHPTVP
NPYNLLSGLPPSHQWYTVLDLKDAFFCLRLHPTSQPLFAFEWRDPEMGISGQLTWTRLPQGFKNS
PTLFDEALHRDLADFRIQHPDLILLQYVDDLLLAATSELDCQQGTRALLQTLGNLGYRASAKKA
QICQKQVKYLGYLLKEGQRWLTEARKETVMGQPTPKTPRQLREFLGTAGFCRLWIPGFAEMAAP
LYPLTKTGTLFNWGPDQQKAYQEIKQALLTAPALGLPDLTKPFELFVDEKQGYAKGVLTQKLGP
WRRPVAYLSKKLDPVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILAEAHGTRPDLTDQPL
PDADHTWYTDGSSLLQEGQRKAGAAVTTETEVIWAKALPAGTSAQRAELIALTQALKMAEGKK
LNVYTDSRYAFATAHIHGEIYRRRGLLTSEGKEIKNKDEILALLKALFLPKRLSIIHCPGHQKGHSA
EARGNRMADQAARKAAITETPDTSTLLIENSSP.
The pRVB_30 engineered protein comprises a modified MMLV
reverse transcriptase comprising SEQ ID NO: 112.
(SEQ ID NO: 112)
MTLNIEDEYRLHETSKEPDVSLGSTWLSDFPQAWAETGGMGLAVRQAPLIIPLKATSTPVSIKQYP
MSQEARLGIKPHIQRLLDQGILVPCQSPWNTPLLPVKKPGTNDYRPVQDLREVNKRVEDIHPTVP
NPYNLLSGLPPSHQWYTVLDLKDAFFCLRLHPTSQPLFAFEWRDPEMGISGQLTWTRLPQGFKNS
PTLFNEALHRDLADFRIQHPDLILLQYVDDLLLAATSELDCQQGTRALLQTLGNLGYRASAKKA
QICQKQVKYLGYLLKEGQRWLTEARKETVMGQPTPKTPRQLREFLGKAGFCRLFIPGFAEMTLN
IEDEYRLHETSKEPDVSLGSTWLSDFPQAWAETGGMGLAVRQAPLIIPLKATSTPVSIKQYPMSQE
ARLGIKPHIQRLLDQGILVPCQSPWNTPLLPVKKPGTNDYRPVQDLREVNKRVEDIHPTVPNPYN
LLSGLPPSHQWYTVLDLKDAFFCLRLHPTSQPLFAFEWRDPEMGISGQLTWTRLPQGFKNSPTLF
NEALHRDLADFRIQHPDLILLQYVDDLLLAATSELDCQQGTRALLQTLGNLGYRASAKKAQICQ
KQVKYLGYLLKEGQRWLTEARKETVMGQPTPKTPRQLREFLGKAGFCRLFIPGFAEMAAPLYPL
TKPGTLFNWGPDQQKAYQEIKQALLTAPALGLPDLTKPFELFVDEKQGYAKGVLTQKLGPWRR
PVAYLSKKLDPVAAGWPPCLRMVAAIAVLTKDAGKLTMGQPLVILAPHAVEALVKQPPDRWLS
NARMTHYQALLLDTDRVQFGPVVALNPATLLPLPEEGLQHNCLDILAEAHGTRPDLTDQPLPDA
DHTWYTDGSSLLQEGQRKAGAAVTTETEVIWAKALPAGTSAQRAELIALTQALKMAEGKKLNV
YTDSRYAFATAHIHGEIYRRRGWLTSEGKEIKNKDEILALLKALFLPKRLSIIHCPGHQKGHSAEA
RGNRMADQAARKAAITETPDTSTLLIENSSPMAAPLYPLTKPGTLFNWGPDQQKAYQEIKQALLT
APALGLPDLTKPFELFVDEKQGYAKGVLTQKLGPWRRPVAYLSKKLDPVAAGWPPCLRMVAAI
AVLTKDAGKLTMGQPLVILAPHAVEALVKQPPDRWLSNARMTHYQALLLDTDRVQFGPVVAL
NPATLLPLPEEGLQHNCLDILAEAHGTRPDLTDQPLPDADHTWYTDGSSLLQEGQRKAGAAVTT
ETEVIWAKALPAGTSAQRAELIALTQALKMAEGKKLNVYTDSRYAFATAHIHGEIYRRRGWLTS
EGKEIKNKDEILALLKALFLPKRLSIIHCPGHQKGHSAEARGNRMADQAARKAAITETPDTSTLLI
ENSSP.

Modified amino acids relative to SEQ ID NO: 111 are show in bold/underlined text above.

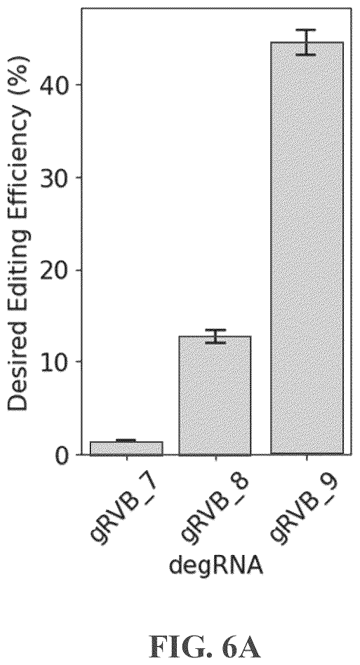

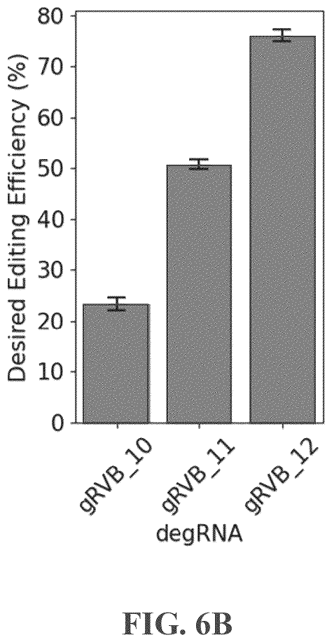

Guide RNAs were engineered to enhance the activity of DdDP genome editors. Various gRNA constructs exhibited a range of precision genome editing activity in HEK293T cells at HEK site 3 (FIG. 6A) and FANCF site 1 (FIG. 6B) using the pRVB_17 DdDP editor construct (SEQ ID NO: 250) and associated nicking gRNAs (HEK site 3: gRVB_21 (SEQ ID NO: 207), FANCF site 1: gRVB_22 (SEQ ID NO: 208)).

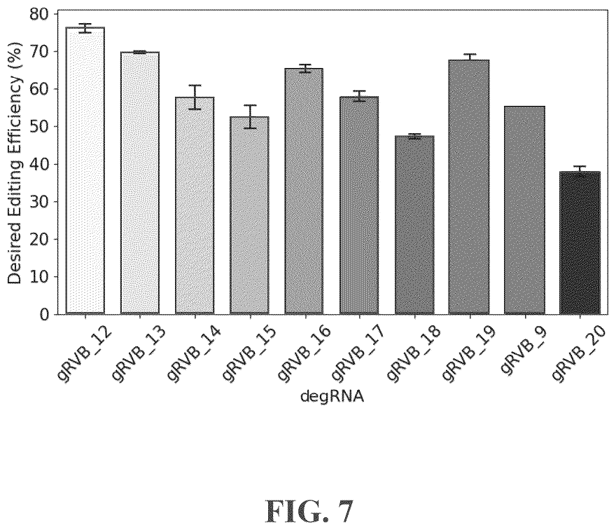

Precision genome editing was performed at genomic target sites FANCF site 1 and HEK site 3 in HEK293T cells using the pRVB_17 DdDP editor construct (SEQ ID NO: 250) and associated nicking gRNAs (HEK site 3: gRVB_21, SEQ ID NO: 207), FANCF site 1: gRVB_22 (SEQ ID NO: 208)). The in vitro transcribed mRNA encoding the DdDP editors and synthetic guide RNAs were delivered to HEK293T cells via electroporation. DdDP genome editors were capable of installing various substitution (transition and transversion edits), insertion, and deletion edits in human cells (FIG. 7).

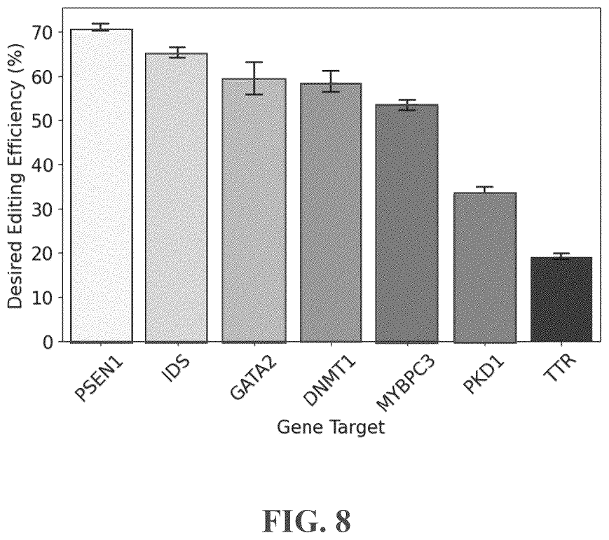

The in vitro transcribed mRNA encoding the DdDP editors and synthetic guide RNAs were delivered to HEK293T cells via electroporation. Assays were performed in HEK293T cells using the pRVB_17 DdDP editor construct (SEQ ID NO: 250) with guide RNAs gRVB_23-36 (SEQ ID NO: 209-SEQ ID NO: 222) to introduce pathogenic variant edits across multiple target genes in the human genome. DdDP genome editors efficiently installed various human pathogenic variants across endogenous genomic loci in human cells (FIG. 8).

To explore alternative mechanisms of genome editing with modified phi29 DNA-dependent DNA polymerases and a SpCas9 of SEQ ID NO: 93, a 3′ exonuclease-deficient DdDP editor (pRVB_15, SEQ ID NO: 248) was engineered to remove the ability to install a point mutation within the hybridization region (HR) of the synthetic gRNA (−2 G>A; gRVB_37 (SEQ ID NO: 223), gRVB_38 (SEQ ID NO: 224)). The pRVB_19 DdDP genome editor (SEQ ID NO: 252) with an intact 3′ exonuclease was compared to the exonuclease deficient editor. The DdDP genome editor, pRVB_19 (SEQ ID NO: 252) facilitated efficient installation of the desired edit using the same guide RNAs (gRVB_37 (SEQ ID NO: 223), gRVB_38 (SEQ ID NO: 224)), signifying the capability of 3′ exonucleases to degrade the target genomic DNA strand to facilitate installation of mutations within the hybridization region (HR) of synthetic guide RNAs. This alternative mechanism of action for DdDP genome editing is dependent on 3′ exonuclease activity, which is only present in certain DNA-dependent DNA polymerases, and increases the editing window for DdDP genome editors with intact 3′ exonuclease activity. The assay conditions are provided in Table 11.

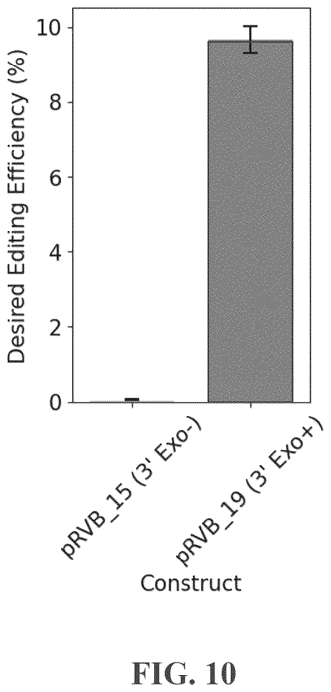

TABLE 11
Assay Conditions in FIG. 10.

| | DdDP | | | |
|---|---|---|---|---|
| SEQ | Genome | | DNA-dependent | Exonuclease |
| ID | Editor | Nickase | polymerase | Activity |
| NO: | Construct | (SEQ ID NO:) | (SEQ ID NO:) | (Yes/No) |
| 248 | pRVB_15 | SpCas9, R221K, | Phi29 DdDP M8R, | No |
| | | N394K, H840A | D12A, V51A, | |
| | | (SEQ ID NO: 93) | D66A, M97T, | |
| | | | G197D, E221K | |
| | | | (SEQ ID NO: 100) | |
| 252 | pRVB_19 | SpCas9, R221K, | Phi29 DdDP M8R, | Yes |
| | | N394K, H840A | V51A, M97T, | |
| | | (SEQ ID NO: 93) | F137C, G197D, | |
| | | | E221K, A377C, | |
| | | | Q497P, K512E, | |
| | | | F526L (SEQ ID | |
| | | | NO: 103) | |

Phi29 DNA polymerase 3′ exonuclease activity enabled improved precision genome editing within the hybridization region (HR) of the guide RNA (FIG. 10).

### Example 9: Targeted Gene Editing in Primary Human Hepatocytes

DdDP editors were assayed for editing efficiency in primary human hepatocytes (PHHs).

PHHs were cultured according to the methods outlined in Example 4.

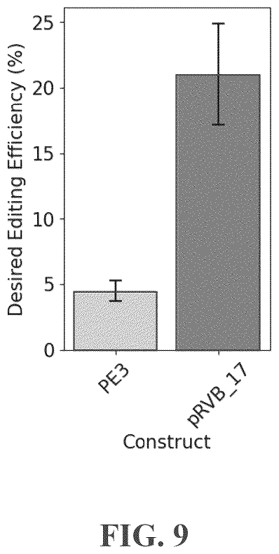

The in vitro transcribed mRNA encoding the DdDP editors and synthetic guide RNAs were delivered to primary human hepatocyte (PHH) cells via transfection using Lipofectamine MessengerMAX. The DdDP editors exhibited enhanced precision genome editing activity at FANCF site 1 (pRVB_17 (SEQ ID NO: 250), gRVB_12 (SEQ ID NO: 198), gRVB_22 (SEQ ID NO: 208)) compared to PE3 (pRVB_30 (SEQ ID NO: 263), gRVB_47 (SEQ ID NO: 223), gRVB_22 (SEQ ID NO: 208)). DdDP genome editors exhibit activity in primary human hepatocytes (FIG. 9).

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. A composition comprising:
(a) a phi29 DNA-dependent DNA polymerase or a variant thereof and a nickase; or
(b) a polynucleotide encoding the phi29 DNA-dependent DNA polymerase or a variant thereof and a polynucleotide encoding the nickase; and, in addition to (a) or (b),
(c) a guide polynucleotide comprising:
(i) a targeting region that has complementarity to a target nucleic acid;
(ii) a protein binding region, wherein the protein binding region comprises a secondary structure that binds to the nickase; and
(iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and
(iv) a hybridization region, wherein the hybridization region comprises:
deoxyribonucleotides and ribonucleotides.

2. The composition of claim 1, wherein the nickase comprises a Cas9, a Cas1, a Cas2, or a Csn2 protein.

3. The composition of claim 2, wherein the Cas9 protein comprises a Streptococcus pyogenes Cas9 protein or a variant thereof, or
a Streptococcus thermophilus Cas9 protein or a variant thereof.

4. The composition of claim 1, wherein the phi29 DNA-dependent DNA polymerase comprises a sequence that is at least 90% identical to SEQ ID NO: 63.

5. The composition of claim 1, wherein the phi29 DNA-dependent DNA polymerase variant comprises a mutation, wherein the mutation comprises an amino acid substitution of an amino acid residue of position: 8, 12, 51, 66, 97, 137, 197, 221, 369, 372, 375, 377, 378, 497, 512, or 526 as compared to SEQ ID NO: 63.

6. The composition of claim 5, wherein the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is a hydrophobic amino acid residue, a hydrophilic amino acid residue, a charged amino acid residue that is a basic amino acid residue or an acidic amino acid residue, or an aliphatic amino acid residue as compared to SEQ ID NO: 63.

7. The composition of claim 5, wherein the amino acid substitution at any one of positions 8, 12, 51, 66, 97, 137, 197, 221, 369, 372, 375, 377, 378, 497, 512, or 526 comprises a substitution of an amino acid residue to a different amino acid residue, wherein the different amino acid residue is an arginine (Arg, R), an alanine (Ala, A), a glutamic acid (Glu, E), a proline (Pro, P), a threonine (Thr, T), an aspartic acid (Asp, D), a cysteine (Cys, C), a leucine (Leu, L) or a lysine (Lys, K) as compared to SEQ ID NO: 63.

8. The composition of claim 6, wherein the amino acid substitution comprises: M8R, D12A, V51A, D66A, M97T, F137C, G197D, E221K, Y369E, T372N, E375D, A377C, I378R, Q497P, K512E, F526L, or any combination thereof as compared to SEQ ID NO: 63.

9. The composition of claim 1, wherein the phi29 DNA-dependent DNA polymerase variant comprises any one of SEQ ID NO: 99-SEQ ID NO: 106.

10. The composition of claim 1, wherein the phi29 DNA-dependent DNA polymerase variant comprises SEQ ID NO: 102.

11. The composition of claim 1, wherein the phi29 DNA-dependent DNA polymerase variant comprises SEQ ID NO: 103.

12. The composition of claim 1, wherein the phi29 DNA-dependent DNA polymerase variant comprises SEQ ID NO: 104.

13. The composition of claim 1, wherein the polynucleotide encoding the phi29 DNA-dependent DNA polymerase or a variant thereof is linked to the polynucleotide encoding the nickase.

14. The composition of claim 1, wherein the phi29 DNA-dependent DNA polymerase or a variant thereof is linked to the nickase.

15. The composition of claim 1, wherein the hybridization region comprises at least about 5 nucleotides up to 10 nucleotides.

16. The composition of claim 1, wherein the hybridization region comprises a ratio of ribonucleic acids (RNAs) to deoxyribonucleic acids (DNAs) of: 1:1, 2:1, 3:1, 3:2, 4:1, 4:3, 4:5, 5:1, 5:2, 5:3, 5:4, 5:6, 6:1, 6:5, 6:7, 7:1, 7:2, 7:3, 7:4, 7:5, 7:6, 7:8, 7:9, 8:1, 8:3, 8:5, 8:7, 8:9, 9:1, 9:2, 9:4, 9:5, 9:7, 9:8, 9:10, 9:11, 10:1, 10:3, 10:7, 10:9, 10:11, 10:13, 11:1, 11:2, 11:3, 11:4, 11:5, 11:6, 11:7, 11:8, 11:9, 11:10, 11:12, 11:13, 12:1, 12:5, 12:7, 12:9, 12:11, 12:13, 13:1, 13:2, 13:3, 13:4, 13:5, 13:6, 13:7, 13:8, 13:9, 13:10, 13:11, 13:12, 13:14, 14:1, 14:3, 14:5, 14:9, 14:11, 14:13, 15:1, 15:2, 15:4, 15:6, 15:8, 15:11, 15:13, 16:1, 16:3, 16:5, 16:7, 16:9, 16:11, 16:13, 16:15, 17:1, 17:2, 17:3, 17:4, 17:5, 17:6, 17:7, 17:8, 17:9, 17:10, 17:11, 17:12, 17:13, 17:14, 17:15, 17:16, 18:1, 18:5, 18:7, 18:11, 18:13, 18:17, 19:1, 19:2, 19:3, 19:4, 19:5, 19:6, 19:7, 19:8, 19:9, 19:10, 19:11, 19:12, 19:13, 19:14, 19:15, 19:16, 19:17, 19:18, 20:1.

17. The composition of claim 1, wherein the DST region comprises at least about 5 nucleotides up to 10,000 nucleotides.

18. The composition of claim 1, further comprising a nicking gRNA.

19. A composition comprising:
(a) a DNA-dependent DNA polymerase or a variant thereof and a Streptococcus pyogenes Cas9 nickase or a variant thereof, wherein the variant comprises an amino acid substitution at position: 61, 221, 394, 840, 1111, 1135, 1136, 1137, 1218, 1219, 1317, 1322, 1333, 1335, or 1337 as compared to SEQ ID NO: 92; or
(b) a polynucleotide encoding the DNA-dependent DNA polymerase or a variant thereof and a polynucleotide encoding the Streptococcus pyogenes Cas9 nickase or the variant thereof, and, in addition to (a) or (b),
(c) a guide polynucleotide comprising:
(i) a targeting region that has complementarity to a target nucleic acid;
(ii) a protein binding region, wherein the protein binding region comprises a secondary structure that binds to a protein; and
(iii) a DNA-dependent DNA polymerase synthesis template (DST), wherein the DNA-dependent DNA polymerase synthesis template (DST) region comprises: a sequence that has complementarity to the target nucleic acid and at least one alteration relative to the target nucleic acid or at least one nucleic acid strand thereof, and
(iv) a hybridization region, wherein the hybridization region comprises:
deoxyribonucleotides and ribonucleotides.

20. A system for synthesizing a nucleic acid sequence, the system comprising:
(a) a polynucleotide encoding an engineered protein construct, wherein the engineered protein construct comprises:
(i) a nickase region or a variant thereof, and
(ii) a DNA-dependent DNA polymerase (DdDP) or a variant thereof, and
(b) a guide polynucleotide, wherein the guide polynucleotide comprises:
(i) a targeting region that has complementarity to a target nucleic acid;
(ii) a protein binding region, wherein the protein binding region comprises a secondary structure that binds to the engineered protein construct comprising a nickase region; and
(iii) a DNA-dependent DNA polymerase synthesis template (DST) region, wherein the DNA-dependent DNA polymerase synthesis template (DST) region comprises a sequence that has complementarity to the target nucleic acid and at least one alteration relative to the target nucleic acid; and
(iv) a hybridization region, wherein the hybridization region comprises: a deoxyribonucleotide and a ribonucleotide, wherein the hybridization region has complementarity to the target nucleic acid,
wherein upon introduction of the system to a cell, a nucleus, or a cell-free system, the system synthesizes a nucleic acid sequence that is incorporated into the target nucleic acid.

21. A method of synthesizing a nucleic acid, the method comprising:
contacting a cell or a cell-free system with:
(a) a guide polynucleotide or a polynucleotide encoding the guide polynucleotide, wherein the guide polynucleotide comprises:
(i) a targeting region that has complementarity to a target nucleic acid;
(ii) a protein binding region, wherein the protein binding region comprises a secondary structure that binds to a nuclease;
(iii) a DNA-dependent DNA polymerase synthesis template (DST) region, wherein the DST region comprises a sequence that has complementarity to a target nucleic acid and at least one alteration relative to the target nucleic acid; and
(iv) a hybridization region, wherein the hybridization region comprises a deoxyribonucleotide and a ribonucleotide; and
(b) an engineered protein or a polynucleotide encoding the engineered protein, wherein the engineered protein comprises:
(i) a nuclease region; and
(ii) a DdDP region;
wherein:
the guide polynucleotide forms a complex with the engineered protein via the protein binding region,
the nuclease region of the engineered protein generates a break in the target nucleic acid to generate a leading strand and a complementary strand,
the targeting sequence forms a complex with a complementary strand of the cleaved target nucleic acid, the hybridization region forms a complex with the leading strand,
and wherein the guide polynucleotide associates with the DdDP region of the engineered protein, thereby synthesizing a new nucleic acid.
